SEQUENCE LISTING (414 sequences)

SEQ ID NO: 1 - DNA, 6222 bp, Homo sapiens
gggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 2 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 3 - DNA, 3926 bp, Artificial Sequence
  Description of Artificial Sequence Synthetic polynucleotide
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 4 - DNA, 4679 bp, Artificial Sequence
  Description of Artificial Sequence Synthetic polynucleotide
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtacttgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 5 - DNA, 2473 bp, Artificial Sequence
  Description of Artificial Sequence Synthetic polynucleotide
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 6 - DNA, 2506 bp, Artificial Sequence
  Description of Artificial Sequence Synthetic polynucleotide
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagcaaaaatgtcaactcgcgaaaaaaacagccaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 7 - DNA, 3409 bp, Artificial Sequence
  Description of Artificial Sequence Synthetic polynucleotide
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagatatgctggatgagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaagaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 8 - DNA, 2014 bp, Artificial Sequence
  Description of Artificial Sequence Synthetic polynucleotide
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 9 - DNA, 2131 bp, Artificial Sequence
  Description of Artificial Sequence Synthetic polynucleotide
actctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggttcagagctcaggggaggatatggagatccagggaggcttcctgtaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 10 - DNA, 3365 bp, Artificial Sequence
  Description of Artificial Sequence Synthetic polynucleotide
tactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggaagtggcctgtgtagtgcttcaagggccaggctgccaggccatgttgcagctgaccacccacctgcagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgg

SEQ ID NO: 11 - DNA, 3435 bp, Artificial Sequence
  Description of Artificial Sequence Synthetic polynucleotide
tactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaggaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtgga

SEQ ID NO: 12 - DNA, 4479 bp, Artificial Sequence
  Description of Artificial Sequence Synthetic polynucleotide
tgcgagaaagatggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgctgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaagcagcctgagggaactggcatgatagatgaagagttcactgttgcaagactctacattagcaaaatgaagtcagaagtaaaaaccatggtgaaacgttgcaagcagttagaaagcacacaaactgagagcaacaaaaaaatggaagaaaatgaaaaggagttagcagcatgtcagcttcgtatctctcaacatgaagccaaaatcaagtcattgactgaataccttcaaaatgtggaacaaaagaaaagacagttggaggaatctgtcgatgccctcagtgaagaactagtccagcttcgagcacaagagaaagtccatgaaatggaaaaggagcacttaaataaggttcagactgcaaatgaagttaagcaagctgttgaacagcagatccagagccatagagaaactcatcaaaaacagatcagtagtttgagagatgaagtagaagcaaaagcaaaacttattactgatcttcaagaccaaaaccagaaaatgatgttagagcaggaacgtctaagagtagaacatgagaagttgaaagccacagatcaggaaaagagcagaaaactacatgaacttacggttatgcaagatagacgagaacaagcaagacaagacttgaagggtttggaagagacagtggcaaaagaacttcagactttacacaacctgcgcaaactctttgttcaggacctggctacaagagttaaaaagagtgctgagattgattctgatgacaccggaggcagcgctgctcagaagcaaaaaatctcctttcttgaaaataatcttgaacagctcactaaagtgcacaaacagttggtacgtgataatgcagatctccgctgtgaacttcctaagttggaaaagcgacttcgagctacagctgagagagtgaaagctttggaatcagcactgaaagaagctaaagaaaatgcatctcgtgatcgcaaacgctatcagcaagaagtagatcgcataaaggaagcagtcaggtcaaagaatatggccagaagagggcattctgcacagattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactca

SEQ ID NO: 13 - DNA, 2412 bp, Artificial Sequence
  Description of Artificial Sequence Synthetic polynucleotide
atgaacggacagttggatctaagtgggaagctaatcatcaaagctcaacttggggaggatattcggcgaattcctattcataatgaagatattacttatgatgaattagtgctaatgatgcaacgagttttcagaggaaaacttctgagtaatgatgaagtaacaataaagtataaagatgaagatggagatcttataacaatttttgatagttctgacctttcctttgcaattcagtgcagtaggatactgaaactgacattatttgttaatggccagccaagaccccttgaatcaagtcaggtgaaatatctccgtcgagaactgatagaacttcgaaataaagtgaatcgtttattggatagcttggaaccacctggagaaccaggaccttccaccaatattcctgaaaatgatactgtggatggtagggaagaaaagtctgcttctgattcttctggaaaacagtctactcaggttatggcagcaagtatgtctgcttttgatcctttaaaaaaccaagatgaaatcaataaaaatgttatgtcagcgtttggcttaacagatgatcaggtttcagggccacccagtgctcctgcagaagatcgttcaggaacacccgacagcattgcttcctcctcctcagcagctcacccaccaggcgttcagccacagcagccaccatatacaggagctcagactcaagcaggtcagattgaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 14 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 15 - DNA, 1251 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggcagccgctctggctctagctccagctccgggaccctctgggaccccccgggacccatgtgacccagcggcccctcgcgctggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 16 - DNA, 4461 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattcttatattca

SEQ ID NO: 17 - DNA, 2366 bp, Homo sapiens
ggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaa

SEQ ID NO: 18 - DNA, 2977 bp, Homo sapiens
ccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcaggagcagtcttccatcatgctgaattttgtcttccaggagctgcccctatggggcgggccgcagggccagcctgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaattt

SEQ ID NO: 19 - DNA, 2458 bp, Homo sapiens
  modified_base (1088)..(1088): a, c, t, g, unknown or other
tgacccaataagggtggaaggctgagtcccgcagagccaataacgagagtccgagaggcgacggaggcggactctgtgaggaaacaagaagagaggcccaagatggagacggcggcggctgtagcggcgtgacaggagccccatggcacctgcccagccccacctcagcccatcttgacaaaatctaaggctccatggagccaccacggggcccccctgccaatggggccgagccatcccgggcagtgggcaccgtcaaagtatacctgcccaacaagcaacgcacggtggtgactgtccgggatggcatgagtgtctacgactctctagacaaggccctgaaggtgcggggtctaaatcaggactgctgtgtggtctaccgactcatcaagggacgaaagacggtcactgcctgggacacagccattgctcccctggatggcgaggagctcattgtcgaggtccttgaagatgtcccgctgaccatgcacaattttgtacggaagaccttcttcagcctggcgttctgtgacttctgccttaagtttctgttccatggcttccgttgccaaacctgtggctacaagttccaccagcattgttcctccaaggtccccacagtctgtgttgacatgagtaccaaccgccaacagttctaccacagtgtccaggatttgtccggaggctccagacagcatgaggctccctcgaaccgccccctgaatgagttgctaaccccccagggtcccagcccccgcacccagcactgtgacccggagcacttccccttccctgccccagccaatgcccccctacagcgcatccgctccacgtccactcccaacgtccatatggtcagcaccacggcccccatggactccaacctcatccagctcactggccagagtttcagcactgatgctgccggtagtagaggaggtagtgatggaaccccccgggggagccccagcccagccagcgtgtcctcggggaggaagtccccacattccaagtcaccagcagagcagcgcgagcggaagtccttggccgatgacaagaagaaagtgaagaacctggggtaccgggantcaggctattactgggaggtaccacccagtgaggtgcagctgctgaagaggatcgggacgggctcgtttggcaccgtgtttcgagggcggtggcatggcgatgtggccgtgaaggtgctcaaggtgtcccagcccacagctgagcaggcccaggctttcaagaatgagatgcaggtgctcaggaagacgcgacatgtcaacatcttgctgtttatgggcttcatgacccggccgggatttgccatcatcacacagtggtgtgagggctccagcctctaccatcacctgcatgtggccgacacacgcttcgacatggtccagctcatcgacgtggcccggcagactgcccagggcatggactacctccatgccaagaacatcatccaccgagatctcaagtctaacaacatcttcctacatgaggggctcacggtgaagatcggtgactttggcttggccacagtgaagactcgatggagcggggcccagcccttggagcagccctcaggatctgtgctgtggatggcagctgaggtgatccgtatgcaggacccgaacccctacagcttccagtcagacgtctatgcctacggggttgtgctctacgagcttatgactggctcactgccttacagccacattggctgccgtgaccagattatctttatggtgggccgtggctatctgtccccggacctcagcaaaatctccagcaactgccccaaggccatgcggcgcctgctgtctgactgcctcaagttccagcgggaggagcggcccctcttcccccagatcctggccacaattgagctgctgcaacggtcactccccaagattgagcggagtgcctcggaaccctccttgcaccgcacccaggccgatgagttgcctgcctgcctactcagcgcagcccgccttgtgccttaggccccgcccaagccaccagggagccaatctcagccctccacgccaaggagccttgcccaccagccaatcaatgttcgtctctgccctgatgctgcctcaggatcccccattccccaccctgggagatgagggggtccccatgtgcttttccagttcttctggaattgggggacccccgccaaagactgagccccctgtctcctccatcatttggtttcctcttggctttggggatacttctaaattttgggagctcctccatctccaatggctgggatttgtggcagggattccactcagaacctctctggaatttgtgcctgatgtgccttccactggattttggggttcccagcaccccatgtggattttggggggtcccttttgtgtctcccccgccattcaaggactcctctctttcttcaccaagaagcacagaattc

SEQ ID NO: 20 - protein, 188 aa, Homo sapiens [sequence omitted]

SEQ ID NO: 21 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 22 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 23 - protein, 766 aa, Homo sapiens [sequence omitted]

SEQ ID NO: 24 - protein, 606 aa, Homo sapiens [sequence omitted]

SEQ ID NO: 25 - protein, 648 aa, Homo sapiens [sequence omitted]

SEQ ID NO: 26 - protein, 465 aa, Homo sapiens
MVSSQKLEKPIEMGSSEPLPIADGDRRRKKKRRGRATDSLPGKFEDMYKLTSELLGEGAYAKVQGAVSLQNGKEYAVKIIEKQAGHSRSRVFREVETLYQCQGNKNILELIEFFEDDTRFYLVFEKLQGGSILAHIQKQKHFNEREASRVVRDVAAALDFLHTKDKVSLCHLGWSAMAPSGLTAAPTSLGSSDPPTSASQVAGTTGIAHRDLKPENILCESPEKVSPVKICDFDLGSGMKLNNSCTPITTPELTTPCGSAEYMAPEVVEVFTDQATFYDKRCDLWSLGVVLYIMLSGYPPFVGHCGADCGWDRGEVCRVCQNKLFESIQEGKYEFPDKDWAHISSEAKDLISKLLVRDAKQRLSAAQVLQHPWVQGQAPEKGLPTPQVLQRNSSTMDLTLFAAEAIALNRQLSQHEENELAEEPEALADGLCSMKLSPPCKSRLARRRALAQAGRGEDRSPPTAL

SEQ ID NO: 27 - protein, 735 aa, Homo sapiens
MPLAQLKEPWPLMELVPLDPENGQTSGEEAGLQPSKDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFYSTIDWNKLYRREITPPFKPAVAQPDDTFYFDTEFTSRTPKDSPGIPPSAGAHQLFRGFSFVATGLMEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQRRVRKLPSTTL

SEQ ID NO: 28 - protein, 733 aa, Homo sapiens
MDLSMKKFAVRRFFSVYLRRKSRSKSSSLSRLEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 29 - protein, 740 aa, Homo sapiens
MPLAQLADPWQKMAVESPSDSAENGQQIMDEPMGEEEINPQTEEVSIKEIAITHHVKEGHEKADPSQFELLKVLGQGSFGKVFLVKKISGSDARQLYAMKVLKKATLKVRDRVRTKMERDILVEVNHPFIVKLHYAFQTEGKLYLILDFLRGGDLFTRLSKEVMFTEEDVKFYLAELALALDHLHSLGIIYRDLKPENILLDEEGHIKLTDFGLSKESIDHEKKAYSFCGTVEYMAPEVVNRRGHTQSADWWSFGVLMFEMLTGTLPFQGKDRKETMTMILKAKLGMPQFLSPEAQSLLRMLFKRNPANRLGAGPDGVEEIKRHSFFSTIDWNKLYRREIHPPFKPATGRPEDTFYFDPEFTAKTPKDSPGIPPSANAHQLFRGFSFVAITSDDESQAMQTVGVHSIVQQLHRNSIQFTDGYEVKEDIGVGSYSVCKRCIHKATNMEFAVKIIDKSKRDPTEEIEILLRYGQHPNIITLKDVYDDGKYVYVVTELMKGGELLDKILRQKFFSEREASAVLFTITKTVEYLHAQGVVHRDLKPSNILYVDESGNPESIRICDFGFAKQLRAENGLLMTPCYTANFVAPEVLKRQGYDAACDIWSLGVLLYTMLTGYTPFANGPDDTPEEILARIGSGKFSLSGGYWNSVSDTAKDLVSKMLHVDPHQRLTAALVLRHPWIVHWDQLPQYQLNRQDAPHLVKGAMAATYSALNRNQSPVLEPVGRSTLAQRRGIKKITSTAL

SEQ ID NO: 30 - protein, 772 aa, Homo sapiens
MGDEDDDESCAVELRITEANLTGHEEKVSVENFELLKVLGTGAYGKVFLVRKAGGHDAGKLYAMKVLRKAALVQRAKTQEHTRTERSVLELVRQAPFLVTLHYAFQTDAKLHLILDYVSGGEMFTHLYQRQYFKEAEVRVYGGEIVLALEHLHKLGIIYRDLKLENVLLDSEGHIVLTDFGLSKEFLTEEKERTFSFCGTIEYMAPEIIRSKTGHGKAVDWWSLGILLFELLTGASPFTLEGERNTQAEVSRRILKCSPPFPPRIGPVAQDLLQRLLCKDPKKRLGAGPQGAQEVRNHPFFQGLDWVALAARKIPAPFRPQIRSELDVGNFAEEFTRLEPVYSPPGSPPPGDPRIFQGYSFVAPSILFDHNNAVMTDGLEAPGAGDRPGRAAVARSAMMQDSPFFQQYELDLREPALGQGSFSVCRRCRQRQSGQEFAVKILSRRLEANTQREVAALRLCQSHPNVVNLHEVHHDQLHTYLVLELLRGGELLEHIRKKRHFSESEASQILRSLVSAVSFMHEEAGVVHRDLKPENILYADDTPGAPVKIIDFGFARLRPQSPGVPMQTPCFTLQYAAPELLAQQGYDESCDLWSLGVILYMMLSGQVPFQGASGQGGQSQAAEIMCKIREGRFSLDGEAWQGVSEEAKELVRGLLTVDPAKRLKLEGLRGSSWLQDGSARSSPPLRTPDVLESSGPAVRSGLNATFMAFNRGKREGFFLKSVENAPLAKRRKQKLRSATASRRGSPAPANPGRAPVASKGAPRRANGPLPPS

SEQ ID NO: 31 - protein, 441 aa, Homo sapiens
MKAAVDLKPTLTIIKTEKVDLELFPSPDMECADVPLLTPSSKEMMSQALKATFSGFTKEQQRLGIPKDPRQWTETHVRDWVMWAVNEFSLKGVDFQKFCMNGAALCALGKDCFLELAPDFVGDILWEHLEILQKEDVKPYQVNGVNPAYPESRYTSDYFISYGIEHAQCVPPSEFSEPSFITESYQTLHPISSEELLSLKYENDYPSVILRDPLQTDTLQNDYFAIKQEVVTPDNMCMGRTSRGKLGGQDSFESIESYDSCDRLTQSWSSQSSFNSLQRVPSYDSFDSEDYPAALPNHKPKGTFKDYVRDRADLNKDKPVIPAAALAGYTGSGPIQLWQFLLELLTDKSCQSFISWTGDGWEFKLSDPDEVARRWGKRKNKPKMNYEKLSRGLRYYYDKNIIHKTAGKRYVYRFVCDLQSLLGYTPEELHAMLDVKPDADE

SEQ ID NO: 32 - protein, 428 aa, Homo sapiens
MDPSVTLWQFLLQLLREQGNGHIISWTSRDGGEFKLVDAEEVARLWGLRKNKTNMNYDKLSRALRYYYDKNIIRKVSGQKFVYKFVSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 33 - protein, 128 aa, Homo sapiens
MDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 34 - DNA, 2043 bp, Artificial Sequence
  Description of Artificial Sequence Synthetic polynucleotide
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 35 - DNA, 37 bp, Artificial Sequence
  Description of Artificial Sequence Synthetic oligonucleotide
gtaaaacgacggccagtttgtggctagaggagtctgc

SEQ ID NO: 36 - DNA, 36 bp, Artificial Sequence
  Description of Artificial Sequence Synthetic oligonucleotide
caggaaacagctatgacctgtaggaagtggcctgtg

SEQ ID NO: 37 - DNA, 38 bp, Artificial Sequence
  Description of Artificial Sequence Synthetic oligonucleotide
gtaaaacgacggccagtaggctgtgagctgagaactgc

SEQ ID NO: 38 - DNA, 39 bp, Artificial Sequence
  Description of Artificial Sequence Synthetic oligonucleotide
caggaaacagctatgaccgcatagcaaagccatgttgag

SEQ ID NO: 39 - DNA, 38 bp, Artificial Sequence
  Description of Artificial Sequence Synthetic oligonucleotide
gtaaaacgacggccagtacaacacgatttcccttggag

SEQ ID NO: 40 - DNA, 39 bp, Artificial Sequence
  Description of Artificial Sequence Synthetic oligonucleotide
caggaaacagctatgaccggtgtatgaaggccaggtgtc

SEQ ID NO: 41 - DNA, 38 bp, Artificial Sequence
  Description of Artificial Sequence Synthetic oligonucleotide
gtaaaacgacggccagtccctgtccaagcctaaagttg

SEQ ID NO: 42 - DNA, 39 bp, Artificial Sequence
  Description of Artificial Sequence Synthetic oligonucleotide
caggaaacagctatgaccgctgcccatgtttacagaatg

SEQ ID NO: 43 - DNA, 37 bp, Artificial Sequence
  Description of Artificial Sequence Synthetic oligonucleotide
gtaaaacgacggccagttactggagcccagaaattcg

SEQ ID NO: 44 - DNA, 38 bp, Artificial Sequence
  Description of Artificial Sequence Synthetic oligonucleotide
caggaaacagctatgacctccttgtgagcactggaagc

SEQ ID NO: 45 - DNA, 38 bp, Artificial Sequence
  Description of Artificial Sequence Synthetic oligonucleotide
gtaaaacgacggccagtatgtaagggacaagcagccac

SEQ ID NO: 46 - DNA, 42 bp, Artificial Sequence
  Description of Artificial Sequence Synthetic oligonucleotide
caggaaacagctatgaccggaaatatagggaagggaaggaac

SEQ ID NO: 47 - DNA, 37 bp, Artificial Sequence
  Description of Artificial Sequence Synthetic oligonucleotide
gtaaaacgacggccagtttgagaaccactgttgtcgg

SEQ ID NO: 48 - DNA, 40 bp, Artificial Sequence
  Description of Artificial Sequence Synthetic oligonucleotide
caggaaacagctatgaccactttctcaactttcccagcag

SEQ ID NO: 49 - DNA, 36 bp, Artificial Sequence
  Description of Artificial Sequence Synthetic oligonucleotide
gtaaaacgacggccagtgaagtcgcagtcacattcg

SEQ ID NO: 50 - DNA, 41 bp, Artificial Sequence
  Description of Artificial Sequence Synthetic oligonucleotide
caggaaacagctatgaccttgaaattgtatgtctgtgtgcc

SEQ ID NO: 51 - DNA, 37 bp, Artificial Sequence
  Description of Artificial Sequence Synthetic oligonucleotide
gtaaaacgacggccagtctctggtttgtgaaggagcc

SEQ ID NO: 52 - DNA, 36 bp, Artificial Sequence
  Description of Artificial Sequence Synthetic oligonucleotide
caggaaacagctatgaccagccacacgacaggggta

SEQ ID NO: 53 - DNA, 36 bp, Artificial Sequence
  Description of Artificial Sequence Synthetic oligonucleotide
gtaaaacgacggccagtaagtgagtgtgcgaccgag

SEQ ID NO: 54 - DNA, 39 bp, Artificial Sequence
  Description of Artificial Sequence Synthetic oligonucleotide
caggaaacagctatgaccgtccacggatccagaaacaag

SEQ ID NO: 55 - DNA, 37 bp, Artificial Sequence
  Description of Artificial Sequence Synthetic oligonucleotide
gtaaaacgacggccagtgttgctggtagccgtaattg

SEQ ID NO: 56 - DNA, 33 bp, Artificial Sequence
  Description of Artificial Sequence Synthetic oligonucleotide
caggaaacagctatgacctcctctggcaaggct

SEQ ID NO: 57 - DNA, 41 bp, Artificial Sequence
  Description of Artificial Sequence Synthetic oligonucleotide
gtaaaacgacggccagtttgtgaatactgggaactatgaaa

SEQ ID NO: 58 - DNA, 40 bp, Artificial Sequence
  Description of Artificial Sequence Synthetic oligonucleotide
caggaaacagctatgacctcatcctaacacatttcaagcc

SEQ ID NO: 59 - DNA, 37 bp, Artificial Sequence
  Description of Artificial Sequence Synthetic oligonucleotide
gtaaaacgacggccagtagggggtgagtcacaggttc

SEQ ID NO: 60 - DNA, 44 bp, Artificial Sequence
  Description of Artificial Sequence Synthetic oligonucleotide
caggaaacagctatgacctcagaagaaatgtttttattccaagg

SEQ ID NO: 61 - DNA, 38 bp, Artificial Sequence
  Description of Artificial Sequence Synthetic oligonucleotide
gtaaaacgacggccagtgcaaatccaattttcccactt

SEQ ID NO: 62 - DNA, 38 bp, Artificial Sequence
  Description of Artificial Sequence Synthetic oligonucleotide
caggaaacagctatgaccgcaggagctctgtgccctat

SEQ ID NO: 63 - DNA, 36 bp, Artificial Sequence
  Description of Artificial Sequence Synthetic oligonucleotide
gtaaaacgacggccagtcccacagcatgacctacca

SEQ ID NO: 64 - DNA, 41 bp, Artificial Sequence
  Description of Artificial Sequence Synthetic oligonucleotide
caggaaacagctatgacctttgcttcttaaggaactgaaaa

SEQ ID NO: 65 - DNA, 36 bp, Artificial Sequence
  Description of Artificial Sequence Synthetic oligonucleotide
gtaaaacgacggccagtgtcacccaaggtcatggag

SEQ ID NO: 66 - DNA, 37 bp, Artificial Sequence
  Description of Artificial Sequence Synthetic oligonucleotide
caggaaacagctatgaccaaaagccaagggcaaagaa

SEQ ID NO: 67 - DNA, 37 bp, Artificial Sequence
  Description of Artificial Sequence Synthetic oligonucleotide
gtaaaacgacggccagtggagtcccaactccttgacc

SEQ ID NO: 68 - DNA, 37 bp, Artificial Sequence
  Description of Artificial Sequence Synthetic oligonucleotide
caggaaacagctatgaccgtcctgcccacacaggatg

SEQ ID NO: 69 - DNA, 37 bp, Artificial Sequence
  Description of Artificial Sequence Synthetic oligonucleotide
gtaaaacgacggccagtgctttccccactcacacaca

SEQ ID NO: 70 - DNA, 37 bp, Artificial Sequence
  Description of Artificial Sequence Synthetic oligonucleotide
caggaaacagctatgaccaaacctcggcaatttgttg

SEQ ID NO: 71 - DNA, 37 bp, Artificial Sequence
  Description of Artificial Sequence Synthetic oligonucleotide
gtaaaacgacggccagtccaccaatccaacatccaga

SEQ ID NO: 72 - DNA, 38 bp, Artificial Sequence
  Description of Artificial Sequence Synthetic oligonucleotide
caggaaacagctatgacctggcccagagccatagaaac

SEQ ID NO: 73 - DNA, 42 bp, Artificial Sequence
  Description of Artificial Sequence Synthetic oligonucleotide
gtaaaacgacggccagttccaagatcattctacaagatgtca

SEQ ID NO: 74 - DNA, 41 bp, Artificial Sequence
  Description of Artificial Sequence Synthetic oligonucleotide
caggaaacagctatgaccgcacattcagagattctttctgc

SEQ ID NO: 75 - DNA, 36 bp, Artificial Sequence
  Description of Artificial Sequence Synthetic oligonucleotide
gtaaaacgacggccagtccaaatgagctggcaagtg

SEQ ID NO: 76 - DNA, 41 bp, Artificial Sequence
  Description of Artificial Sequence Synthetic oligonucleotide
caggaaacagctatgacctcccaaacactcagtgaaacaaa

SEQ ID NO: 77 - DNA, 35 bp, Artificial Sequence
  Description of Artificial Sequence Synthetic oligonucleotide
gtaaaacgacggccagtgcatcgctggtaacatcc

SEQ ID NO: 78 - DNA, 38 bp, Artificial Sequence
  Description of Artificial Sequence Synthetic oligonucleotide
caggaaacagctatgacctgtggagatgagcagggtct

SEQ ID NO: 79 - DNA, 37 bp, Artificial Sequence
  Description of Artificial Sequence Synthetic oligonucleotide
gtaaaacgacggccagtgggtgagtctctgtgtggag

SEQ ID NO: 80 - DNA, 42 bp, Artificial Sequence
  Description of Artificial Sequence Synthetic oligonucleotide
caggaaacagctatgaccattgccatagcaaaaataaacaca

SEQ ID NO: 81 - DNA, 37 bp, Artificial Sequence
  Description of Artificial Sequence Synthetic oligonucleotide
gtaaaacgacggccagtatcgcattcatgcgtcttca

SEQ ID NO: 82 - DNA, 38 bp, Artificial Sequence
  Description of Artificial Sequence Synthetic oligonucleotide
caggaaacagctatgaccatccccatggcaaactcttg

SEQ ID NO: 83 - DNA, 36 bp, Artificial Sequence
  Description of Artificial Sequence Synthetic oligonucleotide
gtaaaacgacggccagtgctcagagcctggcatgaa

SEQ ID NO: 84 - DNA, 36 bp, Artificial Sequence
  Description of Artificial Sequence Synthetic oligonucleotide
caggaaacagctatgaccatcctcccctgcatgtgt

SEQ ID NO: 85 - DNA, 36 bp, Artificial Sequence
  Description of Artificial Sequence Synthetic oligonucleotide
gtaaaacgacggccagtggctcgtctgtgtgtgtca

SEQ ID NO: 86 - DNA, 42 bp, Artificial Sequence
  Description of Artificial Sequence Synthetic oligonucleotide
caggaaacagctatgaccgaaagaaaatacttgcatgtcaga

SEQ ID NO: 87 - DNA, 36 bp, Artificial Sequence
  Description of Artificial Sequence Synthetic oligonucleotide
gtaaaacgacggccagtgaagcaaattgcccaagac

SEQ ID NO: 88 - DNA, 38 bp, Artificial Sequence
  Description of Artificial Sequence Synthetic oligonucleotide
caggaaacagctatgacctgacatttctccagggatgc

SEQ ID NO: 89 - DNA, 37 bp, Artificial Sequence
  Description of Artificial Sequence Synthetic oligonucleotide
gtaaaacgacggccagtaagtgtcgcatcaccaatgc

SEQ ID NO: 90 - DNA, 37 bp, Artificial Sequence
  Description of Artificial Sequence Synthetic oligonucleotide
caggaaacagctatgaccatgcgatctgggacacagg

SEQ ID NO: 91 - DNA, 37 bp, Artificial Sequence
  Description of Artificial Sequence Synthetic oligonucleotide
gtaaaacgacggccagtggcacctgctggcaatagac

SEQ ID NO: 92 - DNA, 45 bp, Artificial Sequence
  Description of Artificial Sequence Synthetic oligonucleotide
caggaaacagctatgacctgacttcatatccatgtgagtttcact

SEQ ID NO: 93 - DNA, 36 bp, Artificial Sequence
  Description of Artificial Sequence Synthetic oligonucleotide
gtaaaacgacggccagtataccctccatgaggcaca

SEQ ID NO: 94 - DNA, 38 bp, Artificial Sequence
  Description of Artificial Sequence Synthetic oligonucleotide
caggaaacagctatgaccgggaaaaacccacacaggaa

SEQ ID NO: 95 - DNA, 37 bp, Artificial Sequence
  Description of Artificial Sequence Synthetic oligonucleotide
gtaaaacgacggccagtcagaaccagcatctcaagga

SEQ ID NO: 96 - DNA, 37 bp, Artificial Sequence
  Description of Artificial Sequence Synthetic oligonucleotide
caggaaacagctatgaccgatgctggagggagcacct

SEQ ID NO: 97 - DNA, 39 bp, Artificial Sequence
  Description of Artificial Sequence Synthetic oligonucleotide
gtaaaacgacggccagtccttgttgaggacattcacagg

SEQ ID NO: 98 - DNA, 38 bp, Artificial Sequence
  Description of Artificial Sequence Synthetic oligonucleotide
caggaaacagctatgaccatgtgcccgaggtggaagta

SEQ ID NO: 99 - DNA, 36 bp, Artificial Sequence
  Description of Artificial Sequence Synthetic oligonucleotide
caggaaacagctatgaccttctccgaggtggaattg

SEQ ID NO: 100 - DNA, 36 bp, Artificial Sequence
  Description of Artificial Sequence Synthetic oligonucleotide
gtaaaacgacggccagtggttcaactgggcgtccta

SEQ ID NO: 101 - DNA, 37 bp, Artificial Sequence
  Description of Artificial Sequence Synthetic oligonucleotide
gtaaaacgacggccagtcgcaccatggcatctcttta

SEQ ID NO: 102 - DNA, 41 bp, Artificial Sequence
  Description of Artificial Sequence Synthetic oligonucleotide
caggaaacagctatgaccaaaacgatctctatgtccgtggt

SEQ ID NO: 103 - DNA, 37 bp, Artificial Sequence
  Description of Artificial Sequence Synthetic oligonucleotide
gtaaaacgacggccagtcagccagccaaacaatcaga

SEQ ID NO: 104 - DNA, 41 bp, Artificial Sequence
  Description of Artificial Sequence Synthetic oligonucleotide
caggaaacagctatgacctctttggagtcttcagagggaaa

SEQ ID NO: 105 - DNA, 36 bp, Artificial Sequence
  Description of Artificial Sequence Synthetic oligonucleotide
gtaaaacgacggccagtgtggtttcgttggaagcaa

SEQ ID NO: 106 - DNA, 38 bp, Artificial Sequence
  Description of Artificial Sequence Synthetic oligonucleotide
caggaaacagctatgaccaattgacagctcccccacag

SEQ ID NO: 107 - DNA, 37 bp, Artificial Sequence
  Description of Artificial Sequence Synthetic oligonucleotide
gtaaaacgacggccagtggctttctgacgggagtcaa

SEQ ID NO: 108 - DNA, 37 bp, Artificial Sequence
  Description of Artificial Sequence Synthetic oligonucleotide
caggaaacagctatgaccacccaaagactctccaaga

SEQ ID NO: 109 - DNA, 37 bp, Artificial Sequence
  Description of Artificial Sequence Synthetic oligonucleotide
gtaaaacgacggccagtcctttccatcacccctcaag

SEQ ID NO: 110 - DNA, 38 bp, Artificial Sequence
  Description of Artificial Sequence Synthetic oligonucleotide
caggaaacagctatgaccagtgccttcccattgcctaa

SEQ ID NO: 111 - DNA, 37 bp, Artificial Sequence
  Description of Artificial Sequence Synthetic oligonucleotide
gtaaaacgacggccagtaccggaattccttcctgctt

SEQ ID NO: 112 - DNA, 40 bp, Artificial Sequence
  Description of Artificial Sequence Synthetic oligonucleotide
caggaaacagctatgaccactgaaacaaacaacagggtga

SEQ ID NO: 113 - DNA, 39 bp, Artificial Sequence
  Description of Artificial Sequence Synthetic oligonucleotide
gtaaaacgacggccagtgacttggagtgagtttggatgg

SEQ ID NO: 114 - DNA, 39 bp, Artificial Sequence
  Description of Artificial Sequence Synthetic oligonucleotide
gtaaaacgacggccagtgggctgcttgaggaagtataag

SEQ ID NO: 115 - DNA, 39 bp, Artificial Sequence
  Description of Artificial Sequence Synthetic oligonucleotide
caggaaacagctatgaccggccagccacgttatagagag

SEQ ID NO: 116 - DNA, 39 bp, Artificial Sequence
  Description of Artificial Sequence Synthetic oligonucleotide
caggaaacagctatgacctccacaacttcgggataggag

SEQ ID NO: 117 - DNA, 38 bp, Artificial Sequence
  Description of Artificial Sequence Synthetic oligonucleotide
gtaaaacgacggccagtcagagcagctccaagtgtttg

SEQ ID NO: 118 - DNA, 39 bp, Artificial Sequence
  Description of Artificial Sequence Synthetic oligonucleotide
caggaaacagctatgaccagtactccctcaggcccaaag

SEQ ID NO: 119 - DNA, 34 bp, Artificial Sequence
  Description of Artificial Sequence Synthetic oligonucleotide
gtaaaacgacggccagtctgcagagtgtgctggg

SEQ ID NO: 120 - DNA, 39 bp, Artificial Sequence
  Description of Artificial Sequence Synthetic oligonucleotide
caggaaacagctatgacctcaagaagggtgcacagagac

SEQ ID NO: 121 - DNA, 37 bp, Artificial Sequence
  Description of Artificial Sequence Synthetic oligonucleotide
gtaaaacgacggccagtctagctgggtcctacctgcc

SEQ ID NO: 122 - DNA, 39 bp, Artificial Sequence
  Description of Artificial Sequence Synthetic oligonucleotide
caggaaacagctatgaccagcctgagagaagggacagtg

SEQ ID NO: 123 - DNA, 37 bp, Artificial Sequence
  Description of Artificial Sequence Synthetic oligonucleotide
gtaaaacgacggccagtcaactgcagccagttccttc

SEQ ID NO: 124 - DNA, 39 bp, Artificial Sequence
  Description of Artificial Sequence Synthetic oligonucleotide
caggaaacagctatgaccagcacaccctactgcatctcg

SEQ ID NO: 125 - DNA, 38 bp, Artificial Sequence
  Description of Artificial Sequence Synthetic oligonucleotide
gtaaaacgacggccagtccaactaagggcctgatccta

SEQ ID NO: 126 - DNA, 40 bp, Artificial Sequence
  Description of Artificial Sequence Synthetic oligonucleotide
caggaaacagctatgaccgggatagaactgctagggcatt

SEQ ID NO: 127 - DNA, 36 bp, Artificial Sequence
  Description of Artificial Sequence Synthetic oligonucleotide
gtaaaacgacggccagttgttgtgaggctggaaagg

SEQ ID NO: 128 - DNA, 38 bp, Artificial Sequence
  Description of Artificial Sequence Synthetic oligonucleotide
caggaaacagctatgacctctagggtgtggagggactg

SEQ ID NO: 129 - DNA, 39 bp, Artificial Sequence
  Description of Artificial Sequence Synthetic oligonucleotide
caggaaacagctatgaccttcctcagctccgtctctttc

SEQ ID NO: 130 - DNA, 38 bp, Artificial Sequence
  Description of Artificial Sequence Synthetic oligonucleotide
gtaaaacgacggccagtatgccaaacaccttcatgtcc

SEQ ID NO: 131 - DNA, 38 bp, Artificial Sequence
  Description of Artificial Sequence Synthetic oligonucleotide
gtaaaacgacggccagtagatccggaagtacacgatgc

SEQ ID NO: 132 - DNA, 39 bp, Artificial Sequence
  Description of Artificial Sequence Synthetic oligonucleotide
caggaaacagctatgaccaaacactgcctccagctcttg

SEQ ID NO: 133 - DNA, 38 bp, Artificial Sequence
  Description of Artificial Sequence Synthetic oligonucleotide
gtaaaacgacggccagtggtgaaggatgtttggaggac

SEQ ID NO: 134 - DNA, 39 bp, Artificial Sequence
  Description of Artificial Sequence Synthetic oligonucleotide
caggaaacagctatgacctaggtttgcgggagtcatatc

SEQ ID NO: 135 - DNA, 36 bp, Artificial Sequence
  Description of Artificial Sequence Synthetic oligonucleotide
gtaaaacgacggccagtggtttgtgatggttgggag

SEQ ID NO: 136 - DNA, 38 bp, Artificial Sequence
  Description of Artificial Sequence Synthetic oligonucleotide
caggaaacagctatgaccatgtagaccttctgggaggg

SEQ ID NO: 137 - DNA, 39 bp, Artificial Sequence; identical to SEQ ID NO: 115
  Description of Artificial Sequence Synthetic oligonucleotide

SEQ ID NO: 138 - DNA, 39 bp, Artificial Sequence; identical to SEQ ID NO: 113
  Description of Artificial Sequence Synthetic oligonucleotide

SEQ ID NO: 139 - DNA, 37 bp, Artificial Sequence
  Description of Artificial Sequence Synthetic oligonucleotide
gtaaaacgacggccagtgaacaggactgctcagtggc

SEQ ID NO: 140 - DNA, 38 bp, Artificial Sequence
  Description of Artificial Sequence Synthetic oligonucleotide
caggaaacagctatgacctaatgcacacaaagcctccc

SEQ ID NO: 141 - DNA, 37 bp, Artificial Sequence
  Description of Artificial Sequence Synthetic oligonucleotide
gtaaaacgacggccagtattgccaaggtatgcacctg

SEQ ID NO: 142 - DNA, 39 bp, Artificial Sequence
  Description of Artificial Sequence Synthetic oligonucleotide
caggaaacagctatgacctcctccaactgtgtgttgtgg

SEQ ID NO: 143 - DNA, 38 bp, Artificial Sequence
  Description of Artificial Sequence Synthetic oligonucleotide
gtaaaacgacggccagtctgggtggagtggtgtctagc

SEQ ID NO: 144 - DNA, 39 bp, Artificial Sequence
  Description of Artificial Sequence Synthetic oligonucleotide
caggaaacagctatgacctacccggtctttccctaatcc

SEQ ID NO: 145 - DNA, 38 bp, Artificial Sequence
  Description of Artificial Sequence Synthetic oligonucleotide
gtaaaacgacggccagtactcctgagcagaacctctgg

SEQ ID NO: 146 - DNA, 39 bp, Artificial Sequence
  Description of Artificial Sequence Synthetic oligonucleotide
caggaaacagctatgaccgttcctcaagagtggctttgg

SEQ ID NO: 147 - DNA, 38 bp, Artificial Sequence
  Description of Artificial Sequence Synthetic oligonucleotide
gtaaaacgacggccagtctctaccacctgagggctttg

SEQ ID NO: 148 - DNA, 38 bp, Artificial Sequence
  Description of Artificial Sequence Synthetic oligonucleotide
caggaaacagctatgacctggactcatctctccttccc

SEQ ID NO: 149 - DNA, 38 bp, Artificial Sequence
  Description of Artificial Sequence Synthetic oligonucleotide
gtaaaacgacggccagtgggaaggagagatgagtccag

SEQ ID NO: 150 - DNA, 39 bp, Artificial Sequence
  Description of Artificial Sequence Synthetic oligonucleotide
caggaaacagctatgaccagaaagggaccctagtccacc

SEQ ID NO: 151 - DNA, 38 bp, Artificial Sequence
  Description of Artificial Sequence Synthetic oligonucleotide
gtaaaacgacggccagtcctgtcaccttccatggagtc

SEQ ID NO: 152 - DNA, 35 bp, Artificial Sequence
  Description of Artificial Sequence Synthetic oligonucleotide
caggaaacagctatgacctctgccactccctctgc

SEQ ID NO: 153 - DNA, 35 bp, Artificial Sequence
  Description of Artificial Sequence Synthetic oligonucleotide
gtaaaacgacggccagtcctctgaagaggaggccc

SEQ ID NO: 154 - DNA, 41 bp, Artificial Sequence
  Description of Artificial Sequence Synthetic oligonucleotide
caggaaacagctatgaccgctggttcacatattctgaaagg

SEQ ID NO: 155 - DNA, 37 bp, Artificial Sequence
  Description of Artificial Sequence Synthetic oligonucleotide
gtaaaacgacggccagtcagagagactgatgggcagg

SEQ ID NO: 156 - DNA, 36 bp, Artificial Sequence
  Description of Artificial Sequence Synthetic oligonucleotide
caggaaacagctatgacctccctttgaaggtgctgg

SEQ ID NO: 157 - DNA, 35 bp, Artificial Sequence
  Description of Artificial Sequence Synthetic oligonucleotide
gtaaaacgacggccagtaaccagccagatgttcgg

SEQ ID NO: 158 - DNA, 38 bp, Artificial Sequence
  Description of Artificial Sequence Synthetic oligonucleotide
caggaaacagctatgaccttgatgccagcagaagtcag

SEQ ID NO: 159 - DNA, 39 bp, Artificial Sequence
  Description of Artificial Sequence Synthetic oligonucleotide
gtaaaacgacggccagtgatagctttctctcctccctgg

SEQ ID NO: 160 - DNA, 39 bp, Artificial Sequence
  Description of Artificial Sequence Synthetic oligonucleotide
caggaaacagctatgaccaggcactgggttgtaagttgg

SEQ ID NO: 161 - DNA, 38 bp, Artificial Sequence
  Description of Artificial Sequence Synthetic oligonucleotide
gtaaaacgacggccagtgggttctctgtcttgtctccc

SEQ ID NO: 162 - DNA, 37 bp, Artificial Sequence
  Description of Artificial Sequence Synthetic oligonucleotide
caggaaacagctatgacctgtatgacacctgcattcc

SEQ ID NO: 163 - DNA, 38 bp, Artificial Sequence
  Description of Artificial Sequence Synthetic oligonucleotide
gtaaaacgacggccagtggataacaggcttgggatgtc

SEQ ID NO: 164 - DNA, 38 bp, Artificial Sequence
  Description of Artificial Sequence Synthetic oligonucleotide
caggaaacagctatgaccatagggcagtaccaggcagg

SEQ ID NO: 165 - DNA, 37 bp, Artificial Sequence
  Description of Artificial Sequence Synthetic oligonucleotide
gtaaaacgacggccagtttgtgcccaatgtgctctac

SEQ ID NO: 166 - DNA, 37 bp, Artificial Sequence
  Description of Artificial Sequence Synthetic oligonucleotide
caggaaacagctatgacccatatgctcccatttacag

SEQ ID NO: 167 - DNA, 38 bp, Artificial Sequence
  Description of Artificial Sequence Synthetic oligonucleotide
gtaaaacgacggccagtatgcgtggtagggcatttaag

SEQ ID NO: 168 - DNA, 39 bp, Artificial Sequence
  Description of Artificial Sequence Synthetic oligonucleotide
caggaaacagctatgaccaggaaggataggacagggtgg

SEQ ID NO: 169 - DNA, 38 bp, Artificial Sequence
  Description of Artificial Sequence Synthetic oligonucleotide
caggaaacagctatgaccacttctgtctcctgccatcc

SEQ ID NO: 170 - DNA, 37 bp, Artificial Sequence
  Description of Artificial Sequence Synthetic oligonucleotide
gtaaaacgacggccagtgggacctagtctctgccttc

SEQ ID NO: 171 - DNA, 37 bp, Artificial Sequence
  Description of Artificial Sequence Synthetic oligonucleotide
gtaaaacgacggccagtttgagtgaaggcattcatgg

SEQ ID NO: 172 - DNA, 38 bp, Artificial Sequence
  Description of Artificial Sequence Synthetic oligonucleotide
caggaaacagctatgaccaggttctggaagacgctgag

SEQ ID NO: 173 - DNA, 38 bp, Artificial Sequence
  Description of Artificial Sequence Synthetic oligonucleotide
gtaaaacgacggccagtatgcagctctttcccagagtc

SEQ ID NO: 174 - DNA, 39 bp, Artificial Sequence
  Description of Artificial Sequence Synthetic oligonucleotide
caggaaacagctatgaccaatatttggagaacgcgatgg

SEQ ID NO: 175 - DNA, 37 bp, Artificial Sequence
  Description of Artificial Sequence Synthetic oligonucleotide
gtaaaacgacggccagtgcctaaggtatcacagcatc

SEQ ID NO: 176 - DNA, 42 bp, Artificial Sequence
  Description of Artificial Sequence Synthetic oligonucleotide
caggaaacagctatgacctgtcttgaaagcagatagaaacca

SEQ ID NO: 177 - DNA, 39 bp, Artificial Sequence
  Description of Artificial Sequence Synthetic oligonucleotide
gtaaaacgacggccagtccccttaaagtagttgtcatgc

SEQ ID NO: 178 - DNA, 40 bp, Artificial Sequence
  Description of Artificial Sequence Synthetic oligonucleotide
caggaaacagctatgaccttgctgcttggaggtattaaag

SEQ ID NO: 179 - DNA, 37 bp, Artificial Sequence
  Description of Artificial Sequence Synthetic oligonucleotide
gtaaaacgacggccagtctgtaagcttcaccgcatcc

SEQ ID NO: 180 - DNA, 39 bp, Artificial Sequence
  Description of Artificial Sequence Synthetic oligonucleotide
caggaaacagctatgacctgaaagcttgacagatcccag

SEQ ID NO: 181 - DNA, 36 bp, Artificial Sequence
  Description of Artificial Sequence Synthetic oligonucleotide
gtaaaacgacggccagtccaccgctcacttaaccag

SEQ ID NO: 182 - DNA, 36 bp, Artificial Sequence
  Description of Artificial Sequence Synthetic oligonucleotide
caggaaacagctatgaccggcattggacaaaatccg

SEQ ID NO: 183 - DNA, 35 bp, Artificial Sequence
  Description of Artificial Sequence Synthetic oligonucleotide
gtaaaacgacggccagtatctgcaccagcctgcaa

SEQ ID NO: 184 - DNA, 39 bp, Artificial Sequence
  Description of Artificial Sequence Synthetic oligonucleotide
caggaaacagctatgaccattgcccagttgatgtcattg

SEQ ID NO: 185 - DNA, 38 bp, Artificial Sequence
  Description of Artificial Sequence Synthetic oligonucleotide
gtaaaacgacggccagtcatccacttaatagcccgagg

SEQ ID NO: 186 - DNA, 39 bp, Artificial Sequence
  Description of Artificial Sequence Synthetic oligonucleotide
caggaaacagctatgaccggcaaaccttgctttatagac

SEQ ID NO: 187 - DNA, 44 bp, Artificial Sequence
  Description of Artificial Sequence Synthetic oligonucleotide
gtaaaacgacggccagtaacaatgcattatagaagatatttggg

SEQ ID NO: 188 - DNA, 37 bp, Artificial Sequence
  Description of Artificial Sequence Synthetic oligonucleotide
caggaaacagctatgaccgcgatgatgaggctgaaga

SEQ ID NO: 189 - DNA, 35 bp, Artificial Sequence
  Description of Artificial Sequence Synthetic oligonucleotide
gtaaaacgacggccagtgccaagtgatcttcccag

SEQ ID NO: 190 - DNA, 40 bp, Artificial Sequence
  Description of Artificial Sequence Synthetic oligonucleotide
caggaaacagctatgacctttagacttgggccttaggttg

SEQ ID NO: 191 - DNA, 36 bp, Artificial Sequence
  Description of Artificial Sequence Synthetic oligonucleotide
gtaaaacgacggccagtcatgcccacccagaaagta

SEQ ID NO: 192 - DNA, 32 bp, Artificial Sequence
  Description of Artificial Sequence Synthetic oligonucleotide
caggaaacagctatgaccagaaatcccgacga

SEQ ID NO: 193 - DNA, 36 bp, Artificial Sequence
  Description of Artificial Sequence Synthetic oligonucleotide
gtaaaacgacggccagtccatgatgaaaactctgcg

SEQ ID NO: 194 - DNA, 40 bp, Artificial Sequence
  Description of Artificial Sequence Synthetic oligonucleotide
caggaaacagctatgaccacatcgatcaagaagagctcaa

SEQ ID NO: 195 - DNA, 40 bp, Artificial Sequence
  Description of Artificial Sequence Synthetic oligonucleotide
gtaaaacgacggccagtgagtcttcgtgtattccaagctg

SEQ ID NO: 196 - DNA, 40 bp, Artificial Sequence
  Description of Artificial Sequence Synthetic oligonucleotide
caggaaacagctatgaccaatacgtcccatcatcttcagg

SEQ ID NO: 197 - DNA, 36 bp, Artificial Sequence
  Description of Artificial Sequence Synthetic oligonucleotide
gtaaaacgacggccagttgtaacagtgctacctgag

SEQ ID NO: 198 - DNA, 40 bp, Artificial Sequence
  Description of Artificial Sequence Synthetic oligonucleotide
caggaaacagctatgacctgaactggcggaagataaagag

SEQ ID NO: 199 - DNA, 39 bp, Artificial Sequence
  Description of Artificial Sequence Synthetic oligonucleotide
gtaaaacgacggccagtccagccacacacaacatagttt

SEQ ID NO: 200 - DNA, 35 bp, Artificial Sequence
  Description of Artificial Sequence Synthetic oligonucleotide
caggaaacagctatgaccgtcttcggaaacatggc

SEQ ID NO: 201 - DNA, 37 bp, Artificial Sequence
  Description of Artificial Sequence Synthetic oligonucleotide
gtaaaacgacggccagtcaatgatcaggaaatgctgt

SEQ ID NO: 202 - DNA, 42 bp, Artificial Sequence
  Description of Artificial Sequence Synthetic oligonucleotide
caggaaacagctatgacctcttaggttctgcctaggtatctg

SEQ ID NO: 203 - DNA, 36 bp, Artificial Sequence
  Description of Artificial Sequence Synthetic oligonucleotide
gtaaaacgacggccagtcaggacaaggtgctccaag

SEQ ID NO: 204 - DNA, 35 bp, Artificial Sequence
  Description of Artificial Sequence Synthetic oligonucleotide
gtaaaacgacggccagtggggatcttccagactga

SEQ ID NO: 205 - DNA, 36 bp, Artificial Sequence
  Description of Artificial Sequence Synthetic oligonucleotide
caggaaacagctatgaccgctggacagcaaacatgg

SEQ ID NO: 206 - DNA, 43 bp, Artificial Sequence
  Description of Artificial Sequence Synthetic oligonucleotide
caggaaacagctatgaccttgggttctacagatttcatttcac

SEQ ID NO: 207 - DNA, 37 bp, Artificial Sequence
  Description of Artificial Sequence Synthetic oligonucleotide
gtaaaacgacggccagttgggaagggatagtactccg

SEQ ID NO: 208 - DNA, 38 bp, Artificial Sequence
  Description of Artificial Sequence Synthetic oligonucleotide
caggaaacagctatgaccaacaaaggccaaaccactcc

SEQ ID NO: 209 - DNA, 39 bp, Artificial Sequence
  Description of Artificial Sequence Synthetic oligonucleotide
gtaaaacgacggccagtgctggaattaggcttgacaatg

SEQ ID NO: 210 - DNA, 36 bp, Artificial Sequence
  Description of Artificial Sequence Synthetic oligonucleotide
caggaaacagctatgaccggggatcctcaagggaaa

SEQ ID NO: 211 - DNA, 35 bp, Artificial Sequence
  Description of Artificial Sequence Synthetic oligonucleotide
gtaaaacgacggccagtacggctggacagccaata

SEQ ID NO: 212 - DNA, 39 bp, Artificial Sequence
  Description of Artificial Sequence Synthetic oligonucleotide
caggaaacagctatgaccaaatagcattaagtcaaatcc

SEQ ID NO: 213 - DNA, 37 bp, Artificial Sequence
  Description of Artificial Sequence Synthetic oligonucleotide
gtaaaacgacggccagtgcaactcgtctcctctatgg

SEQ ID NO: 214 - DNA, 38 bp, Artificial Sequence
  Description of Artificial Sequence Synthetic oligonucleotide
caggaaacagctatgaccaacacaaggacaggagaggg

SEQ ID NO: 215 - DNA, 37 bp, Artificial Sequence
  Description of Artificial Sequence Synthetic oligonucleotide
gtaaaacgacggccagtagctgtcatccttgccactg

SEQ ID NO: 216 - DNA, 38 bp, Artificial Sequence
  Description of Artificial Sequence Synthetic oligonucleotide
caggaaacagctatgaccactgcccaatcatggagatg

SEQ ID NO: 217 - DNA, 37 bp, Artificial Sequence
  Description of Artificial Sequence Synthetic oligonucleotide
gtaaaacgacggccagtggaggctcttcaggtattgc

SEQ ID NO: 218 - DNA, 41 bp, Artificial Sequence
  Description of Artificial Sequence Synthetic oligonucleotide
caggaaacagctatgaccgaactgctttacagacaggatgc

SEQ ID NO: 219 - DNA, 38 bp, Artificial Sequence
  Description of Artificial Sequence Synthetic oligonucleotide
gtaaaacgacggccagtctggaggttccttcactgtgc

SEQ ID NO: 220 - DNA, 39 bp, Artificial Sequence
  Description of Artificial Sequence Synthetic oligonucleotide
caggaaacagctatgaccaagccagggacaatgagattc

SEQ ID NO: 221 - DNA, 38 bp, Artificial Sequence
  Description of Artificial Sequence Synthetic oligonucleotide
gtaaaacgacggccagtgagaatctcattgtccctggc

SEQ ID NO: 222 - DNA, 36 bp, Artificial Sequence
  Description of Artificial Sequence Synthetic oligonucleotide
caggaaacagctatgaccgagccaacatgcaaaggc

SEQ ID NO: 223 - DNA, 37 bp, Artificial Sequence
  Description of Artificial Sequence Synthetic oligonucleotide
gtaaaacgacggccagtggcagaggaagagaaaggtg

SEQ ID NO: 224 - DNA, 39 bp, Artificial Sequence
  Description of Artificial Sequence Synthetic oligonucleotide
caggaaacagctatgaccgttggacaagttccgtgtgtc

SEQ ID NO: 225 - DNA, 37 bp, Artificial Sequence
  Description of Artificial Sequence Synthetic oligonucleotide
gtaaaacgacggccagtgtgtgctctatcccttaggc

SEQ ID NO: 226 - DNA, 39 bp, Artificial Sequence
  Description of Artificial Sequence Synthetic oligonucleotide
caggaaacagctatgaccagcctcctgaacccttacacc

SEQ ID NO: 227 - DNA, 38 bp, Artificial Sequence
  Description of Artificial Sequence Synthetic oligonucleotide
gtaaaacgacggccagtaggtgtaagggttcaggaggc

SEQ ID NO: 228 - DNA, 37 bp, Artificial Sequence
  Description of Artificial Sequence Synthetic oligonucleotide
caggaaacagctatgaccaagcttgaagggtgggaag

SEQ ID NO: 229 - DNA, 40 bp, Artificial Sequence
  Description of Artificial Sequence Synthetic oligonucleotide
gtaaaacgacggccagtcattgttaagaagtgccttgagc

SEQ ID NO: 230 - DNA, 35 bp, Artificial Sequence
  Description of Artificial Sequence Synthetic oligonucleotide
caggaaacagctatgaccagttcccaggaagccct

SEQ ID NO: 231 - DNA, 37 bp, Artificial Sequence
  Description of Artificial Sequence Synthetic oligonucleotide
gtaaaacgacggccagtagctggcagaagacaaggag

SEQ ID NO: 232 - DNA, 39 bp, Artificial Sequence
  Description of Artificial Sequence Synthetic oligonucleotide
caggaaacagctatgaccatgcctcatactgccagttcc

SEQ ID NO: 233 - DNA, 38 bp, Artificial Sequence
  Description of Artificial Sequence Synthetic oligonucleotide
gtaaaacgacggccagtagcacaattagggcttcctgg

SEQ ID NO: 234 - DNA, 36 bp, Artificial Sequence
  Description of Artificial Sequence Synthetic oligonucleotide
caggaaacagctatgaccggagcccacctcgaagat

SEQ ID NO: 235 - DNA, 35 bp, Artificial Sequence
  Description of Artificial Sequence Synthetic oligonucleotide
gtaaaacgacggccagtagcaggtggccaactcag

SEQ ID NO: 236 - DNA, 39 bp, Artificial Sequence
  Description of Artificial Sequence Synthetic oligonucleotide
caggaaacagctatgaccagaggcaagaaggcatgaaac

SEQ ID NO: 237 - DNA, 38 bp, Artificial Sequence
  Description of Artificial Sequence Synthetic oligonucleotide
gtaaaacgacggccagtatgcatggcattagcaaagac

SEQ ID NO: 238 - DNA, 38 bp, Artificial Sequence
  Description of Artificial Sequence Synthetic oligonucleotide
caggaaacagctatgaccgtcatctttggagcaggaac

SEQ ID NO: 239 - DNA, 37 bp, Artificial Sequence
  Description of Artificial Sequence Synthetic oligonucleotide
gtaaaacgacggccagtggattaagaagcaatgccct

SEQ ID NO: 240 - DNA, 45 bp, Artificial Sequence
  Description of Artificial Sequence Synthetic oligonucleotide
caggaaacagctatgacctggtgtagtggaaactaggaattacat

SEQ ID NO: 241 - DNA, 37 bp, Artificial Sequence
  Description of Artificial Sequence Synthetic oligonucleotide
gtaaaacgacggccagtaacagtctgcatggagcagg

SEQ ID NO: 242 - DNA, 42 bp, Artificial Sequence
  Description of Artificial Sequence Synthetic oligonucleotide
caggaaacagctatgacctcagttgcctgaagagaaacataa

SEQ ID NO: 243 - DNA, 35 bp, Artificial Sequence
  Description of Artificial Sequence Synthetic oligonucleotide
gtaaaacgacggccagtggttgccaccttgttacc

SEQ ID NO: 244 - DNA, 40 bp, Artificial Sequence
  Description of Artificial Sequence Synthetic oligonucleotide
caggaaacagctatgaccgaacaaaccaggattctagccc

SEQ ID NO: 245 - DNA, 38 bp, Artificial Sequence
  Description of Artificial Sequence Synthetic oligonucleotide
gtaaaacgacggccagtcactgggtcaaagtctcctgg

SEQ ID NO: 246 - DNA, 43 bp, Artificial Sequence
  Description of Artificial Sequence Synthetic oligonucleotide
caggaaacagctatgaccgtgtcaaatacttacttggcagagg

SEQ ID NO: 247 - DNA, 39 bp, Artificial Sequence
  Description of Artificial Sequence Synthetic oligonucleotide
gtaaaacgacggccagtaaagctcttcctgtttcagtcc

SEQ ID NO: 248 - DNA, 39 bp, Artificial Sequence
  Description of Artificial Sequence Synthetic oligonucleotide
caggaaacagctatgaccgggattatccaattgcttcca

SEQ ID NO: 249 - DNA, 37 bp, Artificial Sequence
  Description of Artificial Sequence Synthetic oligonucleotide
gtaaaacgacggccagtgacaactgaactgctctcgc

SEQ ID NO: 250 - DNA, 36 bp, Artificial Sequence
  Description of Artificial Sequence Synthetic oligonucleotide
caggaaacagctatgaccacacagtcaggacactgg

SEQ ID NO: 251 - DNA, 38 bp, Artificial Sequence
  Description of Artificial Sequence Synthetic oligonucleotide
gtaaaacgacggccagtaaggaccggttcatcaacttc

SEQ ID NO: 252 - DNA, 39 bp, Artificial Sequence
  Description of Artificial Sequence Synthetic oligonucleotide
caggaaacagctatgacctgatagggaatgcacacatgg

SEQ ID NO: 253 - DNA, 37 bp, Artificial Sequence
  Description of Artificial Sequence Synthetic oligonucleotide
caggaaacagctatgaccttcccttcctttcctccag

SEQ ID NO: 254 - DNA, 38 bp, Artificial Sequence
  Description of Artificial Sequence Synthetic oligonucleotide
gtaaaacgacggccagtactccacagaccctctccttg

SEQ ID NO: 255 - DNA, 37 bp, Artificial Sequence
  Description of Artificial Sequence Synthetic oligonucleotide
gtaaaacgacggccagtgccatttgtgtgggtaatgt

SEQ ID NO: 256 - DNA, 42 bp, Artificial Sequence
  Description of Artificial Sequence Synthetic oligonucleotide
caggaaacagctatgaccggtcttgaaacgaacatcaataca

SEQ ID NO: 257 - DNA, 37 bp, Artificial Sequence
  Description of Artificial Sequence Synthetic oligonucleotide
gtaaaacgacggccagttgatgttcgtttcaagacct

SEQ ID NO: 258 - DNA, 41 bp, Artificial Sequence
  Description of Artificial Sequence Synthetic oligonucleotide
caggaaacagctatgaccggtcccttcggtcaagacttaat

SEQ ID NO: 259 - DNA, 38 bp, Artificial Sequence
  Description of Artificial Sequence Synthetic oligonucleotide
gtaaaacgacggccagtgcatggtcttagaaagttccc

SEQ ID NO: 260 - DNA, 39 bp, Artificial Sequence
  Description of Artificial Sequence Synthetic oligonucleotide
caggaaacagctatgaccaaccaaagcagcaggaatagg

SEQ ID NO: 261 - DNA, 36 bp, Artificial Sequence
  Description of Artificial Sequence Synthetic oligonucleotide
gtaaaacgacggccagtgcaaaaacgattttcattg

SEQ ID NO: 262 - DNA, 36 bp, Artificial Sequence
  Description of Artificial Sequence Synthetic oligonucleotide
caggaaacagctatgacctcctcaaggtcttggcgt

SEQ ID NO: 263 - DNA, 36 bp, Artificial Sequence
  Description of Artificial Sequence Synthetic oligonucleotide
gtaaaacgacggccagtaccgcgtccagcctagttc

SEQ ID NO: 264 - DNA, 40 bp, Artificial Sequence
  Description of Artificial Sequence Synthetic oligonucleotide
caggaaacagctatgaccaaccacacaccaaaggaacatc

SEQ ID NO: 265 - DNA, 38 bp, Artificial Sequence
  Description of Artificial Sequence Synthetic oligonucleotide
gtaaaacgacggccagtccctagaggtttgtgttcacc

SEQ ID NO: 266 - DNA, 43 bp, Artificial Sequence
  Description of Artificial Sequence Synthetic oligonucleotide
caggaaacagctatgacctgaagtcaaataaaatacaaaacca

SEQ ID NO: 267 - DNA, 36 bp, Artificial Sequence
  Description of Artificial Sequence Synthetic oligonucleotide
gtaaaacgacggccagttcattatgggagaatgcca

SEQ ID NO: 268 - DNA, 38 bp, Artificial Sequence
  Description of Artificial Sequence Synthetic oligonucleotide
caggaaacagctatgaccagtgtggttctaaggccaaa

SEQ ID NO: 269 - DNA, 37 bp, Artificial Sequence
  Description of Artificial Sequence Synthetic oligonucleotide
gtaaaacgacggccagttcctcttggttgtcagtgct

SEQ ID NO: 270 - DNA, 35 bp, Artificial Sequence
  Description of Artificial Sequence Synthetic oligonucleotide
caggaaacagctatgaccaactctggggcaggaac

SEQ ID NO: 271 - DNA, 39 bp, Artificial Sequence
  Description of Artificial Sequence Synthetic oligonucleotide
gtaaaacgacggccagttgcaaatatatgtcttccaccc

SEQ ID NO: 272 - DNA, 41 bp, Artificial Sequence
  Description of Artificial Sequence Synthetic oligonucleotide
caggaaacagctatgacctttgttcagaaaaggatttcaag

SEQ ID NO: 273 - DNA, 37 bp, Artificial Sequence
  Description of Artificial Sequence Synthetic oligonucleotide
gtaaaacgacggccagtcatttgaagcatttgctctg

SEQ ID NO: 274 - DNA, 39 bp, Artificial Sequence
  Description of Artificial Sequence Synthetic oligonucleotide
caggaaacagctatgaccgggtgtttctgttgctaaggg

SEQ ID NO: 275 - DNA, 44 bp, Artificial Sequence
  Description of Artificial Sequence Synthetic oligonucleotide
caggaaacagctatgaccgctcgtaaacaaaataagattaatgg

SEQ ID NO: 276 - DNA, 36 bp, Artificial Sequence
  Description of Artificial Sequence Synthetic oligonucleotide
gtaaaacgacggccagtgtggttgatgcagttttcc

SEQ ID NO: 277 - DNA, 38 bp, Artificial Sequence
  Description of Artificial Sequence Synthetic oligonucleotide
gtaaaacgacggccagtttgaaacttggctgtagctga

SEQ ID NO: 278 - DNA, 38 bp, Artificial Sequence
  Description of Artificial Sequence Synthetic oligonucleotide
caggaaacagctatgacctgtggctactggtacttgcg

SEQ ID NO: 279 - DNA, 39 bp, Artificial Sequence
  Description of Artificial Sequence Synthetic oligonucleotide
gtaaaacgacggccagtggaagaaatgttggataaagca

SEQ ID NO: 280 - DNA, 38 bp, Artificial Sequence
  Description of Artificial Sequence Synthetic oligonucleotide
caggaaacagctatgaccggtccgtatttgaagtccca

SEQ ID NO: 281 - DNA, 35 bp, Artificial Sequence
  Description of Artificial Sequence Synthetic oligonucleotide
gtaaaacgacggccagtatgcctgtgggtgcactt

SEQ ID NO: 282 - DNA, 41 bp, Artificial Sequence
  Description of Artificial Sequence Synthetic oligonucleotide
caggaaacagctatgaccaacttctggctaccttactgtca

SEQ ID NO: 283 - DNA, 40 bp, Artificial Sequence
  Description of Artificial Sequence Synthetic oligonucleotide
gtaaaacgacggccagtaaggtagccagaagttgtgtacg

SEQ ID NO: 284 - DNA, 38 bp, Artificial Sequence
  Description of Artificial Sequence Synthetic oligonucleotide
caggaaacagctatgacctcctttctaccaataaccgc

SEQ ID NO: 285 - DNA, 38 bp, Artificial Sequence
  Description of Artificial Sequence Synthetic oligonucleotide
gtaaaacgacggccagtgactaaaggtgtgtgtgtggc

SEQ ID NO: 286 - DNA, 42 bp, Artificial Sequence
  Description of Artificial Sequence Synthetic oligonucleotide
caggaaacagctatgacctgacaacactaacttcccaaacat

SEQ ID NO: 287 - DNA, 37 bp, Artificial Sequence
  Description of Artificial Sequence Synthetic oligonucleotide
gtaaaacgacggccagtccccatttgagatgattttg

SEQ ID NO: 288 - DNA, 41 bp, Artificial Sequence
  Description of Artificial Sequence Synthetic oligonucleotide
caggaaacagctatgacctcctatcctagtcctgtcatggg

SEQ ID NO: 289 - DNA, 35 bp, Artificial Sequence
  Description of Artificial Sequence Synthetic oligonucleotide
gtaaaacgacggccagtcagggccattcacaccat

SEQ ID NO: 290 - DNA, 38 bp, Artificial Sequence
  Description of Artificial Sequence Synthetic oligonucleotide
caggaaacagctatgaccttgtctggagatccttgtgg

SEQ ID NO: 291 - DNA, 36 bp, Artificial Sequence
  Description of Artificial Sequence Synthetic oligonucleotide
gtaaaacgacggccagtgccaacgtagacagtggtc

SEQ ID NO: 292 - DNA, 36 bp, Artificial Sequence
  Description of Artificial Sequence Synthetic oligonucleotide
caggaaacagctatgaccaggcacttttcttccccg

SEQ ID NO: 293 - DNA, 39 bp, Artificial Sequence
  Description of Artificial Sequence Synthetic oligonucleotide
gtaaaacgacggccagtgggattgtttgcactaacctga

SEQ ID NO: 294 - DNA, 38 bp, Artificial Sequence
  Description of Artificial Sequence Synthetic oligonucleotide
caggaaacagctatgaccgaagacaatcagccctgcac

SEQ ID NO: 295 - DNA, 39 bp, Artificial Sequence
  Description of Artificial Sequence Synthetic oligonucleotide
gtaaaacgacggccagtgactccatgcagactctcttcc

SEQ ID NO: 296 - DNA, 40 bp, Artificial Sequence
  Description of Artificial Sequence Synthetic oligonucleotide
caggaaacagctatgaccgccttgcttcatgcagtgttag

SEQ ID NO: 297 - DNA, 38 bp, Artificial Sequence
  Description of Artificial Sequence Synthetic oligonucleotide
caggaaacagctatgaccaaattcacaaagcctgccta

SEQ ID NO: 298 - DNA, 36 bp, Artificial Sequence
  Description of Artificial Sequence Synthetic oligonucleotide
gtaaaacgacggccagtgccatttctgtttgcctta

SEQ ID NO: 299 - DNA, 35 bp, Artificial Sequence
  Description of Artificial Sequence Synthetic oligonucleotide
gtaaaacgacggccagtctacacgttgcacttggc

SEQ ID NO: 300 - DNA, 37 bp, Artificial Sequence
  Description of Artificial Sequence Synthetic oligonucleotide
caggaaacagctatgaccatcagcagctagatccttc

SEQ ID NO: 301 - DNA, 39 bp, Artificial Sequence
  Description of Artificial Sequence Synthetic oligonucleotide
gtaaaacgacggccagtttttgttgattccatttgtgtt

SEQ ID NO: 302 - DNA, 39 bp, Artificial Sequence
  Description of Artificial Sequence Synthetic oligonucleotide
caggaaacagctatgaccgcctttgggataaatcaaacc

SEQ ID NO: 303 - DNA, 40 bp, Artificial Sequence
  Description of Artificial Sequence Synthetic oligonucleotide
gtaaaacgacggccagttgggaaggttagaaacactacct

SEQ ID NO: 304 - DNA, 40 bp, Artificial Sequence
  Description of Artificial Sequence Synthetic oligonucleotide
caggaaacagctatgaccatggatttatgtgaaaccgaaa

SEQ ID NO: 305 - DNA, 39 bp, Artificial Sequence
  Description of Artificial Sequence Synthetic oligonucleotide
gtaaaacgacggccagtctcatgttttgggagaagaaaa

SEQ ID NO: 306 - DNA, 40 bp, Artificial Sequence
  Description of Artificial Sequence Synthetic oligonucleotide
caggaaacagctatgacctgaaaatttagttggaagggga

SEQ ID NO: 307 - DNA, 38 bp, Artificial Sequence
  Description of Artificial Sequence Synthetic oligonucleotide
gtaaaacgacggccagtctgggtgtatctggtgttgaa

SEQ ID NO: 308 - DNA, 45 bp, Artificial Sequence
  Description of Artificial Sequence Synthetic oligonucleotide
caggaaacagctatgaccaaaataataataatgaccactggaacc

SEQ ID NO: 309 - DNA, 38 bp, Artificial Sequence
  Description of Artificial Sequence Synthetic oligonucleotide
gtaaaacgacggccagtgctaagggtaagcaattggga

SEQ ID NO: 310 - DNA, 36 bp, Artificial Sequence
  Description of Artificial Sequence Synthetic oligonucleotide
caggaaacagctatgaccggcctggtggcaaactct

SEQ ID NO: 311 - DNA, 38 bp, Artificial Sequence
  Description of Artificial Sequence Synthetic oligonucleotide
gtaaaacgacggccagtgcatgttgccaaattaccctt

SEQ ID NO: 312 - DNA, 39 bp, Artificial Sequence
  Description of Artificial Sequence Synthetic oligonucleotide
caggaaacagctatgaccaggaaacgcaggctatttacc

SEQ ID NO: 313 - DNA, 40 bp, Artificial Sequence
  Description of Artificial Sequence Synthetic oligonucleotide
gtaaaacgacggccagtgccttatttctcagtgtccaaaa

SEQ ID NO: 314 - DNA, 37 bp, Artificial Sequence
  Description of Artificial Sequence Synthetic oligonucleotide
caggaaacagctatgaccagcagccgctcatgatact

SEQ ID NO: 315 - DNA, 35 bp, Artificial Sequence
  Description of Artificial Sequence Synthetic oligonucleotide
gtaaaacgacggccagtccgtcaccaccactttcc

SEQ ID NO: 316 - DNA, 37 bp, Artificial Sequence
  Description of Artificial Sequence Synthetic oligonucleotide
caggaaacagctatgacctaccgtaaactcgggtcag

SEQ ID NO: 317 - DNA, 42 bp, Artificial Sequence
  Description of Artificial Sequence Synthetic oligonucleotide
gtaaaacgacggccagtaaacaacttcatttgtgttttctcc

SEQ ID NO: 318 - DNA, 39 bp, Artificial Sequence
  Description of Artificial Sequence Synthetic oligonucleotide
caggaaacagctatgacctggatttctcaatgtggccta

SEQ ID NO: 319 - DNA, 38 bp, Artificial Sequence
  Description of Artificial Sequence Synthetic oligonucleotide
gtaaaacgacggccagtcaaggactgttctttcttcgc

SEQ ID NO: 320 - DNA, 36 bp, Artificial Sequence
  Description of Artificial Sequence Synthetic oligonucleotide
caggaaacagctatgacctcaatacctgcccaaggc

SEQ ID NO: 321 - DNA, 36 bp, Artificial Sequence
  Description of Artificial Sequence Synthetic oligonucleotide
gtaaaacgacggccagtgccaagtgtcttttctcca

SEQ ID NO: 322 - DNA, 36 bp, Artificial Sequence
  Description of Artificial Sequence Synthetic oligonucleotide
caggaaacagctatgaccagtgctttgcccaatgtg

SEQ ID NO: 323 - DNA, 38 bp, Artificial Sequence
  Description of Artificial Sequence Synthetic oligonucleotide
gtaaaacgacggccagttgattaggctgttccaatgaa

SEQ ID NO: 324 - DNA, 39 bp, Artificial Sequence
  Description of Artificial Sequence Synthetic oligonucleotide
caggaaacagctatgaccttgcacatccttccaataacc

SEQ ID NO: 325 - DNA, 38 bp, Artificial Sequence
  Description of Artificial Sequence Synthetic oligonucleotide
gtaaaacgacggccagtggttattggaaggatgtgcaa

SEQ ID NO: 326 - DNA, 41 bp, Artificial Sequence
  Description of Artificial Sequence Synthetic oligonucleotide
caggaaacagctatgacctttcatgtcattactggaggctt

SEQ ID NO: 327 - DNA, 39 bp, Artificial Sequence
  Description of Artificial Sequence Synthetic oligonucleotide
gtaaaacgacggccagttcaaaatgaaacatggaacttt

SEQ ID NO: 328 - DNA, 37 bp, Artificial Sequence
  Description of Artificial Sequence Synthetic oligonucleotide
caggaaacagctatgaccaataacacagtccatgcaa

SEQ ID NO: 329 - DNA, 38 bp, Artificial Sequence
  Description of Artificial Sequence Synthetic oligonucleotide
gtaaaacgacggccagtggcatgcatgagagatattcc

SEQ ID NO: 330 - DNA, 37 bp, Artificial Sequence
  Description of Artificial Sequence Synthetic oligonucleotide
caggaaacagctatgaccggagaagtgagggcggaac

SEQ ID NO: 331 - DNA, 38 bp, Artificial Sequence
  Description of Artificial Sequence Synthetic oligonucleotide
gtaaaacgacggccagtcctgaattcattccgagattc

SEQ ID NO: 332 - DNA, 42 bp, Artificial Sequence
  Description of Artificial Sequence Synthetic oligonucleotide
caggaaacagctatgacctctttcctttagcactgatgagac

SEQ ID NO: 333 - DNA, 41 bp, Artificial Sequence
  Description of Artificial Sequence Synthetic oligonucleotide
gtaaaacgacggccagtgctaagtaacgttctcagtccagc

SEQ ID NO: 334 - DNA, 40 bp, Artificial Sequence
  Description of Artificial Sequence Synthetic oligonucleotide
caggaaacagctatgacctctaggaacctcaaggcaaagt

SEQ ID NO: 335 - DNA, 38 bp, Artificial Sequence
  Description of Artificial Sequence Synthetic oligonucleotide
gtaaaacgacggccagtgttctgtggttttctgcagtc

SEQ ID NO: 336 - DNA, 44 bp, Artificial Sequence
  Description of Artificial Sequence Synthetic oligonucleotide
caggaaacagctatgaccttgcatttaaagtaagacataagggc

SEQ ID NO: 337 - DNA, 42 bp, Artificial Sequence
  Description of Artificial Sequence Synthetic oligonucleotide
gtaaaacgacggccagtggagttatatttcctttccttgcag

SEQ ID NO: 338 - DNA, 38 bp, Artificial Sequence
  Description of Artificial Sequence Synthetic oligonucleotide
caggaaacagctatgacctgtgaactttctgctctgcc

SEQ ID NO: 339 - DNA, 35 bp, Artificial Sequence
  Description of Artificial Sequence Synthetic oligonucleotide
gtaaaacgacggccagtcctgccctgcctactttg

SEQ ID NO: 340 - DNA, 38 bp, Artificial Sequence
  Description of Artificial Sequence Synthetic oligonucleotide
caggaaacagctatgacctgcttgcctccattagttgg

SEQ ID NO: 341 - DNA, 42 bp, Artificial Sequence
  Description of Artificial Sequence Synthetic oligonucleotide
caggaaacagctatgaccagtcccatgtggatttacacacta

SEQ ID NO: 342 - DNA, 41 bp, Artificial Sequence
  Description of Artificial Sequence Synthetic oligonucleotide
gtaaaacgacggccagtaggtggtgtgtatgtaaggtgttc

SEQ ID NO: 343 - DNA, 35 bp, Artificial Sequence
  Description of Artificial Sequence Synthetic oligonucleotide
gtaaaacgacggccagtgccacttggaaggagcaa

SEQ ID NO: 344 - DNA, 39 bp, Artificial Sequence
  Description of Artificial Sequence Synthetic oligonucleotide
caggaaacagctatgacctgtttcaaggtcccattctca

SEQ ID NO: 345 - DNA, 38 bp, Artificial Sequence
  Description of Artificial Sequence Synthetic oligonucleotide
gtaaaacgacggccagtaaagaaactgctccagggatg

SEQ ID NO: 346 - DNA, 45 bp, Artificial Sequence
  Description of Artificial Sequence Synthetic oligonucleotide
caggaaacagctatgaccttcactctaaagattctaagaaatggc

SEQ ID NO: 347 - DNA, 41 bp, Artificial Sequence
  Description of Artificial Sequence Synthetic oligonucleotide
gtaaaacgacggccagtccaggtgtttgatcacgttaattc

SEQ ID NO: 348 - DNA, 38 bp, Artificial Sequence
  Description of Artificial Sequence Synthetic oligonucleotide
caggaaacagctatgaccacaacactggcctctgctaa

SEQ ID NO: 349 - DNA, 37 bp, Artificial Sequence
  Description of Artificial Sequence Synthetic oligonucleotide
gtaaaacgacggccagtatccaggcgctgcttcttac

SEQ ID NO: 350 - DNA, 39 bp, Artificial Sequence
  Description of Artificial Sequence Synthetic oligonucleotide
caggaaacagctatgacctttgcaaccagtgcacattac

SEQ ID NO: 351 - DNA, 35 bp, Artificial Sequence
  Description of Artificial Sequence Synthetic oligonucleotide
gtaaaacgacggccagtgaagaaatgccccagaaa

SEQ ID NO: 352 - DNA, 43 bp, Artificial Sequence
  Description of Artificial Sequence Synthetic oligonucleotide
caggaaacagctatgacctgtagtcagtgcattctacaacagc

SEQ ID NO: 353 - DNA, 43 bp, Artificial Sequence
  Description of Artificial Sequence Synthetic oligonucleotide
gtaaaacgacggccagtaactgtatgtccaatgtaactggttg

SEQ ID NO: 354 - DNA, 41 bp, Artificial Sequence
  Description of Artificial Sequence Synthetic oligonucleotide
caggaaacagctatgaccacattgtgtgttcttaaagcagg

SEQ ID NO: 355 - DNA, 45 bp, Artificial Sequence
  Description of Artificial Sequence Synthetic oligonucleotide
gtaaaacgacggccagtggaagaaaaatagtaaattaagtccaaa

SEQ ID NO: 356 - DNA, 42 bp, Artificial Sequence
  Description of Artificial Sequence Synthetic oligonucleotide
caggaaacagctatgaccttgctataaactgatcacaaggga

SEQ ID NO: 357 - DNA, 37 bp, Artificial Sequence
  Description of Artificial Sequence Synthetic oligonucleotide
gtaaaacgacggccagtagcgacacatgactgcaatg

SEQ ID NO: 358 - DNA, 39 bp, Artificial Sequence
  Description of Artificial Sequence Synthetic oligonucleotide
caggaaacagctatgaccagcattaaatttaggcaaggc

SEQ ID NO: 359 - DNA, 38 bp, Artificial Sequence
  Description of Artificial Sequence Synthetic oligonucleotide
caggaaacagctatgaccttttgcacaatccacattga

SEQ ID NO: 360 - DNA, 37 bp, Artificial Sequence
  Description of Artificial Sequence Synthetic oligonucleotide
gtaaaacgacggccagtagggcccactctgttactca

SEQ ID NO: 361 - DNA, 34 bp, Artificial Sequence
  Description of Artificial Sequence Synthetic oligonucleotide
caggaaacagctatgacccagttccaaaatgcct

SEQ ID NO: 362 - DNA, 42 bp, Artificial Sequence
  Description of Artificial Sequence Synthetic oligonucleotide
gtaaaacgacggccagtggtgtctttacctttcattgcttac

SEQ ID NO: 363 - DNA, 39 bp, Artificial Sequence
  Description of Artificial Sequence Synthetic oligonucleotide
caggaaacagctatgaccgaagagggttgggcctaattt

SEQ ID NO: 364 - DNA, 37 bp, Artificial Sequence
  Description of Artificial Sequence Synthetic oligonucleotide
gtaaaacgacggccagtttgctgttgttagcatcctg

SEQ ID NO: 365 - DNA, 40 bp, Artificial Sequence
  Description of Artificial Sequence Synthetic oligonucleotide
gtaaaacgacggccagtccagaatgcatttgtgtagttgc

SEQ ID NO: 366 - DNA, 41 bp, Artificial Sequence
  Description of Artificial Sequence Synthetic oligonucleotide
caggaaacagctatgaccaatggttccctgaaatactttgc

SEQ ID NO: 367 - DNA, 41 bp, Artificial Sequence
  Description of Artificial Sequence Synthetic oligonucleotide
gtaaaacgacggccagtgagttttagaggctgttaatttgc

SEQ ID NO: 368 - DNA, 43 bp, Artificial Sequence
  Description of Artificial Sequence Synthetic oligonucleotide
caggaaacagctatgacctgaacttatcaacgaagagtcagaa

SEQ ID NO: 369 - DNA, 37 bp, Artificial Sequence
  Description of Artificial Sequence Synthetic oligonucleotide
gtaaaacgacggccagtttccatcctgcagaagaagc

SEQ ID NO: 370 - DNA, 38 bp, Artificial Sequence
  Description of Artificial Sequence Synthetic oligonucleotide
caggaaacagctatgacctccgtctagccaaacacacc

SEQ ID NO: 371 - DNA, 42 bp, Artificial Sequence
  Description of Artificial Sequence Synthetic oligonucleotide
gtaaaacgacggccagtctgtgatgtataaaccgtgagtttc

SEQ ID NO: 372 - DNA, 42 bp, Artificial Sequence
  Description of Artificial Sequence Synthetic oligonucleotide
caggaaacagctatgacctcacaaagtatctttttctgtggc

SEQ ID NO: 373 - DNA, 38 bp, Artificial Sequence
  Description of Artificial Sequence Synthetic oligonucleotide
gtaaaacgacggccagtctccagctatagtggggaaaa

SEQ ID NO: 374 - DNA, 38 bp, Artificial Sequence
  Description of Artificial Sequence Synthetic oligonucleotide
caggaaacagctatgaccctgaagtccattaggtacgg

SEQ ID NO: 375 - DNA, 46 bp, Artificial Sequence
  Description of Artificial Sequence Synthetic oligonucleotide
gtaaaacgacggccagtcatgattactactctaaacccatagaagg

SEQ ID NO: 376 - DNA, 38 bp, Artificial Sequence
  Description of Artificial Sequence Synthetic oligonucleotide
caggaaacagctatgaccatgaatctgtgccaacaatg

SEQ ID NO: 377 - DNA, 39 bp, Artificial Sequence
  Description of Artificial Sequence Synthetic oligonucleotide
gtaaaacgacggccagttttgttaatggtggctttttgt

SEQ ID NO: 378 - DNA, 39 bp, Artificial Sequence
  Description of Artificial Sequence Synthetic oligonucleotide
caggaaacagctatgacctcaaatatgggctagatgcca

SEQ ID NO: 379 - DNA, 43 bp, Artificial Sequence
  Description of Artificial Sequence Synthetic oligonucleotide
gtaaaacgacggccagtataaagattcaggcaatgtttgttag

SEQ ID NO: 380 - DNA, 37 bp, Artificial Sequence
  Description of Artificial Sequence Synthetic oligonucleotide
caggaaacagctatgaccaactgcctcaaatagtagg

SEQ ID NO: 381 - DNA, 43 bp, Artificial Sequence
  Description of Artificial Sequence Synthetic oligonucleotide
gtaaaacgacggccagtgcaacatttctaaagttacctacttg

SEQ ID NO: 382 - DNA, 39 bp, Artificial Sequence
  Description of Artificial Sequence Synthetic oligonucleotide
caggaaacagctatgacctccaggaagaggaaaggaaaa

SEQ ID NO: 383 - DNA, 37 bp, Artificial Sequence
  Description of Artificial Sequence Synthetic oligonucleotide
gtaaaacgacggccagtagaccataacccaccacagc

SEQ ID NO: 384 - DNA, 45 bp, Artificial Sequence
  Description of Artificial Sequence Synthetic oligonucleotide
caggaaacagctatgacctttacttgtcaattacacctcaataaa

SEQ ID NO: 385 - DNA, 38 bp, Artificial Sequence
  Description of Artificial Sequence Synthetic oligonucleotide
gtaaaacgacggccagtaatggctacgacccagttacc

SEQ ID NO: 386 - DNA, 39 bp, Artificial Sequence
  Description of Artificial Sequence Synthetic oligonucleotide
caggaaacagctatgacctttggcttctttagcccaatg

SEQ ID NO: 387 - DNA, 40 bp, Artificial Sequence
  Description of Artificial Sequence Synthetic oligonucleotide
gtaaaacgacggccagtgcagatacagaatccatatttcg

SEQ ID NO: 388 - DNA, 39 bp, Artificial Sequence
  Description of Artificial Sequence Synthetic oligonucleotide
caggaaacagctatgaccaatgtctcaccaatgccagag

SEQ ID NO: 389 - DNA, 39 bp, Artificial Sequence
  Description of Artificial Sequence Synthetic oligonucleotide
gtaaaacgacggccagtgcaacagataactcagattgcc

SEQ ID NO: 390 - DNA, 38 bp, Artificial Sequence
  Description of Artificial Sequence Synthetic oligonucleotide
caggaaacagctatgaccggagaaaagtatcggttggc

SEQ ID NO: 391 - DNA, 41 bp, Artificial Sequence
  Description of Artificial Sequence Synthetic oligonucleotide
gtaaaacgacggccagtgtcatttcatttctttttcttttc

SEQ ID NO: 392 - DNA, 41 bp, Artificial Sequence
  Description of Artificial Sequence Synthetic oligonucleotide
caggaaacagctatgacctgtcaagcaagttcttcatcagc

SEQ ID NO: 393 - DNA, 39 bp, Artificial Sequence
  Description of Artificial Sequence Synthetic oligonucleotide
caggaaacagctatgacctgacacaatgtcctattgcca

SEQ ID NO: 394 - DNA, 45 bp, Artificial Sequence
  Description of Artificial Sequence Synthetic oligonucleotide
gtaaaacgacggccagtaaagatcatgtttgttacagtgcttaaa

SEQ ID NO: 395 - DNA, 38 bp, Artificial Sequence
  Description of Artificial Sequence Synthetic oligonucleotide
gtaaaacgacggccagtgaagtcggaacacaaggaagg

SEQ ID NO: 396 - DNA, 36 bp, Artificial Sequence
  Description of Artificial Sequence Synthetic oligonucleotide
caggaaacagctatgaccggggatccttcgcaactt

SEQ ID NO: 397 - DNA, 35 bp, Artificial Sequence
  Description of Artificial Sequence Synthetic oligonucleotide
gtaaaacgacggccagtgagctgatgtcggtgggt

SEQ ID NO: 398 - DNA, 39 bp, Artificial Sequence
  Description of Artificial Sequence Synthetic oligonucleotide
caggaaacagctatgaccggtccagctcagggtgttaag

SEQ ID NO: 399 - DNA, 38 bp, Artificial Sequence
  Description of Artificial Sequence Synthetic oligonucleotide
gtaaaacgacggccagtctctctagggaagggaggagg

SEQ ID NO: 400 - DNA, 38 bp, Artificial Sequence
  Description of Artificial Sequence Synthetic oligonucleotide
caggaaacagctatgaccttcaaggagacgggaagagg

SEQ ID NO: 401 - DNA, 38 bp, Artificial Sequence
  Description of Artificial Sequence Synthetic oligonucleotide
gtaaaacgacggccagtgcctggacttctgtgacttcc

SEQ ID NO: 402 - DNA, 36 bp, Artificial Sequence
  Description of Artificial Sequence Synthetic oligonucleotide
caggaaacagctatgaccaagccggagaaggtgtcc

SEQ ID NO: 403 - DNA, 35 bp, Artificial Sequence
  Description of Artificial Sequence Synthetic oligonucleotide
gtaaaacgacggccagtcctgtggtgtttgggagg

SEQ ID NO: 404 - DNA, 36 bp, Artificial Sequence
  Description of Artificial Sequence Synthetic oligonucleotide
caggaaacagctatgaccagatgtccaccttgaagc

SEQ ID NO: 405 - DNA, 37 bp, Artificial Sequence
  Description of Artificial Sequence Synthetic oligonucleotide
gtaaaacgacggccagtgaggtaggcacgtgctaggg

SEQ ID NO: 406 - DNA, 35 bp, Artificial Sequence
  Description of Artificial Sequence Synthetic oligonucleotide
caggaaacagctatgaccaccatctgccgtatgag

SEQ ID NO: 407 - DNA, 38 bp, Artificial Sequence
  Description of Artificial Sequence Synthetic oligonucleotide
gtaaaacgacggccagtgaggtgtccttgagtccacag

SEQ ID NO: 408 - DNA, 38 bp, Artificial Sequence
  Description of Artificial Sequence Synthetic oligonucleotide
caggaaacagctatgaccagtcctctcaatgcctgctg

SEQ ID NO: 409 - DNA, 37 bp, Artificial Sequence
  Description of Artificial Sequence Synthetic oligonucleotide
caggaaacagctatgaccggccggtaacaggacactg

SEQ ID NO: 410 - DNA, 39 bp, Artificial Sequence
  Description of Artificial Sequence Synthetic oligonucleotide
gtaaaacgacggccagtcttaggagcgtccaggtatcac

SEQ ID NO: 411 - DNA, 38 bp, Artificial Sequence
  Description of Artificial Sequence Synthetic oligonucleotide
caggaaacagctatgaccagaagctgtccttgttgcag

SEQ ID NO: 412 - DNA, 37 bp, Artificial Sequence
  Description of Artificial Sequence Synthetic oligonucleotide
gtaaaacgacggccagtagctcctgagtgtgtggcag

SEQ ID NO: 413 - DNA, 39 bp, Artificial Sequence
  Description of Artificial Sequence Synthetic oligonucleotide
caggaaacagctatgaccggtcaccatgactgactagcg

SEQ ID NO: 414 - DNA, 37 bp, Artificial Sequence
  Description of Artificial Sequence Synthetic oligonucleotide
gtaaaacgacggccagtctgggcagcagctgtaagtg

(19) United States
(12) Patent Application Publication
(10) Pub. No.: US 2011/0118298 A1
(43) Pub. Date: May 19, 2011

(54) COMPOSITIONS, KITS, AND METHODS FOR IDENTIFICATION, ASSESSMENT, PREVENTION, AND THERAPY OF CANCER

(75) Inventors: Christian Fritz, Natick, MA (US); Emmanuel Y. Normant, Winchester, MA (US); Juan Guillermo Paez, Spencer, MA (US); Kip A. West, Nahant, MA (US)

(73) Assignee: INFINITY PHARMACEUTICALS, INC., Cambridge, MA (US)

(21) Appl. No.: 12/945,809

(22) Filed: Nov. 12, 2010

Related U.S. Application Data
(60) Provisional application No. 61/261,064, filed on Nov. 13, 2009.
(60) Provisional application No. 61/283,150, filed on Nov. 30, 2009.
(60) Provisional application No. 61/313,364, filed on Mar. 12, 2010.
(60) Provisional application No. 61/313,594, filed on Mar. 12, 2010.
(60) Provisional application No. 61/346,873, filed on May 20, 2010.
(60) Provisional application No. 61/382,447, filed on Sep. 13, 2010.
(60) Provisional application No. 61/390,136, filed on Oct. 5, 2010.
(60) Provisional application No. 61/394,735, filed on Oct. 19, 2010.

(51) Int. Cl. A61K 31/395; C12Q 1/68; G01N 33/53; G01N 33/573; A61K 31/436; A61P 35/00; A61P 35/02

(52) U.S. Cl. 514/291; 435/6; 435/7.1; 435/7.4; 514/183

(57) ABSTRACT

Described herein are compositions, kits, and methods for determining whether subjects having cancer(s) are likely to respond to treatment with an HSP90 inhibitor, as a single agent or in combination therapy. Further described are methods for prognosing a time course of disease in a subject having such cancer.

### CROSS REFERENCE TO RELATED APPLICATIONS

This application claims the benefit of priority to U.S. Provisional Application Ser. No. 61/261,064, filed Nov. 13, 2009; U.S. Provisional Application Ser. No. 61/283,150, filed Nov. 30, 2009; U.S. Provisional Application Ser. No. 61/313,364, filed Mar. 12, 2010; U.S. Provisional Application Ser. No. 61/313,594, filed Mar. 12, 2010; U.S. Provisional Application Ser. No. 61/346,873, filed May 20, 2010; U.S. Provisional Application Ser. No. 61/382,447, filed Sep. 13, 2010; U.S. Provisional Application Ser. No. 61/390,136, filed Oct. 5, 2010; and U.S. Provisional Application Ser. No. 61/394,735, filed Oct. 19, 2010. The contents of all of the aforesaid applications are hereby incorporated by reference in their entirety. A PCT patent application entitled “Compositions, Kits, and Methods for Identification, Assessment, Prevention, and Therapy of Cancer,” filed Nov. 12, 2010 with the U.S. Receiving Office and designating attorney docket number I2041-7008WO is also incorporated by reference.

### SEQUENCE LISTING

The instant application contains a Sequence Listing which has been submitted in ASCII format via EFS-Web and is hereby incorporated by reference in its entirety. Said ASCII copy, created on Nov. 12, 2010, is named I20417US.txt and is 249,163 bytes in size.

### BACKGROUND OF THE INVENTION

Cancer represents the phenotypic end-point of multiple genetic lesions that endow cells with a full range of biological properties required for tumorigenesis. Indeed, a hallmark genomic feature of many cancers, including, for example, B cell cancer, lung cancer, breast cancer, ovarian cancer, pancreatic cancer, and colon cancer, is the presence of numerous complex chromosome structural aberrations—including non-reciprocal translocations, intra-chromosomal inversions, point mutations, deletions, gene copy number changes, gene expression level changes, and germ line mutations.

Karyotype analyses (Johansson, B., et al. (1992) Cancer 69, 1674-81; Bardi, G., et al. (1993) Br J Cancer 67, 1106-12; Griffin, C. A., et al. (1994) Genes Chromosomes Cancer 9, 93-100; Griffin, C. A., et al. (1995) Cancer Res 55, 2394-9; Gorunova, L., et al. (1995) Genes Chromosomes Cancer 14, 259-66; Gorunova, L., et al. (1998) Genes Chromosomes Cancer 23, 81-99), chromosomal CGH and array CGH (Wolf M et al. (2004) Neoplasia 6(3)240; Kimura Y, et al. (2004) Mod. Pathol. 21 May (epub); Pinkel, et al. (1998) Nature Genetics 20:211; Solinas-Toldo, S., et al. (1996) Cancer Res 56, 3803-7; Mahlamaki, E. H., et al. (1997) Genes Chromosomes Cancer 20, 383-91; Mahlamaki, E. H., et al. (2002) Genes Chromosomes Cancer 35, 353-8; Fukushige, S., et al. (1997) Genes Chromosomes Cancer 19:161-9; Curtis, L. J., et al. (1998) Genomics 53, 42-55; Ghadimi, B. M., et al. (1999) Am J Pathol 154, 525-36; Armengol, G., et al. (2000) Cancer Genet Cytogenet 116, 133-41), fluorescence in situ hybridization (FISH) analysis (Nilsson M et al. (2004) Int J Cancer 109(3):363-9; Kawasaki K et al. (2003) Int J Mol. Med. 12(5):727-31) and loss of heterozygosity (LOH) mapping (Wang Z C et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) have been used to identify biomarkers (e.g., chromosomal abnormalities) associated with the etiology of various cancers.

Expression levels of cellular signal transduction components have been found to be useful as biomarkers and predictors of cancer therapeutic efficacy. For example, expression levels of signaling transduction components, such as protein kinases and receptor tyrosine kinases, have been used as biomarkers.

Despite the identification of cancer biomarkers, there is a general lack of understanding between the presence of such biomarkers and the likelihood of cancer therapeutic efficacy, particularly whether a subject with a cancer is likely or unlikely to respond to treatment with an HSP90 inhibitor.

### SUMMARY OF THE INVENTION

The present invention provides, at least in part, compositions, methods, and kits for the identification, assessment and/or treatment of a cancer or tumor (e.g., an oncogene-associated cancer or tumor) responsive to a treatment that includes an HSP90 inhibitor (e.g., a treatment that includes an HSP90 inhibitor as a single agent or in combination, e.g., in combination with an mTOR inhibitor, an ALK inhibitor, and/or other chemotherapeutic agents, such as docetaxel or irinotecan).

In one embodiment, Applicants have discovered that the presence of an alteration in an Anaplastic Lymphoma Kinase (ALK) gene or gene product, e.g., an ALK rearrangement, is indicative of responsiveness to a treatment comprising an HSP90 inhibitor in lung cancer, e.g., non-small cell lung cancer (NSCLC). In other embodiments, the presence of an alteration in a Ras, e.g., K-Ras, gene or gene product, optionally in combination with an alteration in p53, has been identified as being indicative of responsiveness to a combination of an HSP90 inhibitor and an mTOR inhibitor in lung cancer, e.g., NSCLC. In other embodiments, the presence of an alteration (e.g., mutation) in EGFR gene or gene product, e.g., in an NSCLC pre-treated with a tyrosine kinase inhibitor, has been identified as being indicative of responsiveness to an HSP90 inhibitor. In other embodiments, the presence of an alteration (e.g., a mutation) in a Ras, e.g., a K-Ras, gene or gene product, has been identified as being indicative of responsiveness to a treatment comprising an HSP90 inhibitor in colorectal cancer (CRC). In yet other embodiments, the presence of an alteration (e.g., a mutation) in a Raf, e.g., a B-Raf, gene or gene product, has been identified as being indicative of responsiveness to a treatment comprising an HSP90 inhibitor in colorectal cancer.

In other embodiments, the invention further provides a method for identifying or selecting a subject as being likely or unlikely to respond to treatment comprising an HSP90 inhibitor, by evaluating one or more of: the subject's histology (e.g., detecting the presence of NSCLC or squamous cell histology); the subject's smoking status; the level or expression of HSP90, and/or an alteration as described herein (e.g., one or more alterations alteration in an ALK, MAPK pathway and/or EGFR gene or gene product).

In yet other embodiments, the invention includes methods for ameliorating or treating a cancer or tumor harboring an alteration described herein (e.g., one or more oncogenic alterations in an ALK, MAPK pathway and/or EGFR gene or gene product) with an HSP90 inhibitor, alone or in combination, e.g., in combination with an mTOR inhibitor, an ALK inhibitor, and/or other chemotherapeutic agents (e.g., docetaxel or irinotecan). In certain embodiments, the cancer or tumor is present in a subject in need of, being considered, or evaluated for, HSP90 inhibitor therapy (or a combination therapy, e.g., a combination with an mTOR inhibitor, an ALK inhibitor, and/or other chemotherapeutic agents (e.g., docetaxel or irinotecan)).

Thus, the invention provides means to evaluate responsiveness to, or monitor, therapy involving HSP90 inhibition (including combination therapies); stratify patient populations; identify subjects likely to benefit from such agents, predict a time course of disease or a probability of a significant event in the disease for such subjects, and/or more effectively monitor, treat or prevent a cancer or tumor.

Accordingly, in one aspect, the invention features a method of determining the responsiveness of, a tumor or a cancer cell (e.g., a tumor or a cancer cell in vitro, ex vivo), or a subject having said tumor or cancer cell, to a treatment comprising an HSP90 inhibitor (e.g., a treatment comprising an HSP90 inhibitor as a single agent or in combination, e.g., in combination with an mTOR inhibitor, an ALK inhibitor, a tyrosine kinase inhibitor, and/or other chemotherapeutic agents, such as docetaxel or irinotecan). The method includes one or more of the following:

(i) detecting an alteration (e.g., one or more oncogenic alterations) in an ALK, a MAPK pathway, and/or an EGFR gene or gene product; and/or

(ii) evaluating one or more of: a) the subject's histology (e.g., detecting the presence of a cancerous histology, e.g., the presence of a solid tumor, soft tissue tumor, or a metastatic lesion (e.g., detecting the presence of NSCLC, SCC or CRC cells or tissues in the subject's sample); b) the subject's smoking status (e.g., identifying the subject as a smoker or a non-smoker; determining whether the subject has a smoking history of at least 5, 10, 15 or more pack years); or c) the level or expression of HSP90, thereby determining the responsiveness of the tumor, cancer cell or the subject to the treatment comprising the HSP90 inhibitor.

In another aspect, the invention features a method of identifying or selecting a tumor, a cancer cell, or a subject (e.g., a subject having a cancer or tumor, or at risk for developing a cancer or tumor) as having a likelihood (e.g., increased or decreased likelihood), to respond to a treatment comprising an HSP90 inhibitor (e.g., a treatment comprising an HSP90 inhibitor as a single agent or in combination, e.g., in combination with an mTOR inhibitor, an ALK inhibitor, a tyrosine kinase inhibitor, and/or other chemotherapeutic agents, such as docetaxel or irinotecan). The method includes one, two, three or four of the following:

(i) evaluating a sample from the tumor, the cancer cell or the subject, e.g., detecting the presence or absence of an alteration as described herein (e.g., one or more oncogenic alterations in an ALK, a MAPK pathway, and/or an EGFR gene or gene product);

(ii) evaluating the subject's histology (e.g., detecting the presence or absence of a cancerous histology, e.g., the presence or absence of a solid tumor, soft tissue tumor, or a metastatic lesion (e.g., detecting the presence or absence of NSCLC, SCC or CRC cells or tissues in a subject's sample);

iii) evaluating the subject's smoking status (e.g., identifying the subject as a smoker or a non-smoker; determining whether the subject has a smoking history of at least 5, 10, 15 or more pack years); or

iv) determining the level or expression of HSP90 in a sample; and (optionally) identifying the tumor, cancer cell or the subject as being likely or unlikely to respond to the treatment comprising the HSP90 inhibitor.

In yet another aspect, the invention features a method of monitoring the efficacy, or predicting the efficacy, of a treatment comprising an HSP90 inhibitor (e.g., a treatment comprising an HSP90 inhibitor as a single agent or in combination, e.g., in combination with an mTOR inhibitor, an ALK inhibitor, a tyrosine kinase inhibitor, and/or other chemotherapeutic agents, such as docetaxel or irinotecan) to treat a cancer or tumor harboring an alteration as described herein (e.g., one or more oncogenic alterations in an ALK, a MAPK pathway, and/or an EGFR gene or gene product), in a subject. The method includes:

(i) detecting an alteration (e.g., one or more oncogenic alterations in an ALK, a MAPK pathway, and/or an EGFR gene or gene product as described herein), in a sample obtained from the subject; and/or

(ii) evaluating one or more of: a) the subject's histology (e.g., detecting the presence of a cancerous histology, e.g., the presence of a solid tumor, soft tissue tumor, or a metastatic lesion) (e.g., detecting the presence of NSCLC, SCC or CRC cells or tissues in a subject's sample); or b) the level or expression of HSP90;

(iii) comparing the detected alteration in the sample to a pre-determined value, e.g., a reference sample (e.g., a normal control; a blood-matched control sample (e.g., a normal adjacent tumor; or a sample collected from the subject at a different time interval, e.g., before, during or after treatment with the HSP90 inhibitor and/or other anti-cancer therapy). The extent of the difference in the alteration detected in the sample in relation to the pre-determined value is indicative of, or predictive of, the efficacy of the treatment. In one embodiment, the method can further include altering a dose or a therapeutic regimen (e.g., a dose or dosage schedule of an HSP90 inhibitor, alone or in combination, e.g., in combination with an ALK inhibitor, an mTOR inhibitor, a tyrosine kinase inhibitor, and/or a chemotherapeutic agent) in response to the difference detected. For example, the presence of an alteration in the ALK, MAPK pathway, and/or an EGFR gene or gene product (e.g., an ALK rearrangement or an EGFR mutation in a NSCLC and/or SCC sample, or a mutant K-Ras or B-Raf in a colorectal carcinoma sample), or the presence of cancerous cells or tissues, in the sample obtained from a subject during treatment with the HSP90 inhibitor, or after treatment has been discontinued, is indicative of the need to increase in dose or frequency of administration of the HSP90 inhibitor, as a single agent or in combination.

In certain embodiments of the methods of the invention, the presence of an alteration in an ALK, a MAPK pathway, and/or an EGFR gene or gene product is indicative that the tumor or cancer cell has an increased likelihood to respond to a treatment comprising the HSP90 inhibitor. In certain embodiments, the MAPK pathway gene or gene product includes one or more of Ras (e.g., one or more of H-Ras, N-Ras, or K-Ras), Raf (e.g., one or more of A-Raf, B-Raf (BRAF) or C-Raf), Mek, and/or Erk. The methods described herein can, optionally, further include detection of an alteration in one or more gene products chosen from EGFR, PIK3CA, PTEN, AKT, TP53 (p53), CTNNB1 (beta-catenin), APC, KIT, JAK2, NOTCH, FLT3, RSK, ETS, ELK-1, or SAP-1.

In other embodiments, one or more of the following are indicative of an increased likelihood to respond to a treatment comprising the HSP90 inhibitor (e.g., a treatment comprising the combination of the HSP90 inhibitor and docetaxel or irinotecan): (i) detecting presence of a NSCLC, CRC or squamous cell histology (e.g., NSCLC, CRC and/or SCC cells or tissue in the subject's histology); (ii) identifying the subject as a smoker (e.g., a subject who has a smoking history of at least 5, 10, 15 or more pack years); or (iii) detecting an elevated level or expression of HSP90 (e.g., an elevated level of HSP90 gene or gene product). In one embodiment, the method further includes detecting the presence of NSCLC, SCC or colorectal carcinoma (CRC) cells or tissues in a subject's sample, and optionally, comparing the presence of the NSCLC, CRC or SCC histology to other histologies, such as adenocarcinoma for lung tumors).

In one embodiment, the detection of, or the presence of, an alteration in an ALK gene or gene product (e.g., an ALK rearrangement) is indicative of an increased likelihood to respond to a treatment comprising an HSP90 inhibitor, e.g., as a single agent or in combination, to inhibit, reduce, or treat a lung tumor or cancer cell, e.g., NSCLC (e.g., relapsed and/or refractory NSCLC), or SCC, tumor or cancer cell.

In yet other embodiments, detection of, or the presence of, an alteration in a Ras, e.g., K-Ras, gene or gene product, optionally in combination with detection of an alteration in p53 gene or gene product, is indicative of an increased likelihood to respond to a treatment comprising a combination of an HSP90 inhibitor and an mTOR inhibitor, to inhibit, reduce, or treat a lung tumor or cancer cell, e.g., NSCLC (e.g., relapsed and/or refractory NSCLC), or SCC, tumor or cancer cell.

In another embodiment, the detection of, or the presence of, an alteration in a Ras, e.g., K-Ras, gene or gene product, is indicative of an increased likelihood to respond to therapy comprising an HSP90 inhibitor, e.g., as a single agent or in combination, to inhibit, reduce, or treat a colorectal tumor or cancer cell (e.g., a colorectal carcinoma tumor or cancer cell).

In yet another embodiment, the detection of, or the presence of, an alteration in a Raf, e.g., a B-Raf, gene or gene product, is indicative of an increased likelihood to respond to therapy comprising an HSP90 inhibitor, e.g., as a single agent or in combination, to inhibit, reduce, or treat a colorectal tumor or cancer cell (e.g., a colorectal carcinoma tumor or cancer cell).

In yet other embodiments, the methods of the invention further include treating or preventing a cancer or tumor harboring an alteration described herein (e.g., one or more ALK, MAPK pathway or EGFR alterations; the presence of a cancerous histology; or elevated expression of HSP90). The method includes administering to the subject an HSP90 inhibitor, e.g., one or more HSP90 inhibitors as described herein, as a single agent, or in combination, e.g., in combination with an mTOR inhibitor, an ALK inhibitor, and/or other chemotherapeutic agents (e.g., docetaxel or irinotecan), in an amount sufficient to reduce or inhibit the tumor cell growth, and/or treat or prevent the cancer(s), in the subject.

Alternatively, or in combination with the methods described herein, the invention features a method of reducing or inhibiting growth of a cancer or tumor (e.g., one or more cancers or tumors), in a subject. The invention also features a method of treating a subject having, or at risk of having, a cancer or tumor (e.g., one or more cancers or tumors). In certain embodiments, the tumor or cancer harbors an alteration as described herein (e.g., one or more ALK, MAPK pathway, EGFR alterations; the presence of a cancerous histology; or elevated expression of HSP90). The method includes administering to the subject an HSP90 inhibitor, e.g., one or more HSP90 inhibitors as described herein, alone or in combination with an mTOR inhibitor, an ALK inhibitor, a tyrosine kinase inhibitor, and/or other chemotherapeutic agents (e.g., docetaxel or irinotecan), in an amount sufficient to reduce or inhibit the tumor cell growth, and/or treat or prevent the cancer(s), in the subject.

In certain embodiments, the cancer or tumor harboring the alteration is present in a subject, in need of, identified as likely to benefit from, or being considered or evaluated for, HSP90 inhibitor therapy (or combination therapy with, e.g., an mTOR inhibitor, an ALK inhibitor, tyrosine kinase inhibitor and/or a chemotherapeutic agent, e.g., docetaxel or irinotecan). For example, the subject treated by the therapeutic methods of the invention can have, or is identified as having, one or more of: a history of smoking; elevated level or expression of HSP90; NSCLC (e.g., relapsed and/or refractory NSCLC) or SCC cells or tumors; or is experiencing disease progression during or after receiving at least one prior chemotherapeutic regimen; is an NSCLC patient experiencing disease progression during or after receiving at least one prior platinum-containing chemotherapeutic regimen.

In certain embodiments, the subject is previously selected or identified to be treated with a therapy comprising an HSP90 inhibitor, e.g., previously evaluated as having one or more of: a history of smoking; having an NSCLC or SCC; having elevated level or expression of HSP90. In other embodiments, the subject is previously selected to be treated with a therapy comprising an HSP90 inhibitor by evaluating a sample obtained from the subject to detect the presence of one or more oncogenic alterations as described herein (e.g., a mutant ALK, MAPK pathway (e.g., K-Ras), EGFR gene or gene product). In yet other embodiments, the subject has an EGFR mutation (e.g., a T790M) and has been and has been pre-treated with a tyrosine kinase inhibitor, e.g., gefitinib.

In certain embodiments, the methods of treatment (optionally) further includes evaluating a sample from the subject to detect one or more alterations in the gene or gene product described herein, or identifying the subject as having one or more of: a history of smoking; having an NSCLC, SCC or CRC; having elevated level or expression of HSP90.

Treatment can include, but is not limited to, inhibiting tumor growth, reducing tumor mass, reducing size or number of metastatic lesions, inhibiting the development of new metastatic lesions, prolonged survival, prolonged progression-free survival, prolonged time to progression, and/or enhanced quality of life.

In certain embodiments, the cancer or tumor evaluated and/or treated by the methods of the invention includes, but is not limited to, a solid tumor, a soft tissue tumor, and a metastatic lesion (e.g., a cancer or tumor as described herein). In some embodiments, the cancer or tumor evaluated and/or treated harbors an alteration in a gene or gene product chosen from one or more of ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1. In one embodiment, the cancer or tumor evaluated and/or treated has one or more alterations in an ALK gene or gene product, e.g., an ALK rearrangement. In another embodiment, the cancer or tumor evaluated and/or treated has one or more alterations in a MAPK pathway (e.g., K-Ras or B-Raf) gene or gene product. In certain embodiments, the cancer or tumor is chosen from one or more of: lung cancer (e.g., small cell lung cancer (SCLC), non-small cell lung cancer (NSCLC), or squamous cell cancer (SCC)), colorectal cancer, breast cancer, medulloblastoma, chondrosarcoma, osteosarcoma, pancreatic cancer, ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, prostate cancer, anaplastic large cell lymphoma, or neuroblastoma.

In some embodiments, the cancer or tumor evaluated and/or treated is a non-small cell lung cancer (NSCLC) (e.g., relapsed and/or refractory NSCLC), SCC, or a colorectal cancer. In one embodiment, the NSCLC harbors a mutation in an ALK gene or gene product (e.g., the NSCLC has an ALK rearrangement; the NSCLC expresses an EML4-ALK fusion; the NSCLC expresses a nucleophosmin-anaplastic lymphoma kinase fusion (NPM-ALK fusion)). In one embodiment, the tumor or cancer is resistant (e.g., partially or completely resistant) to an ALK inhibitor, but retains sensitivity to an HSP90 inhibitor described herein. In other embodiments, the NSCLC harbors a mutation in a K-Ras gene or gene product. In yet other embodiments, the NSCLC harbors a mutation in a K-Ras gene or gene product, and a p53 gene or gene product. In yet other embodiments, the NSCLC harbors a mutation in a K-Ras gene or gene product, and an EGFR gene or gene product. In yet other embodiments, the NSCLC has a mutation in an EGFR gene or gene product. In yet other embodiments, the NSCLC has a mutation in an EGFR gene or gene product and has been pre-treated with a tyrosine kinase inhibitor. In one embodiment, the tumor or cancer is resistant (e.g., partially or completely resistant) to a tyrosine kinase inhibitor (e.g., gefitinib), but retains sensitivity to an HSP90 inhibitor described herein. In yet other embodiments, the NSCLC has a wild type EGFR and/or K-Ras gene or gene product. In yet other embodiments, the cancer or tumor evaluated or treated, is a squamous cell carcinoma (SCC). In yet other embodiments, the cancer or tumor evaluated and/or treated is a large cell carcinoma or an adenocarcinoma of the lung. In other embodiments, the cancer or tumor evaluated or treated, has at least 20%, 30% 50%, 70% of the cells showing a histology of NSCLC or SCC.

In other embodiments, the cancer or tumor evaluated and/or treated is a colorectal cancer. In one embodiment, the colorectal cancer harbors a mutation in a MAPK pathway gene or gene product (e.g., Ras (e.g., one or more of H-Ras, N-Ras, or K-Ras), Raf (e.g., one or more of A-Raf, B-Raf (BRAF) or C-Raf), Mek, and/or Erk). In one embodiment, the colorectal cancer harbors a mutation in Ras, e.g., K-Ras. In another embodiment, the colorectal cancer harbors a mutation in Raf, e.g., B-Raf.

In certain embodiments, the cancer or tumor identified or treated is a neuroendocrine cancer or a carcinoid tumor (e.g., a functional or non-functional neuroendocrine or carcinoid tumor).

Additional embodiments or features of the present invention are as follows:

In certain embodiments, the alteration (e.g., the one or more oncogenic alterations) of the gene or gene product includes, but is not limited to, cytogenetic abnormalities, non-reciprocal translocations, rearrangements, intra-chromosomal inversions, mutations, point mutations, deletions, changes in gene copy number, mutations in a transcript, and changes in expression of a gene or gene product. In certain embodiments, the mutation in a transcript is an mRNA mutation, rRNA mutation or tRNA mutation. In certain embodiments, the expression level, structure (e.g., post-translational modifications, such as phosphorylation) and/or activity of one or more oncogenic polypeptides is evaluated. In related embodiments, the expression level, structure, and/or activity of one or more mutant oncogenic isoforms, e.g., isoforms arising from one or more of alternative splicing, frameshifting, translational and/or post-translational events, of various proto-oncogene expression products in a cell, e.g., a hyperproliferative cell (e.g., a cancerous or tumor cell) are detected.

In one embodiment, the methods include detecting an alteration in an ALK gene or gene product. In other embodiments, the alteration detected includes one or more alterations in a MAPK pathway gene or gene product (including Ras, Raf, Mek, and/or Erk). In one embodiment, the alteration in the MAPK pathway gene or gene product includes one or more alterations of a Ras (e.g., K-Ras) or Raf (e.g., B-Raf) gene or gene product.

In another embodiment, the methods include detecting an abnormal activation of the MAPK (RAS-RAF-MEK-Erk) pathway (“MAPK pathway activation”), e.g., for example, by detection of mutations in a gene of that pathway (“MAPK pathway gene”) or transcript thereof, by detection of a mutation in a protein of that pathway, or by detection of elevated levels of an unphosphorylated and/or phosphorylated protein of that pathway (“pathway protein”). In certain embodiments, detection of MAPK pathway activation comprises detection of a mutation in a MAPK pathway gene or transcript thereof, detection of a mutation in a MAPK pathway protein or detection of an elevated level of a MAPK pathway protein. In certain embodiments, the MAPK pathway gene is a Ras gene, Raf gene, Mek gene or Erk gene. In certain embodiments, the Ras gene is an H-Ras gene, N-Ras gene or K-Ras gene. In certain embodiments, the Raf gene is an A-Raf gene, B-Raf gene or C-Raf gene.

In other embodiments, the MAPK pathway protein is a Ras protein, a Raf protein, a Mek protein, an Erk protein, a Mk1 protein, an RSK protein, an Ets protein, an Elk-1 protein or a SAP-1 protein. In certain embodiments, the Ras protein is an H-Ras protein, N-Ras protein or K-Ras protein. In certain embodiments, the Raf protein is A-Raf protein, B-Raf protein or C-Raf protein. In certain embodiments, the Mek protein is Mek-1 or Mek-2. In certain embodiments, the Erk protein is Erk-1 or Erk-2. In certain embodiments, the MAPK pathway protein is an unphosphorylated MAPK pathway protein. In certain embodiments, the MAPK pathway protein is a phosphorylated MAPK pathway protein. In certain embodiments, the phosphorylated MAPK pathway protein is a phosphorylated Mek protein.

In other embodiments, the alteration of the gene or gene products evaluated and/or treated is chosen from one or more of ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1. A single gene or gene product, or any combination of two, three, four, five, six, seven, eight, nine, ten or more of the aforesaid gene or gene products can be evaluated or treated. For example, alterations of two of any of the aforesaid gene or gene products can be evaluated (e.g., alterations in ALK and K-Ras, ALK and EGFR, K-Ras and EGFR, EGFR and BRAF, K-Ras and p53 can be evaluated). In other embodiments, alterations of any of three of the aforesaid gene or gene products are evaluated (e.g., alterations in ALK, K-Ras, and EGFR; or EGFR, K-Ras and p53 are evaluated).

In one embodiment, an alteration (e.g., one or more oncogenic alterations) in an ALK gene or gene product is evaluated and/or treated. In certain embodiments, the alteration in a mutant ALK gene or gene product is chosen from a mutant ALK polynucleotide molecules or polypeptides listed in Table 1 (SEQ ID NOs:1-13). Non-limiting examples of alterations in an ALK gene or gene product include EML4-ALK fusions, KIF5B-ALK fusions, TGF-ALK fusions, NPM-ALK fusions, and ALK point mutations including one or more of F1245I/L, L1204F, A1200V, L1196M, 11170S, T1151M, R1275Q, F1174V/C/L, T1087I, and K1062M, as described herein. In one embodiment, the alteration includes an intra-chromosomal inversion between the N-terminus of ELM4 and the C-terminus of ALK, producing an EML4-ALK fusion protein.

In other embodiments, an alteration (e.g., one or more oncogenic alterations) of a Ras gene or gene product is evaluated and/or treated. In certain embodiments, the mutant Ras gene or gene product is chosen from one or more mutant Ras polynucleotide molecules or polypeptides listed in Table 5 (SEQ ID NOs:14-16 and SEQ ID NOs:20-22). In one embodiment, the one or more mutations in any of K-Ras, H-Ras and/or N-Ras include, for example, mutations in codon 12, 13 and/or 61, including but not limited to, G12A, G12N, G12R, G12C, G125, G12V, G13N and Q61R. In one embodiment, one or more alteration of a K-Ras gene or gene product are evaluated or treated. Non-limiting examples of alterations in a KRAS gene is selected from the group consisting of KRAS_G12C, KRAS_G12R, KRAS_G12D, KRAS_G12A, KRAS_G12S, KRAS_G12V, KRAS_G13D, KRAS_G13S, KRAS_G13C, KRAS_G13V, KRAS_Q61H, KRAS_Q61R, KRAS_Q61P, KRAS_Q61L, KRAS_Q61K, KRAS_Q61E, KRAS_A59T and KRAS_G12F.

In yet other embodiments, an alteration (e.g., one or more oncogenic alterations) of a RAF (e.g., one or more of A-Raf, B-Raf (BRAF) or C-Raf) gene or gene product is evaluated or treated. In certain embodiments, the alteration in a mutant Raf gene or gene product is chosen from a mutant Raf polynucleotide molecules or polypeptides listed in Table 5 (SEQ ID NO:17-19 and SEQ ID NOs:23-25), or a mutation in codon 464, 466, 468, 469, 594, 595, 596, 597, 599, 600, or 601, of B-Raf. Exemplary alterations in the RAF gene or gene product, include, but are not limited to, BRAF_D594G, BRAF_D594V, BRAF_F468C, BRAF_F595L, BRAF_G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E, BRAF_K601N, BRAFL597Q, BRAF_L597R, BRAF_L597S, BRAF L597V, BRAF_T5991, BRAF_V600E, BRAF_V600K, BRAF_V600L, and BRAF_V600R.

In other embodiments, an alteration (e.g., one or more oncogenic alterations) of an EGFR gene or gene product is evaluated or treated. Exemplary alterations in an EGFR gene or gene product, include but are not limited to, an EGFR exon deletion (e.g., EGFR exon 19 Deletion), and/or exon mutation (e.g., an L858R/T790M EGFR mutation). Other exemplary alterations include, but are not limited to, EGFR_D770_N771>AGG; EGFR_D770_N771insG; EGFR_D770_N771insG; EGFR_D770_{—}771insN: EGFR_E709A; EGFR_E709G: EGFR_{—}709H; EGFR_E709K: EGFR_E709V; EGFR_E746_A750del: EGFR_E746_A750del, T751A; EGFR_E746_A750del, V ins; EGFR_E746_T751del, I ins; EGFR_E746_T751del, S752A; EGFR_E746_T751del, S752D; EGFR_E746_T751 del, V ins; EGFR_G719A; EGFR_G719C; EGFR_G719S: EGFR_H773_V774insH; EGFR_H773_V774insNPH; EGFR_H773_V774insPH; EGFR_H773>NPY; EGFR_L747_E749del; EGFR_L747_E749del, A750P; EGFR_L747_S752del: EGFR_L747_S752del, P753S; EGFR_L747_S752del, Q ins; EGFR_L747_T750del, P ins; EGFR_L747_T751del; EGFR_L858R; EGFR_L861Q; EGFR_M766_A767insAI; EGFR_P772_H773insV; EGFR S752_{—}1759del; EGFR_S7681; EGFR_T790M: EGFR_V769_D770insASV; EGFR_V769_D770insASV: and EGFR_V774_C775insHV.

In certain embodiments, the subject evaluated and/or treated is a mammal, e.g., a primate, typically a human (e.g., a patient having, or at risk of having, a cancer or tumor described herein). The subject can be one at risk of having the disorder, e.g., a subject having a relative afflicted with the cancer, or a subject having a genetic trait associated with risk for the cancer. In one embodiment, the subject can be symptomatic or asymptomatic. In certain embodiments, the subject is a patient having an oncogenic alteration in a gene or gene product. For example, the subject can have one or more alterations in a gene or gene product chosen from one or more of ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1. In one embodiment, the subject is a patient having an alteration in an ALK gene or gene product, e.g., an ALK gene rearrangement (e.g., an EML4-ALK fusion); or an alteration in a MAPK pathway gene or gene product, such as Ras (e.g., K-Ras) or Raf (e.g., B-Raf) gene or gene product (e.g., an activating mutation in K-Ras or B-Raf gene). In one embodiment, the subject has, or is diagnosed with, NSCLC (e.g., relapsed and/or refractory NSCLC) or SCC. In other embodiments, the subject has, or is diagnosed with, a colorectal cancer. In certain embodiments, the subject identified or treated has, or is currently being treated, with an HSP90 inhibitor as a single agent or in combination, e.g., alone or in combination with an mTOR inhibitor, an ALK inhibitor and/or other chemotherapeutic agents. In certain embodiments, the subject is in need of, is identified as likely to benefit from, or is being considered for, HSP90 inhibitor therapy (or combination therapy with another chemotherapeutic agent, e.g., docetaxel or irinotecan). For example, the subject can be a patient with one or more of: a history of smoking; a patient having an NSCLC or SCC; or a patient having elevated level or expression of HSP90. In one embodiment, the subject is resistant (e.g., partially or completely resistant) to an ALK kinase inhibitor. In another embodiment, the subject is resistant (e.g., partially or completely resistant) to a prior chemotherapeutic regimen (e.g., a platinum-containing chemotherapeutic regimen). In yet another embodiment, the subject has a mutation in an EGFR gene or gene product. In yet another embodiment, the subject (e.g., an NSCLC patient) has a mutation in an EGFR gene or gene product, and has been pre-treated with a tyrosine kinase inhibitor. In one embodiment, the subject is resistant (e.g., partially or completely resistant) to a tyrosine kinase inhibitor, e.g., geftinib.

In one embodiment, the sample evaluated in the methods of the invention is collected or obtained from the subject, or alternatively, the method further includes obtaining or collecting a sample from the subject. The sample can be chosen from one or more of: tissue (e.g., a tissue biopsy), whole blood, serum, plasma, buccal scrape, sputum, saliva, cerebrospinal fluid, urine, stool, circulating tumor cells, circulating nucleic acids, or bone marrow.

In other embodiments, the alteration is detected by any method of detection available in the art, including but not limited to, one or more of nucleic acid hybridization assay, amplification-based assays (e.g., polymerase chain reaction (PCR)), PCR-RFLP assay, real-time PCR, sequencing, screening analysis (including metaphase cytogenetic analysis by standard karyotype methods, FISH, spectral karyotyping or MFISH, comparative genomic hybridization), in situ hybridization, SSP, HPLC or mass-spectrometric genotyping.

In yet other embodiment, the expression level of the one or more oncogenic polypeptides described herein, e.g., ALK, MAPK pathway, or EGFR polypeptides, is detected. For example, the polypeptide can be detected using a reagent which specifically binds to an ALK, MAPK pathway, or EGFR polypeptide. In another embodiment, the reagent is selected from the group consisting of an antibody, and antibody derivative, and an antibody fragment. In yet another embodiment, the amount, structure and/or activity of the oncogenic polypeptide, e.g., ALK, MAPK pathway or EGFR polypeptide, is compared to a pre-determined value, e.g., a reference value (e.g., a control sample).

In one embodiment, the method includes: contacting a sample, e.g., a genomic DNA sample (e.g., a chromosomal sample or a fractionated, enriched or otherwise pre-treated sample) or a gene product (mRNA, cDNA), obtained from the subject, with a probe (e.g., an exon-specific probe, a probes specific for the desired sequence) under conditions suitable for hybridization, and determining the presence or absence of one or more of the abnormalities in the gene or gene product (e.g., genomic DNA in chromosomal regions associated with cytogenetic abnormalities (e.g., one or more of the ALK, MAPK or EGFR pathway mutations described herein)). The method can, optionally, include enriching a sample for the gene or gene product.

In yet another embodiment, the alteration, e.g., the one or more alterations in ALK, MAPK pathway (e.g., K-Ras or B-Raf) or EGFR, tumor histology, or HSP90 levels, are assessed at a pre-determined interval, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to metastatic disease. In another embodiment, the significant event is the progression from primary diagnosis to relapse. In another embodiment, the significant event is the progression from metastatic disease to death. In another embodiment, the significant event is the progression from metastatic disease to relapse. In another embodiment, the significant event is the progression from relapse to death. In certain embodiments, the time course is measured with respect to one or more of overall survival rate, time to progression and/or using the RECIST or other response criteria.

In certain embodiments, a pre-determined value is created by dividing patient samples into at least two patient subgroups. In certain embodiments, the number of subgroups is two so that the patient sample is divided into a first subgroup of patients having the oncogenic alteration, e.g., an ALK, MAPK pathway or EGFR (e.g., K-Ras) mutation(s); or one or more of a positive smoking status, tumor histology, or elevated expression of HSP90; and a second subgroup not having the oncogenic abnormalities, non-smokers, benign tumor histology or control levels of HSP90 expression. In certain embodiments, the ALK mutation, MAPK pathway (e.g., K-Ras or B-Raf) or EGFR status, or one or more smoking status, tumor histology, elevated expression of HSP90, in the subject is compared to either the first or second subgroup; if the patient has one or more of: a mutation(s) in an ALK, MAPK pathway (e.g., K-Ras or B-Raf) or EGFR, is a smoker, has elevated HSP90 levels, or a cancer histology, then the patient is likely to respond to an HSP90 inhibitor (e.g., IPI-493 and/or IPI-504), as a single agent or in combination. In certain embodiments, the responders have an increased likelihood, or are likely, to have a long time course. In certain embodiments, the number of subgroups is greater than two, including, without limitation, three subgroups, four subgroups, five subgroups and six subgroups, depending on stratification of predicted HSP90 and/or mTOR, ALK, tyrosine kinase inhibitor efficacy as correlated with particular oncogenic alterations, smoking status, histology and HSP90 levels described herein. In certain embodiments, likelihood to respond is measured with respect to overall survival rate, time to progression and/or using the RECIST criteria.

In other embodiments, the methods further include one or more of: determining whether a subject with a cancer or tumor having an alteration described herein, or smoking status, histology and HSP90 levels described herein, is likely to respond to treatment with an HSP90 inhibitor (e.g., IPI-493 and/or IPI-504), as a single agent or in combination, e.g., alone or in combination with an ALK inhibitor, an mTOR inhibitor, a tyrosine kinase inhibitor or other chemotherapeutic agent (e.g., docetaxel or irinotecan); determining a treatment regimen (e.g., altering the course of therapy, dosing, treatment schedule or time course, combination therapies). The method can be used to predict a time course of the cancer in a subject. In other embodiments, the method is used to predict the probability of a significant event in the subject with cancer.

In one embodiment, the HSP90 inhibitor is a geldanamycin derivative, e.g., a benzoquinone or hygroquinone ansamycin HSP90 inhibitor (e.g., IPI-493 and/or IPI-504). For example, the HSP90 inhibitor can be chosen from one or more of IPI-493, IPI-504, 17-AAG (also known as tanespimycin or CNF-1010), BIIB-021 (CNF-2024), BBB-028, AUY-922 (also known as VER-49009), SNX-5422, STA-9090, AT-13387, XL-888, MPC-3100, CU-0305, 17-DMAG, CNF-1010, Macbecin (e.g., Macbecin I, Macbecin II), CCT-018159, CCT-129397, PU-H71, or PF-04928473 (SNX-2112).

In one embodiment, the Hsp90 inhibitor is a compound of formula 1:

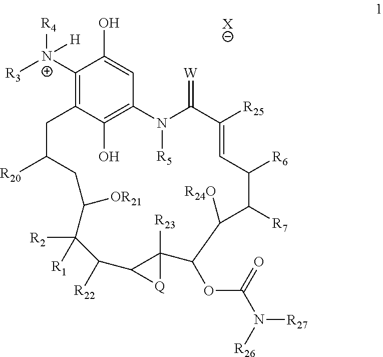

or the free base thereof;

wherein independently for each occurrence:

W is oxygen or sulfur;

Q is oxygen, NR, N(acyl) or a bond;

X⁻ is a conjugate base of a pharmaceutically acceptable acid;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₃ taken together with R₄ represent a 4-8 membered optionally substituted heterocyclic ring;

R₅ is selected from the group consisting of H, alkyl, aralkyl, and a group having the formula 1a:

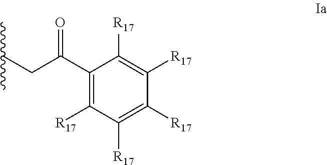

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₉ is alkyl, alkenyl, alkynyl, aryl, cycloalkyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(CR₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl;

provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and

the absolute stereochemistry at a stereogenic center of formula 1 can be R or S or a mixture thereof and the stereochemistry of a double bond can be E or Z or a mixture thereof.

In other embodiments, the Hsp90 inhibitor is a compound of formula 3:

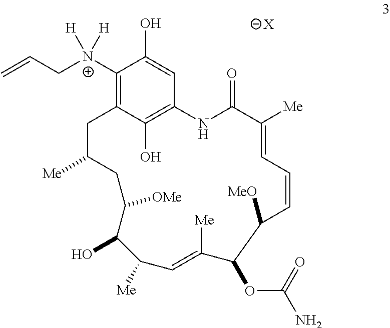

or the free base thereof;

wherein X⁻ is the conjugate base of a pharmaceutically acceptable acid. In certain embodiments, the pharmaceutically acceptable acid has a pKa of between about −10 and about 3. X⁻ can be selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamate, thiocyanate, naphthalene-2-sulfonate, and oxalate. In one embodiment, X⁻ is chloride.

In certain embodiments, the Hsp90 inhibitor is 17-AG. In other embodiments, the HSP90 inhibitor is IPI-493. In other embodiments, the HSP90 inhibitor is IPI-504.

In certain embodiments, one or more HSP90 inhibitors are administered as monotherapy or as a single agent, e.g., present in a composition, e.g., a pharmaceutical composition composition including one HSP90 inhibitor.

In other embodiments, the HSP90 inhibitor is administered in combination with a second therapeutic agent or a different therapeutic modality, e.g., anti-cancer agents, and/or in combination with surgical and/or radiation procedures.

In other embodiments, the HSP90 inhibitor is administered in combination with another HSP inhibitor, e.g., IPI-493 and/or IPI-504, in combination with one or more of 17-AAG (also known as tanespimycin or CNF-1010), BIIB-021 (CNF-2024), BIIB-028, AUY-922 (also known as VER-49009), SNX-5422, STA-9090, AT-13387, XL-888, MPC-3100, CU-0305, 17-DMAG, CNF-1010, Macbecin (e.g., Macbecin I, Macbecin II), CCT-018159, CCT-129397, PU-H71, or PF-04928473 (SNX-2112).

The HSP90 inhibitors described herein can be administered to the subject systemically (e.g., orally, parenterally, subcutaneously, intravenously, rectally, intramuscularly, intraperitoneally, intranasally, transdermally, or by inhalation or intracavitary installation). Typically, the HSP90 inhibitors are administered subcutaneously, intravenously or orally.

In one embodiment, the HSP90 inhibitor is IPI-504. IPI-504 can be administered intravenously weekly at a dose of about 300 to 500 mg/m², typically about 350 to 500 mg/m², and more typically 450 mg/m², alone or in combination with a second agent as described herein.

In one embodiment, the second agent or the anti-cancer agent used in combination with the HSP90 inhibitor is a cytotoxic or a cytostatic agent. Exemplary cytotoxic agents include antimicrotubule agents, topoisomerase inhibitors (e.g., irinotecan), or taxanes (e.g., docetaxel), antimetabolites, mitotic inhibitors, alkylating agents, intercalating agents, agents capable of interfering with a signal transduction pathway, agents that promote apoptosis and radiation. In yet other embodiments, the methods can be used in combination with immunodulatory agents, e.g., IL-1, 2, 4, 6, or 12, or interferon alpha or gamma, or immune cell growth factors such as GM-CSF. In one embodiment, the anti-cancer agent is a topoisomerase inhibitor, e.g., irinotecan.

In other embodiments, the anti-cancer agent used in combination with the HSP90 inhibitor is a tyrosine kinase inhibitor (e.g., a receptor tyrosine kinase (RTK) inhibitor, e.g., gefitinib), a topoisomerase inhibitor (e.g., irinotecan), or a taxane (e.g., docetaxel). In other embodiments, a combination of an HSP90 inhibitor, alone or combination with an mTOR inhibitor, a tyrosine kinase inhibitor (e.g., a receptor tyrosine kinase (RTK) inhibitor, e.g., gefitinib), a topoisomerase inhibitor (e.g., irinotecan), and/or a taxane (e.g., docetaxel), can be used.

Any combination of the HSP90 inhibitor, alone or combination with an mTOR inhibitor or an ALK inhibitor, and other therapeutic modalities can be used. For example, the HSP90 inhibitor and other therapeutic modalities can be administered during periods of active disorder, or during a period of remission or less active disease. The HSP90 inhibitor, alone or combination with an mTOR inhibitor or an ALK inhibitor, a tyrosine kinase inhibitor, and other therapeutic modalities can be administered before treatment, concurrently with treatment, post-treatment, or during remission of the disorder. In one embodiment, the anti-cancer agent is administered simultaneously or sequentially with the HSP90 inhibitor and/or the mTOR inhibitor or the ALK inhibitor.

In other embodiments, the HSP90 inhibitor, the mTOR inhibitor, the ALK inhibitor, and/or the anti-cancer agent are administered as separate compositions, e.g., pharmaceutical compositions. In other embodiments, the HSP90 inhibitor, the mTOR inhibitor, the ALK inhibitor, the tyrosine kinase inhibitor, and/or the anti-cancer agent are administered separately, but via the same route (e.g., both orally or both intravenously). In still other instances, the HSP90 inhibitor, the mTOR inhibitor, the tyrosine kinase inhibitor, and/or the anti-cancer agent are administered in the same composition, e.g., the same pharmaceutical composition.

In one embodiment, the HSP90 inhibitor is administered in combination with an mTOR inhibitor, e.g., one or more mTOR inhibitors chosen from one or more of rapamycin, temsirolimus (TORISEL®), everolimus (RAD001, AFINITOR®), ridaforolimus, AP23573, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, or PKI-587. In one embodiment, the mTOR inhibitor inhibits TORC1 and TORC2. Examples of TORC1 and TORC2 dual inhibitors include, e.g., OSI-027, XL765, Palomid 529, and INK128. The HSP90 inhibitor can be administered via the same or a different route than the mTOR inhibitor. In one embodiment, the mTOR inhibitor is administered systemically, e.g., orally, subcutaneously, or intravenously.

In yet another embodiment, the HSP90 inhibitor is administered in combination with an ALK kinase inhibitor(s). Exemplary ALK inhibitors include TAE-684 (also referred to herein as “NVP-TAE694”), PF02341066 (also referred to herein as “crizotinib” or “1066”), and AP26113. Additional examples of ALK kinase inhibitors are described in example 3-39 of WO 2005016894 by Garcia-Echeverria C, et al.

In some embodiments, the HSP90 inhibitor is administered in combination with folfirinox. Folfirinox comprises oxaliplatin 85 mg/m2 and irinotecan 180 mg/m2 plus leucovorin 400 mg/m2 followed by bolus fluorouracil (5-FU) 400 mg/m2 on day 1, then 5-FU 2,400 mg/m2 as a 46-hour continuous infusion.

In some embodiments, the HSP90 inhibitor is administered in combination with a tyrosine kinase inhibitor, e.g., gefitinib. In some embodiments, the HSP90 inhibitor is administered after treatment with the tyrosine kinase inhibitor.

In some embodiments, the HSP90 inhibitor is administered in combination with a PI3K inhibitor. In one embodiment, the PI3K inhibitor is an inhibitor of delta and gamma isoforms of PI3K. Exemplary PI3K inhibitors that can be used in combination with the HSP90 inhibitors, include but are not limited to, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof.

In some embodiments, the HSP90 inhibitor is administered in combination with a BRAF inhibitor, e.g., GSK2118436, RG7204, PLX4032, GDC-0879, PLX4720, and sorafenib tosylate (Bay 43-9006).

In some embodiments, the HSP90 inhibitor is administered in combination with a MEK inhibitor, e.g., ARRY-142886, GSK1120212, RDEA436, RDEA119/BAY 869766, AS703026, AZD6244 (selumetinib), BIX 02188, BIX 02189, CI-1040 (PD184352), PD0325901, PD98059, and U0126.

In some embodiments, the HSP90 inhibitor is administered in combination with a JAK2 inhibitor, e.g., CEP-701, INCB18424, CP-690550 (tasocitinib).

In other embodiments, the HSP90 inhibitor is administered in combination with a vascular disrupting agent (e.g., DMXAA, vadimezan).

In some embodiments, the HSP90 inhibitor is a first line treatment for the cancer or tumor, i.e., it is used in a subject who has not been previously administered another drug intended to treat the cancer.

In other embodiments, the HSP90 inhibitor is a second line treatment for the cancer, i.e., it is used in a subject who has been previously administered another drug intended to treat the cancer.

In other embodiments, the HSP90 inhibitor is a third or fourth line treatment for the cancer, i.e., it is used in a subject who has been previously administered two or three other drugs intended to treat the cancer.

In some embodiments, the HSP90 inhibitor is administered to a subject prior to, or following surgical excision/removal of the cancer.

In some embodiments, the HSP90 inhibitor is administered to a subject before, during, and/or after radiation treatment of the cancer.

In some embodiments, the HSP90 inhibitor is administered to a subject, e.g., a cancer patient who is undergoing or has undergone cancer therapy (e.g., treatment with a chemotherapeutic, radiation therapy and/or surgery). For example, the HSP90 inhibitor can be administered to a patient undergoing therapy with a second agent, e.g., an mTOR inhibitor, and/or a tyrosine kinase inhibitor, a topoisomerase inhibitor (e.g., irinotecan), or a taxane (e.g., docetaxel). In other embodiments, the HSP90 inhibitor is administered concurrently with the second agent. In instances of concurrent administration, the HSP90 inhibitor can continue to be administered after treatment with the second agent has ceased. In other embodiments, the HSP90 is administered after treatment with the second agent has ceased (i.e., with no period of overlap with the cancer treatment).

In one embodiment, the second agent used in combination with the HSP90 inhibitor is a tyrosine kinase inhibitor (e.g., a receptor tyrosine kinase (RTK) inhibitor).

In yet other embodiments, the HSP90 inhibitor, alone or combination with the mTOR inhibitor, the ALK inhibitor, the tyrosine kinase inhibitor, and/or the anti-cancer agent (e.g., a topoisomerase inhibitor or RTK inhibitor, a topoisomerase inhibitor (e.g., irinotecan), or a taxane (e.g., docetaxel)) is administered in a therapeutically effective amount, e.g., at a predetermined dosage schedule.

For example, for treatment of a colorectal cancer, an HSP90 inhibitor can be administered in combination with a topoisomerase inhibitor, e.g., irinotecan.

For example, for treatment of a NSCLC or SCC cancer, an HSP90 inhibitor can be administered in combination with a taxane, e.g., docetaxel (e.g., as a Docetaxel injection (Taxotere®)). In one embodiment, the HSP90 inhibitor is IPI-504. IPI-504 can be administered weekly at a dose of 450 mg/m², alone or in combination with the standard second line dose of docetaxel (75 mg/m²). Docetaxel (Taxotere®) can be administered by intravenous (IV) infusion every 3 weeks (Day 1 of each 21-day cycle) at a dose of 75 mg/m² over approximately 60 minutes.

In other embodiments, treatment of a breast cancer can be effected by administering to a subject (e.g., a patient with advanced or metastatic breast cancer; a patient with HER2-positive breast cancer) an HSP90 inhibitor in combination with a HER2 inhibitor, e.g., an anti-HER2 antibody such as trastuzumab (HERCEPTIN®). In one embodiment, the HSP-90 inhibitor, e.g., IPI-504 is administered to a patient with metastatic HER2-positive breast cancer weekly (e.g., at a dose of about 300 mg/m2) and the HER2 inhibitor (e.g., trastuzumab) is administered every 3 weeks.

In other embodiments, the methods and/or kits described herein further include providing and/or transmitting information, e.g., a report, containing a parameter of the evaluation or treatment determined by the methods and/or kits as described herein to a report-receiving party or entity, e.g., a patient, a health care provider, a diagnostic provider, and/or a regulatory agency, e.g., the FDA, or otherwise submitting information about the methods and kits disclosed herein to another party. The method can relate to compliance with a regulatory requirement, e.g., a pre- or post approval requirement of a regulatory agency, e.g., the FDA.

In one embodiment, the report-receiving party or entity can determine if a predetermined requirement or reference value is met by the data, and, optionally, a response from the report-receiving entity or party is received, e.g., by a physician, patient, diagnostic provider.

In another aspect, the invention features kits for determining the chemosensitivity of a cancer patient to treatment with an HSP90 inhibitor, comprising a reagent that specifically binds to one or more oncogenic alterations, e.g., mutant ALK, MAPK pathway (e.g., K-Ras), EGFR polynucleotide molecules or polypeptides. In certain embodiments, the kits include an HSP90 inhibitor, alone or in combination with an mTOR inhibitor, ALK inhibitor, a tyrosine kinase inhibitor. In one embodiment, the reagent comprises one or more polynucleotide probes. In one embodiment, each of the probes comprises a polynucleotide sequence which is complementary to a nucleotide sequence listed in Table 1 or Table 5, or a sequence disclosed herein, or a complementary sequence thereto. In another embodiment, the probes comprise polynucleotides from 50 to 10⁷ nucleotides in length. In still another embodiment, the probes comprise polynucleotides from about 10 to 10⁷ nucleotides in length. In yet another embodiment, the probes are selected from the group consisting of oligonucleotides, cDNA molecules, RNA molecules, and synthetic gene probes comprising nucleobases. In other embodiment, the probes include exonic sequence, or sequences complemetary thereto. In still another embodiment, the reagent comprises an antibody, and antibody derivative, and an antibody fragment to a polypeptide encoded by one or more polynucleotide sequences listed in Table 1 or Table 5, or a sequence disclosed herein. In embodiments, the sample is evaluated in relation to a reference value, e.g., a control sample. The kit can optionally include instructions for use in detecting the oncogenic alterations, and/or evaluating the results.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE FIGURES

FIG. 1 depicts a waterfall plot showing the best percent change in size of target lesions responses according to ALK status. The y axis represents % tumor volume change from baseline. For each patient (each bar) the percent change in measurable tumor at best response is displayed by the genotype of the patient, i.e. ALK rearrangement status. Black (also indicated with an arrow): ALK mutant; dark grey (also indicated with an asterisk): ALK wild type; light grey: ALK status unknown.

FIG. 2 depicts the number of days on study. The y axis represents the number of days from first dose. Each bar represents a patient. Black (also indicated with an arrow): ALK mutant; dark grey (also indicated with an asterisk): ALK wild type; light grey: ALK status unknown.

FIG. 3A depicts a waterfall plot showing responses according to EGFR status. The y axis represents % tumor volume change from baseline. Each bar represents a patient. EGFR mutant is indicated by an arrow.

FIG. 3B depicts a waterfall plot showing responses according to KRAS status. The y axis represents % tumor volume change from baseline. Each bar represents a patient. KRAS mutant is indicated by an arrow.

FIG. 3C depicts a waterfall plot showing responses according to ALK FISH status. The y axis represents % tumor volume change from baseline. Each bar represents a patient. ALK rearrangement is indicated by an arrow.

FIG. 4 depicts change in size of target lesions over time for patients tested for ALK rearrangement. The y axis represents % tumor volume change from baseline; the x axis represents months on study. Each dot represents a patient.

FIG. 5 depicts the relative dose-response curves of cell growth inhibition (% of control) in H3122 cells treated with IPI-504 (open circles) or Pf-02341066 (solid circles).

FIG. 6A is a bar graph showing the percentage of viable H3122 cells treated or not treated with IPI-504.

FIG. 6B depicts the relative dose-response curves of cell viability in H3122 cells treated with IPI-504 or Pf-02341066.

FIGS. 7A-7C demonstrate that EML4-ALK is an Hsp90 client protein more sensitive to Hsp90 inhibition than Her2 or mEGFR

FIG. 7A depicts the relative dose-response curves of the level of EML-ALK (open circles) and phospho-EML4-ALK (solid squares) monitored using an ELISA in H3122 cells treated with increasing concentration of IPI-504 for 72 hr. Results are shown as percentage of untreated cells.

FIG. 7B is panel of immunoblot images showing the levels of ALK, phospho-ALK and cleaved-PARP in H3122 cells, HER2 in BT474 cells, and EGFR in H1650 cells at various time points after IPI-504 (1 uM) treatment. Proteins levels were monitored by immunoblotting.

FIG. 7C shows the results of co-immunoprecipitation of EML4-ALK with Hsp90 in different cell lysates.

FIGS. 8A-8B show that IPI-504 treatment induced EML4-ALK degradation, downstream pathway inhibition and cell growth arrest.

FIG. 8A is immunoblot images showing the levels of ALK, phospho-ALK, AKT, phospho-ERK1/2, ERK1/2, phospho-STAT3, and STAT3 in H3122 cells at various time points after IPI-504 (1 uM) treatment.

FIG. 8B is a linear graph showing the effects in growth of H3122 cells incubated with increasing concentrations of IPI-504 for 72 h. Cell growth was monitored using Cell Titer Glo.

FIGS. 9A-9C show that EML4-ALK expression in 293FT confers sensitivity to IPI-504 both in vitro and in vivo.

FIG. 9A is immunoblot images showing the levels of total and phospho-EML4-ALK in lysates from 293FT parental cells (293FT^{wt}), 293FT cells over-expressing EML4-ALK (293FT^{ALK}) and 293FT over-expressing kinase dead EML4-ALK (293FT^{ALK-KD}) in response to IPI-504 treatment. Lysates were separated by SDS-PAGE and immunoblotted using ALK or pALK antibodies.

FIG. 9B is bar graph showing the percentage of viable 293FT^{ALK-KD} and 293FT^{ALK} cells after IPI-504.

FIG. 9C is bar graph showing changes in tumor volume of 293FT cells over-expressing either EML4-ALK (293FT^{ALK}) or YFP (293FT^{YFP}) after injection in the right flank of nude mice and tumor bearing animals and treatment with either vehicle or IPI-504 100 mg/kg, twice weekly for 2 weeks. Results are presented as means and SEM (n=8).

FIGS. 10A-10D show that IPI-504 treatment leads to tumor regression in vivo.

FIG. 10A is a linear graph showing the effects of IPI-504 treatment in tumor regressions in the H3122 xenograft model in samples treated with IPI-504, PF02341066 or vehicle-treated controls. H3122 xenografts (n=10 per arm) were treated with 75 mg/kg IPI-504 i.p. twice weekly (open circles), vehicle (open squares) or PF-1066 50 mg/kg, p.o., QD (solid triangles).

FIG. 10B is an enlargement of the box in FIG. 10A. Results are presented as means and SEM.

FIG. 10C is a linear graph depicting the effects of the combination of IPI-504 and PF-1066 in tumor size in H3122 xenograft model. Tumor volume (in mm³) is shown as a function of days of treatment. The combination of IPI-504 and PF-1066 resulted in 66% regression in tumor size. Combination of IPI-504 and PF-1066. H3122 xenografts (n=10 per arm) were treated with IPI-504 50 mg/kg BIW, (open circles), PF-1066 37.5 mg/kg, QD (solid triangles) or a combination of both (solid squares).

FIG. 10D is an enlargement of the box in FIG. 10C showing the regression of the tumor in the combination arm.

FIG. 11 is a bar graph showing the tumor size in nude mice implanted with 293FTEML4ALKv1 or 293FT-YFP cells after IPI-504 treatment.

FIGS. 12A-12B depict a PD time course after IPI-504 treatment. After a single injection of 100 mg/kg IPI-504 tumors were collected at various times and ALK (FIG. 12A) and cleaved PARP (FIG. 12B) levels were monitored using ELISA and immunoblotting respectively.

FIGS. 13A-13B depict waterfall plots showing responses to IPI-504 according to cancer subtypes analyzed by histology. The cancers examined were adenocarcinoma (shown as #1), bronchioloalveolar carcinoma (BAC) (shown as #2), large cell lung carcinoma (shown as #3), squamous cell carcinoma (shown as #4), unknown (shown as #5) and control (shown as #6). Each bar represents one patient.

FIG. 14 depicts a waterfall plot showing responses to IPI-504 according to smoking status. Non-smokers are shown as #1 and smokers are shown as #2. The y-axis represents % of tumor volume change from baseline. Each bar represents one patient.

FIG. 15 depicts a graph showing increased efficacy of IPI-504 determined by % decrease in tumor volume as the tobacco exposure (assessed by number of pack years) increased in patients with NSCLC. The y-axis represents % of tumor volume change from baseline.

FIG. 16 depicts a graph showing increased efficacy of IPI-504 determined by % decrease in tumor volume as the tobacco exposure (assessed by number of pack years) increased in patients with SCC and other lung cancer histologies. The y-axis represents % of tumor volume change from baseline.

FIG. 17 is a bar graph summarizing the efficacy of the combination of IPI-504 and docetaxel in patients with NSCLC.

FIGS. 18A-18B are flow charts summarizing the study designs of two clinical trials evaluating the combination of IPI-504 and docetaxel.

FIG. 19 depicts the MAPK (Ras-Raf-Mek-Erk) pathway.

FIGS. 20A-20D depict efficacy of the Hsp90 inhibitor 17-AG (also referred to herein as “IPI-493”) in mutant B-Raf colorectal cancer models: Colo205 (FIG. 20A), Colo201 (FIG. 20B), Colo741 (FIG. 20C) and HT55 (FIG. 20D).

FIGS. 21A-21C depict efficacy of the Hsp90 inhibitor 17-AG in mutant K-Ras colorectal cancer models: HCT-116 (FIG. 21A), SW480 (FIG. 21B) and DuDu-1 (FIG. 21C).

FIGS. 22A-22D depict the lack of efficacy of the Hsp90 inhibitor 17-AG (IPI-493) in colorectal cancer models wild type (wt) for both K-Ras and B-Raf: Colo320HSR (FIG. 22A), NCI-H716 (FIG. 22B), SNU-C1 (FIG. 22C) and C2BBe1 (FIG. 22D).

FIG. 23A shows a panel of immunoblots depicting a time dependent decrease in phosphorylated BRAF in mutant Colo 201 and Colo 205 xenografts upon a single dose of IPI-493 (100 mpk). Similar changes were observed in KRAS mutant models. Minimal changes in phosphorylated BRAF activity were detected in wild type Colo320HSR.

FIG. 23B shows a panel of bar graphs depicting a time dependent decrease in phosphorylated MEK in mutant Colo 201 and Colo 205 xenografts. Similar changes were observed in KRAS mutant models. Minimal changes in phosphorylated BRAF activity were detected in wild type Colo320HSR upon a single dose of IPI-493 (100 mpk).

FIG. 23C shows a panel of bar graphs depicting a time dependent increase in cleaved caspase 3 activity in mutant Colo 201 and Colo 205 xenografts (correlating with the decrease on phosphor MEK). Minimal changes were detected in wild type Colo320HSR upon a single dose of IPI-493 (100 mpk).

FIGS. 24A-24B depict the efficacy of the Hsp90 inhibitor 17-AG in primary models of wild-type (wt/wt) and mutant (mut) K-Ras models: CXF-1729 (FIG. 24A) and CXF-260 (FIG. 24B).

FIG. 25 demonstrates activation of the MAPK pathway predicts sensitivity to the Hsp90 inhibitor 17-AG.

FIGS. 26A-26B depict the efficacious combination of the Hsp90 inhibitor 17-AG and irinotecan in a mutant B-Raf colorectal cancer model (Colo-201). FIG. 26B is a zoomed-in section of FIG. 26A.

FIGS. 27A-27B depict the efficacious combination of the Hsp90 inhibitor 17-AG and irinotecan in a mutant K-Ras colorectal cancer model (HCT-116). FIG. 27B is a zoomed-in section of FIG. 27A.

FIGS. 28A-28B depict the efficacious combination of the Hsp90 inhibitor 17-AG and irinotecan in a mutant K-Ras colorectal cancer model (DuDu-1). FIG. 21B is a zoomed-in section of FIG. 21A.

FIG. 29A is a graph depicting the percent growth inhibition for three cell lines (BON-1, QGP-1 and H-720) incubated with various concentrations of 17-AG.

FIG. 29B is a graph depicting the percent growth inhibition for three cell lines (BON-1, QGP-1 and H-720) incubated with various concentrations of IPI-504.

FIG. 30 is a graph depicting the change in BON-1 xenograph tumor size in mice treated with IPI-504 (15 mg/kg) and vehicle administered i.p. twice per week (n=10 per arm).

FIG. 31 is a graph depicting phospho-IGF-1R degradation in BON-1 cells upon treatment with IPI-504.

FIG. 32 is a Western blot of BON-1 cells incubated for 6 or 24 hours with 1 uM IPI-504, 100 nM rapamycin or the combination of both. 50 ug of cell lysate was immunoblotted for pAKT, total AKT, pS6, total S6, pERK 1/2, IGF-1Rb, Hsp70, and b-actin.

### BRIEF DESCRIPTION OF THE TABLES

Table 1 depicts the nucleotide and amino acid sequences of various wild type or mutant ALK or ALK fusions.

Table 2 depicts demographics, baseline characteristics and chemotherapy treatment history by EGFR, KRAS and ALK genotypes.

Table 3 depicts the most commonly reported adverse events.

Table 4 depicts the efficacy of IPI-504 by EGFR, KRAS and ALK genotypes.

Table 5 depicts the nucleotide and amino acid sequences of various wild type or mutant MAPK pathway gene and gene products.

Tables 6-7 are set forth in the appended examples.

Table 8 summarizes the activity of IPI-504 and IPI-493 in CRC cell lines in vitro.

Supplemental Table 1 is a table summarizing the genetic results from snapshot, Oncomap, DxS and Sanger sequencing for mutations in EGFR, KRAS, BRAF, ALK, PIK3CA, TP53 and CTNNB1.

### DETAILED DESCRIPTION OF THE INVENTION

The present invention provides, at least in part, compositions, methods, and kits for the identification, assessment and/or treatment of a cancer or tumor (e.g., an oncogene-associated cancer or tumor) responsive to a treatment that includes an HSP90 inhibitor (e.g., an HSP90 inhibitor as a single agent or in combination, e.g., alone or in combination with an mTOR inhibitor, an ALK inhibitor, a tyrosine kinase inhibitor, and/or other chemotherapeutic agents (e.g., docetaxel or irinotecan).

In one embodiment, the invention provides a method for evaluating the responsiveness of, a tumor, a cancer cell, and/or a subject having said tumor or cancer cell, to a treatment that includes an HSP90 inhibitor by detecting an alteration in an ALK, a MAPK pathway, and/or EGFR gene or gene product (e.g., by detecting one or more of: a gene mutation; a change in gene expression, a transcript or protein level of an an ALK, a MAPK pathway and/or EGFR gene or gene product, such as Ras, Raf, Mek, and/or Erk). In one embodiment, the presence of an alteration in an ALK gene or gene product, e.g., an ALK rearrangement, is indicative of responsiveness to a treatment comprising an HSP90 inhibitor in lung cancer, e.g., non-small cell lung cancer (NSCLC). In other embodiments, the presence of an alteration in a Ras, e.g., K-Ras, gene or gene product, optionally in combination with an alteration in p53, is indicative of responsiveness to a combination of an HSP90 inhibitor and an mTOR inhibitor in lung cancer, e.g., NSCLC. In other embodiments, the presence of an alteration (e.g., a mutation) in a Ras, e.g., a K-Ras, gene or gene product, is indicative of responsiveness to a treatment comprising an HSP90 inhibitor in colorectal cancer (CRC). In yet other embodiments, the presence of an alteration (e.g., a mutation) in a Raf, e.g., a B-Raf, gene or gene product, is indicative of responsiveness to a treatment comprising an HSP90 inhibitor in colorectal cancer.

In another embodiment, the invention further provides a method for identifying or selecting a subject as being likely or unlikely to respond to treatment comprising an HSP90 inhibitor, by evaluating one or more of: the subject's histology (e.g., detecting the presence of NSCLC or squamous cell histology (e.g., detecting NSCLC or SCC cells or tissues in a sample from the subject); the subject's smoking status (e.g., subjects having a smoking history of at least 5, 10, 15 or more pack years); the level or expression of HSP90 gene or gene product, and/or an alteration described herein (e.g., one or more alterations alteration in an ALK, a MAPK pathway and/or EGFR gene or gene product).

In yet other embodiments, the invention includes methods for ameliorating or treating a cancer or tumor harboring an oncogenic alteration described herein (e.g., one or more alterations in an an ALK, a MAPK pathway and/or EGFR gene or gene product) with an HSP90 inhibitor, alone or in combination with an mTOR inhibitor, an ALK inhibitor, a tyrosine kinase inhibitor (e.g., gefitinib), and/or other chemotherapeutic agents (e.g., docetaxel or irinotecan). In certain embodiments, the cancer or tumor is present in a subject in need of, being considered, or evaluated for, HSP90 inhibitor therapy (or a combination therapy with an mTOR inhibitor, an ALK inhibitor, a tyrosine kinase inhibitor, and/or other chemotherapeutic agents (e.g., docetaxel or irinotecan)).

Thus, the invention can, therefore, be used as a means to evaluate responsiveness to, or monitor, therapy including HSP90 inhibition, and/or TOR and/or ALK inhibition; stratify patient populations, identify patients likely to benefit from such agents, predict a time course of disease or a probability of a significant event in the disease for such patients, and/or more effectively monitor, treat or prevent a cancer or tumor.

Certain embodiments of the invention disclosed in the appended Examples are summarized below.

In certain embodiments, methods for identifying specific genomic regions are disclosed. Such methods use techniques known in the art, including, but not limited to, oligonucleotide-based microarrays (Brennan, et al. (2004) Cancer Res. 64(14):4744-8; Lucito, et al. (2003) Genome Res. 13:2291-2305; Bignell et al. (2004) Genome Res. 14:287-295; Zhao, et al (2004) Cancer Research, 64(9):3060-71), and other methods as described herein including, for example, hybridization methods (such as, for example, FISH and FISH plus spectral karotype (SKY)). Moreover, compositions and kits are provided for carrying out the methods of the present invention.

For example, the invention provides methods for evaluation of genomic rearrangements in the ALK locus, of the presence, absence or copy number changes of the ALK gene, mutations and/or gene products identified herein (e.g., the markers set forth in Table 1), or by evaluating the copy number, expression level, protein level, protein activity, presence of mutations (e.g., substitution, deletion, or addition mutations) which affect activity of the ALK gene products (e.g., the markers set forth in Table 1).

In other embodiments, the invention provides methods for detection of abnormal activation of the MAPK (RAS-RAF-MEK-Erk) pathway (“MAPK pathway activation”), e.g., for example, by detection of mutations in a gene of that pathway (“MAPK pathway gene”) or transcript thereof, by detection of mutations in a protein of that pathway, or by detection of elevated levels of an unphosphorylated and/or phosphorylated protein of that pathway (“pathway protein”).

In other embodiments, Applicants have discovered that: 1) EML4-ALK is a highly sensitive Hsp90 client protein; 2) expression of EML4-ALK can sensitize cells to IPI-504 treatment; 3) combinations of IPI-504 and ALK kinase inhibitors lead to pronounced tumor regressions in xenograft models of human NSCLC; 4) cells selected for resistance to ALK kinase inhibitors retain sensitivity to IPI-504; and 5) in patients, rearrangements in the ALK locus are associated with responses to IPI-504 as a single agent. Thus, the present invention provides methods and compositions for treating patients with NSCLC and an ALK rearrangement with an HSP-90 inhibitor as a single agent or in combination therapy, e.g., in combination with an ALK kinase inhibitor.

In another embodiment, Applicants have discovered that detecting the presence of a mutation in K-Ras, alone or in combination with p53, is indicative of responsiveness to the combination therapy of an HSP90 inhibitor and an mTOR inhibitor, but not predictive of responsiveness to HSP90 inhibitor therapy as a single agent.

In yet another embodiment, Applicants have discovered that the Hsp90 inhibitor 17-AG demonstrates a dramatic efficacy in both in vitro and in vivo models of KRAS and BRAF mutant CRC. In contrast, the majority of the models wt/wt for both KRAS and BRAF exhibited little to no sensitivity to Hsp90 inhibition. It was also observed that the combination of the Hsp90 inhibitor 17-AG and irinotecan (SOC in CRC) demonstrates efficacy over either agent administered alone. Furthermore, pathway analysis of tumors from mutant K-Ras/B-Raf and wt/wt models demonstrated that MAPK pathway activity is a good predictor of Hsp90 sensitivity. Thus, HSP90 inhibition is comparable to SOC and the combination of an HSP90i with SOC can be a more efficacious approach for treatment of CRC.

Various aspects of the invention are described in further detail in the following subsections.

### I. Definitions

As used herein, each of the following terms has the meaning associated with it in this section.

### Chemical Definitions

Definitions of specific functional groups and chemical terms are described in detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in, for example, Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, i.e., stereoisomers (enantiomers, diastereomers, cis-trans isomers, E/Z isomers, etc.). Thus, inventive compounds and pharmaceutical compositions thereof can be in the form of an individual enantiomer, diastereomer or other geometric isomer, or can be in the form of a mixture of stereoisomers. Enantiomers, diastereomers and other geometric isomers can be isolated from mixtures (including racemic mixtures) by any method known to those skilled in the art, including chiral high pressure liquid chromatography (HPLC) and the formation and crystallization of chiral salts or prepared by asymmetric syntheses; see, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Carbon atoms, unless otherwise specified, can optionally be substituted with one or more substituents. The number of substituents is typically limited by the number of available valences on the carbon atom, and can be substituted by replacement of one or more of the hydrogen atoms that would be available on the unsubstituted group. Suitable substituents are known in the art and include, but are not limited to, alkyl, alkenyl, alkynyl, alkoxy, alkoxy, aryl, aryloxy, arylthio, aralkyl, heteroaryl, heteroaralkyl, cycloalkyl, heterocyclyl, halo, azido, hydroxyl, thio, alkthiooxy, amino, nitro, nitrile, imino, amido, carboxylic acid, aldehyde, carbonyl, ester, silyl, alkylthio, haloalkyl (e.g., perfluoroalkyl such as —CF₃), ═O, ═S, and the like.

When a range of values is listed, it is intended to encompass each value and sub-range within the range. For example, an alkyl group containing 1-6 carbon atoms (C₁₋₆ alkyl) is intended to encompass, C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

The term “alkyl,” as used herein, refers to saturated, straight- or branched-chain hydrocarbon radical containing between one and thirty carbon atoms. In certain embodiments, the alkyl group contains 1-20 carbon atoms. Alkyl groups, unless otherwise specified, can optionally be substituted with one or more substituents. In certain embodiments, the alkyl group contains 1-10 carbon atoms. In certain embodiments, the alkyl group contains 1-6 carbon atoms. In certain embodiments, the alkyl group contains 1-5 carbon atoms. In certain embodiments, the alkyl group contains 1-4 carbon atoms. In certain embodiments, the alkyl group contains 1-3 carbon atoms. In certain embodiments, the alkyl group contains 1-2 carbon atoms. In certain embodiments, the alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a straight- or branched-chain hydrocarbon radical having at least one carbon-carbon double bond by the removal of a single hydrogen atom, and containing between two and thirty carbon atoms. Alkenyl groups, unless otherwise specified, can optionally be substituted with one or more substituents. In certain embodiments, the alkenyl group contains 2-20 carbon atoms. In certain embodiments, the alkenyl group contains 2-10 carbon atoms. In certain embodiments, the alkenyl group contains 2-6 carbon atoms. In certain embodiments, the alkenyl group contains 2-5 carbon atoms. In certain embodiments, the alkenyl group contains 2-4 carbon atoms. In certain embodiment, the alkenyl group contains 2-3 carbon atoms. In certain embodiments, the alkenyl group contains 2 carbon atoms. Alkenyl groups include, for example, ethenyl, propenyl, butenyl, 1-methyl-2-buten-1-yl, and the like.

The term “alkynyl,” as used herein, denotes a straight- or branched-chain hydrocarbon radical having at least one carbon-carbon triple bond by the removal of a single hydrogen atom, and containing between two and thirty carbon atoms. Alkynyl groups, unless otherwise specified, can optionally be substituted with one or more substituents. In certain embodiments, the alkynyl group contains 2-20 carbon atoms. In certain embodiments, the alkynyl group contains 2-10 carbon atoms. In certain embodiments, the alkynyl group contains 2-6 carbon atoms. In certain embodiments, the alkynyl group contains 2-5 carbon atoms. In certain embodiments, the alkynyl group contains 2-4 carbon atoms. In certain embodiments, the alkynyl group contains 2-3 carbon atoms. In certain embodiments, the alkynyl group contains 2 carbon atoms. Representative alkynyl groups include, but are not limited to, ethynyl, 2-propynyl (propargyl), 1-propynyl, and the like.

The terms “cycloalkyl”, used alone or as part of a larger moiety, refer to a saturated monocyclic or bicyclic hydrocarbon ring system having from 3-15 carbon ring members. Cycloalkyl groups, unless otherwise specified, can optionally be substituted with one or more substituents. In certain embodiments, cycloalkyl groups contain 3-10 carbon ring members. In certain embodiments, cycloalkyl groups contain 3-9 carbon ring members. In certain embodiments, cycloalkyl groups contain 3-8 carbon ring members. In certain embodiments, cycloalkyl groups contain 3-7 carbon ring members. In certain embodiments, cycloalkyl groups contain 3-6 carbon ring members. In certain embodiments, cycloalkyl groups contain 3-5 carbon ring members. Cycloalkyl groups include, without limitation, cyclopropyl, cyclobutyl, cyclopentyl, cyclohexyl, cycloheptyl, and cyclooctyl. The term “cycloalkyl” also includes saturated hydrocarbon ring systems that are fused to one or more aryl or heteroaryl rings, such as decahydronaphthyl or tetrahydronaphthyl, where the point of attachment is on the saturated hydrocarbon ring.

The term “aryl” used alone or as part of a larger moiety (as in “aralkyl”), refers to an aromatic monocyclic and bicyclic hydrocarbon ring system having a total of 6-10 carbon ring members. Aryl groups, unless otherwise specified, can optionally be substituted with one or more substituents. In certain embodiments of the present invention, “aryl” refers to an aromatic ring system which includes, but not limited to, phenyl, biphenyl, naphthyl, anthrancyl and the like, which can bear one or more substituents. Also included within the scope of the term “aryl”, as it is used herein, is a group in which an aryl ring is fused to one or more non-aromatic rings, such as indanyl, phthalimidyl or tetrahydronaphthalyl, and the like, where the point of attachment is on the aryl ring.

The term “aralkyl” refers to an alkyl group, as defined herein, substituted by aryl group, as defined herein, wherein the point of attachment is on the alkyl group.

The term “heteroatom” refers to boron, phosphorus, selenium, nitrogen, oxygen, or sulfur, and includes any oxidized form of nitrogen or sulfur, and any quaternized form of a basic nitrogen.

The terms “heteroaryl” used alone or as part of a larger moiety, e.g., “heteroaralkyl”, refer to an aromatic monocyclic or bicyclic hydrocarbon ring system having 5-10 ring atoms wherein the ring atoms comprise, in addition to carbon atoms, from one to five heteroatoms. Heteroaryl groups, unless otherwise specified, can optionally be substituted with one or more substituents. When used in reference to a ring atom of a heteroaryl group, the term “nitrogen” includes a substituted nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaryl ring is fused to one or more aryl, cycloalkyl or heterocycloalkyl rings, wherein the point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl.

The term “heteroaralkyl” refers to an alkyl group, as defined herein, substituted by a heteroaryl group, as defined herein, wherein the point of attachment is on the alkyl group.

As used herein, the terms “heterocycloalkyl” or “heterocyclyl” refer to a stable non-aromatic 5-7 membered monocyclic hydrocarbon or stable non-aromatic 7-10 membered bicyclic hydrocarbon that is either saturated or partially unsaturated, and having, in addition to carbon atoms, one or more heteroatoms. Heterocycloalkyl or heterocyclyl groups, unless otherwise specified, can optionally be substituted with one or more substituents. When used in reference to a ring atom of a heterocycloalkyl group, the term “nitrogen” includes a substituted nitrogen. The point of attachment of a heterocycloalkyl group can be at any of its heteroatom or carbon ring atoms that results in a stable structure. Examples of heterocycloalkyl groups include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. “Heterocycloalkyl” also include groups in which the heterocycloalkyl ring is fused to one or more aryl, heteroaryl or cycloalkyl rings, such as indolinyl, chromanyl, phenanthridinyl, or tetrahydroquinolinyl, where the radical or point of attachment is on the heterocycloalkyl ring.

The term “unsaturated”, as used herein, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but is not intended to include aromatic groups, such as aryl or heteroaryl moieties, as defined herein.

The term “diradical” as used herein refers to an alkyl, alkenyl, alkynyl, aryl, cycloalkyl, heterocycloalkyl, aralkyl, heteroaryl, and heteroaralkyl groups, as described herein, wherein 2 hydrogen atoms are removed to form a divalent moiety. Diradicals are typically end with a suffix of “-ene”. For example, alkyl diradicals are referred to as alkylenes (for example:

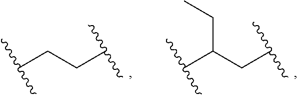

and —(CR′₂)ₓ— wherein R′ is hydrogen or other substituent and x is 1, 2, 3, 4, 5 or 6); alkenyl diradicals are referred to as “alkenylenes”; alkynyl diradicals are referred to as “alkynylenes”; aryl and aralkyl diradicals are referred to as “arylenes” and “aralkylenes”, respectively (for example:

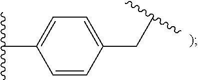

heteroaryl and heteroaralkyl diradicals are referred to as

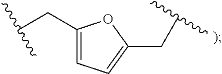

“heteroarylenes” and “heteroaralkylenes”, respectively (for example: cycloalkyl diradicals are referred to as “cycloalkylenes”; heterocycloalkyl diradicals are referred to as “heterocycloalkylenes”; and the like.

The terms “halo”, “halogen” and “halide” as used herein refer to an atom selected from fluorine (fluoro, F), chlorine (chloro, Cl), bromine (bromo, Br), and iodine (iodo, I).

As used herein, the term “haloalkyl” refers to an alkyl group, as described herein, wherein one or more of the hydrogen atoms of the alkyl group is replaced with one or more halogen atoms. In certain embodiments, the haloalkyl group is a perhaloalkyl group, that is, having all of the hydrogen atoms of the alkyl group replaced with halogens (e.g., such as the perfluoroalkyl group —CF₃).

As used herein, the term “azido” refers to the group —N₃.

As used herein, the term “nitrile” refers to the group —CN.

As used herein, the term “nitro” refers to the group —NO₂.

As used herein, the term “hydroxyl” or “hydroxy” refers to the group —OH.

As used herein, the term “thiol” or “thio” refers to the group —SH.

As used herein, the term “carboxylic acid” refers to the group —CO₂H.

As used herein, the term “aldehyde” refers to the group —CHO.

As used herein, the term “alkoxy” refers to the group —OR′, wherein R′ is an alkyl, alkenyl or alkynyl group, as defined herein.

As used herein, the term “aryloxy” refers to the group —OR′, wherein each R′ is an aryl or heteroaryl group, as defined herein.

As used herein, the term “alkthiooxy” refers to the group —SR', wherein each R′ is, independently, a carbon moiety, such as, for example, an alkyl, alkenyl, or alkynyl group, as defined herein.

As used herein, the term “arylthio” refers to the group —SR', wherein each R′ is an aryl or heteroaryl group, as defined herein.

As used herein, the term “amino” refers to the group —NR'₂, wherein each R′ is, independently, hydrogen, a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “carbonyl” refers to the group —C(═O)R′, wherein R′ is, independently, a carbon moiety, such as, for example, an alkyl, alkenyl, alkynyl, aryl or heteroaryl group, as defined herein.

As used herein, the term “ester” refers to the group —C(═O)OR′ or —OC(═O)R′ wherein each R′ is, independently, a carbon moiety, such as, for example, an alkyl, alkenyl, alkynyl, aryl or heteroaryl group, as defined herein.

As used herein, the term “amide” or “amido” refers to the group —C(═O)N(R′)₂ or —NR′C(═O)R′ wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfonamido” or “sulfonamide” refers to the group —N(R′)SO₂R′ or —SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfamido” or “sulfamide” refers to the group —NR′SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “imide” or “imido” refers to the group —C(═NR′)N(R′)₂ or —NR′C(═NR')R′ wherein each R′ is, independently, hydrogen or a carbon moiety, such as, for example, an alkyl, alkenyl, alkynyl, aryl or heteroaryl group, as defined herein, or wherein two R′ groups together with the nitrogen atom to which they are bound form a 5-8 membered ring.

As used herein “silyl” refers to the group —SiR′ wherein R′ is a carbon moiety, such as, for example, an alkyl, alkenyl, alkynyl, aryl or heteroaryl group.

In some cases, the HSP90 inhibitor can contain one or more basic functional groups (e.g., such as an amino group), and thus is capable of forming pharmaceutically acceptable salts with pharmaceutically acceptable acids. The term “pharmaceutically acceptable salts” in these instances refers to the relatively non-toxic, inorganic and organic acid addition salts. These salts can be prepared in situ in the administration vehicle or the dosage form manufacturing process, or by separately treating the compound in its free base form with a suitable acid. Examples of pharmaceutically acceptable, nontoxic acid addition salts from inorganic acids include, but are not limited to, hydrochloric, hydrobromic, phosphoric, sulfuric, nitric and perchloric acid or from organic acids include, but are not limited to, acetic, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic, and valeric acid addition salts, and the like. In other cases, the HSP90 inhibitor can contain one or more acidic functional groups, and thus is capable of forming pharmaceutically acceptable salts with pharmaceutically acceptable bases. The term “pharmaceutically acceptable salts” in these instances refers to the relatively non-toxic, inorganic and organic base addition salts. These salts can likewise be prepared in situ in the administration vehicle or the dosage form manufacturing process, or by separately treating the compound in its free acid form with a suitable base. Examples of suitable bases include, but are not limited to, metal hydroxides, metal carbonates or metal bicarbonates, wherein the metal is an alkali or alkaline earth metal such as lithium, sodium, potassium, calcium, magnesium, or aluminum. Suitable bases can also include ammonia or organic primary, secondary or tertiary amines. Representative organic amines useful for the formation of base addition salts include, for example, ethylamine, diethylamine, ethylenediamine, ethanolamine, diethanolamine, piperazine and the like (see, e.g., Berge et al., supra).

The term “solvate” refers to a compound of the present invention having either a stoichiometric or non-stoichiometric amount of a solvent associated with the compound. The solvent can be water (i.e., a hydrate), and each molecule of inhibitor can be associated with one or more molecules of water (e.g., monohydrate, dihydrate, trihydrate, etc.). The solvent can also be an alcohol (e.g., methanol, ethanol, propanol, isopropanol, etc.), a glycol (e.g., propylene glycol), an ether (e.g., diethyl ether), an ester (e.g., ethyl acetate), or any other suitable solvent. The HSP90 inhibitor can also exist as a mixed solvate (i.e., associated with two or more different solvents).

The term “sugar” as used herein refers to a natural or an unnatural monosaccharide, disaccharide or oligosaccharide comprising one or more pyranose or furanose rings. The sugar can be covalently bonded to the steroidal alkaloid of the present invention through an ether linkage or through an alkyl linkage. In certain embodiments the saccharide moiety can be covalently bonded to a steroidal alkaloid of the present invention at an anomeric center of a saccharide ring. Sugars can include, but are not limited to ribose, arabinose, xylose, lyxose, allose, altrose, glucose, mannose, gulose, idose, galactose, talose, glucose, and trehalose.

### Non-Chemical Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term or “alteration” or “altered structure” of a marker, gene or gene product refers to the presence of mutations or mutations within the marker gene or maker protein, e.g., mutations which affect expression or activity of the marker, as compared to the normal or wild-type gene or protein. For example, mutations include, but are not limited to inter- and intra-chromosomal rearrangement, substitutions, deletions, and insertion mutations. Mutations can be present in the coding or non-coding region of the marker.

The term “altered amount” of a marker or “altered level” of a marker refers to increased or decreased copy number of a marker or chromosomal region, such as gene mutations and/or gene products described herein (e.g., the markers set forth in Table 1 or Table 5), or one or more gene mutations and/or gene products chosen from ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1, and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample.

The term “altered level of expression” of an oncogenic alteration, e.g., ALK gene mutations and/or gene products described herein (e.g., the markers set forth in Table 1 or Table 5), or one or more gene mutations and/or gene products chosen from ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1, refers to an expression level or copy number of a marker in a test sample, such as a sample derived from a patient suffering from cancer, that is greater or less than the standard error of the assay employed to assess expression or copy number. In embodiments, the alteration can be at least twice, at least twice three, at least twice four, at least twice five, or at least twice ten or more times the expression level or copy number of the alterations, e.g., gene mutations and/or gene products described herein, in a control sample (e.g., a sample from a healthy subject not having the associated disease), or the average expression level or copy number of the alterations, e.g., gene mutations and/or gene products described herein, in several control samples. The altered level of expression is greater or less than the standard error of the assay employed to assess expression or copy number. In embodiments, the alteration is at least twice, at least three, at least four, at least five, at least ten or more times the expression level or copy number of the alterations, e.g., gene mutations and/or gene products described herein, in a control sample (e.g., a sample from a healthy subject not having the associated disease), or the average expression level or copy number of the alterations, e.g., gene mutations and/or gene products described herein, in several control samples.

The term “altered activity” of a marker refers to an activity of a marker which is increased or decreased in a disease state, e.g., in a cancer sample, as compared to the activity of the marker in a normal, control sample. Altered activity of a marker can be the result of, for example, altered expression of the marker, altered protein level of the marker, altered structure of the marker, or, e.g., an altered interaction with other proteins involved in the same or different pathway as the marker.

“Anaplastic lymphoma kinase” and “ALK” are used interchangeably herein and refer to native anaplastic lymphoma kinase, and certain mutations thereof, derived from any source (e.g., rodents, humans, and other mammals), as described herein. In some embodiments, ALK protein is represented by NCBI Ref Seq identification number NP_{—}004295. Unless indicated otherwise, the terms refer to the human protein. The gene encoding ALK can also be referred to herein as “ALK”. In some embodiments, ALK nucleotide sequences are represented by NCBI Ref Seq identification number NM_{—}004304.3 and GenBank accession number 29029631, relevant sequences therein (e.g., the coding, 5′ UTR, 3′UTR, transcription start, translation start, transcription stop, translation stop, etc. sequences) of which can readily be identified by a skilled artisan. By contrast “ALK mutations” refer to mutations and mutants predictive of positive response to treatment with HSP90 inhibiting agents (e.g., IPI-493 and/or IPI-504), as described herein. Representative, non-limiting examples of cytogenetic abnormalities that are screened include EML4-ALK fusions, KIF5B-ALK fusions, TGF-ALK fusions, NPM-ALK fusions, and ALK point mutations comprising one or more of F1245I/L, L1204F, A1200V, L1196M, I1170S, T1151M, R1275Q, F1174V/C/L, T1087I, and K1062M, as described herein.

“Binding compound” shall refer to a binding composition, such as a small molecule, an antibody, a peptide, a peptide or non-peptide ligand, a protein, an oligonucleotide, an oligonucleotide analog, such as a peptide nucleic acid, a lectin, or any other molecular entity that is capable of specifically binding to a target protein or molecule or stable complex formation with an analyte of interest, such as a complex of proteins.

“Binding moiety” means any molecule to which molecular tags can be directly or indirectly attached that is capable of specifically binding to an analyte. Binding moieties include, but are not limited to, antibodies, antibody binding compositions, peptides, proteins, nucleic acids and organic molecules having a molecular weight of up to about 1000 daltons and containing atoms selected from the group consisting of hydrogen, carbon, oxygen, nitrogen, sulfur and phosphorus.

A “biomarker” or “marker” is a gene, mRNA, or protein which can be altered, wherein said alteration is associated with cancer. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, a marker of the invention which is associated with cancer or predictive of responsiveness to anti-cancer therapeutics can have an altered nucleotide sequence, amino acid sequence, chromosomal translocation, intra-chromosomal inversion, copy number, expression level, protein level, protein activity, or methylation status, in a cancer tissue or cancer cell as compared to a normal, healthy tissue or cell. Furthermore, a “marker” includes a molecule whose structure is altered, e.g., mutated (contains an mutation), e.g., differs from the wild type sequence at the nucleotide or amino acid level, e.g., by substitution, deletion, or insertion, when present in a tissue or cell associated with a disease state, such as cancer.

The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, mu chain disease, benign monoclonal gammopathy, immunocytic amyloidosis, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), Waldenstrom's macroglobulinemia, heavy chain disease, soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

“Chemotherapeutic agent” means a chemical substance, such as a cytotoxic or cytostatic agent, that is used to treat a condition, particularly cancer.

As used herein, “cancer” and “tumor” are synonymous terms.

As used herein, “cancer therapy” and “cancer treatment” are synonymous terms.

As used herein, “chemotherapy” and “chemotherapeutic” and “chemotherapeutic agent” are synonymous terms.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

The “copy number of a gene” or the “copy number of a marker” refers to the number of DNA sequences in a cell encoding a particular gene product. Generally, for a given gene, a mammal has two copies of each gene. The copy number can be increased, however, by gene amplification or duplication, or reduced by deletion.

A marker is “fixed” to a substrate if it is covalently or non-covalently associated with the substrate such that the substrate can be rinsed with a fluid (e.g., standard saline citrate, pH 7.4) without a substantial fraction of the marker dissociating from the substrate.

“Hazard ratio”, as used herein, refers to a statistical method used to generate an estimate for relative risk. “Hazard ratio” is the ratio between the predicted hazard of one group versus another group. For example, patient populations treated with an HSP90 inhibiting agent versus without an HSP90 inhibiting agent can be assessed for whether or not the HSP90 inhibiting agent is effective in increasing the time to distant recurrence of disease, particularly with regard to ALK mutation status. For example, treating subjects harboring ALK mutations in cancerous tissue, as described herein, results in increased therapeutic benefit from HSP90 inhibiting agents relative to subjects not having said ALK mutations in cancerous tissue.

“Heat shock protein (Hsp) 90” or “HSP90”, as used herein, includes each member of the family of heat shock proteins having a mass of about 90-kiloDaltons. For example, in humans the highly conserved Hsp90 family includes cytosolic Hsp90α and Hsp90β isoforms, as well as GRP94, which is found in the endoplasmic reticulum, and HSP75/TRAP1, which is found in the mitochondrial matrix. Hsp90 pay an integral role in protein homeostasis and regulates the stability of key proteins involved in oncogenesis, cancer cell proliferation, and survival through its role as a protein chaperone (Kanelakis K. C. et al. (2003) Methods Enzymol. 364:159-173; Hanahan D. et al. (2000) Cell. 100(1):57-70). Hsp90 can preferentially chaperone mutant oncoproteins over wild-type versions, further increasing its attractiveness as a therapeutic target (Nathan D. F. et al. (1995) Mol Cell Biol. 15(7):3917-3925; Rutherford S. L. et al. (1998) Nature 396(6709):336-342; Grbovic O. M. et al. (2006) Proc Natl Acad Sci USA. 103(1):57-62; Shimamura T. et al. (2005) Cancer Res. 65(14):6401-6408).

“HSP90 inhibiting agent” or “HSP90 inhibitor,” as used herein, refers to a compound that can inhibit the biological activity of HSP90. Biological activities can also include patient response as set forth in this application. Exemplary HSP90 inhibiting agents include, but are not limited to, IPI-493 (Infinity Pharm.), IPI-504 (Infinity Pharm.), 17-AAG (also known as tanespimycin or CNF-1010; BMS), BIIB-021 (also known as CNF-2024, Biogen IDEC), BIIB-028 (Biogen IDEC), AUY-922 (also known as VER-49009, Novartis), SNX-5422 (Pfizer), STA-9090, AT-13387 (Astex), XL-888 (Exelixis), MPC-3100 (Myriad), CU-0305 (Curis), 17-DMAG, CNF-1010, a Macbecin (e.g., Macbecin I, Macbecin II), CCT-018159, CCT-129397, PU-H71 (Memorial Sloan Kettering Cancer Center), PF-04928473 (SNX-2112), TAE684, and PF-02341066. Other HSP90 inhibitors are disclosed in Zhang, M-Q. et al., J. Med. Chem. 51(18):5494-5497 (2008) and Menzella, H. et al., J. Med. Chem., 52(6):15128-1521 (2009), the entire contents of which are incorporated herein by reference.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, at least 60%, at least 65%, at least 70%, at least 75%, at least 80%, at least 85%, at least 90%, at least 95% or more.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

“Likely to” or “increased likelihood,” as used herein, refers to an increased probability that an item, object, thing or person will occur. Thus, in one example, a subject that is likely to respond to treatment with an HSP90 inhibiting agent, alone or in combination with an mTOR inhibitor, has an increased probability of responding to treatment with an HSP90 inhibiting agent, alone or in combination with an mTOR inhibitor, relative to a reference subject or group of subjects.

“Long,” as used herein, refers to a time measure that is greater than normal, greater than a standard such as a predetermined measure or a subgroup measure that is relatively longer than another subgroup measure. For example, with respect to a patient's longevity, a long time progression refers to time progression that is longer than a normal time progression. Whether a time progression is long or not can be determined according to any method available to one skilled in the art. Long could include, for example, no progression.

A “marker nucleic acid” is a nucleic acid (e.g., DNA, mRNA, cDNA) encoded by or corresponding to a marker of the invention. For example, such marker nucleic acid molecules include DNA (e.g., genomic DNA and cDNA) comprising the entire or a partial sequence of any of the nucleic acid sequences set forth herein (e.g., in Table 1 or Table 5), or the complement or hybridizing fragment of such a sequence. The marker nucleic acid molecules also include RNA comprising the entire or a partial sequence of any of the nucleic acid sequences set forth herein (e.g., in Table 1 or Table 5), or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth herein (e.g., in Table 1 or Table 5), or a fragment thereof. The terms “protein” and “polypeptide” are used interchangeably herein.

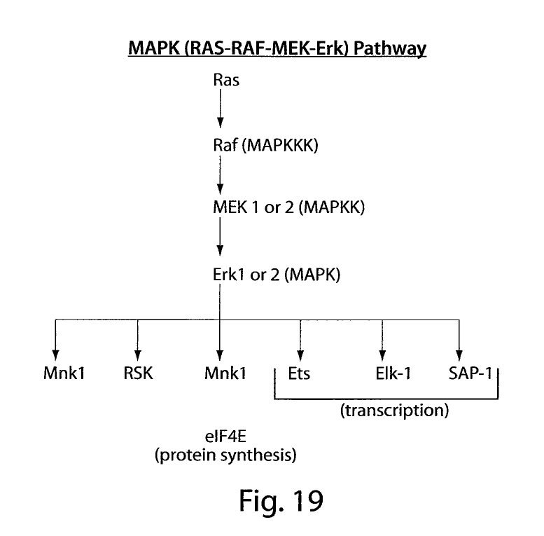

“MAPK pathway gene(s),” as used herein, refers to genes that are directly and/or indirectly involved in intracellular signaling via mitogen activated protein kinases (MAPK). In some embodiments, this direct and/or indirect involvement can comprise genes upstream and/or downstream of MAPK. MAP kinases are well known in the art to comprise important mediators of cancer-related disease mechanisms (Chen et al., Chem Rev (2001) 101:2449-76; Pearson et al., Endocr Rev (2001) 22:153-83; English et al., Trends Pharmacol Sci (2002) 23:40-45; Kohno et al., Prog Cell Cycle Res (2003) 5:219-24; and Sebolt-Leopold, Oncogene (2000) 19:6594-99). One of the MAPK pathways enables the transmission of signals from extracellular signals, such as epidermal growth factor (EGF) and vascular endothelial derived growth factor (VEGF), which bind to a corresponding receptor in the cell membrane, EGFR, HER, and VEGFR, respectively, which sends the signal on to the cell nucleus via intermediary kinases and kinase targets. In one embodiment, a MAPK pathway comprises RAS, RAF, MEK, and ERK (MAPK) (e.g., Ras, Raf-1, A-Raf, B-Raf (BRAF), MEK1 and/or MEK2, which are collectively referred to herein as MEK1/2, and ERK1 and/or ERK2, which are collectively referred to herein as ERK1/2. In some embodiments, such MAPK pathways further comprise MAPK target genes as Mnk1, Rsk, Ets, Elk-1, and Sap-1 (see, for example, FIG. 19). The latter proteins ultimately govern expression of genes that, for example, control vital cell functions such as proliferation, growth, motility and survival. Nucleic acid and protein sequences for MAPK pathway genes are well known to a skilled artisan and representative, non-limiting examples of gene and protein accession numbers for the specific MAPK pathway genes include: Kras (NM_{—}033360.2; NP_{—}203524.1), Hras (NM_{—}176795.3; NP_{—}789765.1), Nras (NM_{—}002524.3; NP_{—}002515.1), Braf (BC101757.1; AAI01758), Craf (X03484.1; CAA27204.1), Araf (X04790.1; CAA28476.1), Nk1 (NM_{—}003684.4; NP_{—}003675.2), Rsk (NM_{—}002953.3; NP_{—}002944.2; NM_{—}021135.4; NP_{—}066958.2; NM_{—}004586.2; NP_{—}004577.1; NM_{—}003942.2; and NP_{—}003933.1), Ets (NM_{—}005238.3; NP_{—}005229.1), Elk1 (NM_{—}005229.3; NP_{—}005220.2), and Sap-1 (NM_{—}002351.3; NP_{—}002342.1). In some embodiments, MAPK pathway gene(s) can also refer to either or both of the wild type or native gene, as well as or alternatively, certain mutations thereof, and derived from any source (e.g., rodents, humans, and other mammals), as described herein. In some embodiments, MAPK pathway gene product(s) refer to polypeptides and/or fragments thereof, of the encoding MAPK pathway gene(s). Table 5 provides a non-limiting listing of MAPK pathway gene(s) and/or gene product(s). In some embodiments, MAPK pathway gene(s) and/or gene product(s) are represented by NCBI Ref Seq identification numbers, from which relevant sequences (e.g., the coding, 5′ UTR, 3′UTR, transcription start, translation start, transcription stop, translation stop, mutation sites, etc. sequences) can readily be identified by a skilled artisan. In some embodiments, “MAPK pathway gene(s) and/or gene product(s)” specifically refers to mutations and mutants predictive of positive response to treatment with Hsp90 inhibitors (e.g., compounds of the present invention), alone or in combination with mTOR inhibitors as described herein. Representative, non-limiting examples of such mutations are provided throughtout the specification and in Table 5.

“mTOR inhibitor” as used herein refers to an agent that directly or indirectly target, decreases or inhibits the activity/function of an mTOR kinase (mammalian Target Of Rapamycin). Exemplary mTOR inhibitors include, but are not limited to, compounds, proteins or antibodies that target members of the mTOR kinase family, e.g., one or more mTOR inhibitors chosen from one or more of rapamycin (sirolimus), temsirolimus (TORISEL®), everolimus (RAD001, AFINITOR®), ridaforolimus, AP23573, AP23841, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, PKI-587, ABT578, SAR543, and ascomycin. In one embodiment, the mTOR inhibitor inhibits TORC1 and TORC2. Examples of TORC1 and TORC2 dual inhibitors include, e.g., OSI-027, XL765, Palomid 529, and INK128.

The “normal” copy number of a marker or “normal” level of expression of a marker is the level of expression, copy number of the marker, in a biological sample, e.g., a sample containing tissue, whole blood, serum, plasma, buccal scrape, saliva, cerebrospinal fluid, urine, stool, and bone marrow, from a subject, e.g., a human, not afflicted with cancer.

An “overexpression” or “significantly higher level of expression or copy number” of the gene (e.g., ALK or MAPK pathway gene) mutations and/or gene products (e.g., the markers set forth in Table 1 and Table 5) refers to an expression level or copy number in a test sample that is greater than the standard error of the assay employed to assess expression or copy number. In embodiments, the overexpression can be at least two, at least three, at least four, at least five, or at least ten or more times the expression level or copy number of the gene (e.g., ALK or MAPK pathway gene) mutations and/or gene products (e.g., the markers set forth in Table 1 and Table 5) in a control sample (e.g., a sample from a healthy subject not afflicted with cancer), or the average expression level or copy number of the gene (e.g., ALK or MAPK pathway gene) mutations and/or gene products (e.g., the markers set forth in Table 1 and Table 5) in several control samples.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

“RECIST” shall mean an acronym that stands for “Response Evaluation Criteria in Solid Tumours” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “PR,” “CR,” “SD” and “PD.”

“Responsiveness,” to “respond” to treatment, and other forms of this verb, as used herein, refer to the reaction of a subject to treatment with an HSP90 inhibitor, alone or in combination, e.g., in combination with an mTOR, an ALK inhibitor, or a chemotherapeutic agent. As an example, a subject responds to treatment with an HSP90 inhibiting agent if growth of a tumor in the subject is retarded about 10%, 20%, 30%, 40%, 50%, 60%, 70%, 80%, 90% or more. In another example, a subject responds to treatment with an HSP90 inhibitor, alone or in combination, if a tumor in the subject shrinks by about 5%, 10%, 20%, 30%, 40%, 50% or more as determined by any appropriate measure, e.g., by mass or volume. In another example, a subject responds to treatment with an HSP90 inhibitor, alone or in combination, if the subject experiences a life expectancy extended by about 5%, 10%, 20%, 30%, 40%, 50% or more beyond the life expectancy predicted if no treatment is administered. In another example, a subject responds to treatment with an HSP90 inhibitor, alone or in combination, if the subject has an increased disease-free survival, overall survival or increased time to progression. Several methods can be used to determine if a patient responds to a treatment including the RECIST criteria, as set forth above.

“Sample,” “tissue sample,” “patient sample,” “patient cell or tissue sample” or “specimen” each refers to a collection of similar cells obtained from a tissue of a subject or patient. The source of the tissue sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like.

“Short,” as used herein, refers to a time measure that is shorter than normal, shorter than a standard such as a predetermined measure or a subgroup measure that is relatively shorter than another subgroup measure. For example, with respect to a patient's longevity, a short time progression refers to time progression that is shorter than a normal time progression. Whether a time progression is short or not can be determined according to any method available to one skilled in the art.

The amount of a marker, e.g., expression or copy number of gene (e.g., ALK or MAPK pathway gene) mutations and/or gene products (e.g., one or more the markers set forth in Table 1, Table 5, or described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about two, at least about three, at least about four, or at least about five times, higher or lower, respectively, than the normal amount of the marker.

As used herein, “significant event” shall refer to an event in a patient's disease that is important as determined by one skilled in the art. Examples of significant events include, for example, without limitation, primary diagnosis, death, recurrence, the determination that a patient's disease is metastatic, relapse of a patient's disease or the progression of a patient's disease from any one of the above noted stages to another. A significant event can be any important event used to assess OS, TTP and/or using the RECIST or other response criteria, as determined by one skilled in the art.

As used herein, “time course” shall refer to the amount of time between an initial event and a subsequent event. For example, with respect to a patient's cancer, time course can relate to a patient's disease and can be measured by gauging significant events in the course of the disease, wherein the first event can be diagnosis and the subsequent event can be metastasis, for example.

“Time to progression” or “TTP” refers to a time as measured from the start of the treatment to progression or a cancer or censor. Censoring can come from a study end or from a change in treatment. Time to progression can also be represented as a probability as, for example, in a Kaplein-Meier plot where time to progression can represent the probability of being progression free over a particular time, that time being the time between the start of the treatment to progression or censor.

A “transcribed polynucleotide” is a polynucleotide (e.g., an RNA, a cDNA, or an analog of one of an RNA or cDNA) which is complementary to or homologous with all or a portion of a mature RNA made by transcription of a marker of the invention and normal post-transcriptional processing (e.g., splicing), if any, of the transcript, and reverse transcription of the transcript.

An “underexpression” or “significantly lower level of expression or copy number” of gene (e.g., ALK or MAPK pathway gene) mutations and/or gene products (e.g., the markers set forth in Table 1 or Table 5) refers to an expression level or copy number in a test sample that is greater than the standard error of the assay employed to assess expression or copy number, for example, at least twice, at least three, at least four, at least five, or at least ten or more times less than the expression level or copy number of the gene (e.g., ALK or MAPK pathway gene) mutations and/or gene products (e.g., the markers set forth in Table 1 or Table 5) in a control sample (e.g., a sample from a healthy subject not afflicted with cancer), or the average expression level or copy number of the gene (e.g., ALK or MAPK pathway gene) mutations and/or gene products (e.g., the markers set forth in Table 1 or Table 5) in several control samples.

“Unlikely to” refers to a decreased probability that an event, item, object, thing or person will occur with respect to a reference. Thus, a subject that is unlikely to respond to treatment with an HSP90 inhibiting agent has a decreased probability of responding to treatment with an HSP90 inhibiting agent relative to a reference subject or group of subjects.

Various aspects of the invention are described in further detail below. Additional definitions are set out throughout the specification.

### II. Methods of the Present Invention

Analysis of activating mutations, copy number and/or levels of expression and/or activity of ALK and MAPK pathway gene and/or gene products has led to the identification of individual biomarkers and combinations of biomarkers described herein, which correlate with the efficacy of HSP90 inhibitors, alone or in combination, e.g., in combination with mTOR inhibitors, in treating cancer, in a subject. For example, the present invention provides methods for evaluation of copy number, expression level, protein level, protein activity, presence of mutations (e.g., inter- and intra-chromosomal rearrangements, substitutions, deletions, insertions, or addition mutations) of the ALK or MAPK pathway gene mutations and/or gene products identified herein (e.g., the markers set forth in Table 1 and Table 5), one or more gene mutations and/or gene products chosen from ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1.

In some embodiments, methods of the present invention can be used to monitor the progression of cancer in a subject, wherein if a sample in a subject has a significant increase in the amount, e.g., expression, and/or activity of a marker disclosed herein (e.g., listed in Table 1 or Table 5) during the progression of cancer, e.g., at a first point in time and a subsequent point in time, then the cancer is more likely to respond to treatment with an HSP90 inhibitor, alone or in combination, and vice versa. In yet another embodiment, between the first point in time and a subsequent point in time, the subject has undergone treatment, e.g., chemotherapy, radiation therapy, surgery, or any other therapeutic approach useful for inhibiting cancer, has completed treatment, or is in remission.

### III. Isolated Nucleic Acid Molecules

One aspect of the invention pertains to isolated nucleic acid molecules that correspond to a marker of the invention, including nucleic acids which encode a polypeptide corresponding to a marker of the invention or a portion of such a polypeptide. The nucleic acid molecules of the invention include those nucleic acid molecules which reside in genomic regions identified herein. Isolated nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

A nucleic acid molecule of the present invention, e.g., ALK or MAPK activating gene mutations and/or gene products identified herein (e.g., the markers set forth in Table 1 or Table 5), can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, at least about 15, at least about 25, at least about 50, at least about 75, at least about 100, at least about 125, at least about 150, at least about 175, at least about 200, at least about 250, at least about 300, at least about 350, at least about 400, at least about 500, at least about 600, at least about 700, at least about 800, at least about 900, at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 15 kb, at least about 20 kb, at least about 25 kb, at least about 30 kb, at least about 35 kb, at least about 40 kb, at least about 45 kb, at least about 50 kb, at least about 60 kb, at least about 70 kb, at least about 80 kb, at least about 90 kb, at least about 100 kb, at least about 200 kb, at least about 300 kb, at least about 400 kb, at least about 500 kb, at least about 600 kb, at least about 700 kb, at least about 800 kb, at least about 900 kb, at least about 1 mb, at least about 2 mb, at least about 3 mb, at least about 4 mb, at least about 5 mb, at least about 6 mb, at least about 7 mb, at least about 8 mb, at least about 9 mb, at least about 10 mb or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that are substantially homologous to the gene mutations and/or gene products described herein, e.g., ALK or MAPK activating gene mutations and/or gene products identified herein (e.g., the markers set forth in Table 1 or Table 5) such that they are at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or greater. In other embodiments, the invention further encompasses nucleic acid molecules that are substantially homologous to the gene mutations and/or gene products described herein, e.g., ALK or MAPK activating gene mutations and/or gene products identified herein (e.g., the markers set forth in Table 1 or Table 5) such that they differ by only or at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1 kb, at least 2 kb, at least 3 kb, at least 4 kb, at least 5 kb, at least 6 kb, at least 7 kb, at least 8 kb, at least 9 kb, at least 10 kb, at least 15 kb, at least 20 kb, at least 25 kb, at least 30 kb, at least 35 kb, at least 40 kb, at least 45 kb, at least 50 kb nucleotides or any range in between.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's can occur in protein-coding nucleic acid sequences, in which case they can give rise to a defective or otherwise variant protein, or genetic disease. Such a SNP can alter the coding sequence of the gene and therefore specify another amino acid (a “missense” SNP) or a SNP can introduce a stop codon (a “nonsense” SNP). When a SNP does not alter the amino acid sequence of a protein, the SNP is called “silent.” SNP's can also occur in noncoding regions of the nucleotide sequence. This can result in defective protein expression, e.g., as a result of alternative spicing, or it can have no effect on the function of the protein.

In another embodiment, an isolated nucleic acid molecule of the invention is at least 7, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 550, at least 650, at least 700, at least 800, at least 900, at least 1000, at least 1200, at least 1400, at least 1600, at least 1800, at least 2000, at least 2200, at least 2400, at least 2600, at least 2800, at least 3000, at least 3500, at least 4000, at least 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60%, at least 65%, at least 70%, at least 75%, at least 80%, or at least 85% identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). Another, non-limiting example of stringent hybridization conditions are hybridization in 6× sodium chloride/sodium citrate (SSC) at about 45° C., followed by one or more washes in 0.2×SSC, 0.1% SDS at 50-65° C.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### IV. Isolated Proteins and Antibodies

One aspect of the invention pertains to isolated proteins which correspond to individual markers of the invention, and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, less than about 20%, less than about 10%, or less than about 5% (by dry weight) of heterologous protein (also referred to herein as a “contaminating protein”). When the protein or biologically active portion thereof is recombinantly produced, it can be substantially free of culture medium, i.e., culture medium represents less than about 20%, less than about 10%, or less than about 5% of the volume of the protein preparation. When the protein is produced by chemical synthesis, it can substantially be free of chemical precursors or other chemicals, i.e., it is separated from chemical precursors or other chemicals which are involved in the synthesis of the protein. Accordingly such preparations of the protein have less than about 30%, less than about 20%, less than about 10%, less than about 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the gene mutations and/or gene products described herein, e.g., ALK or MAPK activating gene mutations and/or gene products identified herein (e.g., the markers set forth in Table 1 or Table 5) of the present invention, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

In certain embodiments, the polypeptide has an amino acid sequence of a protein encoded by a nucleic acid molecule disclosed herein. Other useful proteins are substantially identical (e.g., at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 99.5% or greater) to one of these sequences and retain the functional activity of the protein of the corresponding full-length protein yet differ in amino acid sequence.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. Another, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (or at least 10, at least 15, at least 20, or at least 30 or more) amino acid residues of the amino acid sequence of one of the polypeptides of the invention, and encompasses an epitope of the protein such that an antibody raised against the peptide forms a specific immune complex with a marker of the invention to which the protein corresponds. Exemplary epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to, for example, determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

### V. Kits

A kit is any manufacture (e.g., a package or container) comprising at least one reagent, e.g., a probe, for specifically detecting a marker of the invention, the manufacture being promoted, distributed, or sold as a unit for performing the methods of the present invention. When the compositions, kits, and methods of the invention are used for carrying out the methods of the invention, the gene mutations and/or gene products described herein, e.g., ALK or MAPK activating gene mutations and/or gene products identified herein (e.g., the markers set forth in Table 1 or Table 5) of the invention can be selected such that a positive result is obtained in at least about 20%, at least about 40%, at least about 60%, at least about 80%, at least about 90%, at least about 95%, at least about 99% or in 100% of subjects afflicted with cancer, of the corresponding stage, grade, histological type, or benign/premaligant/malignant nature. In certain embodiments, the marker or panel of markers of the invention can be selected such that a PPV (positive predictive value) of greater than about 10% is obtained for the general population (e.g., coupled with an assay specificity greater than 99.5%).

When a plurality of gene mutations and/or gene products described herein, e.g., ALK or MAPK activating gene mutations and/or gene products identified herein (e.g., the markers set forth in Table 1 or Table 5) are used in the compositions, kits, and methods of the invention, the amount, structure, and/or activity of each marker or level of expression or copy number can be compared with the normal amount, structure, and/or activity of each of the plurality of markers or level of expression or copy number, in non-cancerous samples of the same type, either in a single reaction mixture (i.e., using reagents, such as different fluorescent probes, for each marker) or in individual reaction mixtures corresponding to one or more of the gene mutations and/or gene products described herein, e.g., ALK or MAPK activating gene mutations and/or gene products identified herein (e.g., the markers set forth in Table 1 or Table 5). If a plurality of gene (e.g., ALK gene) mutations and/or gene products (e.g., the markers set forth in Table 1 or described herein) is used, then 2, 3, 4, 5, 6, 7, 8, 9, 10, or more individual markers can be used or identified.

The invention includes compositions, kits, and methods for assaying cancer cells in a sample (e.g., an archived tissue sample or a sample obtained from a subject). These compositions, kits, and methods are substantially the same as those described above, except that, where necessary, the compositions, kits, and methods are adapted for use with certain types of samples. For example, when the sample is a parafinized, archived human tissue sample, it can be necessary to adjust the ratio of compounds in the compositions of the invention, in the kits of the invention, or the methods used. Such methods are well known in the art and within the skill of the ordinary artisan.

The invention thus includes a kit for assessing the presence of cancer cells (e.g., in a sample such as a subject sample). The kit can comprise one or more reagents capable of identifying gene mutations and/or gene products described herein, e.g., ALK or MAPK activating gene mutations and/or gene products identified herein (e.g., the markers set forth in Table 1 or Table 5), e.g., binding specifically with a nucleic acid or polypeptide corresponding to gene mutations and/or gene products described herein, e.g., ALK or MAPK activating gene mutations and/or gene products identified herein (e.g., the markers set forth in Table 1 or Table 5). Suitable reagents for binding with a polypeptide corresponding to a marker of the invention include antibodies, antibody derivatives, antibody fragments, and the like. Suitable reagents for binding with a nucleic acid (e.g., a genomic DNA, an mRNA, a spliced mRNA, a cDNA, or the like) include complementary nucleic acids. For example, the nucleic acid reagents can include oligonucleotides (labeled or non-labeled) fixed to a substrate, labeled oligonucleotides not bound with a substrate, pairs of PCR primers, molecular beacon probes, and the like.

The kit of the invention can optionally comprise additional components useful for performing the methods of the invention. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of cancer cells, and the like.

A kit of the invention can comprise a reagent useful for determining protein level or protein activity of a marker. In another embodiment, a kit of the invention can comprise a reagent for determining methylation status of a marker, or can comprise a reagent for determining alteration of structure of a marker, e.g., the presence of a mutation.

### VI. Predictive Medicine

The present invention also pertains to the field of predictive medicine in which diagnostic assays, pharmacogenomics, and monitoring clinical trials are used for predictive purposes to thereby treat an individual prophylactically. Accordingly, one aspect of the present invention relates to assays for determining the amount, structure, and/or activity of polypeptides or nucleic acids corresponding to one or more markers of the invention, in order to determine whether an individual having cancer or at risk of developing cancer will be more likely to respond to HSP90 inhibitor-mediated therapy.

Accordingly, in one aspect, the invention is drawn to a method for determining whether a subject with a cancer is likely to respond to treatment with an HSP90 inhibiting agent, alone or in combination. In another aspect, the invention is drawn to a method for predicting a time course of disease. In still another aspect, the method is drawn to a method for predicting a probability of a significant event in the time course of the disease. In certain embodiments, the method comprises detecting a biomarker or combination of biomarkers associated with responsiveness to treatment with an HSP90 inhibiting agent as described herein, alone or in combination, and determining whether the subject is likely to respond to treatment with the HSP90 inhibiting agent, alone or in combination.

In some embodiments, the methods involve evaluation, e.g., cytogenetic screening, of biological tissue sample from a subject, e.g., a patient who has been diagnosed with or is suspected of having cancer (e.g., presents with symptoms of cancer) to detect one or more ALK alterations, e.g., ALK mutations. Representative, non-limiting examples of cytogenetic abnormalities that are screened include one or more of the following: EML4-ALK fusions, KIF5B-ALK fusions, TGF-ALK fusions, NPM-ALK fusions, ALK gene copy number changes, and ALK point mutations comprising one or more of F1245I/L, L1204F, A1200V, L1196M, I1170S, T1151M, R1275Q, F1174V/C/L, T1087I, and K1062M, as described herein.

In other embodiments, the methods involve evaluation, e.g., cytogenetic screening, of biological tissue sample from a subject, e.g., a patient who has been diagnosed with or is suspected of having cancer (e.g., presents with symptoms of cancer) to detect one or more alteration in RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1. Examples of gene mutations are described in e.g., The Catalogue of Somatic Mutations in Cancer (COSMIC) (http://www.sanger.ac.uk/genetics/CGP/cosmic/).

Examples of EGFR mutations are described in e.g., Couzin J., (2004) Science 305:1222-1223; Fukuoka, M. et al., (2003) J. Clin. Oncol. 21:2237-46; Lynch et al., (2004) NEJM 350(21):2129-2139; Paez et al. (2004) Science 304:1497-1500; Pao, W. et al. Proc Natl Acad Sci USA. (2004) 101(36):13306-11; Gazdar A. F. et al., Trends Mol. Med. (2004) 10(10):481-6; Huang S. F. et al. (2004) Clin Cancer Res. 10(24):8195-203; Couzin J. Science (2004) 305(5688):1222-3; Sordella R. et al. (2004) 305(5687):1163-7; Kosaka T. et al. (2004) Cancer Res. 64(24):8919-23; Marchetti A. et al. J Clin Oncol. (2005) 23(4):857-65; Tokumo M. et al. (2005) Clin Cancer Res. 11(3):1167-1173; Han S. W. et al. (2005) J Clin Oncol. 23(11):2493-501; Mitsudomi T. et al. (2005) J Clin Oncol. 23(11):2513-20; Shigematsu H. et al. J Natl Cancer Inst. 97(5):339-46; Kim K. S. et al., (2005) Clin Cancer Res. 11(6):2244-51; Cappuzzo F. et al. (2005) J Natl Cancer Inst. 97(9):643-55; Cortes-Funes H. et al. Ann Oncol. (2005) 16(7):1081-6; Sasaki H. et al. (2005) Clin Cancer Res. 11(8):2924-9; Chou T. Y. et al., (2005) Clin Cancer Res. 11(10):3750-7; Pao W. et al. (2005) PLoS Med. 2(3):e73; Sasaki H. et al. (2005) Int J Cancer. 118(1):180-4; Eberhard D. A. et al. (2005) J Clin Oncol. 23(25):5900-9; Takano T. et al. (2005) J Clin Oncol. 23(28):6829-37; Tsao M. S. et al., (2005) N Engl J Med. 353(2):133-44; Mu X. L. et al. (2005) Clin Cancer Res. 11(12):4289-94; Sonobe M. et al. (2005) Br J. Cancer. 93(3):355-63; Taron M. et al. (2005) Clin Cancer Res. 11(16):5878-85; Mukohara T. et al., (2005) J Natl Cancer Inst. 97(16):1185-94; Zhang X. T. et al. (2005) Oncol. 16(8):1334-42. Exemplary alterations in an EGFR gene or gene product, include but are not limited to, an EGFR exon deletion (e.g., EGFR exon 19 Deletion), and/or exon mutation (e.g., an L858R/T790M EGFR mutation). Other exemplary alterations include, but are not limited to, EGFR_D770_N771>AGG; EGFR_D770_N771insG; EGFR_D770_N771insG; EGFR_D770_N771insN; EGFR_E709A; EGFR E709G; EGFR_{—}709H; EGFR_E709K; EGFR_E709V; EGFR_E746_A750del; EGFR_E746_A750del, T751A; EGFR_E746_A750del, V ins; EGFR_E746_T751del, I ins; EGFR_E746_T751del, S752A; EGFR_E746_T751del, S752D; EGFR_E746_T751 del, V ins; EGFR_G719A; EGFR_G719C; EGFR_G719S: EGFR_H773_V774insH; EGFR_H773_V774insNPH; EGFR_H773_V774insPH; EGFR_H773>NPY; EGFR_L747_E749del; EGFR_L747_E749del, A750P; EGFR_L747_{—}5752del; EGFR_L747_{—}5752del, P753S; EGFR_L747_{—}5752del, Q ins; EGFR_L747_T750del, P ins; EGFR_L747_T751del; EGFR_L858R; EGFR_L861Q; EGFR_M766_A767insAI; EGFR_P772_H773insV; EGFR S752_{—}1759del; EGFR_{—}5768I; EGFR_T790M; EGFR_V769_D770insASV; EGFR_V769_D770insASV: and EGFR_V774_C775insHV.

Examples of Ras mutations, include but are not limited to, K-Ras, H-Ras and/or N-Ras include, for example, mutations in codon 12, 13 and/or 61, including but not limited to, G12A, G12N, G12R, G12C, G125, G12V, G13N and Q61R. Examples of NRAS mutations are described in e.g., Bacher U. et al. (2006) Blood 107:3847-53; Banerji U. et al. (2008) Mol Cancer Ther. 7:737-9. Examples of KRAS mutations are described in e.g., Tang W. Y. et al. (1999) Br J Cancer 81(2):237-41; Burmer G. C. et al. (1989) Proc. Natl. Acad. Sci. U.S.A. 86(7): 2403-7; Almoguera C. et al. (1988) Cell 53(4): 549-54; Tam I. Y. et al. (2006), Clin. Cancer Res. 12(5): 1647-53; and Ratner, E. et al. (2010) Cancer Res 70(16): OF1-OF7. Non-limiting examples of alterations in a KRAS gene is selected from the group consisting of KRAS_G12C, KRAS_G12R, KRAS_G12D, KRAS_G12A, KRAS_G12S, KRAS_G12V, KRAS_G13D, KRAS_G13S, KRAS_G13C, KRAS_G13V, KRAS_Q61H, KRAS_Q61R, KRAS_Q61P, KRAS_Q61L, KRAS_Q61K, KRAS_Q61E, KRAS_A59T and KRAS_G12F.

Examples of PIK3CA mutations are described in e.g., Samuels Y. et al. (2004) Science 304(5670):554; Kurds E. et al. (2004) Cancer Biology & Therapy 3(8):772-775; Stemke-Hale K. et al. (2008) Cancer Res. 68(15):6084-91.

Examples of mutations in RAF (e.g., one or more of A-Raf, B-Raf (BRAF) or C-Raf) gene or gene product include, but are not limited to, a mutation in codon 600 of B-Raf. Examples of BRAF mutations are described in e.g., Davies H. et al. (2002) Nature 417: 949-954. Exemplary alterations in the BRAF gene or gene product, include but are not limited to, BRAF_D594G, BRAF_D594V, BRAF_F468C, BRAF_F595L, BRAF G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E, BRAF_K601N, BRAF L597Q, BRAF_L597R, BRAF_L597S, BRAF_L597V, BRAF_T599I, BRAF_V600E, BRAF_V600K, BRAF_V600L, and BRAF_V600R.

Examples of PTEN mutations are described in, e.g., Minaguchi T. et al. (2001) Clin Cancer Res. 7(9):2636-42; Latta E. et al. (2002) Curr Opin Obstet. Gynecol. 14(1):59-65; Eng C. (2003) Hum Mutat. 22(3):183-98; Konopka B. et al. (2002) Cancer Lett. 178(1):43-51; Stemke-Hale K. et al. (2008) Cancer Res. 68(15):6084-91.

Examples of AKT mutations are described in, e.g., Stemke-Hale K. et al. (2008) Cancer Res. 68(15):6084-91; Davies M. A. et al. (2008) Br J. Cancer. 99(8):1265-8; Askham J. M. (2010) Oncogene 29(1):150-5; Shoji K. et al (2009) Br J. Cancer. 101(1):145-8.

Examples of TP53 mutations are described in, e.g., Soussi T. (2007) Cancer Cell 12(4):303-12; Cheung K. J. (2009) Br J Haematol. 146(3):257-69; Pfeifer G. P. et al. (2009) Hum Genet. 125(5-6):493-506; Petitjean A. et al. (2007) Oncogene 26(15):2157-65.

Examples of CTNNB1 (beta-catenin)mutations are described in, e.g., Polakis P. et al. (2000) Genes Dev. 14(15):1837-51; Miyaki M. et al. (1999) Cancer Res. 59(18):4506-9; Tejpar S. et al. (1999) Oncogene 18(47):6615-20; Garcia-Rostan G. et al. (1999) Cancer Res. 59(8):1811-5; Chan E. F. et al. (1999) Nat. Genet. 21(4):410-3; Legoix P. et al. (1999) Oncogene 18(27):4044-6; Mirabelli-Primdahl L. et al. (1999) Cancer Res. 59(14):3346-51.

Examples of NOTCH mutations are described in, e.g., Collins B. J. et al. (2004) Semin Cancer Biol. 14(5):357-64; Callahan R. et al. (2001) J Mammary Gland Biol Neoplasia. 6(1):23-36; Mansour M. R. et al. (2006) Leukemia 20:537-539; de Celis J. F. et al. (1993) Proc Natl Acad Sci USA. 90(9):4037-41.

Examples of FLT3 mutations are described in, e.g., Kiyoi H. et al. (2006) Methods Mol. Med. 125:189-97; Small D. (2006) Hematology Am Soc Hematol Educ Program. 2006:178-84; Kiyoi H. et al. (2006) Int J Hematol. 2006 May; 83(4):301-8; Schnittger S. et al. (2004) Acta Haematol. 112(1-2):68-78.

Examples of ERBB2 mutations are described in, e.g., U.S. Patent Application Publication Number 2008/0206248; Lee J. W. et al. (2006) Clin Cancer Res. 12(1):57-61; Lee J. W. et al. (2006) Cancer Lett. 237(1):89-94; Cancer Genome Atlas Research Network (2008) Nature 455(7216):1061-8.

Examples of HSP90AA1 mutations are described in, e.g., Cancer Genome Atlas Research Network (2008) Nature 455(7216):1061-8; Parsons D. W. et al. (2008) Science 321; 1807-12; Sjöblom T. et al. (2006) Science 314; 268-74.

Examples of HSP90AB1 mutations are described in, e.g., Dalgliesh G. L. et al. (2010) Nature 463; 360-3; Parsons D. W. et al. (2008) Science 321; 1807-12; Sjöblom T. et al. (2006) Science 314; 268-74.

Examples NF1 mutations are described in, e.g., Thomson S. A. et al. (2002) J Child Neurol. 17(8):555-61; Bottillo I. et al. (2009) J. Pathol. 217(5):693-701; Kluwe L. et al. (2003) J Med. Genet. 40(5):368-71.

Examples of STK11 mutations are described in, e.g., Resta N. et al. (1998) Cancer Res. 58(21):4799-801; Nishioka Y. et al. (1999) Jpn J Cancer Res. 90(6):629-32; Marignani P. A. (2005) J Clin Pathol. 58(1):15-9; Katajisto P. et al. (2007) Biochim Biophys Acta. 1775(1):63-75.

Any oncogenic alteration known in the art can be evaluated or treated using the methods of the invention are known in the art.

The results of the screening method and the interpretation thereof are predictive of the patient's response to treatment with HSP90 inhibiting agents (e.g., IPI-493 and/or IPI-504), alone or in combination. According to the present invention, the presence of one or oncogenic alterations in a gene or gene product, e.g., an ALK and/or a MAPK pathway mutation, is indicative that treatment with HSP90 inhibiting agents (e.g., IPI-493 and/or IPI-504), alone or in combination, will provide enhanced therapeutic benefit against the cancer cells relative to those of patients not having the mutation.

As discussed further herein, a variety of methods and techniques that are well known in the art can be used for the screening analysis, including metaphase cytogenetic analysis by standard karyotype methods, FISH, spectral karyotyping or MFISH, and comparative genomic hybridization.

In one embodiment, the methods of the present invention comprise contacting a DNA sample, e.g., a genomic DNA sample, such as a chromosomal sample, obtained from cells isolated from the patient to polynucleotide probes that are specific for and hybridize under stringent conditions with genomic DNA in chromosomal regions associated with cytogenetic abnormalities (e.g., the mutations described herein) to determine the presence or absence of one or more of the abnormalities in the cells of the patient. The results of the analysis are predictive of the patient's likely response to treatment with therapeutic agents, particularly agents that inhibit HSP90 (e.g., IPI-493 and/or IPI-504), alone or in combination with an mTOR inhibitor.

In yet another embodiment, the one or more alterations, e.g., alterations in ALK or MAPK pathway (e.g., K-Ras) are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to metastatic disease. In another embodiment, the significant event is the progression from primary diagnosis to relapse. In another embodiment, the significant event is the progression from metastatic disease to death. In another embodiment, the significant event is the progression from metastatic disease to relapse. In another embodiment, the significant event is the progression from relapse to death. In certain embodiments, the time course is measured with respect to one or more overall survival rate, time to progression and/or using the RECIST or other response criteria.

In certain embodiments, a pre-determined measure or value is created by dividing patient samples into at least two patient subgroups. In certain embodiments, the number of subgroups is two so that the patient sample is divided into a subgroup of patients having the one or more oncogenic abnormalities, e.g., an ALK or MAPK pathway (e.g., K-Ras) mutation(s), and a subgroup not having the oncogenic abnormalities. In certain embodiments, the ALK mutation or MAPK pathway (e.g., K-Ras) status in the subject is compared to either the subgroup having or not having an ALK or MAPK pathway (e.g., K-Ras) mutation(s); if the patient has a mutation(s) in an ALK or MAPK pathway (e.g., K-Ras), then the patient is likely to respond to an HSP90 inhibitor (e.g., IPI-493 and/or IPI-504), alone or in combination, and/or the patient has an increased likelihood, or is likely, to have a long time course. In certain embodiments, the number of subgroups is greater than two, including, without limitation, three subgroups, four subgroups, five subgroups and six subgroups, depending on stratification of predicted HSP90 inhibitor efficacy as correlated with particular oncogenic abnormalities, e.g., ALK or MAPK pathway (e.g., K-Ras) mutations. In certain embodiments, likelihood to respond is measured with respect to overall survival rate, time to progression and/or using the RECIST criteria.

In other embodiments, the methods further include one or more of: determining whether a subject with a cancer or tumor having an alteration described herein, e.g., an alteration in an ALK or MAPK pathway (e.g., K-Ras), is likely to respond to treatment with an HSP90 inhibitor (e.g., IPI-493 and/or IPI-504), alone or in combination; determining a treatment regimen (e.g., altering the course of therapy, dosing, treatment schedule or time course, combination therapies). The method can be used to predict a time course of the cancer in a subject. In other embodiments, the method is used to predict the probability of a significant event in the subject with cancer.

1. Methods for Detection of Gene Mutations

Methods of evaluating gene, mutations and/or gene products (e.g., one or more of the markers set forth in Table 1, Table 5, or disclosed herein) are well known to those of skill in the art, including hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the presence/absence and relative copy number of the target nucleic acid. Alternatively, a Northern blot can be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the presence/absence and relative copy number of the target nucleic acid.

An alternative means for determining the copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

Exemplary hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g., membrane or glass) bound methods or array-based approaches.

In one aspect, FISH analysis is used. Cell samples are obtained from patients according to methods well known in the art in order to be tested by an appropriate cytogenetic testing method known in the art, for example, the FISH method. In one embodiment, FISH can be performed according to the Vysis™ system (Abbott Molecular), whose manufacturer's protocols are incorporated herein by reference.

Probes are used that contain DNA segments that are essentially complementary to DNA base sequences existing in different portions of chromosomes. Examples of probes useful according to the invention, and labeling and hybridization of probes to samples are described in two U.S. patents to Vysis, Inc. U.S. Pat. Nos. 5,491,224 and 6,277,569 to Bittner, et al.

Chromosomal probes are typically about 50 to about 10⁵ nucleotides in length. Longer probes typically comprise smaller fragments of about 100 to about 500 nucleotides in length. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromsome) along with the normal chromosome complement of the host, and chromosomes purified by flow cytometry or microdis section. The region of interest can be isolated through cloning, or by site-specific amplification via the polymerase chain reaction (PCR). See, for example, Nath and Johnson, Biotechnic Histochem., 1998, 73(1):6-22, Wheeless et al., Cytometry 1994, 17:319-326, and U.S. Pat. No. 5,491,224.

The probes to be used hybridize to a specific region of a chromosome to determine whether a cytogenetic abnormality is present in this region. One type of cytogenetic abnormality is a deletion. Although deletions can be of one or more entire chromosomes, deletions normally involve loss of part of one or more chromosomes. If the entire region of a chromosome that is contained in a probe is deleted from a cell, hybridization of that probe to the DNA from the cell will normally not occur and no signal will be present on that chromosome. If the region of a chromosome that is partially contained within a probe is deleted from a cell, hybridization of that probe to the DNA from the cell can still occur, but less of a signal can be present. For example, the loss of a signal is compared to probe hybridization to DNA from control cells that do not contain the genetic abnormalities which the probes are intended to detect. In some embodiments, at least 1, 5, 10, 20, 30, 40, 50, 60, 70, 80, 90, 100, 110, 120, 130, 140, 150, 160, 170, 180, 190, 200, or more cells are enumerated for presence of the cytogenetic abnormality.

Cytogenetic abnormalities to be detected can include, but are not limited to, non-reciprocal translocations, intra-chromosomal inversions, point mutations, deletions, gene copy number changes, gene expression level changes, and germ line mutations. In particular, one type of cytogenetic abnormality is a duplication. Duplications can be of entire chromosomes, or of regions smaller than an entire chromosome. If the region of a chromosome that is contained in a probe is duplicated in a cell, hybridization of that probe to the DNA from the cell will normally produce at least one additional signal as compared to the number of signals present in control cells with no abnormality of the chromosomal region contained in the probe. Although any probes that detect human chromosome 2p23 or ortholog thereof or any chromosomal region comprising a translocation with the ALK gene of 2p23 or ortholog thereof can be used. Suitable probes are well known in the art (e.g., available from Vysis, Inc. (Downers Grove, Ill.).

Chromosomal probes are labeled so that the chromosomal region to which they hybridize can be detected. Probes typically are directly labeled with a fluorophore, an organic molecule that fluoresces after absorbing light of lower wavelength/higher energy. The fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, U.S. Pat. No. 5,491,224.

U.S. Pat. No. 5,491,224 describes probe labeling as a number of the cytosine residues having a fluorescent label covalently bonded thereto. The number of fluorescently labeled cytosine bases is sufficient to generate a detectable fluorescent signal while the individual so labeled DNA segments essentially retain their specific complementary binding (hybridizing) properties with respect to the chromosome or chromosome region to be detected. Such probes are made by taking the unlabeled DNA probe segment, transaminating with a linking group a number of deoxycytidine nucleotides in the segment, covalently bonding a fluorescent label to at least a portion of the transaminated deoxycytidine bases.

Probes can also be labeled by nick translation, random primer labeling or PCR labeling. Labeling is done using either fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP or Dinitrophenyl (DNP)-11-dUTP.

Probes also can be indirectly labeled with biotin or digoxygenin, or labeled with radioactive isotopes such as ³²P and .³H, although secondary detection molecules or further processing then is required to visualize the probes. For example, a probe labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Probes can also be prepared such that a fluorescent or other label is not part of the DNA before or during the hybridization, and is added after hybridization to detect the probe hybridized to a chromosome. For example, probes can be used that have antigenic molecules incorporated into the DNA. After hybridization, these antigenic molecules are detected using specific antibodies reactive with the antigenic molecules. Such antibodies can themselves incorporate a fluorochrome, or can be detected using a second antibody with a bound fluorochrome.

However treated or modified, the probe DNA is commonly purified in order to remove unreacted, residual products (e.g., fluorochrome molecules not incorporated into the DNA) before use in hybridization.

Prior to hybridization, chromosomal probes are denatured according to methods well known in the art. In general, hybridization steps comprise adding an excess of blocking DNA to the labeled probe composition, contacting the blocked probe composition under hybridizing conditions with the chromosome region to be detected, e.g., on a slide where the DNA has been denatured, washing away unhybridized probe, and detecting the binding of the probe composition to the chromosome or chromosomal region.

Probes are hybridized or annealed to the chromosomal DNA under hybridizing conditions. “Hybridizing conditions” are conditions that facilitate annealing between a probe and target chromosomal DNA. Since annealing of different probes will vary depending on probe length, base concentration and the like, annealing is facilitated by varying probe concentration, hybridization temperature, salt concentration and other factors well known in the art.

Hybridization conditions are facilitated by varying the concentrations, base compositions, complexities, and lengths of the probes, as well as salt concentrations, temperatures, and length of incubation. For example, in situ hybridizations are typically performed in hybridization buffer containing 1-2×SSC, 50-65% formamide and blocking DNA to suppress non-specific hybridization. In general, hybridization conditions, as described above, include temperatures of about 25° C. to about 55° C., and incubation lengths of about 0.5 hours to about 96 hours.

Non-specific binding of chromosomal probes to DNA outside of the target region can be removed by a series of washes. Temperature and concentration of salt in each wash are varied to control stringency of the washes. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes. In some applications it is necessary to block the hybridization capacity of repetitive sequences. Thus, in some embodiments, tRNA, human genomic DNA, or Cot-I DNA is used to block non-specific hybridization.

After washing, the slide is allowed to drain and air dry, then mounting medium, a counterstain such as DAPI, and a coverslip are applied to the slide. Slides can be viewed immediately or stored at −20° C. before examination.

For fluorescent probes used in fluorescence in situ hybridization (FISH) techniques, fluorescence can be viewed with a fluorescence microscope equipped with an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the chromosomal probes. FISH can be used to detect chromosome copy number or rearrangement of regions of chromosomes. These probes hybridize, or bind, to the complementary DNA and, because they are labeled with fluorescent tags, allow researchers to see the location of those sequences of DNA using a fluorescence microscope. Unlike most other techniques used to study chromosomes, which require that the cells be actively dividing, FISH can also be performed on non-dividing cells, making it a highly versatile procedure. Therefore, FISH can be performed using interphase cells, or cells in metaphase of the cell division cycle. Many of the techniques involved in FISH analysis are described in U.S. Pat. No. 5,447,841 by Gray and Pinkel.

FISH results can be interpreted with reference to control cells that are known not to contain the specific cytogenetic abnormality the probe is designed to detect. The FISH hybridization pattern of the probe to DNA from the control cells is compared to hybridization of the same probe to the DNA from cells that are being tested or assayed for the specific cytogenetic abnormality. When a probe is designed to detect a deletion of a chromosome or chromosomal region, there normally is less hybridization of the probe to DNA from the cells being tested than from the control cells. Normally, there is absence of a probe signal in the tested cells, indicative of loss of the region of a chromosome the probe normally hybridizes to. When a probe is designed to detect a chromosomal duplication or addition, there normally is more hybridization of the probe to DNA from the cells being tested than from the control cells. Normally, there is addition of a probe signal in the tested cells, indicative of the presence of an additional chromosomal region that the probe normally hybridizes to.

In CGH methods, a first collection of nucleic acids (e.g., from a sample, e.g., a possible tumor) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH can also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl. Acad Sci USA 89:5321-5325 (1992) is used. Array-based CGH is described in U.S. Pat. No. 6,455,258, the contents of each of which are incorporated herein by reference.

In still another embodiment, amplification-based assays can be used to measure presence/absence and copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g., healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR can also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) can also be used to identify regions of amplification or deletion.

2. Methods for Detection of Gene Expression

Marker expression level can also be assayed. Expression of a marker of the invention can be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that can contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

In another embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Exemplary nucleic acid probes are 20 bases or longer in length (See, e.g., Sambrook et al. for methods of selecting nucleic acid probe sequences for use in nucleic acid hybridization). Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a marker, the level of expression of the marker is determined for 10 or more samples of normal versus cancer cell isolates, or even 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In certain embodiments, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

In another embodiment, expression of a marker is assessed by preparing genomic DNA or mRNA/cDNA (i.e., a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g., at least 7, at least 10, at least 15, at least 20, at least 25, at least 30, at least 40, at least 50, at least 100, at least 500, or more nucleotide residues) of a marker of the invention. If polynucleotides complementary to or homologous with a marker of the invention are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, the hybridization can be performed under stringent hybridization conditions.

In another embodiment, a combination of methods to assess the expression of a marker is utilized.

Because the compositions, kits, and methods of the invention rely on detection of a difference in expression levels or copy number of one or more markers of the invention, in certain embodiments the level of expression or copy number of the marker is significantly greater than the minimum detection limit of the method used to assess expression or copy number in at least one of normal cells and cancerous cells.

3. Methods for Detection of Expressed Protein

The activity or level of a marker protein can also be detected and/or quantified by detecting or quantifying the expressed polypeptide. The polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining whether cells express a marker of the present invention.

Another agent for detecting a polypeptide of the invention is an antibody capable of binding to a polypeptide corresponding to a marker of the invention, e.g., an antibody with a detectable label. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Immunohistochemistry or IHC refers to the process of localizing antigens (e.g. proteins) in cells of a tissue section exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used in research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as fluorescein, rhodamine, DyLight Fluor or Alexa Fluor.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies can be directly labeled or alternatively can be subsequently detected using labeled antibodies (e.g., labeled sheep anti-human antibodies) that specifically bind to the anti-polypeptide.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In another embodiment, the capture agent is an antibody that specifically binds a polypeptide. The antibody (anti-peptide) can be produced by any of a number of means well known to those of skill in the art.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one embodiment, the labeling agent is a second human antibody bearing a label. Alternatively, the second antibody can lack a label, but it can, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Exemplary immunoassays for detecting a polypeptide can be competitive or noncompetitive. Noncompetitive immunoassays are assays in which the amount of captured analyte is directly measured. In one “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In another embodiment, the antibody is immobilized on a solid substrate. The amount of polypeptide bound to the antibody can be determined either by measuring the amount of polypeptide present in a polypeptide/antibody complex, or alternatively by measuring the amount of remaining uncomplexed polypeptide. The amount of polypeptide can be detected by providing a labeled polypeptide.

The assays described herein are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described herein.

In another embodiment, level (activity) is assayed by measuring the enzymatic activity of the gene product. Methods of assaying the activity of an enzyme are well known to those of skill in the art.

In vivo techniques for detection of a marker protein include introducing into a subject a labeled antibody directed against the protein. For example, the antibody can be labeled with a radioactive marker whose presence and location in a subject can be detected by standard imaging techniques.

Certain markers identified by the methods of the invention can be secreted proteins. It is a simple matter for the skilled artisan to determine whether any particular marker protein is a secreted protein. In order to make this determination, the marker protein is expressed in, for example, a mammalian cell, e.g., a human cell line, extracellular fluid is collected, and the presence or absence of the protein in the extracellular fluid is assessed (e.g., using a labeled antibody which binds specifically with the protein).

The following is an example of a method which can be used to detect secretion of a protein. About 8×10⁵ 293T cells are incubated at 37° C. in wells containing growth medium (Dulbecco's modified Eagle's medium {DMEM} supplemented with 10% fetal bovine serum) under a 5% (v/v) CO2, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵ S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

It will be appreciated that subject samples, e.g., a sample containing tissue, whole blood, serum, plasma, buccal scrape, saliva, cerebrospinal fluid, urine, stool, and bone marrow, can contain cells therein, particularly when the cells are cancerous, and, more particularly, when the cancer is metastasizing, and thus can be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the marker in the sample. Thus, the compositions, kits, and methods of the invention can be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods can be used to detect such proteins on whole cells, or well known computer-based sequence analysis methods (e.g., the SIGNALP program; Nielsen et al., 1997, Protein Engineering 10:1-6) can be used to predict the presence of at least one extracellular domain (i.e., including both secreted proteins and proteins having at least one cell-surface domain). Expression of a marker corresponding to a protein having at least one portion which is displayed on the surface of a cell which expresses it can be detected without necessarily lysing the cell (e.g., using a labeled antibody which binds specifically with a cell-surface domain of the protein).

The invention also encompasses kits for detecting the presence of a polypeptide or nucleic acid corresponding to a marker of the invention in a biological sample, e.g., a sample containing tissue, whole blood, serum, plasma, buccal scrape, saliva, cerebrospinal fluid, urine, stool, and bone marrow. Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

4. Method for Detecting Structural Alterations

The invention also provides a method for assessing the presence of a structural alteration, e.g., mutation.

Another detection method is allele specific hybridization using probes overlapping the polymorphic site and having about 5, about 10, about 20, about 25, or about 30 nucleotides around the polymorphic region. In another embodiment of the invention, several probes capable of hybridizing specifically to mutations are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the mutations of at least one polymorphic region of a gene. The solid phase support is then contacted with a test nucleic acid and hybridization to the specific probes is detected. Accordingly, the identity of numerous mutations of one or more genes can be identified in a simple hybridization experiment. For example, the identity of the mutation of the nucleotide polymorphism in the 5′ upstream regulatory element can be determined in a single hybridization experiment.

In other detection methods, it is necessary to first amplify at least a portion of a marker prior to identifying the mutation. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In certain embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a marker and detect mutations by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci. 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled DNA Sequencing by Mass Spectrometry Via Exonuclease Degradation by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

Other sequencing methods include, but not limited to, in vitro clonal amplification (e.g., as described in Margulies M. et al. (2005) Nature 437 (7057):376-380; Shendure J. (2005) Science 309:1728 (also known as Polony sequencing); SOLid™ sequencing (Applied Biosystem http://www.appliedbiosystems.com/absite/us/en/home/applications-technologies/solid-next-generation-sequencing.html); bridge amplification (Illumina http://www.illumina.com/technology/sequencing_technology.html); Braslaysky I. et al. (2003) Proc. Natl. Acad. Sci. U.S.A. 100(7):3960-3964), parallelized sequencing (e.g., as described in Margulies M. et al. (2005) Nature 437 (7057):376-380; Ronaghi M. et al. (1996) Analytical Biochemistry 242(1):84-89; reversible terminator methods (e.g., used by Illumina and Helicos); pyrosuencing (e.g., used by 454 Life Sciences), sequencing by ligation (e.g., as described in Shendure J. (2005) Science 309:1728; SOLid™ sequencing (Applied Biosystem http://www.appliedbiosystems.com/absite/us/en/home/applications-technologies/solid-next-generation-sequencing.html); U.S. Pat. No. 5,750,341 entitled “DNA sequencing by parallel oligonucleotide extentions”), microfluidic Sanger sequencing, sequencing by hybridization (e.g., non-enzymatic method that uses a DNA microarray as described in Hanna G. J. et al. (2000) J. Clin. Microbiol. 38(7):2715-2721); microscopy-based techniques (e.g., as described in U.S. Patent Application Publication Number 2006/0029957).

In some cases, the presence of a specific allele of a marker in DNA from a subject can be shown by restriction enzyme analysis. For example, a specific nucleotide polymorphism can result in a nucleotide sequence comprising a restriction site which is absent from the nucleotide sequence of another mutation.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a marker mutation with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl. Acad Sci USA 85:4397; Saleeba et al (1992) Methods Enzymol. 217:286-295. In another embodiment, the control or sample nucleic acid is labeled for detection.

In another embodiment, an mutation can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility are used to identify the type of marker mutation. For example, single strand conformation polymorphism (SSCP) can be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of a mutation of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes can be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci. USA 86:6230; and Wallace et al. (1979) Nucl. Acids Res. 6:3543). Such allele specific oligonucleotide hybridization techniques can be used for the simultaneous detection of several nucleotide changes in different polymorphic regions of marker. For example, oligonucleotides having nucleotide sequences of specific mutations are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it can be desirable to introduce a novel restriction site in the region of the mutation to create cleavage-based detection (Gasparini et al (1992) Mol. Cell. Probes 6:1).

In another embodiment, identification of the mutation is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

The invention further provides methods for detecting single nucleotide polymorphisms in a marker. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087), the method of Goelet, P. et al. is a heterogeneous phase assay, in which the primer or the target molecule is immobilized to a solid phase.

Several primer-guided nucleotide incorporation procedures for assaying polymorphic sites in DNA have been described (Komher, J. S. et al., (1989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mutat. 1:159-164; Ugozzoli, L. et al., (1992) GATA 9:107-112; Nyren, P. (1993) et al., Anal. Biochem. 208:171-175). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the mutation of a polymorphic region located in the coding region of a marker, yet other methods than those described above can be used. For example, identification of a mutation which encodes a mutated marker can be performed by using an antibody specifically recognizing the mutant protein in, e.g., immunohistochemistry or immunoprecipitation. Antibodies to wild-type markers or mutated forms of markers can be prepared according to methods known in the art.

Alternatively, one can also measure an activity of a marker, such as binding to a marker ligand. Binding assays are known in the art and involve, e.g., obtaining cells from a subject, and performing binding experiments with a labeled ligand, to determine whether binding to the mutated form of the protein differs from binding to the wild-type of the protein.

### VII. HSP90-Inhibiting Therapeutic Agents, Compositions and Administration

HSP90-inhibiting agents for therapeutic purposes are known in the art. HSP90-inhibiting agents include each member of the family of heat shock proteins having a mass of about 90-kiloDaltons. For example, in humans the highly conserved Hsp90 family includes cytosolic Hsp90α and Hsp90β isoforms, as well as GRP94, which is found in the endoplasmic reticulum, and HSP75/TRAP1, which is found in the mitochondrial matrix.

Representative, non-limiting examples include HSP90 inhibitors selected from the group consisting of IPI-493 (Infinity Pharm.), IPI-504 (Infinity Pharm.), 17-AAG (also known as tanespimycin or CNF-1010; BMS), BIIB-021 (also known as CNF-2024, Biogen IDEC), BIIB-028 (Biogen IDEC), AUY-922 (also known as VER-49009, Novartis), SNX-5422 (Pfizer), STA-9090, AT-13387 (Astex), XL-888 (Exelixis), MPC-3100 (Myriad), CU-0305 (Curis), 17-DMAG, CNF-1010, a Macbecin (e.g., Macbecin I, Macbecin II), CCT-018159, CCT-129397, PU-H71 (Memorial Sloan Kettering Cancer Center), and PF-04928473 (SNX-2112). Other HSP90 inhibitors are disclosed in Zhang, M-Q. et al., J. Med. Chem. 51(18):5494-5497 (2008) and Menzella, H. et al., J. Med. Chem., 52(6):15128-1521 (2009), the entire contents of which are incorporated herein by reference.

1. IPI-504

Compositions, methods of synthesis, methods of administration, etc. for IPI-504 can be found in the art in PCT application WO2005/063714, the entire contents of which is incorporated by reference.

The present invention also provides the isolated analogs of benzoquinone-containing ansamycins, wherein the benzoquinone is reduced to a hydroquinone and trapped as the ammonium salt by reaction of the hydroquinone with a suitable organic or inorganic acid.

In one embodiment, the present invention provides a pure and isolated compound of formula 1:

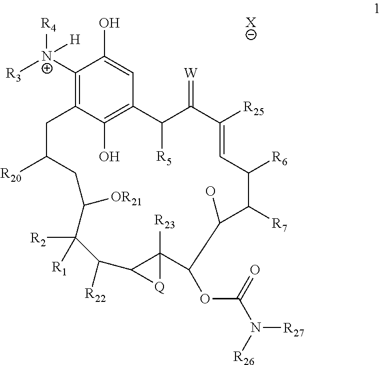

or the free base thereof;

wherein independently for each occurrence:

W is oxygen or sulfur;

Q is oxygen, NR, N(acyl) or a bond;

X⁻ is a conjugate base of a pharmaceutically acceptable acid;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₃ taken together with R₄ represent a 4-8 membered optionally substituted heterocyclic ring;

R₅ is selected from the group consisting of H, alkyl, aralkyl, and a group having the formula 1a:

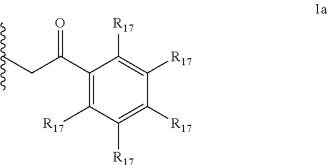

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₉ is alkyl, alkenyl, alkynyl, aryl, cycloalkyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(CR₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —C(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(R₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl;

provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and

the absolute stereochemistry at a stereogenic center of formula 1 can be R or S or a mixture thereof and the stereochemistry of a double bond can be E or Z or a mixture thereof.

In certain embodiments, the present invention relates to the aforementioned compound and the attendant definitions, provided that when R₁ is hydroxyl, R₂ is hydrogen, R₅ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine.

In certain embodiments, the present invention relates to the aforementioned compound and the attendant definitions, wherein R₂₀, R₂₁, R₂₂, R₂₃, R₂₄, and R₂₅ are methyl; R₂₆ is hydrogen, Q is a bond; and W is oxygen.

In certain embodiments, the present invention relates to the aforementioned compound and the attendant definitions, wherein said pharmaceutically acceptable acid has a pKa between about −10 and about 7 in water.

In certain embodiments, the present invention relates to the aforementioned compound and the attendant definitions, wherein said pharmaceutically acceptable acid has a pKa between about −10 and about 4 in water.

In certain embodiments, the present invention relates to the aforementioned compound and the attendant definitions, wherein said pharmaceutically acceptable acid has a pKa between about −10 and about 1 in water.

In certain embodiments, the present invention relates to the aforementioned compound and the attendant definitions, wherein said pharmaceutically acceptable acid has a pKa between about −10 and about −3 in water.

In certain embodiments, the present invention relates to the aforementioned compound and the attendant definitions, wherein X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

In certain embodiments, the present invention relates to the aforementioned compound and the attendant definitions, wherein R₁ is hydroxyl or —OC(O)R₈.

In certain embodiments, the present invention relates to the aforementioned compound and the attendant definitions, wherein R₂ is hydrogen.

In certain embodiments, the present invention relates to the aforementioned compound and the attendant definitions, wherein R₃ and R₄ are independently hydrogen, alkyl, alkenyl, alkynyl, cycloalkyl, aralkyl, heteroaralkyl, or —[(CR₂)ₚ]—R₁₆.

In certain embodiments, the present invention relates to the aforementioned compound and the attendant definitions, wherein R₅ is hydrogen or has a formula 1a:

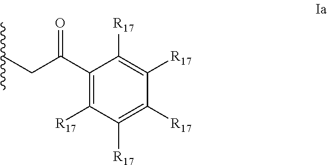

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl.

In certain embodiments, the present invention relates to the aforementioned compound and the attendant definitions, wherein R₆ and R₇ taken together form a double bond.

In certain embodiments, the present invention relates to the aforementioned compound and the attendant definitions, wherein R₂₇ is hydrogen.

In certain embodiments, the present invention relates to the aforementioned compound and the attendant definitions, wherein R₁ is hydroxyl or —OC(O)R₈; and R₂ is hydrogen.

In certain embodiments, the present invention relates to the aforementioned compound and the attendant definitions, wherein R₁ is hydroxyl or —OC(O)R₈; R₂ is hydrogen; and R₃ and R₄ are independently hydrogen, alkyl, alkenyl, alkynyl, cycloalkyl, aralkyl, heteroaralkyl, or —[(CR₂)ₚ]—R₁₆.

In certain embodiments, the present invention relates to the aforementioned compound and the attendant definitions, wherein R₁ is hydroxyl or —OC(O)R₈; R₂ is hydrogen; R₃ and R₄ are independently hydrogen, alkyl, alkenyl, alkynyl, cycloalkyl, aralkyl, heteroaralkyl, or —[(CR₂)ₚ]—R₁₆; and R₅ is hydrogen or has a formula 1a:

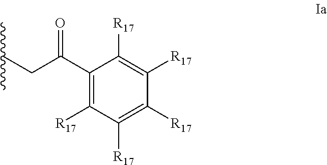

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl.

In certain embodiments, the present invention relates to the aforementioned compound and the attendant definitions, wherein R₁ is hydroxyl or —OC(O)R₈; R₂ is hydrogen; R₃ and R₄ are independently hydrogen, alkyl, alkenyl, alkynyl, cycloalkyl, aralkyl, heteroaralkyl, or —[(CR₂)ₚ]—R₁₆; R₅ is hydrogen or has a formula 1a:

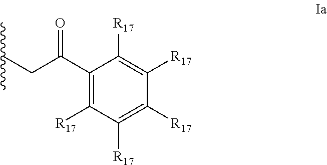

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; and R₆ and R₇ taken together form a double bond.

In certain embodiments, the present invention relates to the aforementioned compound and the attendant definitions, wherein R₁ is hydroxyl or —OC(O)R₈; R₂ is hydrogen; R₃ and R₄ are independently hydrogen, alkyl, alkenyl, alkynyl, cycloalkyl, aralkyl, heteroaralkyl, or —[(CR₂)ₚ]—R₁₆; R₅ is hydrogen or has a formula 1a:

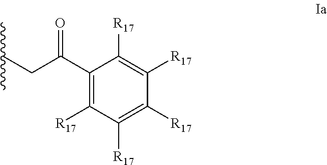

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; R₆ and R₇ taken together form a double bond; and R₂₇ is hydrogen.

In certain embodiments, the present invention relates to the aforementioned compound and the attendant definitions, wherein R₁ is hydroxyl or —OC(O)R₈; R₂ is hydrogen; R₃ and R₄ are independently hydrogen, alkyl, alkenyl, alkynyl, cycloalkyl, aralkyl, heteroaralkyl, or —[(CR₂)ₚ]—R₁₆; R₅ is hydrogen or has a formula 1a:

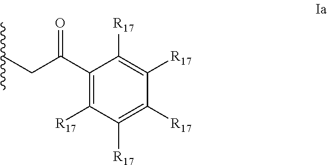

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₉)SO₂R₁₉, —N(R₁₈)C(O)N(R₁₈)(R₁₉), and —CH₂O-heterocyclyl; R₆ and R₇ taken together form a double bond; R₂₇ is hydrogen; and X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

In certain embodiments, the present invention relates to the aforementioned compound and the attendant definitions, wherein R₁ is hydroxyl or —OC(O)R₈; R₂ is hydrogen; R₃ and R₄ are independently hydrogen, alkyl, alkenyl, alkynyl, cycloalkyl, aralkyl, heteroaralkyl, or —[(CR₂)ₚ]—R₁₆; R₅ is hydrogen or has a formula 1a:

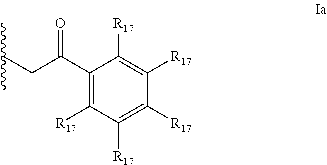

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; R₆ and R₇ taken together form a double bond; R₂₇ is hydrogen; and X⁻ is selected from the group consisting of chloride and bromide.

In one embodiment the present invention provides a pure and isolated compound with absolute sterochemistry as shown in formula 2:

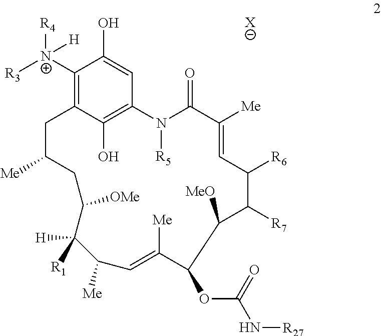

or the free base thereof;

wherein independently for each occurrence:

X⁻ is selected from the group consisting of chloride, bromide, iodide, H₂PO₄⁻, HSO₄⁻, methylsulfonate, benzenesulfonate, p-toluenesulfonate, trifluoromethylsulfonate, 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

R₁ is hydroxyl or —OC(O)R₈;

R₃ and R₄ are hydrogen, alkyl, alkenyl, alkynyl, cycloalkyl, aralkyl, heteroaralkyl, or —[(CR₂)ₚ]—R₁₆; or R₃ taken together with R₄ represent a 4-8 membered optionally substituted heterocyclic ring;

R₅ is hydrogen or has a formula 1a:

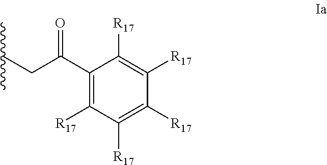

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₇ is hydrogen, alkyl, aryl, cycloalkyl, heterocycloalkyl, aralkyl, heteroaryl, or heteroaralkyl;

provided that when R₁ is hydroxyl, R₂ is hydrogen, R₆ and R₇ taken together form a double bond, R₂₇ is hydrogen; R₃ and R₄ are not both hydrogen nor when taken together represent an unsubstituted azetidine; and

the stereochemistry of a double bond can be E or Z or a mixture thereof.

In certain embodiments, the present invention relates to the aforementioned compound and the attendant definitions, provided that when R₁ is hydroxyl, R₅ is hydrogen, R₆ and R₇ taken together form a double bond, R₂₇ is hydrogen; R₃ and R₄ are not both hydrogen nor when taken together represent an unsubstituted azetidine.

In certain embodiments, the present invention relates to the aforementioned compound and the attendant definitions, wherein R₁ is hydroxyl.

In certain embodiments, the present invention relates to the aforementioned compound and the attendant definitions, wherein R₃ is allyl.

In certain embodiments, the present invention relates to the aforementioned compound and the attendant definitions, wherein R₃ has formula 9

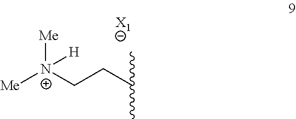

or the free base thereof;

wherein X₁⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

In certain embodiments, the present invention relates to the aforementioned compound and the attendant definitions, wherein R₄ is hydrogen.

In certain embodiments, the present invention relates to the aforementioned compound and the attendant definitions, wherein R₅ is hydrogen.

In certain embodiments, the present invention relates to the aforementioned compound and the attendant definitions, wherein R₆ and R₇ taken together form a bond.

In certain embodiments, the present invention relates to the aforementioned compound and the attendant definitions, wherein R₂₇ is hydrogen.

In certain embodiments, the present invention relates to the aforementioned compound and the attendant definitions, wherein R₁ is hydroxyl; R₃ is allyl; and R₄ is hydrogen.

In certain embodiments, the present invention relates to the aforementioned compound and the attendant definitions, wherein R₁ is hydroxyl; R₃ has formula 9

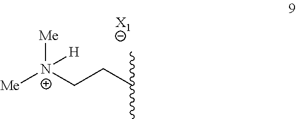

or the free base thereof;

wherein X₁⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate; and R₄ is hydrogen.

In certain embodiments, the present invention relates to the aforementioned compound and the attendant definitions, wherein R₁ is hydroxyl; R₃ is allyl; R₄ is hydrogen; and R₅ is hydrogen.

In certain embodiments, the present invention relates to the aforementioned compound and the attendant definitions, wherein R₁ is hydroxyl; R₃ has formula 9

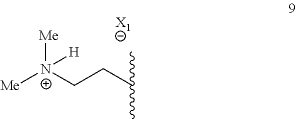

or the free base thereof;

wherein X₁⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate; R₄ is hydrogen; and R₅ is hydrogen.

In certain embodiments, the present invention relates to the aforementioned compound and the attendant definitions, wherein R₁ is hydroxyl; R₃ is allyl; R₄ is hydrogen; R₅ is hydrogen; and R₆ and R₇ taken together form a bond.

In certain embodiments, the present invention relates to the aforementioned compound and the attendant definitions, wherein R₁ is hydroxyl; R₃ has formula 9

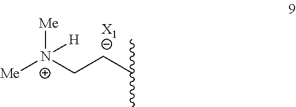

or the free base thereof;

wherein X₁⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate; R₄ is hydrogen; R₅ is hydrogen; and R₆ and R₇ taken together form a bond.

In certain embodiments, the present invention relates to the aforementioned compound and the attendant definitions, wherein R₁ is hydroxyl; R₃ is allyl; R₄ is hydrogen; R₅ is hydrogen; R₆ and R₇ taken together form a bond; and R₂₇ is hydrogen.

In certain embodiments, the present invention relates to the aforementioned compound and the attendant definitions, wherein R₁ is hydroxyl; R₃ has formula 9

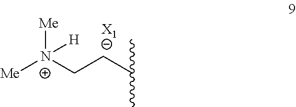

or the free base thereof;

wherein X₁⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate; R₄ is hydrogen; R₅ is hydrogen; R₆ and R₇ taken together form a bond; and R₂₇ is hydrogen.

In one embodiment the present invention provides a pure and isolated compound with absolute sterochemistry as shown in formula 3:

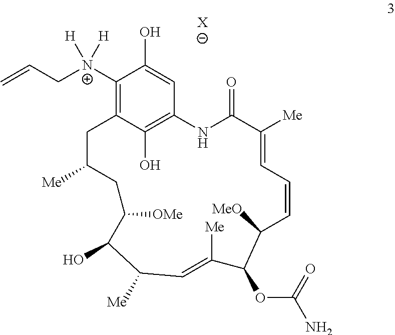

wherein X⁻ is selected from the group consisting of chloride, bromide, iodide, H₂PO₄⁻, HSO₄⁻, methylsulfonate, benzenesulfonate, p-toluenesulfonate, trifluoromethylsulfonate, and 10-camphorsulfonate, naphthalene-1-sulfonic acid-5-sulfonate, ethan-1-sulfonic acid-2-sulfonate, cyclamic acid salt, thiocyanic acid salt, naphthalene-2-sulfonate, and oxalate.

In certain embodiments, the present invention relates to the aforementioned compound and the attendant definitions, wherein X⁻ is chloride.

In certain embodiments, the present invention relates to the aforementioned compound and the attendant definitions, wherein X⁻ is bromide.

In one embodiment, the present invention relates to a composition comprising a compound of any one of the aforementioned compounds and an amino acid.

In certain embodiments, the present invention relates to the aforementioned composition and the attendant definitions, wherein the amino acid is selected from the group consisting of:

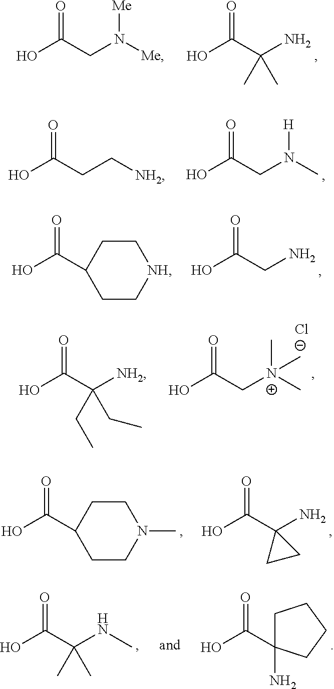

In one embodiment the present invention provides a compound of formula 4:

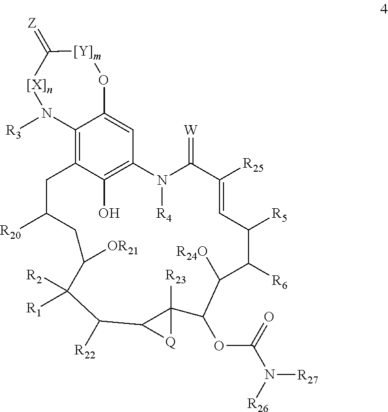

or a pharmaceutically acceptable salt thereof;

wherein, independently for each occurrence,

W is oxygen or sulfur;

Z is oxygen or sulfur;

Q is oxygen, NR, N(acyl) or a bond;

n is equal to 0, 1, or 2;

m is equal to 0, 1, or 2;

X and Y are independently C(R₃₀)₂; wherein R₃₀ for each occurrence is independently selected from the group consisting of hydrogen, alkyl, cycloalkyl, heterocycloalkyl, aralkyl, heteroaryl, and heteroaralkyl; or —[(CR₂)ₚ]—R₁₆;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆;

R₄ is selected from the group consisting of H, alkyl, aralkyl, and a group having the Formula 4a:

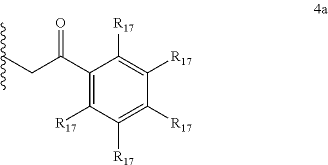

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;

R₅ and R₆ are both hydrogen; or R₅ and R₆ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₉ is alkyl, alkenyl, alkynyl, aryl, cycloalkyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(CR₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl; and

the absolute stereochemistry at a stereogenic center of formula 4 can be R or S or a mixture thereof and the stereochemistry of a double bond can be E or Z or a mixture thereof.

In certain embodiments, the present invention relates to the aforementioned compound and the attendant definitions, wherein R₂₀, R₂₁, R₂₂, R₂₃, R₂₄, R₂₅ are methyl; R₂₆ is hydrogen; Q is a bond; and Z and W are oxygen.

In certain embodiments, the present invention relates to the aforementioned compound and the attendant definitions, wherein R₁ is hydroxyl or —OC(O)R₈.

In certain embodiments, the present invention relates to the aforementioned compound and the attendant definitions, wherein R₂ is hydrogen.

In certain embodiments, the present invention relates to the aforementioned compound and the attendant definitions, wherein R₃ is hydrogen, alkyl, alkenyl, cycloalkyl, aralkyl, heteroaralkyl, or —[(CR₂)ₚ]—R₁₆.

In certain embodiments, the present invention relates to the aforementioned compound and the attendant definitions, wherein R₄ is hydrogen or has a formula 1a:

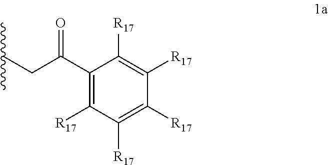

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl.

In certain embodiments, the present invention relates to the aforementioned compound and the attendant definitions, wherein R₅ and R₆ taken together form a bond.

In certain embodiments, the present invention relates to the aforementioned compound and the attendant definitions, wherein X and Y are —CH₂—.

In certain embodiments, the present invention relates to the aforementioned compound and the attendant definitions, wherein n is equal to 0; and m is equal to 0 or 1.

In certain embodiments, the present invention relates to the aforementioned compound and the attendant definitions, wherein R₁ is hydroxyl or —OC(O)R₈; and R₂ is hydrogen.

In certain embodiments, the present invention relates to the aforementioned compound and the attendant definitions, wherein R₁ is hydroxyl or —OC(O)R₈; R₂ is hydrogen; and R₃ is hydrogen, alkyl, alkenyl, cycloalkyl, aralkyl, heteroaralkyl, or —[(CR₂)ₚ]—R₁₆.

In certain embodiments, the present invention relates to the aforementioned compound and the attendant definitions, wherein R₁ is hydroxyl or —OC(O)R₈; R₂ is hydrogen; R₃ is hydrogen, alkyl, alkenyl, cycloalkyl, aralkyl, heteroaralkyl, or —[(CR₂)ₚ]—R₁₆; and R₄ is hydrogen or has a formula 1a:

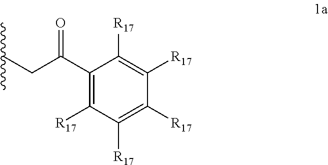

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl.

In certain embodiments, the present invention relates to the aforementioned compound and the attendant definitions, wherein R₁ is hydroxyl or —OC(O)R₈; R₂ is hydrogen; R₃ is hydrogen, alkyl, alkenyl, cycloalkyl, aralkyl, heteroaralkyl, or —[(CR₂)ₚ]—R₁₆; R₄ is hydrogen or has a formula 1a:

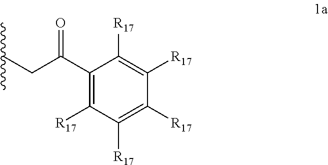

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; and R₅ and R₆ taken together form a bond.

In certain embodiments, the present invention relates to the aforementioned compound and the attendant definitions, wherein R₁ is hydroxyl or —OC(O)R₈; R₂ is hydrogen; R₃ is hydrogen, alkyl, alkenyl, cycloalkyl, aralkyl, heteroaralkyl, or —[(CR₂)ₚ]—R₁₆; R₄ is hydrogen or has a formula 1a:

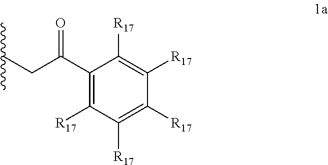

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; R₅ and R₆ taken together form a bond; and X and Y are —CH₂—.

In certain embodiments, the present invention relates to the aforementioned compound and the attendant definitions, wherein R₁ is hydroxyl or —OC(O)R₈; R₂ is hydrogen; R₃ is hydrogen, alkyl, alkenyl, cycloalkyl, aralkyl, heteroaralkyl, or —[(CR₂)ₚ]—R₁₆; R₄ is hydrogen or has a formula 1a:

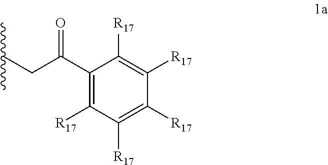

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl; R₅ and R₆ taken together form a bond; X and Y are —CH₂—; n is equal to 0; and m is equal to 0 or 1.

In one embodiment the present invention provides a compound with absolute sterochemistry as shown in formula 5:

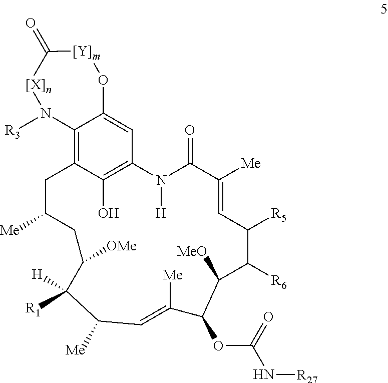

wherein independently for each occurrence:

n is equal to 0, 1, or 2;

m is equal to 0, 1, or 2;

X and Y are independently C(R₃₀)₂; wherein R₃₀ for each occurrence is independently selected from the group consisting of hydrogen, alkyl, cycloalkyl, heterocycloalkyl, aralkyl, heteroaryl, and heteroaralkyl; or —[(CR₂)ₚ]—R₁₆;

R₁ is hydroxyl or —OC(O)R₈;

R₃ is hydrogen, alkyl, alkenyl, alkynyl, cycloalkyl, aralkyl, heteroaralkyl, or —[(CR₂)ₚ]—R₁₆;

R₅ and R₆ are both hydrogen; or R₅ and R₆ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₇ is hydrogen, alkyl, aryl, cycloalkyl, heterocycloalkyl, aralkyl, heteroaryl, or heteroaralkyl; and

the stereochemistry of a double bond can be E or Z or a mixture thereof.

In certain embodiments, the present invention relates to the aforementioned compound and the attendant definitions, wherein R₁ is hydroxyl.

In certain embodiments, the present invention relates to the aforementioned compound and the attendant definitions, wherein R₃ is allyl.

In certain embodiments, the present invention relates to the aforementioned compound and the attendant definitions, wherein R₅ and R₆ taken together form a bond.

In certain embodiments, the present invention relates to the aforementioned compound and the attendant definitions, wherein R₂₇ is hydrogen.

In certain embodiments, the present invention relates to the aforementioned compound and the attendant definitions, wherein X and Y are —CH₂—.

In certain embodiments, the present invention relates to the aforementioned compound and the attendant definitions, wherein n is equal to 0; and m is equal to 0 or 1.

In certain embodiments, the present invention relates to the aforementioned compound and the attendant definitions, wherein R₁ is hydroxyl; and R₃ is allyl.

In certain embodiments, the present invention relates to the aforementioned compound and the attendant definitions, wherein R₁ is hydroxyl; R₃ is allyl; and R₅ and R₆ taken together form a bond.

In certain embodiments, the present invention relates to the aforementioned compound and the attendant definitions, wherein R₁ is hydroxyl; R₃ is allyl; R₅ and R₆ taken together form a bond; and R₂₇ is hydrogen.

In certain embodiments, the present invention relates to the aforementioned compound and the attendant definitions, wherein R₁ is hydroxyl; R₃ is allyl; R₅ and R₆ taken together form a bond; R₂₇ is hydrogen; and X and Y are —CH₂—.

In certain embodiments, the present invention relates to the aforementioned compound and the attendant definitions, wherein R₁ is hydroxyl; R₃ is allyl; R₅ and R₆ taken together form a bond; R₂₇ is hydrogen; X and Y are —CH₂—; n is equal to 0; and m is equal to 0 or 1.

In one embodiment the present invention provides a compound selected from the group consisting of:

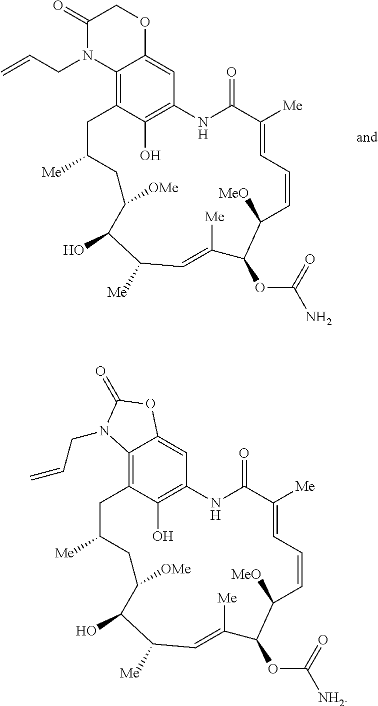

The embodiments described above and in the following sections encompass hydroquinone analogs of the geldanamycin family of molecules. In addition to reduced forms of 17-AAG (17-allylamino-18,21-dihydro-17-demethoxygeldanamycin), other compounds of the present invention relates to 18,21-dihydro-geldanamycin family including, but not limited to, 18,21-dihydro analogs of 17-Amino-4,5-dihydro-17-demethoxy-geldanamycin; 17-Methylamino-4,5-dihydro-17-demethoxygeldanamycin; 17-Cyclopropylamino-4,5-dihydro-17-demethoxygeldanarnycin; 17-(2′-Hydroxyethylamino)-4,5-dihydro-17-demethoxygelclanamycin; 17-(2-Methoxyethylamino)-4,5-dihydro-17-demethoxygeldanamycin; 17-(2′-Fluoroethylamino)-4,5-dihydro-17-demethoxygeldanamycin; 17-(S)-(+)-2-Hydroxypropylamino-4,5-dihydro-17-demethoxygeldanamycin; 17-Azetidin-1-yl-4,5-dihydro-17-demethoxygeldanamycin; 17-(3-Hydroxyazetidin-1-yl)-4,5-dihydro-17-demethoxygeldanamycin; 17-Azetidin-1-yl-4,5-dihydro-11-alpha-fluoro-17-demethoxygeldanamycin; 17-(2′-Cyanoethylamino)-17-demethoxygeldanamycin; 17-(2′-Fluoroethylamino)-17-demethoxygeldanamycin; 17-Amino-22-(2′-methoxyphenacyl)-17-demethoxygeldanamycin; 17-Amino-22-(3′-methoxyphenacyl)-17-demethoxygeldanetmycin; 17-Amino-22-(4′-chlorophenacyl)-17-demethoxygeldanamycin; 17-Amino-22-(3′,4′-dichlorophenacyl)-17-demethoxygeldanamycin; 17-Amino-22-(4′-amino-3′-iodophenacyl)-17-demethoxygeldanamycin; 17-Amino-22-(4′-azido-3′-iodophenacyl)-17-demethoxygeldanamycin; 17-Amino-1′-alpha-fluoro-17-demethoxygeldanamycin; 17-Allylamino-1′-alpha-fluoro-17-demethoxygeldanamycin; 17-Propargylamino-1′-alpha-fluoro-17-demethoxygeldanamycin; 17-(2′-Fluoroethylamino)-11-alpha-fluoro-17-demethoxygeldanamycin; 17-Azetidin-1-yl-11-(4′-azidophenyl)sulfamylcarbonyl-17-demethoxygeldanamycin; 17-(2′-Fluoroethylamino)-11-keto-17-demethoxygeldanamycin; 17-Azetidin-1-yl-11-keto-17-demethoxygeldanamycin; and 17-(3′-Hydroxyazetidin-1-yl)-11-keto-17-demethoxygeldanamycin.

It will be understood by one skilled in the art that the methodology outlined herein can be used with any amino substituted benzoquinone ansamycin.

The compositions of the present invention exists as salts of the reduced ansamycin, e.g., HCl or H₂SO₄ salts. In another embodiment the compounds are co-crystallized with another salt, such as an amino acid, e.g., glycine. In general, in these embodiments, the ratio of amino acid to ansamycin can vary, but is often from 2:1 to 1:2 amino acid:ansamycin.

2. IPI-493

Compositions, methods of synthesis, methods of administration, etc. for IPI-493 can be found in PCT application WO2008/073424, the entire contents of which is incorporated by reference.

In some embodiments, a pharmaceutical composition for oral administration is provided, comprising a crystallization inhibitor and a compound of formula 1:

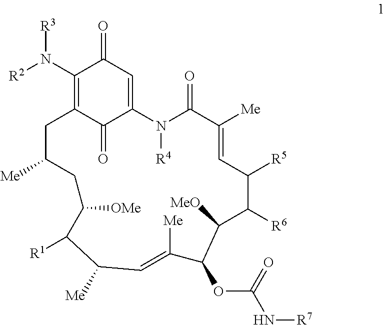

or a pharmaceutically acceptable salt thereof;
wherein;
R¹ is H, —OR⁸, —SR⁸—N(R⁸)(R⁹), —N(R⁸)C(O)R⁹, —N(R⁸)C(O)OR⁹, —N(R⁸)C(O)N(R⁸)(R⁹), —OC(O)R⁸, —OC(O)OR⁸, —OS(O)₂R⁸, —OS(O)₂OR⁸, —OP(O)₂OR⁸, CN or a carbonyl moiety;
each of R² and R³ independently is H, alkyl, alkenyl, alkynyl, cycloalkyl, cycloalkenyl, heterocycloaklyl, aryl, aralkyl, heteroaryl, heteroaralkyl, —C(═O)CH₃ or —[(C(R¹⁰)₂)ₚ]—R¹¹;
or R² and R³ taken together with the nitrogen to which they are bonded represent a 3-8 membered optionally substituted heterocyclic ring which contains 1-3 heteroatoms selected from O, N, S, and P;
p independently for each occurrence is 0, 1, 2, 3, 4, 5, or 6;
R⁴ is H, alkyl, akenyl, or aralkyl;
R⁵ and R⁶ are each H; or R⁵ and R⁶ taken together form a bond;
R⁷ is hydrogen alkyl, alkenyl, alkynyl, cycloalkyl, cycloalkenyl, heterocycloaklyl, aryl, aralkyl, heteroaryl, heteroaralkyl, or —[(CR₂)ₚ]—R₁₆;
each of R⁸ and R⁹ independently for each occurrence is H, alkyl, alkenyl, alkynyl, cycloalkyl, cycloalkenyl, heterocycloaklyl, aryl, aralkyl, heteroaryl, heteroaralkyl, or —[(C(R¹⁰)₂)ₚ]—R¹¹; or R⁸ and R⁹ taken together represent a 3-8 membered optionally substituted heterocyclic ring which contains 1-3 heteroatoms selected from O, N, S, and P;
R¹⁰ for each occurrence independently is H, alkyl, alkenyl, alkynyl, cycloalkyl, cycloalkenyl, heterocycloaklyl, aryl, aralkyl, heteroaryl, or heteroaralkyl; and
R¹¹ for each occurrence independently is H, cycloalkyl, aryl, heteroaryl, heterocyclyl, —OR⁸, —SR⁸, —N(R⁸)(R⁹), —N(R⁸)C(O)R⁹, —N(R⁸)C(O)OR⁹, —N(R⁸)C(O)N(R⁸)(R⁹), —OC(O)R⁸, —OC(O)OR⁸, —OS(O)₂R⁸, —OS(O)₂OR⁸, —OP(O)₂OR⁸, —C(O)R⁸, —C(O)₂R⁸, —C(O)N(R⁸)(R⁹), halide, or CN.

In some embodiments R¹ is OH, R⁴ is H, and R⁵ and R⁶ taken together form a bond.

In some embodiments, a pharmaceutical composition for oral administration is provided, comprising a crystallization inhibitor and a compound of formula 1:

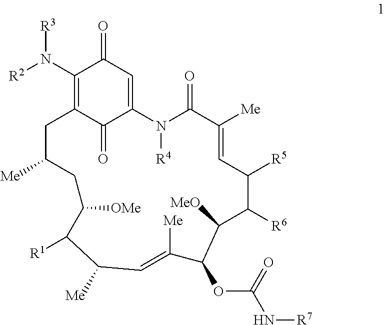

In certain embodiments, a pharmaceutical composition for oral administration is provided, comprising a crystallization inhibitor and a compound of formula 1:

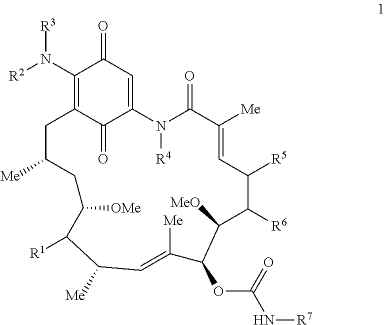

or a pharmaceutically acceptable salt thereof;
wherein;
R¹ is —OR⁸, —C(═O)CH₃, or a carbonyl moiety;
each of R² and R³ independently is H, alkyl, alkenyl or)-[(C(R¹⁰)₂)ₚ]—R¹¹; or R² and R³ taken together with the nitrogen to which they are bonded represent a 3-8 membered optionally substituted heterocyclic ring which contains 1-3 heteroatoms selected from O, N, S, and P;
p independently for each occurrence is 0, 1 or 2;

### R⁴ is H;

R⁵ and R⁶ are each H; or R⁵ and R⁶ taken together form a bond;
R⁷ is hydrogen or —[(C(R¹⁰)₂)ₚ]—R¹¹;
each of R⁸ and R⁹ independently are H; or R⁸ and R⁹ taken together represent a 3-8 membered optionally substituted heterocyclic ring which contains 1-3 heteroatoms selected from O, N, S, and P;
R¹⁰ for each occurrence independently is H; and
R^{H} for each occurrence independently is H, —N(R⁸)(R⁹) or halide.

Examples of benzoquinone ansamycin compounds include those having the following structures:

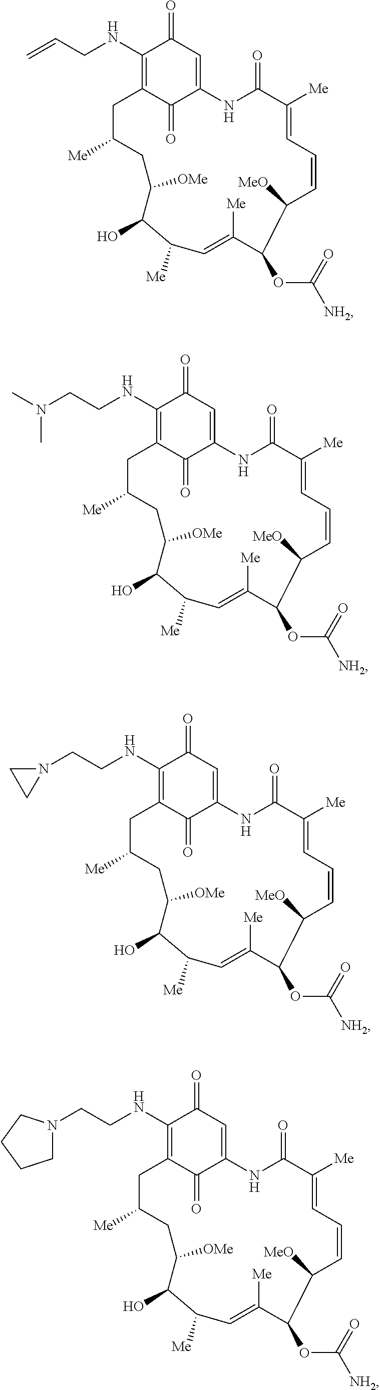

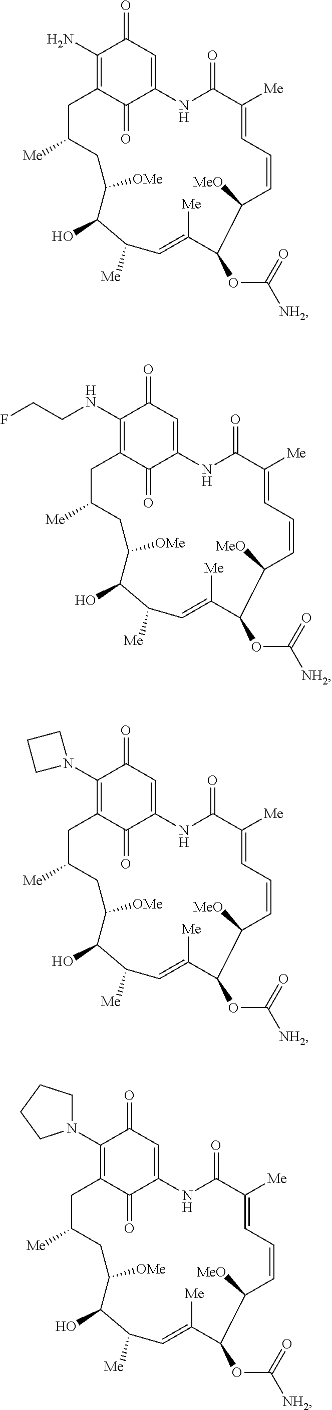

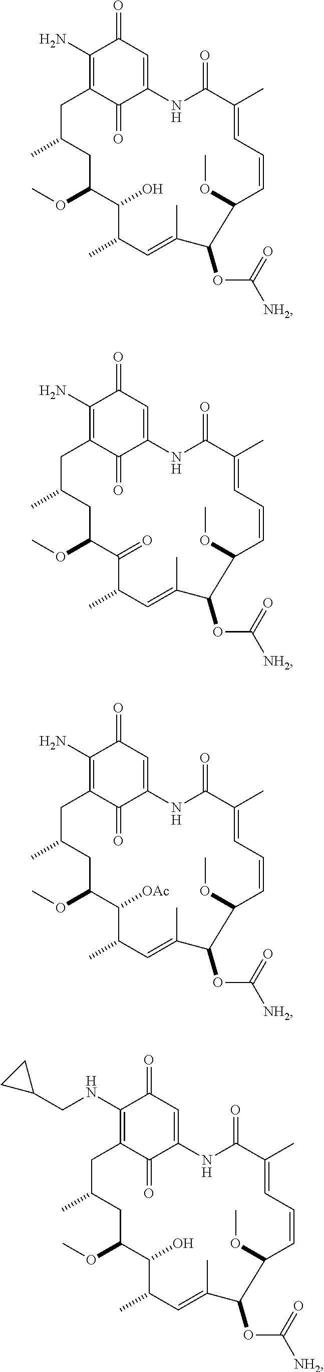

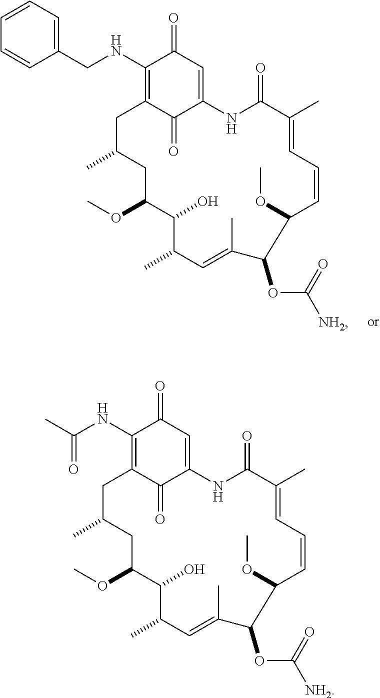

In some embodiments, compositions provided herein containing amorphous 17-AG resulted in a surprising finding of improved bioavailability relative to crystalline 17-AG even when no crystallization inhibitor was used; such compositions are therefore useful for administration, such as oral administration.

In some of the foregoing embodiments, the compound is present in substantially amorphous form.

Similarly, in some embodiments, the composition contains an amount of crystallization inhibitor of at least about 10%, at least about 25%, at least about 50%, at least about 75% (w/w), based on the total weight of the composition.

In some of the foregoing embodiments, the crystallization inhibitor is PVP. In some of the foregoing embodiments, the 17-AG is substantially amorphous.

In certain embodiments, the pharmaceutical composition can be in the form of a paste, solution, slurry, ointment, emulsion or dispersion. In certain embodiments, the pharmaceutical composition is, or comprises, a molecular dispersion.

In certain embodiments, the crystallization inhibitor can be selected from polyvinylpyrrolidone (PVP) (including homo- and copolymers of polyvinylpyrrolidone and homopolymers or copolymers of N-vinylpyrrolidone); crospovidone; gums; cellulose derivatives (including hydroxypropyl methylcellulose (HPMC), hydroxypropyl methylcellulose phthalate, hydroxypropyl cellulose, ethyl cellulose, hydroxyethylcellulose, sodium carboxymethyl cellulose, calcium carboxymethyl cellulose, sodium carboxymethyl cellulose, and others); dextran; acacia; homo- and copolymers of vinyllactam, and mixtures thereof; cyclodextrins; gelatins; hypromellose phthalate; sugars; polyhydric alcohols; polyethylene glycol (PEG); polyethylene oxides; polyoxyethylene derivatives; polyvinyl alcohol; propylene glycol derivatives and the like, SLS, Tween, Eudragit; and combinations thereof. The crystallization inhibitor can be water soluble or water insoluble.

HPMCs vary in the chain length of their cellulosic backbone and consequently in their viscosity as measured for example at a 2% (W/W) in water. HPMC used in the pharmaceutical compositions provided herein can have a viscosity in water (at a concentration of 2% (w/w)), of about 100 to about 100,000 cP, about 1000 to about 15,000 cP, for example about 4000 cP. In certain embodiments, the molecular weight of HPMC used in the pharmaceutical compositions provided herein can have greater than about 10,000, but not greater than about 1,500,000, not greater than about 1,000,000, not greater than about 500,000, or not greater than about 150,000.

HPMCs also vary in the relative degree of substitution of available hydroxyl groups on the cellulosic backbone by methoxy and hydroxypropoxy groups. With increasing hydroxypropoxy substitution, the resulting HPMC becomes more hydrophilic in nature. In certain embodiments, the HPMC has about 15% to about 35%, about 19% to about 32%, or about 22% to about 30%, methoxy substitution, and having about 3% to about 15%, about 4% to about 12%, or about 7% to about 12%, hydroxypropoxy substitution.

HPMCs which can be used in the pharmaceutical compositions are illustratively available under the brand names Methocel™ of Dow Chemical Co. and Metolose™ of Shin-Etsu Chemical Co. Examples of suitable HPMCs having medium viscosity include Methocel™ E4M, and Methocel™ K4M, both of which have a viscosity of about 4000cP at 2% (w/w) water. Examples of HPMCs having higher viscosity include Methocel™ E10M, Methocel™ K15M, and Methocel™ K100M, which have viscosities of about 10,000 cP, 15,000 cP, and 100,000 cP respectively viscosities at 2% (w/w) in water. An example of an HPMC is HPMC-acetate succinate, i.e., HPMC-AS.

In certain embodiments the PVPs used in pharmaceutical compositions provided herein have a molecular weight of about 2,500 to about 3,000,000 Daltons, about 8,000 to about 1,000,000 Daltons, about 10,000 to about 400,000 Daltons, about 10,000 to about 300,000 Daltons, about 10,000 to about 200,000 Daltons, about 10,000 to about 100,000 Daltons, about 10,000 to about 80,000 Daltons, about 10,000 to about 70,000 Daltons, about 10,000 to about 60,000 Daltons, about 10,000 to about 50,000 Daltons, or about 20,000 to about 50,000 Daltons. In certain instances the PVPs used in pharmaceutical compositions provided herein have a dynamic viscosity, 10% in water at 20° C., of about 1.3 to about 700, about 1.5 to about 300, or about 3.5 to about 8.5 mPas.

When PEGs are used they can have an average molecular about 5,000-20,000 Dalton, about 5,000-15,000 Dalton, or about 5,000-10,000 Dalton.

Also provided herein is a pharmaceutical composition for oral delivery, comprising 17-AG and at least one pharmaceutically acceptable excipient, wherein said pharmaceutical composition is substantially free of crystalline 17-AG. In certain instances, the 17-AG in such a pharmaceutical composition includes less than about 15% (w/w), less than about 10% (w/w), less than about 5% (w/w), less than about 3% (w/w), or less than about 1% (w/w) crystalline 17-AG. Such a pharmaceutical composition can be formulated as a solid dosage form (e.g., a tablet or capsule), a paste, emulsion, slurry, or ointment.

Also provided herein is a pharmaceutical composition for oral delivery, comprising 17-AAG and at least one pharmaceutically acceptable excipient, wherein said pharmaceutical composition is substantially free of crystalline 17-AAG. In certain instances, the 17-AAG in such a pharmaceutical composition includes less than about 15% (w/w), less than about 10% (w/w), less than about 5% (w/w), less than about 3% (w/w), or less than about 1% (w/w) crystalline 17-AAG. Such a pharmaceutical composition can be formulated as a solid dosage form (e.g., a tablet or capsule), a paste, emulsion, slurry, or ointment.

As described above, benzoquinone ansamycins and pharmaceutical compositions of the present invention can additionally comprise pharmaceutically acceptable carriers and excipients according to conventional pharmaceutical compounding techniques to form a pharmaceutical composition or dosage form. Suitable pharmaceutically acceptable carriers and excipients include, but are not limited to, those described in Remington's, The Science and Practice of Pharmacy, (Gennaro, A. R., ed., 19^{th} edition, 1995, Mack Pub. Co.), which is herein incorporated by reference. The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human). For oral liquid pharmaceutical compositions, pharmaceutical carriers and excipients can include, but are not limited to water, glycols, oils, alcohols, flavoring agents, preservatives, coloring agents, and the like. Oral solid pharmaceutical compositions can include, but are not limited to starches, sugars, microcrystalline cellulose, diluents, granulating agents, lubricants, binders and disintegrating agents. The pharmaceutical composition and dosage form can also include a benzoquinone ansaymyscin compound or solid form thereof as discussed above.

The solid forms described herein can be useful for making pharmaceutical compositions suitable for oral administration. Such pharmaceutical compositions can contain any of the benzoquinone ansamycin compounds described herein, for example, in an amorphous form and no crystallization inhibitor, or an amorphous form in combination with a crystallization inhibitor. Examples of such benzoquinone ansamycins are described in Schnur et al., J. Med. Chem. 1995, 38: 3806-12.

### X. Therapeutic Methods

Alternatively, or in combination with the methods described herein, the invention features a method of treating a cancer or tumor harboring an oncogenic alteration described herein, e.g., one or more ALK, MAPK pathway (e.g., K-Ras), and/or EGFR alterations as described herein, with one or more HSP90 inhibitors, alone or in combination, e.g., in combination with one or more mTOR inhibitors; an ALK inhibitor; a tyrosine kinase inhibitor and/or other chemotherapeutic agents. The method includes administering to the subject an HSP inhibitor, e.g., one or more HSP90 inhibitors as described herein, alone or in combination with an mTOR inhibitor, an ALK inhibitor a tyrosine kinase inhibitor, and/or other chemotherapeutic agents, in an amount sufficient to reduce or inhibit the tumor cell growth, and/or treat or prevent the cancer(s), in the subject.

“Treat,” “treatment,” and other forms of this word refer to the administration of an HSP90 inhibiting agent, alone or in combination with a second agent to impede growth of a cancer, to cause a cancer to shrink by weight or volume, to extend the expected survival time of the subject and or time to progression of the tumor or the like. In those subjects, treatment can include, but is not limited to, inhibiting tumor growth, reducing tumor mass, reducing size or number of metastatic lesions, inhibiting the development of new metastatic lesions, prolonged survival, prolonged progression-free survival, prolonged time to progression, and/or enhanced quality of life.

As used herein, unless otherwise specified, the terms “prevent,” “preventing” and “prevention” contemplate an action that occurs before a subject begins to suffer from the regrowth of the cancer and/or which inhibits or reduces the severity of the cancer.

As used herein, and unless otherwise specified, the terms “manage,” “managing” and “management” encompass preventing the recurrence of the cancer in a patient who has already suffered from the cancer, and/or lengthening the time that a patient who has suffered from the cancer remains in remission. The terms encompass modulating the threshold, development and/or duration of the cancer, or changing the way that a patient responds to the cancer.

As used herein, and unless otherwise specified, a “therapeutically effective amount” of a compound is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent.

As used herein, and unless otherwise specified, a “prophylactically effective amount” of a compound is an amount sufficient to prevent regrowth of the cancer, or one or more symptoms associated with the cancer, or prevent its recurrence. A prophylactically effective amount of a compound means an amount of the compound, alone or in combination with other therapeutic agents, which provides a prophylactic benefit in the prevention of the cancer. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g, infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug.

In some embodiments, the HSP90 inhibitor is a first line treatment for the cancer, i.e., it is used in a patient who has not been previously administered another drug intended to treat the cancer.

In other embodiments, the HSP90 inhibitor is a second line treatment for the cancer, i.e., it is used in a patient who has been previously administered another drug intended to treat the cancer.

In other embodiments, the HSP90 inhibitor is a third or fourth line treatment for the cancer, i.e., it is used in a patient who has been previously administered two or three other drugs intended to treat the cancer.

In some embodiments, the HSP90 inhibitor is administered to a patient following surgical excision/removal of the cancer.

In some embodiments, the HSP90 inhibitor is administered to a patient before, during, and/or after radiation treatment of the cancer.

In one embodiment, the cancer evaluated and/or treated has one or more alterations in an ALK gene or gene product, e.g., an ALK rearrangement.

In another embodiment, the cancer evaluated and/or treated has one or more alterations in a MAPK pathway (e.g., K-Ras) gene or gene product. MAPK pathway activation has been detected in a wide variety of cancers. For example, Ras and Raf mutations have been detected in cancers including, but not limited to:
(i) bladder cancer (H-Ras mutations: Malone et al., Br. J. Urol. (1985) 57:664-667, Fujita et al., Gastroenterology (1987) 6:1339-1345, Vis Vanathan et al., Oncogene Res. (1988) 3:77-86);
(ii) brain cancer (C-Raf mutations: LaRocca et al., J. Neurosci. Res. (1989) 24:97-106; Fukui et al., Mol. Cell. Biol. (1987) 7:1776-1781),
(iii) breast cancer (C-Raf mutations: Callans et al., Ann. Surg. Oncol. (1995) 2:38-42; McFarlin et al., Carcinogenesis (2003) 24:1149-1153; Ras mutations: Miyakis et al., Biochem. Biophys Res. Commun. (1998) 251:609-612 (K-Ras), Spandidos et al., Anticancer Res. (1987) 7:991-996 (H-Ras));
(iv) biliary cancer (B-Raf mutations in cholangiocarcinoma: Tannapfel et al., Gut (2003) 52:706-712; K-Ras mutations: Hidaka et al., Cancer Res. (2000) 60:522-524, Laghi et al., Oncogene (2002 21:4301-4306);
(v) cervical cancer (H-Ras mutations: Riou et al., Oncogene (1988) 3:329-333);
(vi) colorectal cancer (B-raf mutations: Rajagopalan et al., Nature (2002) 418:934; B-Raf and K-Ras mutations: Yuen et al., Cancer Res. (2002) 62:6451-6455; K-Ras mutations: Vogelstein et al., N. Engl. J. Med. (1988) 319:525-532, Bos et al., Nature (London) (1987) 327:293-297, Forrester et al., Nature (London) (1987) 327:298-303, Farr et al., Oncogene (1988) 3:673-678);
(vii) endometrial cancer (K-Ras mutations: Lagarda et al., J. Pathol. (2001) 193-199);
(viii) esophageal cancer (B-raf mutations in Barrett's adenocarinoma: Sommerer et al., Oncogene (2004) 23:554-558);
(ix) ependymoma (C-Raf mutations: Korshunov et al., Am. J. Pathol. (2003) 163:1721-1727);
(x) leukemia (B-raf mutations in AML: Lee et al., Leukemia (2004) 18:170-172; N-Ras mutations in AML: Needleman et al., Blood (1986) 67:753-757, Bos et al., Blood (1987) 69:1237-1241, Janssen et al., Proc. Natl. Acad. Sci. USA (1987) 84:9228-9232, Toksoz et al., Oncogene (1987) 1:409-413, Farr et al., Proc. Natl. Adac. Sci. USA (1988) 1629-1633, Senn et al., Int. J. Cancer (1988) 41:59-64, Bos et al., Nature (London) (1985) 315:726-730, Bartram et al., Leukemia (Baltimore) (1989) 3:247-256, Hirai et al., Biochim. Biophys. Res. Commun. (1987) 147:108-114; N-Ras mutations in CML: Liu et al. Proc. Natl. Acad. Sci. USA (1988) 85:1952-1956);
(xi) lymphoma (B-raf mutations in NHL: Lee et al., Br. J. Cancer (2003) 89:1958-1960; C-Raf mutations in NHL: Storm et al., Toxicol. Letters (1993) 67:201-210);
(xii) liver cancer (C-Raf mutations: Ting et al., Xue. Za. Zhi. (1988) 21:141-150; Jenke et al., Xenobiotica (1994) 24:569-580; Beer et al., Cancer Res. (1988) 48:1610-1617; N-Ras mutations: Gu et al., J. Cell. Physiol. Suppl. (1986) 4:13-20);
(xiii) lung cancer (B-raf and K-Ras mutations in NSCLC: Brose et al., Cancer Res. (2002) 62:6997-7000; C-Raf mutations in NSCLC: Kerkhoff et al., Cell Growth Differ. (2000) 11:185-190; C-Raf mutations in SCLC: Graziano et al., Chromosomes Cancer (1991) 3:283-293; K-Ras mutations: Rodenhuis et al., Cancer Res. (1988) 48:5738-5741),
(xiv) head and neck cancer (B-raf mutations: Cohen et al., Surgery (2002) 132:960-967; Weber et al., Oncogene (2003) 22:4757-4759; C-Raf mutations: Patel et al., Mol. Carcinog. (1997) 18:1-6; Riva et al., Eur. J. Cancer. B. Oral. Oncol. (1995) 31B:384-391);
(xv) kidney cancer (C-Raf mutations in renal cell carcinoma: Oka et al., Cancer Res. (1995) 55:4182-4187; H-Ras mutations: Fujita et al., Cancer Res. (1988) 48:5251-5255);
(xvi) gastric cancer (B-raf and K-Ras mutations: Lee et al., Oncogene (2003) 22:6942-6945);
(xvii) multiple myeloma (N-Ras mutations: Kalakonda et al., Blood (2001) 98:1555-1560);
(xviii) myeloproliferative disorders (N-Ras mutations in idiopathic myelofibrosis (IMF): Buschle et al., Leukemia (Baltimore) (1988) 2:658-660; N-Ras mutations in CML: Liu et al. Proc. Natl. Acad. Sci. USA (1988) 85:1952-1956);
(xix) myelodysplatic syndrome (N-Ras and/or K-Ras mutations: Yunis et al., Oncogene (1988) 4:609-614, Hirai et al., Nature (London) (1987) 327:430-432, Paudua et al., Leukemia (Baltimore) (1988) 2:503-510, Lyons et al., Blood (1988) 71:1707-1712);
(xx) ovarian cancer (B-raf and K-Ras mutations: Singer et al., J. Natl. Cancer Inst. (2003) 95:484-486; Gemignani et al., Gynecol. Oncol. (2003) 90:378-381), K-Ras 3′ UTR variant: Ratner, E. et al. (2010) Cancer Res. 70(16): OF1-7;
(xxi) osteosarcoma (C-Raf mutations: Ikeda et al., Jpn. J. Cancer Res. (1989) 80:6-9);
(xxii) pancreatic cancer (C-Raf mutations: Berger et al., J. Surg. Res. (1997) 69:199-204; K-Ras mutations: Almoquera et al., Cell (1988) 53:549-554, Smith et al., Nucleic Acids Res. (1988) 16:7773-7782, Grunewald et al., Int. J. Cancer (1989) 43:1037-1041, Laghi et al., Oncogene (2002 21:4301-4306);
(xxiii) salivary gland cancer (H-Ras mutations: Yoo et al., Arch Pathol Lab Med (2000) 124:836-839)
(xxiv) skin cancer (B-raf mutations in melanoma: Davies et al., Nature (2002) 417:949-954; Pollock et al., Cancer Cell (2002) 2:5-7; B-raf and K-ras mutations in melanoma: Brose et al., Cancer Res. (2002) 62:6997-7000; H-Ras mutations in keratoacanthoma: Leon et al., Mol. Cell. Biol. (1988) 8:786-793; N-Ras mutations in melanoma: Van't Veer et al., Mol. Cell. Biol. (1989) 9:3114-3116);
(xxv) soft tissue sarcoma (C-Raf mutations: Mitsunobu et al., Oncogene (1989) 4:437-442); and
(xxvi) thyroid cancer (B-raf mutations: Nikiforova et al., J. Clin. Endocrinol. Metab. (2003) 88:5399-5404; Kimura et al., Cancer Res. (2003) 63:1454-1457; Cohen et al., J. Natl. Cancer Inst. (2003) 95:625-627; C-Raf mutations: Carson et al., Cancer Res. (1995) 555:2048-2052; H-Ras, K-Ras and N-Ras mutations: Lemoine et al., Cancer Res. (1988) 48:4459-4463; Lemoine et al., Oncogene (1989) 4:159-164).

In certain embodiments, the cancer or tumor identified or treated by the methods of the invention includes, but is not limited to, a solid tumor, a soft tissue tumor, and a metastatic lesion (e.g., a cancer as described herein). In some embodiments, the cancer identified or treated harbors one or more alterations in a gene or gene product chosen from one or more of ALK, RAS (e.g., one or more of H-Ras, N-Ras, or K-Ras), EGFR, PIK3CA, RAF (e.g., one or more of A-Raf, B-Raf (BRAF) or C-Raf), PTEN, AKT, TP53 (p53), CTNNB1 (beta-catenin), APC, KIT, JAK2, NOTCH, FLT3, MEK, ERK, RSK, ETS, ELK-1, or SAP-1.

Proliferative disorders and cancers that can be treated using the methods disclosed herein include, for example, lung cancer (including small cell lung cancer and non small cell lung cancer), other cancers of the pulmonary system, medulloblastoma and other brain cancers, pancreatic cancer, basal cell carcinoma, breast cancer, prostate cancer and other genitourinary cancers, gastrointestinal stromal tumor (GIST) and other cancers of the gastrointestinal tract, colon cancer, colorectal cancer, ovarian cancer, and cancers of the hematopoietic system.

In certain embodiments, the cancer is chosen from one or more of lung cancer (e.g., small cell lung cancer, non-small cell lung cancer, SCC, adenocarcinoma of the lung, bronchogenic carcinoma), bladder cancer, neuroblastoma, breast cancer, colorectal cancer, colon cancer, inflammatory myofibroblastic tumors, multiple myeloma, leukemia (e.g., acute lymphocytic leukemia (ALL), acute myelocytic leukemia (AML), chronic myelocytic leukemia (CML), chronic lymphocytic leukemia (CLL)), lymphoma (e.g., anaplastic large cell lymphoma, Hodgkin lymphoma, non-Hodgkin lymphoma (NHL)), pancreatic cancer (e.g., pancreatic andenocarcinoma, intraductal papillary mucinous neoplasm (IPMN)), prostate cancer, medulloblastoma, chondrosarcoma, osteosarcoma, ovarian cancer (e.g., cystadenocarcinoma, ovarian embryonal carcinoma, ovarian adenocarcinoma), head and neck squamous cell carcinoma (HNSCC), brain cancer (e.g., meningioma; glioma, e.g., astrocytoma, oligodendroglioma; medulloblastoma), kidney cancer, liver cancer, gastric cancer (e.g., stomach adenocarcinoma), gastrointestinal stromal tumor (GIST), skin cancer (e.g., squamous cell carcinoma (SCC), keratoacanthoma (KA), melanoma, basal cell carcinoma) and neuroendocrine cancer.

In one embodiment, the cancer treated is a non-small cell lung cancer (NSCLC) (e.g., a relapsed and/or refractory NSCLC), or SCC.

In certain embodiments, the cancer is colorectal cancer (e.g., colorectal adenocarcinoma).

In certain embodiments, the cancer is breast cancer (e.g., adenocarcinoma of the breast, papillary carcinoma of the breast, mammary cancer, medullary carcinoma of the breast).

In certain embodiments, the cancer is multiple myeloma.

In certain embodiments, the cancer is a neuroendocrine cancer (e.g., gastroenteropancreatic neuroendoctrine tumor (GEP-NET), carcinoid tumor).

In certain embodiments, the cancer is lung cancer. In certain embodiments, the lung cancer is small cell lung cancer (SCLC). In certain embodiments, the lung cancer is non-small cell lung cancer (NSCLC). As of 2009, non small cell lung cancer accounts for approximately 85% of all lung cancers. Approximately 262,000 stage IIIB/IV are diagnosed every year. In 2009, the % of NSCLC patients is distributed as follows: approx. 18% patients have large cell carcinoma, 47% of the patients have adenocarcinoma, and 35% of the patients have squamous cell carcinoma. With respect to the smoking status, approx. 70% of the patient are smokers with greater that 15 pack-years, 13% of the patients have less or equal to 15 pack-years; 15% of the patients are non-smokers; and 2% of the patients have a history of second hand smoking.

Other exemplary cancers include, but are not limited to, acoustic neuroma, adenocarcinoma, adrenal gland cancer, angiosarcoma (e.g., lymphangiosarcoma, lymphangioendotheliosarcoma, hemangiosarcoma), benign monoclonal gammopathy, biliary cancer (e.g., cholangiocarcinoma), bronchus cancer, cervical cancer (e.g., cervical adenocarcinoma), choriocarcinoma, chordoma, craniopharyngioma, epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, hypereosinophilic syndrome (HES)), neuroblastoma, neurofibroma (e.g., neurofibromatosis (NF) type 1 or type 2, schwannomatosis), osteosarcoma, oral cancer (e.g., oral squamous cell carcinoma (OSCC)), Paget's disease of the vulva, Paget's disease of the penis, papillary adenocarcinoma, pinealoma, primitive neuroectodermal tumor (PNT), prostate cancer (e.g., prostate adenocarcinoma), rhabdomyosarcoma, retinoblastoma, salivary gland cancer, small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenström's macroglobulinemia.

Neuroendocrine cancers (also known as gastroenteropancreatic tumors or gastroenteropancreatic neuroendocrine cancers), are cancers derived from cells at the interface between the endocrine (hormonal) system and the nervous system. The majority of neuroendocrine cancers fall into two categories: carcinoids and pancreatic endocrine tumors (also known as endocrine pancreatic tumors or islet cell tumors). In addition to the two main categories, other forms of neuroendocrine cancers exist, including neuroendocrine lung tumors, which arise from the respiratory rather than the gastro-entero-pancreatic system. Neuroendocrine cancers can originate from endocrine glands such as the adrenal medulla, the pituitary, and the parathyroids, as well as endocrine islets within the thyroid or the pancreas, and dispersed endocrine cells in the respiratory and gastrointestinal tract. The total incidence of neuroendocrine cancers in the United States is about 9,000 new cases per year.

For example, the cancer treated can be a neuroendocrine cancer chosen from one or more of, e.g., a neuroendocrine cancer of the pancreas, lung, appendix, duodenum, ileum, rectum or small intestine. In other embodiments, the neuroendocrine cancer is chosen from one or more of: a pancreatic endocrine tumor; a neuroendocrine lung tumor; or a neuroendocrine cancer from the adrenal medulla, the pituitary, the parathyroids, thyroid endocrine islets, pancreatic endocrine islets, or dispersed endocrine cells in the respiratory or gastrointestinal tract.

Pancreatic endocrine tumors can secrete biologically active peptides (e.g., hormones) that can cause various symptoms in a subject. Such tumors are referred to functional or secretory tumors. Functional tumors can be classified by the hormone most strongly secreted. Examples of functional pancreatic endocrine tumors include gastrinoma (producing excessive gastrin and causing Zollinger-Ellison Syndrome), insulinoma (producing excessive insulin), glucagonoma (producing excessive glucagon), vasoactive intestinal peptideoma (VIPoma, producing excessive vasoactive intestinal peptide), PPoma (producing excessive pancreatic polypeptide), somatostatinoma (producing excessive somatostatin), watery diarrhea hypokalemia-achlorhydria (WDHA), CRHoma (producing excessive corticotropin-releasing hormonse), calcitoninoma (producing excessive calcitonin), GHRHoma (producing excessive growth-hormone-releasing hormone), neurotensinoma (producing excessive neurotensin), ACTHoma (producing excessive adrenocorticotropic hormone), GRFoma (producing excessive growth hormone-releasing factor), and parathyroid hormone-related peptide tumor. In some instances, pancreatic endocrine tumors can arise in subjects who have multiple endocrine neoplasia type 1 (MEN1); such tumors often occur in the pituitary gland or pancreatic islet cells. Pancreatic endocrine tumors that do not secrete peptides (e.g., hormones) are called nonfunctional (or nonsecretory or nonfunctional) tumors.

In other embodiments, the cancer treated is a carcinoid tumor, e.g., a carcinoid neuroendocrine cancer. Carcinoid tumors tend to grow more slowly than pancreatic endocrine tumors. A carcinoid tumor can produce biologically active molecules such as serotonin, a biogenic molecule that causes a specific set of symptoms called carcinoid syndrome. Carcinoid tumors that produce biologically active molecules are often referred to as functional carcinoid tumors, while those that do not are referred to as nonfunctional carcinoid tumors. In some embodiments, the neuroendocrine cancer is a functional carcinoid tumor (e.g., a carcinoid tumor that can produce biologically active molecules such as serotonin). In other embodiments, the neuroendocrine cancer is a non-functional carcinoid tumor. In certain embodiments, the carcinoid tumor is a tumor from the thymus, stomach, small intestine (duodenum, jejunum, ileum), large intestine (cecum, colon), rectal, pancreatic, appendix, ovarian or testicular carcinoid.

Carcinoid tumors can be further classified depending on the point of origin, such as lung, thymus, stomach, small intestine (duodenum, jejunum, ileum), large intestine (cecum, colon), rectum, pancreas, appendix, ovaries and testes.

In some embodiments, the neuroendocrine cancer is a carcinoid tumor. In other embodiments, the neuroendocrine cancer is a pancreatic endocrine tumor. In still other embodiments, the neuroendocrine cancer is a neuroendocrine lung tumor. In certain embodiments, the neuroendocrine cancers originate from the adrenal medulla, the pituitary, the parathyroids, thyroid endocrine islets, pancreatic endocrine islets, or dispersed endocrine cells in the respiratory or gastrointestinal tract.

Further examples of neuroendocrine cancers that can be treated include, but are not limited to, medullary carcinoma of the thyroid, Merkel cell cancer (trabecular cancer), small-cell lung cancer (SCLC), large-cell neuroendocrine carcinoma (of the lung), extrapulmonary small cell carcinomas (ESCC or EPSCC), neuroendocrine carcinoma of the cervix, Multiple Endocrine Neoplasia type 1 (MEN-1 or MEN1), Multiple Endocrine Neoplasia type 2 (MEN-2 or MEN2), neurofibromatosis type 1, tuberous sclerosis, von Hippel-Lindau (VHL) disease, neuroblastoma, pheochromocytoma (phaeochromocytoma), paraganglioma, neuroendocrine cancer of the anterior pituitary, and/or Carney's complex.

In yet other embodiments, the cancer or tumor evaluated and/or treated is a hematologic malignancy, e.g., a malignancy that contains the BCR-ABL fusion gene (Ph+ such as chronic myelogeneous leukemia (CML) and acute lymphocytic leukemia (ALL); a malignancy that contains a mutation or internal tandem duplication of Flt3 (Flt3 such as acute myelogeneous leukemia (AML); a malignancy that contains a mutation of JAK2 (JAK2+ such as polycethemia vera, essential thrombocytopenia, and myelofibrosis (MF). In one embodiment, the subject with the hematologic malignancy is treated with IPI-493 at a dose of about 100-200 mg (e.g., 100, 125, 150, 175 or 200 mg) weekly. Parameters evaluated in the subject after treatment include reduced blood counts and bone marrow recovery without blasts. In other embodiments, the subject treated with IPI-493 has a solid tumor. In such subjects, IPI-493 is administed at a dose of about 100-200 mg (e.g., 100, 125, 150, 175 or 200 mg) twice a week.

The invention also relates to methods of extending relapse free survival in a cancer patient who is undergoing or has undergone cancer therapy (for example, treatment with a chemotherapeutic (including small molecules and biotherapeutics, e.g., antibodies), radiation therapy, surgery, RNAi therapy and/or antisense therapy) by administering a therapeutically effective amount of a HSP90 inhibitor to the patient. “Relapse free survival”, as understood by those skilled in the art, is the length of time following a specific point of cancer treatment during which there is no clinically-defined relapse in the cancer. In some embodiments, the HSP90 inhibitor is administered concurrently with the cancer therapy. In instances of concurrent administration, the HSP90 inhibitor can continue to be administered after the cancer therapy has ceased. In other embodiments, the HSP90 inhibitor is administered after cancer therapy has ceased (i.e., with no period of overlap with the cancer treatment). The HSP90 inhibitor can be administered immediately after cancer therapy has ceased, or there can be a gap in time (e.g., up to about a day, a week, a month, six months, or a year) between the end of cancer therapy and the administration of the HSP90 inhibitor. Treatment with the HSP90 inhibitor can continue for as long as relapse-free survival is maintained (e.g., up to about a day, a week, a month, six months, a year, two years, three years, four years, five years, or longer).

In one aspect, the invention relates to a method of extending relapse free survival in a cancer patient who had previously undergone cancer therapy (for example, treatment with a chemotherapeutic (including small molecules and biotherapeutics, e.g., antibodies), radiation therapy, surgery, RNAi therapy and/or antisense therapy) by administering a therapeutically effective amount of a HSP90 inhibitor to the patient after the cancer therapy has ceased. The HSP90 inhibitor can be administered immediately after cancer therapy has ceased, or there can be a gap in time (e.g., up to about a day, a week, a month, six months, or a year) between the end of cancer therapy and the administration of the HSP90 inhibitor.

Certain methods of the current invention can be especially effective in treating cancers that respond well to existing chemotherapies, but suffer from a high relapse rate. In these instances, treatment with the HSP90 inhibitor can increase the relapse-free survival time or rate of the patient. Examples of such cancers include lung cancer (e.g., small cell lung cancer or non-small cell lung cancer), pancreatic cancer, bladder cancer, ovarian cancer, breast cancer, colon cancer, multiple myeloma, acute myelogenous leukemia (AML), chronic myelogenous leukemia (CML) and neuroendocrine cancer.

The invention also encompasses the use of a chemotherapeutic agent and a HSP90 inhibitor for preparation of one or more medicaments for use in a method of extending relapse free survival in a cancer patient. The invention also relates to the use of a HSP90 inhibitor in the preparation of a medicament for use in a method of extending relapse free survival in a cancer patient who had previously been treated with a chemotherapeutic.

### Combination Therapy

It will be appreciated that the HSP90 inhibitor, as described above and herein, can be administered in combination with one or more additional therapies, e.g., such as radiation therapy, surgery and/or in combination with one or more therapeutic agents, to treat the cancers described herein.

By “in combination with,” it is not intended to imply that the therapy or the therapeutic agents must be administered at the same time and/or formulated for delivery together, although these methods of delivery are within the scope of the invention. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination will be lower than those utilized individually.

In certain embodiments, the cancer treated by the methods described herein can be selected from, for example, medulloblastoma, chondrosarcoma, osteosarcoma, pancreatic cancer, lung cancer (e.g., small cell lung cancer (SCLC) or non-small cell lung cancer (NSCLC)), colorectal cancer, ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, and prostate cancer.

An example of suitable therapeutics for use in combination with the HSP90 inhibitors for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., IPI-493, IPI-504, tanespimycin, STA-9090); mTOR inhibitor (e.g., everolimus); Ep-CAM-/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

An example of suitable therapeutics for use in combination with the HSP90 inhibitors for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, RO5083945), MET inhibitor (e.g., PF-02341066, ARQ 197), PI3K kinase inhibitor (e.g., XL147, GDC-0941), Raf/MEK dual kinase inhibitor (e.g., RO5126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bc1-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., IPI-493, IPI-504, tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., RO4929097), epigenetic therapy (e.g., 5-azacitidine), nitroglycerin, MEK inhibitor (e.g., AZD6244), cyclin-dependent kinase inhibitor (e.g., UCN-01), cholesterol-Fust, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination with the HSP90 inhibitors for treatment of colorectal cancer includes, but is not limited to, 5-Fluorouracil (5FU-TS inhibitor); Irinotecan (Topo I poison); Oxaliplatin (DNA adducts), Erbitux and Vectabix (monoclonal Abs against EGFR), FOLFOX: 5-Fluorouracil+Leucovorin+Oxaliplatin; FOLFIRI: 5-Fluorouracil+Leucovorin+Irinotecan, and a combination thereof.

An example of suitable therapeutics for use in combination with the HSP90 inhibitors for treatment of medulloblastoma includes, but is not limited to, a chemotherapeutic agent (e.g., lomustine, cisplatin, carboplatin, vincristine, and cyclophosphamide), radiation therapy, surgery, and a combination thereof.

An example of suitable therapeutics for use in combination with the HSP90 inhibitors for treatment of chondrosarcoma includes, but is not limited to, a chemotherapeutic agent (e.g., trabectedin), radiation therapy (e.g., proton therapy), surgery, and a combination thereof.

An example of suitable therapeutics for use in combination with the HSP90 inhibitors for treatment of osteosarcoma includes, but is not limited to, a chemotherapeutic agent (e.g., methotrexate (e.g., alone or in combination with leucovorin rescue), cisplatin, adriamycin, ifosfamide (e.g., alone or in combination with mesna), BCG (Bacillus Calmette-Guerin), etoposide, muramyl tri-peptite (MTP)), radiation therapy, surgery, and a combination thereof.

An example of suitable therapeutics for use in combination with the HSP90 inhibitors for treatment of pancreatic cancer includes, but is not limited to, a chemotherapeutic agent, e.g., paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., R05126766); Notch signaling inhibitor (e.g., MK0752); monoclonal antibody-antibody fusion protein (e.g., L19IL2); curcumin; HSP90 inhibitor (e.g., IPI-493, IPI-504, tanespimycin, STA-9090); rIL-2; denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., RO4929097); ribonucleotide reductase inhibitor (e.g., 3-AP); immunotoxin (e.g., HuC242-DM4); PARP inhibitor (e.g., KU-0059436, veliparib); CTLA-4 inhibitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof. In certain embodiments, a combination of paclitaxel or a paclitaxel agent, and gemcitabine can be used with the HSP90 inhibitors.

An example of suitable therapeutics for use in combination with the HSP90 inhibitors for treatment of ovarian cancer includes, but is not limited to, a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agnet (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy; and combinations thereof.

An example of suitable therapeutics for use in combination with the HSP90 inhibitors for treatment of chronic myelogenous leukemia (AML) according to the invention includes, but is not limited to, a chemotherapeutic (e.g., cytarabine, hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib, RGB-286638)), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., IPI-493, IPI-504, tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., RO5045337), Aurora B kinase inhibitor (e.g., TAK-901), radioimmunotherapy (e.g., actinium-225-labeled anti-CD33 antibody HuM195), Hedgehog inhibitor (e.g., PF-04449913), STAT3 inhibitor (e.g., OPB-31121), KB004, cancer vaccine (e.g., AG858), bone marrow transplantation, stem cell transplantation, radiation therapy, and combinations thereof.

An example of suitable therapeutics for use in combination with the HSP90 inhibitors for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bc1-2 inhibitor (e.g., ABT-263), immunotherapy (e.g., allogeneic CD4+ memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., RO05045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination with the HSP90 inhibitors for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., RO5045337), immunotoxin (e.g., CAT-8015, DT2219ARL), HDAC inhibitor (e.g., JNJ-26481585), JVRS-100, paclitaxel or a paclitaxel agent, STAT3 inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination with the HSP90 inhibitors for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT3881L3 fusion protein, HDAC inhibitor (e.g., vorinostat, LBH589), plerixafor, mTOR inhibitor (e.g., everolimus), SRC inhibitor (e.g., dasatinib), HSP90 inhibitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., RO5045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of suitable therapeutics for use in combination with the HSP90 inhibitors for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3 inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of suitable therapeutics for use in combination with the HSP90 inhibitors for treatment of head and neck cancer includes, but is not limited to, a chemotherapeutic (e.g., paclitaxel or a paclitaxel agent, carboplatin, docetaxel, amifostine, cisplantin, oxaliplatin, docetaxel), tyrosine kinase inhibitors (e.g., EGFR inhibitor (e.g., erlotinib, gefitinib, icotinib, cetuximab, panitumumab, zalutumumab, nimotuzumab, necitumumab, matuzumab, cetuximab), dual inhibitor (e.g., lapatinib, neratinib, vandetanib, BIBW 2992, multikinase inhibitor (e.g., XL-647)), VEGF inhibitor (e.g., bevacizumab), reovirus, radiation therapy, surgery, and a combination thereof.

An example of suitable therapeutics for use in combination with the HSP90 inhibitors for treatment of prostate cancer includes, but is not limited to, a chemotherapeutic agent (e.g., docetaxel, carboplatin, fludarabine), hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

In some embodiments, the HSP90 inhibitor is used in combination with an mTOR inhibitor. mTOR inhibitors suitable for use in the invention are described in numerous references, including but not limited to: WO 94/02136 (16-O-substituted derivatives); U.S. Pat. No. 5,258,389 (40-O-substituted derivatives); WO 94/9010 (O-aryl and O-alkyl derivatives); WO 92/05179 (carboxylic acid esters); U.S. Pat. Nos. 5,118,677 and 5,118,678 (amide esters); U.S. Pat. No. 5,118,678 (carbamates); U.S. Pat. No. 5,100,883 (fluorinated esters); U.S. Pat. No. 5,151,413 (acetals); U.S. Pat. No. 5,120,842 (silyl esters); WO 93,11130 (methylene derivatives); WO 94/02136 (methoxy derivatives); WO 94/02385 and WO 95/14023 (alkenyl derivatives); U.S. Pat. No. 5,256,790 (32-O-dihydro or substituted derivatives); EP 96/02441; U.S. 2004/023562 (carbohydrate derivatives); U.S. Pat. No. 4,316,885 (mono and diacylated derivatives); U.S. Pat. No. 5,120,725 (bicylic derivatives); U.S. Pat. No. 5,120,727 (rapamycin dimers); EP 467606 (27-oximes of rapamycin); U.S. Pat. No. 5,023,262 (42-oxo analogs); U.S. Pat. No. 5,177,203 (arylsulfonates and sulfamates); U.S. Pat. No. 5,177,203. In addition, various rapamycin prodrugs have been described in U.S. Pat. Nos. 4,650,803; 5,672,605; 5,583,189; 5,527,906; 5,457,111; 5,995,100; and 6,146,658. Of particular interest for use in treatment methods are derivatives described in patents owned by Novartis (U.S. Pat. Nos. 5,665,772; 5,912,253; 5,985,890; 5,912,253; 6,200,985; 6,384,046; and 6,440,990), Ariad (WO 96/41865); and Wyeth (U.S. Pat. Nos. 5,362,718; 6,399,625; 6,399,627; 6,432,973; 6,440,991; 6,677,357; and 6,680,718). Exemplary mTOR inhibitors, include, but are not limited to, rapamycin, temsirolimus (TORISEL®), everolimus (RAD001, AFINITOR®), ridaforolimus, AP23573, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, or PKI-587. In one embodiment, the mTOR inhibitor inhibits TORC1 and TORC2. Examples of TORC1 and TORC2 dual inhibitors include, e.g., OSI-027, XL765, Palomid 529, and INK128.

In some embodiments, the HSP90 inhibitor is used in combination with an inhibitor of insulin-like growth factor receptor (IGF-1R), e.g., BMS-536924, GSK1904529A, AMG 479, MK-0646, cixutumumab, OSI 906, figitumumab (CP-751,871), or BIIB022.

In some embodiments, the HSP90 inhibitor is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD 173074,nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In some embodiments, the HSP90 inhibitor is used in combination with folfirinox comprising oxaliplatin 85 mg/m2 and irinotecan 180 mg/m2 plus leucovorin 400 mg/m2 followed by bolus fluorouracil (5-FU) 400 mg/m2 on day 1, then 5-FU 2,400 mg/m2 as a 46-hour continuous infusion.

In some embodiments, the HSP90 inhibitor is used in combination with a PI3K inhibitor. In one embodiment, the PI3K inhibitor is an inhibitor of delta and gamma isoforms of PI3K. Exemplary PI3K inhibitors that can be used in combination are described in, e.g., WO 2010/036380; WO 2010/006086, WO 09/114,870, WO 05/113556. Additional PI3K inhibitors that can be used in combination with the pharmaceutical compositions, include but are not limited to, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof.

In some embodiments, the HSP90 inhibitor is administered in combination with a BRAF inhibitor, e.g., GSK2118436, RG7204, PLX4032, GDC-0879, PLX4720, and sorafenib tosylate (Bay 43-9006).

In some embodiments, the HSP90 inhibitor is administered in combination with a MEK inhibitor, e.g., ARRY-142886, GSK1120212, RDEA436, RDEA119/BAY 869766, AS703026, AZD6244 (selumetinib), BIX 02188, BIX 02189, CI-1040 (PD184352), PD0325901, PD98059, and U0126.

In some embodiments, the HSP90 inhibitor is administered in combination with a JAK2 inhibitor, e.g., CEP-701, INCB18424, CP-690550 (tasocitinib)

In some embodiments, the HSP90 inhibitor is administered in combination with paclitaxel or a paclitaxel agent, e.g., TAXOL®, protein-bound paclitaxel (e.g., ABRAXANE®). A “paclitaxel agent” as used herein refers to a formulation of paclitaxel (e.g., for example, TAXOL) or a paclitaxel equivalent (e.g., for example, a prodrug of paclitaxel). Exemplary paclitaxel equivalents include, but are not limited to, nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620). In certain embodiments, the paclitaxel agent is a paclitaxel equivalent. In certain embodiments, the paclitaxel equivalent is ABRAXANE.

In certain embodiments, the HSP90 inhibitor and the second agent, e.g., the mTOR inhibitor, the ALK inhibitor and/or the additional anti-cancer agent, are administered concurrently (i.e., administration of the two agents at the same time or day, or within the same treatment regimen) or sequentially (i.e., administration of one agent over a period of time followed by administration of the other agent for a second period of time, or within different treatment regimens).

In certain embodiments, the HSP90 inhibitor and the second agent, e.g., the mTOR inhibitor, the ALK inhibitor and/or the additional anti-cancer agent, are administered concurrently. For example, in certain embodiments, the HSP90 inhibitor and the second agent(s) are administered at the same time. In certain embodiments, the HSP90 inhibitor and the second agent(s) are administered on the same day. In certain embodiments, the HSP90 inhibitor is administered after the second agent(s) on the same day or within the same treatment regimen. In certain embodiments, the HSP90 inhibitor is administered before the second agent(s) on the same day or within the same treatment regimen.

In certain embodiments, a HSP90 inhibitor is concurrently administered with the second agent(s) for a period of time, after which point treatment with the additional anti-cancer agent is stopped and treatment with the HSP90 inhibitor continues.

In other embodiments, a HSP90 inhibitor is concurrently with the second agent(s) for a period of time, after which point treatment with the HSP90 inhibitor is stopped and treatment with the additional anti-cancer agent continues.

In certain embodiments, the HSP90 inhibitor and the second agent(s) are administered sequentially. For example, in certain embodiments, the HSP90 inhibitor is administered after the treatment regimen of the mTOR inhibitor, and/or additional anti-cancer agent has ceased. In certain embodiments, the mTOR inhibitor, and/or additional anti-cancer agent is administered after the treatment regimen of the HSP90 inhibitor has ceased.

Cancer therapies that can be combined with HSP90 inhibitors according to the invention include surgical treatments, radiation therapy, and chemotherapeutic agents such as biotherapeutics. Exemplary anti-cancer agents, include, but are not limited to, radiation therapy, interferon (e.g., interferon α, interferon γ), antibodies (e.g. HERCEPTIN (trastuzumab), T-DM1, AVASTIN (bevacizumab), ERBITUX (cetuximab), VECTIBIX (panitumumab), RITUXAN (rituximab) BEXXAR (tositumomab)), anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids or taxanes (e.g. paclitaxel, albumin-bound paclitaxel (ABRAXANE), nab-paclitaxel, docetaxel (e.g., as an injectable Docetaxel (Taxotere)), taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase Inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin-aminopterin, and hexamethyl melamine.

In other embodiments, a HSP90 inhibitor and the second agent(s), e.g., the mTOR inhibitor, the ALK inhibitor, or the chemotherapeutic agent, can be used in combination with one or more of: other chemotherapeutic agents, radiation, or surgical procedures.

### EXEMPLIFICATION

This invention is further illustrated by the following examples which should not be construed as limiting. The contents of all references, figures, sequence listing, patents and published patent applications cited throughout this application are hereby incorporated by reference.

### Example 1

### Preparation of the Hydrochloride Salt of the Hydroquinone of 17-AAG

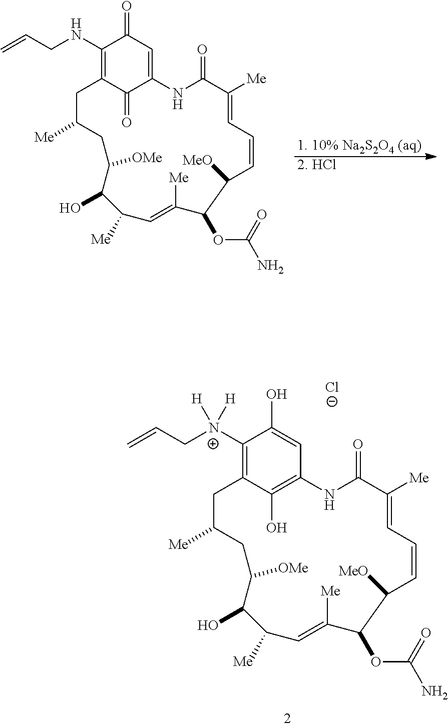

17-AAG (0.450 g, 0.768 mmol, 1.0 equiv) is dissolved in dichloromethane (50 mL) and stirred with a 10% aqueous solution of sodium hydrosulfite (50 mL). The solution is stirred for 30 minutes. The organic layer was collected, dried over Na₂SO₄, filtered and transferred to a round bottom flask. To this solution was added a solution of HCl in dioxane (4 N, 0.211 mL, 1.1 equiv.). The resulting mixture was allowed to stir under nitrogen for 30 minutes. A yellow solid slowly crashed out of solution. The yellow solid was purified by recrystallization form MeOH/EtOAc to yield 0.386 g of the hydroquinone HCl salt (2).

Compound 2 is also referred to herein as IPI-504. IPI-504 (retaspimycin hyrdrochloride) is a water-soluble, potent inhibitor of Hsp90.

Additional salts of 17-AAG can be prepared following the procedures described herein, and/or known in the art (see e.g., US 2006/0019941, U.S. Pat. No. 7,375,217 and U.S. Pat. No. 7,767,663, the contents of which are hereby incorporated by reference). For example, US 2006/0019941 discloses hydrobromide salts, p-toluenesulfonate salts, d-camphorsulfonate salts, hydrogen phosphate salts, methylsulfonate salts, benzenesulfonate salts, of 17-AAG. U.S. Pat. No. 7,767,663 discloses the preparation of salts of 17-AAG, including dimethylamino acetate co-salts (disclosed in Example 3 of U.S. Pat. No. 7,767,663), α-aminoisobutyrate co-salts (Example 4), β-alanine co-salts (Example 5), N-methyl glycine co-salts (Example 6), piperidine carboxylate co-salts (Example 7), glycine co-salts (Example 8), 2-amino-2-ethyl-butyrate co-salts (Example 9), 1-amino-cyclopropanecarboxylate co-salts (Example 10), 1-amino-cyclopentanecarboxylate co-salts (Example 12), N-methyl piperidinecarboxylate co-salts (Example 13), N,N,N-trimethylammonium acetate co-salts (Example 14).

The preparation of exemplary solid and liquid formulations of IPI-504 is disclosed in Examples 32-35 of US 2006/0019941.

### Example 2

### ALK Mutations Predict Response and Clinical Benefit to Treatment with HSP90 Inhibitors, Including IPI-504

Retrospective molecular characterization was performed on tumor samples from patients having relapsed and/or refractory stage IIIb or stage 1V non-small cell lung cancer (NSCLC) and who were enrolled in a phase 1/2 study testing safety, tolerability and activity of IPI-504 (Infinity Pharm.). ALK status was determined by the dual-color, break-apart fluorescence in situ hybridization (FISH) using probes developed by Vysis™ following the manufacturer's protocol. The test was scored as negative when the probes were either overlapping (yellow) or within 2 probe lengths from each other. The test was scored as positive when the probes were isolated or the distance between them was greater than 2 probe lengths in >15% of cells or >8/50 of nuclei. For example, in an ALK FISH from a patient with a partial response, wild-type ALK was represented by colocalization of the two (green and red) fluorescent probes and ALK gene re-arrangement was indicated by split FISH signal.

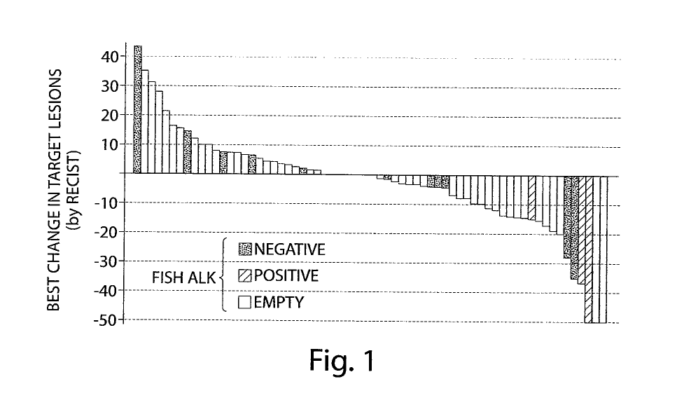

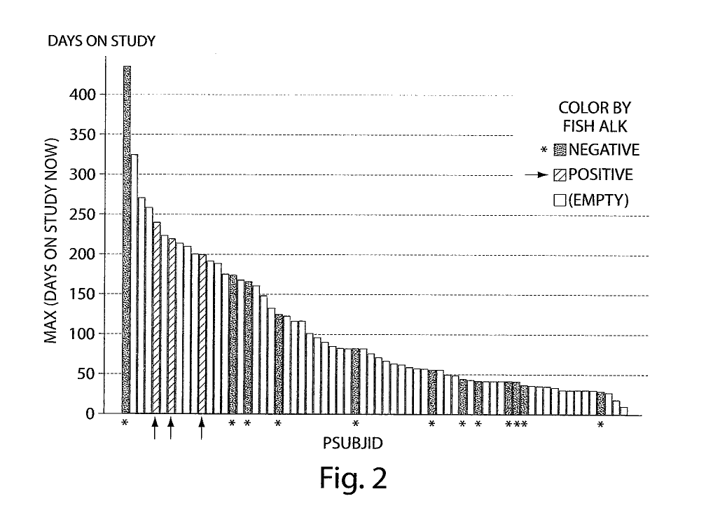

Two of five patients with partial response with IPI-504, as defined by RECIST (>30 tumor shrinkage), tested positive for ALK FISH (FIGS. 1-2). A third ALK positive patient by FISH had prolonged stable disease. Additional molecular studies including EGFR and KRAS genotyping by DxS and general oncogene and tumor suppressor gene genotyping by Oncomap have shown no other genomic alterations in these patients. Accordingly, the results indicate that ALK mutations identify patients most likely to benefit from HSP90 inhibitors, including IPI-504.

### Example 3A

### Association Between ALK Rearrangements (RALK) and the Clinical Activity of IPI-504 (Retaspimycin Hydrochloride) in Patients with Non-Small Cell Lung Cancer (NSCLC)

### Summary

This example describes the results from a clinical trial that assessed the efficacy of IPI-504 (a potent inhibitor of Hsp90 described herein) after EGFR tyrosine kinase inhibitor (TKI) therapy in patients with advanced, molecularly-defined non-small cell lung cancer (NSCLC).

Patients with advanced NSCLC, prior treatment with EGFR TKIs, and tumor tissue available for molecular genotyping were enrolled in this prospective, non-randomized, multicenter, phase II study of IPI-504 monotherapy. The primary outcome was objective response rate. Secondary aims included safety, progression-free survival (PFS), and analysis of activity by molecular subtypes.

Seventy-six patients were enrolled between December 2007 and May 2009 from ten US cancer centers. An overall response rate of 7% was observed in the overall study population, 10% in patients who were EGFR wild-type, 4% in patients with EGFR mutations, and 12% among KRAS wild-type patients. Among the 3 patients with an ALK gene rearrangement, 2 had partial responses and the third had prolonged (7.2 months) stable diseases (24% reduction in tumor size). The common adverse events included fatigue, nausea, and diarrhea, which were mostly grades 1 and 2. Grade 3 or higher liver function abnormalities were observed in less than 10% of patients.

In conclusion, IPI-504 has clinical activity patients with NSCLC, and in particular among patients with ALK rearrangements. NSCLC patients with ALK rearrangement can preferentially response to Hsp90 inhibition.

### Background and Study Design

Heat shock protein (Hsp) 90 is integral in protein homeostasis and regulates the stability of key proteins involved in oncogenesis, proliferation, and survival through its role as a protein chaperone (Whitesell L. et al. Nat Rev Cancer. (2005) 5(10):761-772). Hsp90 is an emerging focus of cancer therapy by virtue of its ability to inhibit multiple vital signaling pathways simultaneously (Xu W. et al. Clin Cancer Res (2007)13(6):1625-1629; Workman P. et al. Ann N Y Acad. Sci. (2007) 1113:202-216). Furthermore, mutated oncoproteins, including epidermal growth factor receptor (EGFR), can preferentially rely on Hsp90 chaperones more than their wild-type counterparts, further increasing the appeal of Hsp90 as a therapeutic target for cancers defined by such mutations (Nathan D. F. et al. Mol Cell Biol. (1995) 15(7):3917-3925; Rutherford S. L. et al. Nature (1998) 396(6709):336-342; Grbovic O. M. et al. Proc Natl Acad Sci USA. (2006) 103(1):57-62; Shimamura T. et al. Cancer Res. (2005) 65(14):6401-6408).

Non-small cell lung cancer (NSCLC) is a heterogeneous disease that can be sub-classified based on “driver mutations,” in which specific oncogene mutations result in dependence upon the driver's signaling pathway, or “oncogene addiction.” Common driver mutations in NSCLC appear to involve the genes for KRAS, epidermal growth factor receptor (EGFR), and anaplastic lymphoma kinase (ALK) (Suda K. et al. (2010) Cancer Metastasis Rev. 29(1):49-60; Sharma S. V. et al. (2007) Nat Rev Cancer. 7(3):169-181; Shaw A. T. et al. (2009) 27(26):4247-4253). When potent and specific inhibitors are used to block the signal from the driver oncogene, treatment can be effective, as demonstrated in the case of EGFR tyrosine kinase inhibitors (TKIs) in EGFR-mutant NSCLC (Mok T. S. et al. (2009) N Engl J. Med. 361(10):947-957; Kobayashi K. et al. (2009) J Clin Oncol. 27(15s):suppl abst 8016; Mitsudomi T. et al. (2010) Lancet Oncol. 11(2):121-128). This success can be mirrored with ALK TKI therapy in ALK-rearranged NSCLC (Kwak E. L. et al. (2009) J Clin Oncol. 27(15s):suppl; abstr 3509).

An analog of 17-AAG, IPI-504's potential anti-cancer activity has been validated in pre-clinical in vitro and in vivo models (Ge J. et al. (2006) J Med. Chem. 49(15):4606-4615; Sydor J. R. et al. (2006) Proc Natl Acad Sci USA. 103(46):17408-17413). In particular, the biological and anti-neoplastic effects of IPI-504 have been demonstrated in multiple human xenograft and murine orthotopic models of cancer. The free base of IPI-504 inter-converts with 17-AAG and exists in a pH and enzyme-mediated dynamic redox equilibrium in humans (Ge J. et al. (2006) J Med. Chem. 49(15):4606-4615; Demetri G. D. et al. (2007) J Clin Oncol. ASCO Annual Meeting Proc. 2007; 25:10024). In addition, ALK is a client protein of the Hsp90 chaperone inhibited by IPI-504.

Preclinical studies suggested that mutant EGFR can be a stronger client of Hsp90 than wild-type EGFR. A phase I dose-escalation study of intravenous IPI-504 monotherapy in patients with NSCLC demonstrated a favorable side-effect profile and evidence of clinical benefit (Sequist L. V. et al. (2007) AACR-NCI-EORTC International Conference on Molecular Targets and Cancer Therapeutics, San Francisco, Calif.). Therefore this multicenter phase II study was conducted to prospectively assess the efficacy of IPI-504 after EGFR TKI therapy in advanced NSCLC patients. EGFR genotype was mandatory so that difference in activity by mutation status could be observed. Other biomarkers were retrospectively assessed to identify the groups more likely to respond to therapy.

### Study Design and Patient Selection

This was a non-randomized phase II clinical trial to assess the objective response rate (ORR) to IPI-504 monotherapy in patients with advanced NSCLC who either had an activating EGFR mutation or were EGFR wild-type (Therasse, P., et al. (2000) J Natl Cancer Inst 92, 205-216). Each genotype-defined arm of the trial (mutant EGFR cohort or wild-type EGFR cohort) functioned as a Simon two-stage study with planned interim evaluation after 10 patients and expanded enrollment of an additional 19 patients (a total of 29 patients) if there was at least one patient with a complete response (CR), partial response (PR) or stable disease (SD) lasting 3 months. As pre-defined EGFR genotype was not a requirement at study entry, the trial remained open until both cohorts fully enrolled, which led to over-enrollment of the wild-type arm. Secondary aims included describing the safety and progression-free survival (PFS) of the regimen, and examining molecular markers associated with response.

Patients were recruited between December 2007 and May 2009 from ten US cancer centers. To be eligible, patients had to have stage IIIB (with pleural effusion), or stage 1V NSCLC with progression on EGFR TKI therapy at some point in their history; adequate baseline renal, hepatic, and bone marrow function; Eastern Cooperative Oncology Group performance status (PS) of 0-2; measurable disease by RECIST 1.0; no active or untreated central nervous system (brain) metastases; no significant cardiac conduction abnormalities; no ongoing keratoconjunctivitis; and either previously defined EGFR genotype or sufficient tumor tissue to undergo genotype assessment, for example, EGFR mutation analysis (EGFR status not required for study entry) (Therasse, P. et al. (2000) J Natl Cancer Inst 92, 205-216; Oken, M. M. et al. (1982) Am J Clin Oncol. 5:649-655). Patients had experienced prior EGFR TKI therapy and there was no limit on prior therapies. All patients signed a written informed consent and the study was monitored by all local institutional review boards. Funding for the trial was provided by Infinity Pharmaceuticals, Inc.

### Treatment and Evaluation

Treatment consisted of a 30-minute infusion of intravenous IPI-504 on days 1, 4, 8 and 11 of a 21-day cycle. Therapy continued until progressive disease (PD), intolerable side effects, or elective withdrawal. A total of 76 patients were enrolled. The starting dose was 400 mg/m² for 75 patients. In April 2009, the dose for patients who were on study (19 patients) was lowered to 225 mg/m², due to hepatotoxicities observed at the 400 mg/m² dose in a separate trial of IPI-504 in patients with gastrointestinal stromal tumors (GIST) (Demetri G. D. et al. Final results from a phase III study of IPI-504 (retaspimycin hydrochloride) versus placebo in patients with gastrointestinal stromal tumors (GIST) following failure of kinase inhibitor therapies. Paper presented at: Gastrointestinal Cancers Symposium; Jan. 22-24, 2010, 2010; Orlando, Fla.), and the last enrolled patient started at a dose of 225 mg/m².

All patients were assessed for safety by history, physical examination, blood chemistries, liver function tests and blood counts, at baseline and prior to each infusion. Amylase and lipase were assessed if there were symptoms of pancreatitis. Adverse events were graded using the NCl common toxicity criteria version 3.0 (NCl. Common terminology criteria for adverse events (CTCAE) version 3.0 Aug. 9, 2006). Slit lamp eye examinations were performed during screening and electrocardiograms were obtained during screening and before and after the first infusion, to evaluate for keratitis and QTc prolongation, respectively. Radiographic evaluation of tumor response by CT-scan was performed after every 2 cycles. All films were reviewed and independently assessed by a central radiology core laboratory.

### Patient Tumor Molecular Analyses

Tumor tissue specimens from all patients were assessed for EGFR mutations via direct sequencing of exons 18-21, using standard methods. EGFR sequencing was performed at participating institutions' CLIA-certified internal laboratories or at Genzyme (Cambridge, Mass.). A subset of patients also underwent EGFR (n=25), KRAS (n=30) and BRAF (n=5) genotyping analysis with the allele-specific ARMS assay (DxS, United Kingdom) at Infinity Pharmaceuticals, Inc (Cambridge, Mass.). Patients who underwent successful testing via both direct sequencing and the allele-specific ARMS assay were classified using the result of the more sensitive assay (allele-specific ARMS assay). Post-hoc analyses of other molecular markers of interest were performed for all patients for whom sufficient tissue was available. The primary post-hoc analyses were performed in CLIA-certified laboratories and consisted of the SNaPshot assay (Applied Biosystems, Foster City, Calif.), adapted to detect key oncogenic mutations in EGFR, KRAS, PIK3CA, BRAF, PTEN, AKT, TP53, NRAS, CTNNB1 (beta-catenin), APC, KIT, JAK2, NOTCH1, and FLT3 (n=19); and the fluorescence in situ hybridization (FISH) break-apart assay for detection of ALK gene rearrangements, using methods previously described (n=15) (Shaw A. T. et al. (2009) J Clin Oncol. 27: 4247-4253; Dias-Santagada D. et al. (2010) EMB Mol. Med. (in press)). Other post-hoc analyses were performed on a subset of samples, which included genotyping by Oncomap analysis (Dana-Farber Cancer Institute; Boston, Mass.) covering 1155 mutations in 114 cancer genes (n=10). In addition, 11 genes (ALK, BRAF, EGFR, ERBB2, HSP90AA1, HSP90AB1, KRAS, MET, NF1, PTEN and STK11) were sequenced by the Sanger method at Functional Biosciences Inc. (Madison, Wis., n=12)) and Genewiz (South Plainfield, N.J.). The nucleotide sequences used for the sequencing analysis of ALK, BRAF, EGFR, ERBB2, HSP90AA1, HSP90AB1, KRAS, MET, NF1, PTEN and STK11 are shown as SEQ ID NOs:35-56 (ALK), SEQ ID NOs:57-58 (BRAF), SEQ ID NOs:59-112 (EGFR), SEQ ID NOs:113-172 (ERBB2), SEQ ID NOs:173-236 (HSP90AB1), SEQ ID NOs:237-244 (KRAS), SEQ ID NOs:245-252 (MET), SEQ ID NOs:253-368 (NF1), SEQ ID NOs:369-394 (PTEN), and SEQ ID NOs:395-414 (SKT-11). Copy number was evaluated using a CGH and SNP arrays.

### Western Blot Analyses

In order to explore further the significance of the clinical molecular observations, laboratory models assessing sensitivity of EGFR and ALK-mutant cancer cell lines to varying concentrations of 17-AAG were derived. H1975 (EGFR L858R/T790M), HCC827 (EGFR del 19), H3122 (EML4-ALK), and MGH006 (EML4-ALK derived from a patient who was sensitive to PF-02341066) cells were treated with increasing doses of 17-AAG for 24 hours. Western blotting was performed using previously described methods (Faber A. C. et al. Proc Natl Acad Sci USA. (2009) 106(46):19503-19508). Membranes were probed with antibodies against P-ALK (Cell Signaling, Inc.), ALK (Cell Signaling, Inc.), P-EGFR (Biosource), and EGFR (Santa Cruz). Chemiluminescence was detected using the Syngene G:Box camera (Synoptics), and signal intensity was quantified using Syngene Genetools software (Synoptics). All measurements were performed in the linear range without saturation and were normalized to ERK loading control.

### Cell Survival Assays

Cells were seeded at 2,000 cells per well of a 96-well plate. After overnight incubation, the cells were treated in triplicate with serial dilutions of 17-AAG for 72 h. Viable cell titer relative to untreated cells was determined using Syto60 assays as previously described (Faber A. C. et al. Proc Natl Acad Sci USA. (2009) 106(46):19503-19508). Membranes were probed with antibodies against P-ALK (Cell Signaling, Inc.).

### Statistical Considerations

The primary endpoint of the study was ORR, calculated as the sum of patients with confirmed complete or partial responses divided by the number of treated patients. Each arm (EGFR mutant and wild-type) was analyzed independently. The study was powered assuming a null ORR of 5% and a target ORR of 20%.

Summary statistics were used to describe safety and included all patients treated with IPI-504. PFS was defined as the time from enrollment to progressive disease or death, censored at the last known follow-up, and was calculated with the Kaplan-Meier method, following the intent-to-treat principle.

### Results

### Patients

Seventy-six patients were enrolled in the study. EGFR genotype analysis did not need to be completed prior to start of study treatment, consequently 8 (10%) patients with indeterminate genotype were not assigned to either the EGFR mutant or wild-type arms. The median age was 64 (range 31-82) years and was similar between the genotypes (Table 2). The entire study population had an over-representation of women (63%) and never-smokers (45%), which was even more pronounced among the EGFR mutants (71% women and 61% never-smokers). The study cohort was also heavily pre-treated with a median of 4 prior systemic regimens and a median time since diagnosis of 27.5 months. Prior EGFR TKI therapy had yielded a 54% response rate and lasted a median of 10.5 months among EGFR mutation-positive patients.

### Toxicity

IPI-504 was well tolerated. Most adverse events were grades 1 or 2 and 9 (12%) patients had dose reductions for toxicity, while 11 (14%) discontinued therapy for adverse events. The most commonly reported adverse events were fatigue, nausea, diarrhea, vomiting, cough, anorexia and joint/muscle aches, Table 3. About a third of patients had transient, non-toxic purple-colored urine due to a renal clearance of an IPI-504 chromometabolite. In terms of laboratory abnormalities, aspartate aminotransferase (AST), alanine aminotransferase (ALT), and alkaline phosphatase elevations were common (49%, 41% and 62%, respectively), but Grade 3 or greater elevations were infrequent (9%, 7% and 5%, respectively). No Grade 3 or 4 bilirubin elevation was noted. Three patients died while on study, all three were considered related to study drug. Two died of complications from pneumonia, including sepsis and respiratory failure or distress, and had renal failure and ALT and AST elevations prior to death. The third patient died from complications following respiratory distress, lactic acidosis, nausea, and vomiting.

### Response and Molecular Analyses

Sixty-eight (89%) patients had successful EGFR genotype analyses, with 28 (37% of the 76 enrolled) patients assigned to the EGFR mutation-positive arm and 40 (53%) to the wild-type arm. Of the EGFR mutant patient, 16 (57%) had exon 19 deletions, 6 (21%) had an exon 21 L858R point mutation, 2 (7%) had exon 20 insertions, and 4 (14%) had two mutations each (2 patients with exon 18 G719S and exon 21 L861Q mutations; 2 with exon 19 deletion and exon 20 T790M mutations). Reasons for indeterminate EGFR genotype included lack of tumor tissue or poor quality or quantity of available tissue. Thirty-eight (50%) patients underwent KRAS mutation testing and 12 (16%) had a mutation; 15 (20%) patients underwent ALK rearrangement testing and 3 (4%) were positive. Demographic characteristics of the patients who were positive for a KRAS mutation were notable for a positive smoking history, and those of the ALK rearranged patients were notable for young age, male predominance, and never-smoking (Table 2).

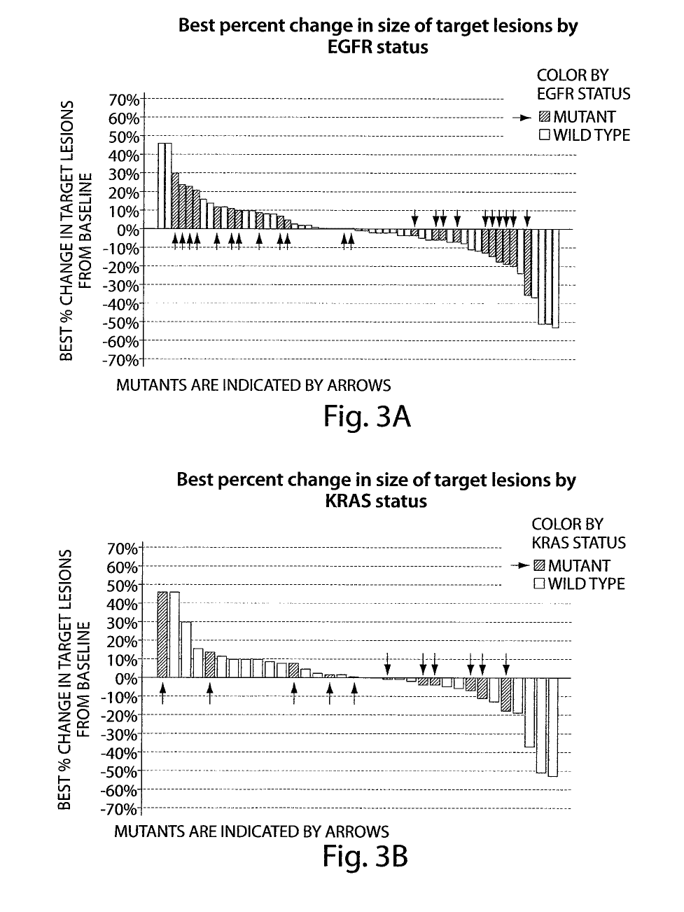

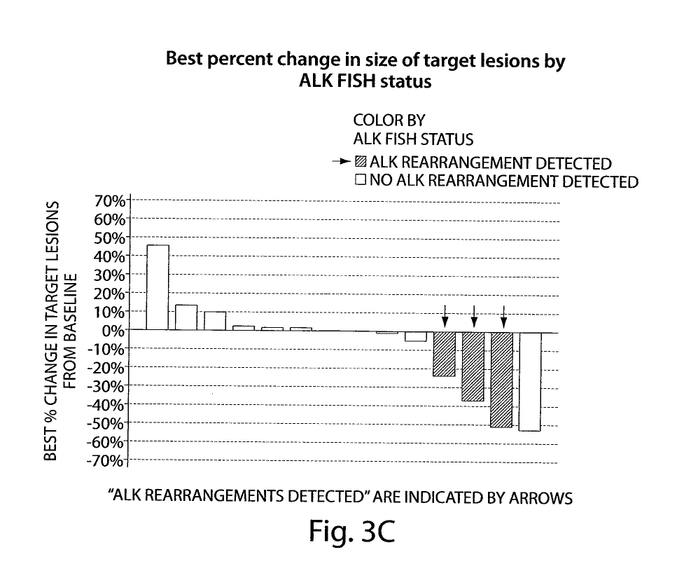

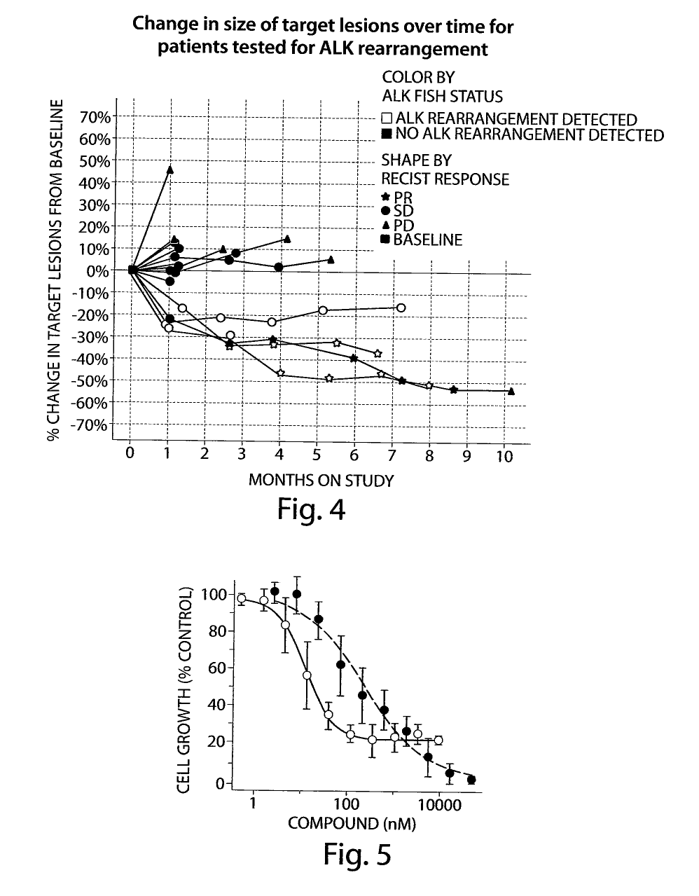

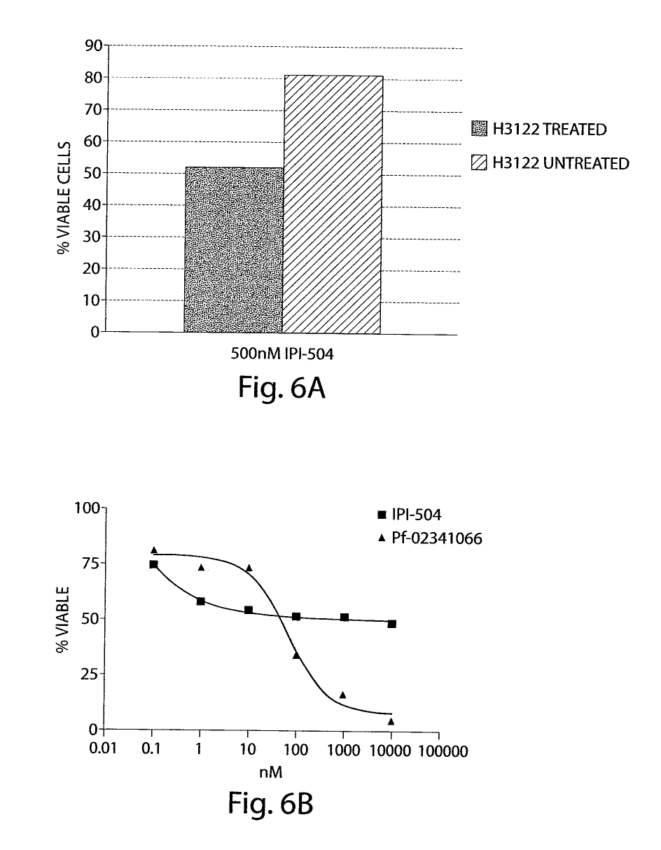

The ORR to IPI-504 was 7% overall, 10% in EGFR wild-type patients and 4% in EGFR mutants. The EGFR mutation-positive patient with a RECIST PR had an L858R mutation, had previously had a PR to the combination of erlotinib and enzastaurin lasting for approximately 8 months and transitioned directly from erlotinib to IPI-504. Responses were also seen in 12% of KRAS wild-type patients, and in 67% of patients with an ALK rearrangement (Table 4, FIGS. 3A-3C). FISH break-apart assay was used to detect a patient positive for ALK rearrangement. For example, wild-type allele was shown as one yellow signal and ALK rearranged allele was shown as separated red and green probe signals. Note that 2 of the 3 KRAS wild-type responders had ALK rearrangement, but the third was confirmed ALK wild-type. At the time of analysis, 35 (46%) patients had a PFS event (progressed or died), and 41 (54%) were censored. The median PFS was 2.86 months (95% CI-2.43, 4.18) for all patients, though the 3 patients with ALK rearrangements received IPI-504 for approximately 7 months and have not yet progressed or died on study (FIG. 4). Images from ALK translocation positive patient with partial response indicate a partial reduction in tumor size in the lung after cycle 12 compared to the baseline. Additional genetic results from snapshot, Oncomap, DxS and Sanger sequencing are summarized in Supplemental Table 1.

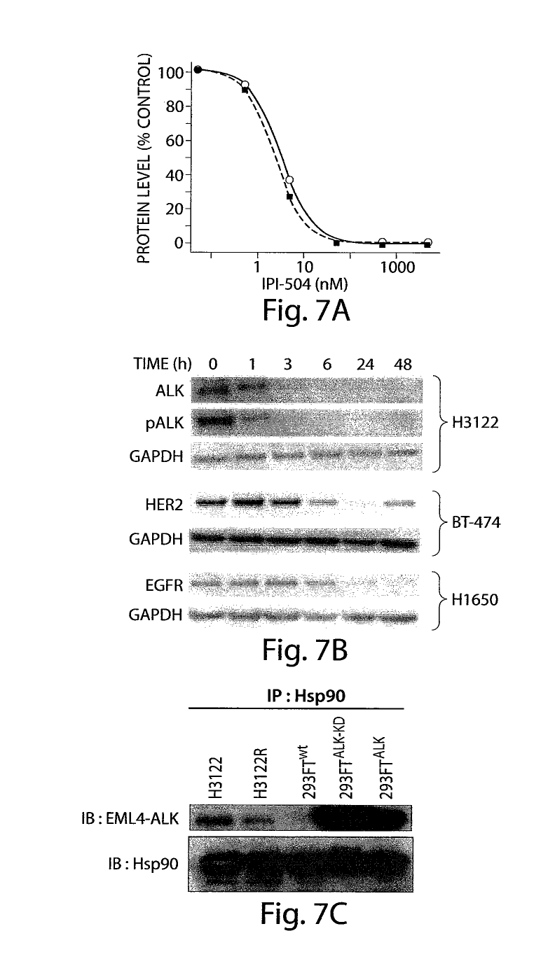

Laboratory assessments of lung cancer models harboring EGFR mutations and ALK rearrangments confirmed that the EGFR mutant models were sensitive to Hsp90 inhibition with 17-AAG, as previously demonstrated, but also revealed that ALK-rearranged models were highly sensitive. Indeed, both the stability of the ALK-rearranged protein and the viability of the cancer cells were highly sensitive to Hsp90 inhibition, for example, as shown by immunobloting (FIG. 7B) and relative dose-response curve of cell survival. Therefore, ALK is a more sensitive client protein than EGFR. In one embodiment, the invention discloses a relationship between the sensitivity of client protein degradation and clinical response to treatment with HSP90 inhibitors.

### Discussion

This trial is the first of an Hsp90 inhibitor in molecularly-defined cohorts of patients with advanced NSCLC. This study has demonstrated that IPI-504 is active in NSCLC, with a response rate of 7% in the overall study population, 10% in patients who were EGFR wild-type, 4% in patients with EGFR mutations and acquired resistance to TKIs, and 12% among KRAS wild-type patients. The intriguing finding is the post-hoc analysis demonstrated that 2 of 3 patients known to have ALK rearrangements had a PR to IPI-504 and the third patient had SD (24% reduction) for 7.2 months. This is the first demonstration of clinical activity of an Hsp90 inhibitor in patients with ALK rearrangements, suggesting that NSCLC patients with rALK can preferentially respond to Hsp90 inhibition.

ALK is a member of the insulin superfamily of receptor tyrosine kinases and was initially associated with anaplastic large cell lymphoma, which commonly has ALK oncogenic signaling mediated by fusion between the ALK kinase domain and the partner protein nucleophosmin (NPM) (Morris S. W. et al. Science (1994) 263(5151):1281-1284). More recently, EML4-ALK and other rearrangements involving the ALK locus have been described in NSCLC as transforming driver mutations conferring sensitivity to therapy with ALK TKIs (Shaw A. T. et al. J Clin Oncol. (2009) 27(26):4247-4253; Kwak E. L. et al. J Clin Oncol. (2009) 27(15s):suppl; abstr 3509; Soda M. et al. Nature (2007) 448(7153):561-566). The prevalence of ALK translocations in NSCLC is approximately 5%. The prevalence in patients who have never smoked or are light smokers without EGFR mutations is approximately 33%. ALK translocations in NSCLC is associated with adenocarcinoma, signet ring cell subtype. Pre-clinical models have demonstrated that NPM-ALK is a client of Hsp90 (Bonvini P. et al. Cancer Res. (2002) 62(5):1559-1566) and FIG. 7B indicates that EML4-ALK is also a potent client. As shown in FIG. 7B, EML4-ALK is a more sensitive client protein than mutant EGFR or HER2.

Overall, the study confirms that Hsp90, by virtue of its chaperone role for multiple oncoproteins and pervasive effect on key signaling pathways, has the potential to be an effective cancer therapy against multiple types of oncogene-addicted cancers, including those that have developed resistance to receptor-specific targeted treatments. TKIs that inhibit “driver mutations” in such cancers have been effective, including imatinib in chronic myelogenous leukemia and GIST (targets BCR-ABL and c-KIT, respectively), gefitinib and erlotinib in NSCLC (targets EGFR), and PF-02341066 in NSCLC (targets ALK) (Mok T. S. et al. (2009) N Engl J. Med. 361:947-957; Kwak E. L. et al. (2009) J Clin Oncol. 27(15s):suppl; abstr 3509; Druker B. J. et al. (2001) N Engl J Med. 344:1038-1042; Demetri G. D. et al. (2002) N Engl J. Med. 347:472-480). This study confirms that inhibition of a driver mutation need not be via a receptor-specific molecule in order to be highly effective; inhibition of Hsp90, with subsequent impact on multiple signaling pathways, is now a bona fide approach to cancer therapy.

It is notable that despite extensive pre-clinical evidence that Hsp90 inhibition, and specifically IPI-504 treatment, leads to effective suppression of tumor growth in EGFR mutation-positive models, including those with acquired resistance to EGFR TKIs, few responses in patients with EGFR mutations were observed (Shimamura T. et al. Cancer Res. (2008) 68(14):5827-5838; Sawai A. et al. Cancer Res. (2008) 68(2):589-596; Brain J. et al. IPI-504, a novel orally administered Hsp90 inhibitor, demonstrates anti-tumor effects in EGFR mutant, kinase inhibitor resistant NSCLC. Paper presented at: American Association of Cancer Research Annual Meeting; Apr. 14-18, 2007; Los Angeles, Calif.). There could be several reasons for this observation. The population of patients was atypical in that the median time from diagnosis among patients with EGFR mutations was two years and 56% had been treated with at least two prior EGFR TKI agents. Since their cancers bad become resistant to EGFR TKIs, the biology of their tumors can have changed from being dependent on a single oncogene to a more heterogeneous state. The dose of IPI-504 could have been a factor in the modest response rate among EGFR mutants. The analysis of cancer cell lines suggests that lower concentration of Hsp90 inhibitors can be required to downregulate expression of EML4-ALK compared to mutant EGFR. The dose-response curve for EGFR mutant cancers was modestly shifted to the right compared to the dose-response curve for ALK-rearranged cancers. The potentially wider therapeutic window can have also contributed to the higher response rate observed in the patients with an ALK rearrangement. Importantly, the lack of acquired resistance to ALK-specific therapy among the patients with an ALK rearrangement can imply a discrete molecular biology that was more susceptible to Hsp90 inhibition than the patients with EGFR mutations, all of whom had previously received and acquired resistance to EGFR TKIs. None of the patients on the trial (regardless of genotype) had previously been treated with an ALK-specific therapy.

In this study, IPI-504 was generally well tolerated, with low rates of grade 3 or higher adverse events. The most common adverse events included fatigue, nausea, and diarrhea, and these were mostly grades 1 and 2. Grade 3 or higher liver function abnormalities were observed in <10% of patients and drug-related deaths were infrequent and complicated by patients underlying lung cancer. This is in contrast to observations in late-stage GIST patients treated with IPI-504, in which life-threatening liver toxicity was seen (Demetri G. D. et al. Final results from a phase III study of IPI-504 (retaspimycin hydrochloride) versus placebo in patients with gastrointestinal stromal tumors (GIST) following failure of kinase inhibitor therapies. Paper presented at: Gastrointestinal Cancers Symposium; Jan. 22-24, 2010, 2010; Orlando, Fla.).

Both the EGFR mutation-positive and wild-type cohorts had a long interval since diagnosis, a high number of prior therapies, and a low proportion of smokers. Furthermore, tumor tissue available for genetic analysis was primarily from diagnostic biopsies, prior to any targeted therapy or development of resistance that might have altered the genetic signature. However, the fact that tumor tissue for genotyping was collected from 100% of participants due to eligibility mandate is a credit to the study. This study not only provides specific observations regarding response by EGFR genotype, but also includes careful examination of the minority of patients with robust responses, enabling the key observation of activity in ALK-rearranged NSCLC. It is believed that all studies of targeted therapies should require tissue from all participants. It is not uncommon for studies of novel agents to show activity among only a minority of patients, and this study effectively illustrates how post-hoc molecular analysis of the best responding patients can provide direction for avenues of further research. Of note, it was confirmed that ALK rearrangement in the patients with the current standard “break-apart” FISH assay that detects rearrangement in chromosome 2 but does not identify the specific variant of EML4-ALK present (EML4 has multiple break-points at which it can partner with ALK) (Horn L. et al. J Clin Oncol. (2009) 27(26):4232-4235). Therefore, it is currently unknown if IPI-504 has a range of expected activity dependent on the oncogenic EML4-ALK variant.

In summary, IPI-504 is a novel inhibitor of Hsp90 with activity in patients with NSCLC, in particular those with ALK rearrangements. It is notable that unlike the positive association between detection of ALK rearrangements and clinical activity activity of IPI-504 monotherapy in patients with NSCLC, few responses were observed to IPI-504 monotherapy in patients with K-Ras or EGFR mutations. Further study can be conducted to prospectively evaluate the efficacy of Hsp90 inhibition in patients with ALK rearrangements and other oncogenic driver mutations.

### Example 3B

### Hsp90 Inhibition Results in a Significant Delay in Tumor Progression in a Model of Emerging EGFR TKI Resistance in Non-Small Cell Lung Cancer

Heat-shock protein 90 (Hsp90) has emerged as an attractive target in cancer due to its role in maintaining the activity and stability of a variety of oncoproteins, including HER2, BCR-ABL, EML4-ALK and mutant EGFR. Infinity is developing two novel Hsp90 inhibitors, IPI-504 (IV administered) and IPI-493 (orally administered). IPI-504 is currently being evaluated in multiple phase 2 clinical trials; IPI-493 is being evaluated in two phase 1 trials.

EGFR tyrosine kinase inhibitors (TKIs) are an effective treatment for lung cancer patients with activating mutations in EGFR. After a dramatic initial response, however, most patients become resistant to drug treatment and progress. In about half of these cases, resistance is due to a second site point mutation in EGFR (T790M). It is believed that in at least some of these cases the TKI resistance mutations are pre-existing and that treatment with TKIs selects for the resistant cells.

In an effort to model the emergence of resistance to TKIs from pre-existing mutations, we developed an in vivo model, where gefitinib treatment initially leads to tumor regression followed by rebound of tumor growth and outgrowth of drug resistant clones containing the T790M mutation. In this model, treatment with IPI-493 alone or IPI-493 following gefitinib resulted in a tumor growth inhibition of 61 and 77%, respectively, when compared with gefitinib treatment alone. Treatment with IPI-493 alone also resulted in a significant delay in time to tumor progression with ˜40% of animals still on study on day 45; all animals treated with either vehicle or gefitinib had been removed due to tumor progression. Interestingly, treatment with IPI-493 following gefitinib resulted in an even more impressive delay in time to progression, with >50% of animals still on study on day 65.

These studies suggest that further studies with Hsp90 inhibitors in EGFR mutant NSCLC patients who have been pre-treated with a TKI may be warranted.

### Example 4

### Pre-Clinical Evaluation of Hsp90 Inhibitors in NSCLC

This example describes in vitro and in vivo studies showing the inhibition of tumor cell growth after treatment with an HSP90 inhibitor, alone or in combination with other agents. More specifically, this example shows that the Hsp90 inhibitor, IPI-504, rapidly lowers EML4-ALK levels and induces tumor regression in ALK-driven NSCLC models.

### Summary

Hsp90 is an emerging target for cancer therapy due to its important role in maintaining the activity and stability of key oncogenic signaling proteins. This example shows that the EML4-ALK fusion protein, presumed to be an “oncogenic driver” in about 5% of patients with NSCLC, is associated with Hsp90 in cells and is rapidly degraded upon exposure of cells to IPI-504. EML4-ALK is shown to be more sensitive to Hsp90 inhibition than either HER2 or mutant EGFR with an IC₅₀ for protein degradation in the low nanomolar range. This degradation leads to a potent inhibition of downstream signaling pathways and to the induction of growth arrest and apoptosis in cells carrying the EML4-ALK fusion. To generate a causative link between the expression of EML4-ALK and sensitivity to IPI-504, an EML4-ALK cDNA was introduced into HEK293 cells and shown that expression of the fusion protein sensitizes cells to IPI-504 both in vitro and in vivo. In a xenograft model of a human NSCLC cell line containing the ALK rearrangement, tumor regression was observed at clinically relevant doses of IPI-504. Finally, cells that have been selected for resistance to ALK kinase inhibitors retain their sensitivity to IPI-504. This study provides a molecular explanation for the clinical observations discussed in Example 3 showing partial responses to IPI-504 in NSCLC, specifically in patients that carry an ALK rearrangement.

### Background

Heat shock protein 90 (Hsp90) is an abundant cellular chaperone protein that maintains the stability, activity and sorting of its protein substrates also called client proteins Pearl, et al. (2006) Annu. Rev. Biochem. 75: 271-294; Young J C, et al. (2001) J Cell Biol 154: 267-73. Upon inhibition of Hsp90, client proteins are rapidly degraded through the proteasome Connell P, et al. (2001) Nat Cell Biol 3: 93-6. Hsp90 has recently become an emerging target for cancer therapeutics Neckers L. (2007) J. Biosci. 32: 517-530 since many of its client proteins are oncoproteins such as HER2Munster P N, et al. (2001) Cancer Res. 61: 2945-2952, mutant cKIT Dewaele B, et al. (2008) Clin. Cancer Res. 14: 5749-5758; Fumo G, et al. (2004) Blood 103: 1078-1084, mutant EGFR (Shimamura T, et al. (2008) Cancer Res. 68: 5827-5838; Shimamura T, Lowell A M, Engelman J A, Shapiro G I. (2005). Epidermal growth factor receptors harboring kinase domain mutations associate with the heat shock protein 90 chaperone and are destabilized following exposure to geldanamycins. Cancer Res. 65: 6401-6408

Shimamura T, et al. (2005) Cancer Res. 65: 6401-6408) or BCR-ABL An W G, et al. (2000) Cell Growth Differ 11: 355-60; Nimmanapalli R, et al. (2001) Cancer Res. 61: 1799-1804; Peng C, et al. (2007) Blood 110: 678-685. Consistent with this supportive role in malignant transformation and maintenance of oncogene addiction in some cancers, Hsp90 is overexpressed in cancer cells and overexpression is correlated with disease progression in melanoma McCarthy M M, et al. (2008) Ann Oncol 19: 590-4 and associated with decreased survival in breast Pick E, et al. (2007) Cancer Res 67: 2932-7, lung Gallegos Ruiz M I, et al. (2008) PLoS One 3: e0001722 and gastrointestinal stromal tumors L1 CF, et al. (2008) Clin Cancer Res 14: 7822-31.

Oncogenic activation of the anaplastic lymphoma kinase (ALK) occurs in various cancer types. In NSCLC, an intra-chromosomal rearrangement results in the fusion of the N-terminus of the echinoderm microtubule-associated protein-like 4 (EML4) with the C-terminal tyrosine kinase domain of ALK Soda M, et al. (2007) Nature 448: 561-6. To date, multiple EML4-ALK variants have been identified Choi Y L, et al. (2008) Cancer Res 68: 4971-6; Takeuchi K, et al. (2009) Clin Cancer Res 15: 3143-9. All fusion oncoproteins comprise the entire tyrosine kinase domain of ALK and variable portions of the EML4 protein. Dimerization of the fusion protein through the EML4 domain leads to constitutive activation of the ALK kinase and to cellular transformation Choi Y L, et al. (2008) Cancer Res 68: 4971-6; Koivunen J P, et al. (2008) Clin Cancer Res 14: 4275-83. IPI-504 (retaspimycin hydrochloride), a water soluble derivative of 17-AAG, is a novel, potent inhibitor of Hsp90 Ge J, et al. (2006) J. Med. Chem. 49: 4606-4615; Sydor J R, et al. (2006) Proc. Natl. Acad. Sci. U.S.A. 103: 17408-17413. The biological and anti-neoplastic effects of IPI-504 have been demonstrated in multiple in vitro and in vivo models of cancer Bauer S, et al. (2006) Cancer Res. 66: 9153-9161; Dewaele B, et al. (2008) Clin. Cancer Res. 14: 5749-5758; Leow C C, et al. (2009) Mol Cancer Ther 8: 2131-41; Peng C, et al. (2007) Blood 110: 678-685; Song D, et al. (2008) Mol. Cancer. Ther. 7: 3275-3284 leading to its clinical development in various phase 2 studies (*Sequist et al., 2010). While there is good preclinical rationale for the use of Hsp90 inhibitors in cancer and multiple inhibitors are in clinical development, none of the inhibitors have yet shown clinical proof of concept. This might be due, in part, to the difficulty of finding the right clinical indication for these inhibitors. It is not clear whether Hsp90 inhibitors in the clinic ultimately act through a sensitive client protein that is also an oncoprotein or whether cell killing is mediated through the simultaneous degradation of multiple clients and a more general effect on protein homeostasis. In a phase 2 trial of IPI-504 in NSCLC, we have recently observed partial responses in patients whose tumor cells contain rearrangements of the ALK locus (2010) J Clin Oncol 28: abstr 7517; Example 3). In this example, the sensitivity of EML4-ALK as an Hsp90 client protein, was examined whether EML4-ALK is causatively involved in the sensitivity of cancer cells to Hsp90 inhibition and the activity of IPI-504 in xenograft models that express the EML4-ALK fusion protein.

### Results

Observation of Clinical Benefit in Nsclc Patients Positive for Alk Gene Rearrangements with IPI-504 Treatment

In the phase 2 study of IPI-504 in NSCLC described in Example 3, an overall response rate of 7% was observed. Upon molecular characterization of patient tumors, we determined that the response rate was 4% in patients with mutant EGFR and 10% in patients with wt EGFR (Sequist L et al. (2010) J Clin Oncol 28: abstr 7517; Example 3). This finding was surprising mutant EGFR was expected to be a sensitive Hsp90 client protein mediating clinical responses to Hsp90 inhibition in this population. When patient tumors were further analyzed for genetic abnormalities, 3 samples (of 15 available for testing) scored positive for rearrangements involving the ALK locus. Interestingly, all three of these patients showed some degree of tumor shrinkage. Two patients reached >30% tumor shrinkage (PR) and one patient had a 24% tumor shrinkage and stable disease for greater than 7 months (FIG. 1).

### EML4-ALK is a Sensitive Client Protein of Hsp90

Rearrangements at the ALK locus have been reported in about 5% of NSCLC patients, forming an oncogenic fusion of the N-terminus of EML4 with the C-terminal kinase domain of ALK (Soda, M. et al. (2007) Nature 448: 561-6). To determine whether the EML4-ALK fusion protein is a client protein of Hsp90, we incubated the EML4-ALK-expressing NSCLC cell line H3122 with increasing concentrations of IPI-504 and measured the abundance of total and phosphorylated ALK protein by ELISA (FIG. 7a). In this experiment, virtually all ALK protein is degraded at concentrations above 50 nM IPI-504. The degradation IC₅₀ measured (4 nM) makes this the most sensitive Hsp90 client protein we have encountered. To directly compare the sensitivity of EML4-ALK to other well known Hsp90 client proteins, we incubated H3122, BT-474 and H1650 cells with 1 uM IPI-504 for different times and probed for EML4-ALK, HER2 and mutant EGFR in the relevant cell lines. We have previously shown in Tillotson B. et al. (2010) J Biol Chem that time to degradation is the most sensitive measure of client protein dependency on Hsp90. In this analysis, EML4-ALK is completely degraded by 3 h, whereas it takes about 24 h for most of the HER2 and mutEGFR to be degraded (FIG. 7b). To further confirm that EML4-ALK is indeed a client of Hsp90, we performed immunoprecipitations with an antibody directed against Hsp90a, and detected a protein band at the predicted molecular weight (90 kDa) with an ALK antibody in Hsp90 immunoprecipitates from H3122 cells but not control cells (FIG. 7c, lane 1 and 3).

### IPI-504 Treatment Induces EML4-ALK Degradation, Inhibition of Downstream Pathways and Inhibits Cell Growth

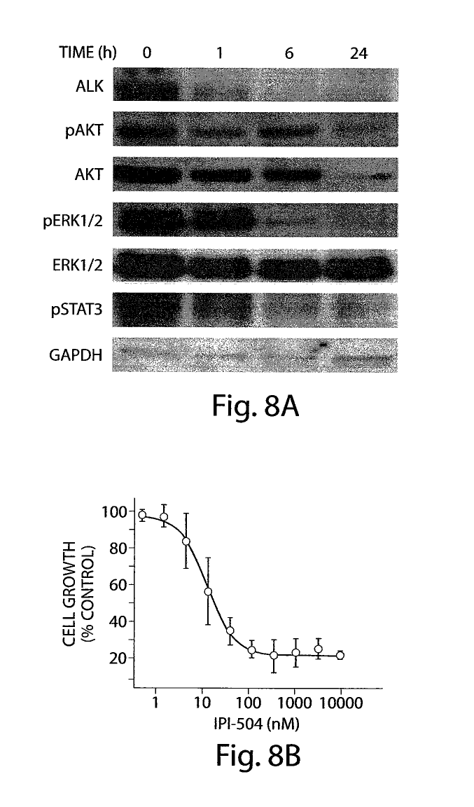

To investigate the cellular consequences of degrading EML4-ALK, we probed lysates from H3122 cells that had been incubated with IPI-504 for different times with antibodies against ALK and different downstream signaling proteins (FIG. 8a). Upon IPI-504 treatment and EML4-ALK degradation, the active (phosphorylated) forms of ERK and STAT3 are rapidly depleted whereas AKT and phospho-AKT are depleted on a different time scale. These results argue that EML4-ALK in the NSCLC cell line H3122 signals through the ERK and STAT pathways and that this signaling is effectively disrupted by Hsp90 inhibitor treatment. To investigate what effect the degradation of EML4-ALK and the inhibition of downstream signaling pathways have on cell growth, H3122 cells were incubated for 72 h with increasing concentrations of IPI-504 and cell growth monitored by measuring cellular ATP levels. IPI-504 has a potent effect on cell growth (FIG. 8b) with an IC50 value for growth inhibition of 22 nM. This value matches well with the IC50 of ALK degradation, consistent with ALK degradation being the cause for the cell growth inhibition observed.

Expression of EML4-ALK sensitizes cells to Hsp90 inhibition

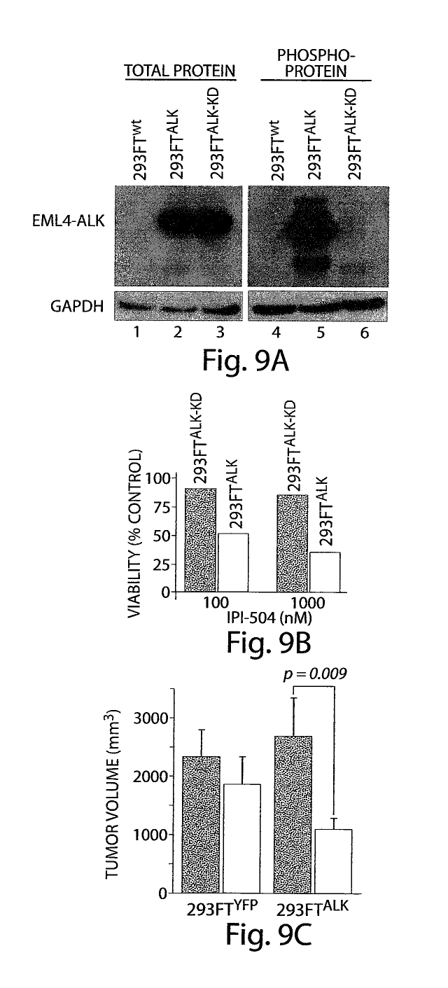

The fact that so many proteins within the cell depend on Hsp90 for their stability makes it hard to mechanistically pinpoint the client protein responsible for cell growth effects after Hsp90 inhibition. It is often assumed that when cells contain a sensitive client that is also the oncoprotein believed to drive that cancer subtype (e.g. HER2 in breast cancer) the growth inhibitory effects of Hsp90 inhibitors on such cells are due to the degradation of this oncoprotein. While this is a reasonable assumption, cells often contain multiple ‘driver’ mutations and hundreds of Hsp90 clients. Therefore, a mere matching of IC50s for client protein degradation and cell growth inhibition is correlative but does not present proof of mechanism. To obtain a mechanistic connection between the expression of EML4-ALK and sensitivity to Hsp90 inhibition, we asked whether transfection of an EML4-ALK cDNA could sensitize a cell to Hsp90 inhibition. Two cDNAs were constructed, encoding the EML4-ALK fusion protein and a mutant version of the fusion where the kinase domain of ALK is inactivated by a point mutation (ALK-KD). cDNAs were transfected into HEK293 cells and the expression (FIG. 9a, lanes 1 to 3) and activity (FIG. 9a, lanes 4 to 6) of the fusion proteins monitored by Western blot. Large amounts of EML4-ALK fusion proteins are expressed from both constructs, and as expected, the kinase dead mutant (ALK-KD) while expressed, has no enzymatic activity. We then asked how the expression of the active or inactive fusion protein modulates sensitivity to IPI-504. While treatment with either 100 or 1000 nM IPI-504 has very little effect on the growth of 293FT cells expressing the kinase dead mutant (293FT^{ALK-KD}), expression of the active EML4-ALK fusion protein (293FT^{ALK}) significantly sensitizes 293FT cells to treatment with IPI-504 (FIG. 9b). This sensitizing effect could also be observed in vivo. When tumors formed from EML4-ALK expressing 293FT cells or control 293FT tumors in nude mice were treated with 100 mg/kg IPI-504 twice weekly for 2 weeks, the drug caused a significant growth inhibition of the EML4-ALK containing tumors but not the control tumors (FIG. 9c).

### IPI-504 Treatment Causes Regression in a Xenograft Model of EML4-ALK Containing NSCLC Cells

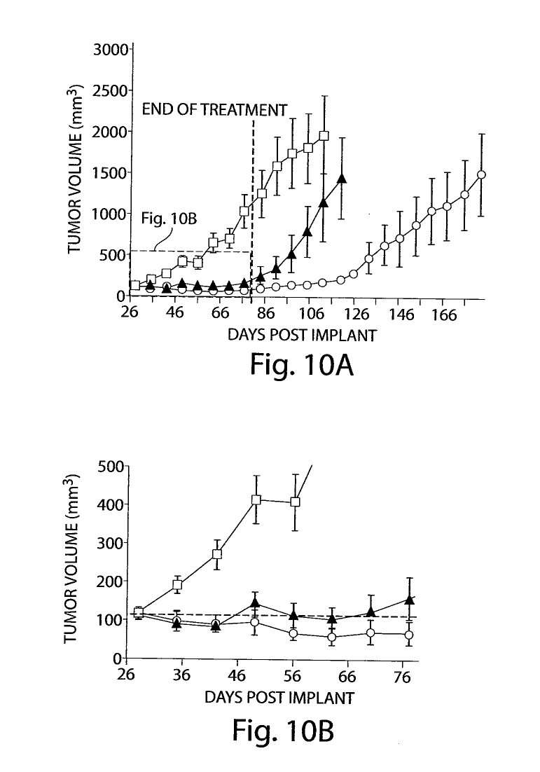

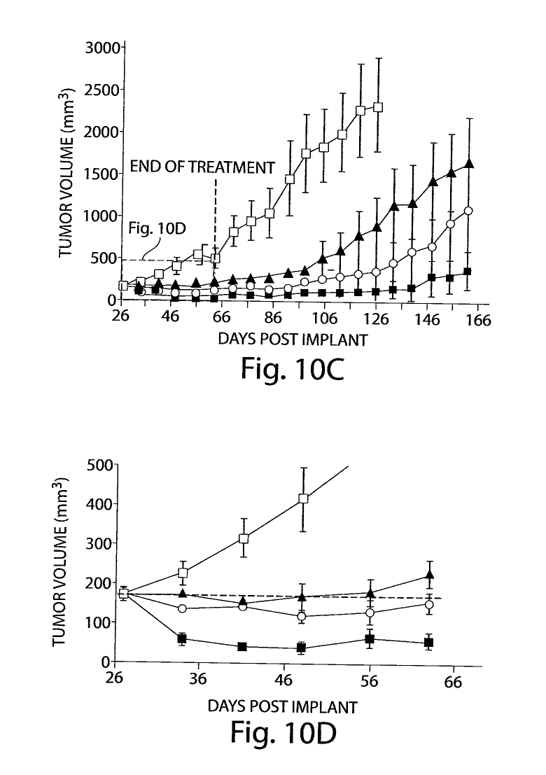

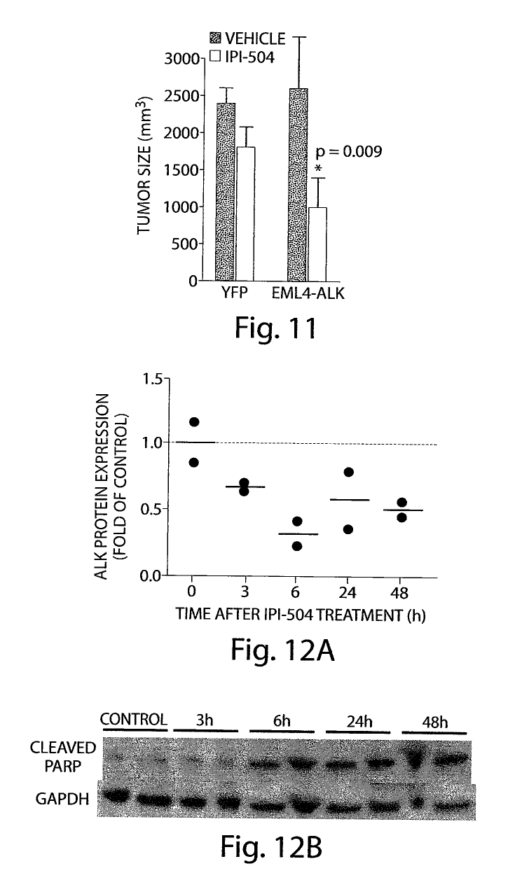

In most xenograft models, Hsp90 inhibitors cause tumor growth inhibition but no tumor regression. Consistent with this, the responses to Hsp90 inhibitors in the clinic have so far mostly consisted of induction of stable disease. To determine whether the extreme sensitivity of the EML4-ALK fusion protein to Hsp90 inhibition would translate into tumor regression of the ALK dependent H3122 cell line in vivo. H3122 cells were injected into the flanks of nude mice and animals were treated with 75 mg/kg of IPI-504 twice weekly. This treatment led to tumor regression, comparable to treatment with the ALK kinase inhibitor PF-1066 at 50 mg/kg every day (FIGS. 10a and 10b). We also tested a combination of IPI-504 and PF-1066 and this treatment led to even more profound tumor regression (FIGS. 10b and 10d). Interestingly, once treatment was stopped, tumors from the PF-1066 arm re-grew more rapidly than tumors in the IPI-504 arm; additionally, an even longer tumor growth delay was observed with the combination of IPI-504 and PF-1066. To confirm degradation of EML4-ALK within these tumors, a separate arm was included in the study where tumors were harvested at different time points after a single injection of IPI-504 and the abundance of the fusion protein over time was monitored using an ALK specific ELISA (FIG. 12A). The result shows that the ALK fusion protein is depleted for more than 48 h after a single injection of IPI-504. The depletion of EML4-ALK coincides with the appearance of PARP cleavage, an indication of caspase 3 activation and the induction of apoptosis (FIG. 12B).

### NSCLC Cells Selected for Resistance to PF-1066 Remain Sensitive to IPI-504

While tyrosine kinase inhibitors have shown impressive response rates in selected patient populations in NSCLC, clinical resistance to these inhibitors often emerges. To ask whether cells resistant to PF-1066 would remain sensitive to IPI-504, we conducted an in vitro experiment to select H3122 cells resistant to PF-1066 by incubating cells with increasing concentrations of the inhibitor. This treatment resulted in a pool of cells (H3122R) that were 12 times less sensitive to PF-1066 than the parental cells. These H3122R cells were also resistant to a structurally different ALK inhibitor (TAE-684) but remained sensitive to IPI-504 (Table 7). Sequencing of the EML4-ALK gene in the H3122R cells did not reveal any secondary mutations (data not shown), indicating that the resistance might be caused by the activation of alternative signaling pathways that remain sensitive to Hsp90 inhibition.

Table 7 summarizes the results of in vitro studies showing that H3122 NSCLC cells selected for resistance to an ALK kinase inhibitor retain sensitivity to IPI-504. More specifically, with type (wt) and resistant (res) H3122 cells had the indicated changes in the GI50 value in response to the HSP90 inhibitor shown below (IPI-504). Resistance to ALK inhibitors is shown by comparing the change in GI50 value in samples treated with the ALK kinase inhibitors, PF-1066 and TAE-684. NSCLC cells selected for resistance to ALK kinase inhibitors retain sensitivity to IPI-504.

TABLE 7

| | Wt H3122 | Res. H3122 | |
|---|---|---|---|
| | (GI50, nM) | (GI50, nM) | Fold change |
| IPI-504 | 22 | 76 | 3.4 |
| PF-1066 | 166 | 2000 | 12.0 |
| TAE-684 | 50 | 3141 | 63.0 |

In sum, the experiments described in this example demonstrate that:

1) EML4-ALK is a highly sensitive Hsp90 client protein.

2) Expression of EML4-ALK can sensitize cells to IPI-504 treatment.

3) Combinations of IPI-504 and ALK kinase inhibitors lead to pronounced tumor regressions in xenograft models of human NSCLC.

4) Cells selected for resistance to ALK kinase inhibitors retain sensitivity to IPI-504.

5) In patients, rearrangements in the ALK locus are associated with responses to IPI-504 as a single agent.

6) Further validation of these findings is ongoing in a prospective trial of IPI-504 in patients with NSCLC and an ALK rearrangement.

### Discussion

Personalized cancer medicine relies on the ability to match the right drug with the right patient. To that end, linking somatic genetic alterations in the tumor and response to targeted therapies has become a critical and integral step in cancer drug development. The advent of novel genetic technologies, including high throughput cancer gene genotyping (Oncomap (Thomas et al., 2007, supra) and Snapshot) as well as FISH, have enabled the identification of these alterations and hence patient subpopulations most likely to benefit from novel therapies. Using these strategies in a retrospective analysis of tumor specimens from our study of IPI-504 in NSCLC, we have discovered an association between ALK rearrangements and response to IPI-504 (Sequist et al., 2010, supra). Here we present evidence that this clinical correlation is likely due to the sensitive, rapid and sustained degradation of oncogenic ALK fusions upon treatment with IPI-504. The results herein show that both in terms of dose response and time to degradation, EML4-ALK is a more sensitive client than either mutant EGFR or HER2, a protein previously believed to be one of the most sensitive Hsp90 clients in the cell. Since there are more than 200 Hsp90 client proteins in the cell, it has been hard to narrow down the exact mechanism of cancer cell growth inhibition after Hsp90 inhibitor treatment. It is not clear whether the cellular effects of Hsp90 inhibition are brought about by general effects on protein homeostasis through the simultaneous inhibition of multiple client proteins or whether they can be traced back to the degradation of a single oncogenic driver protein. The results herein show through the use of isogenic cell lines that at least in the case of EML4-ALK the latter seems to be the case. This has implications for the future clinical development of Hsp90 inhibitors since our results suggest that patients with cancer subtypes driven by oncoproteins that are very sensitive Hsp90 client proteins might benefit the most from Hsp90 inhibitor treatment.

The exquisite sensitivity of EML4-ALK as a client protein also translates to the in vivo setting. Different from most other in vivo models, we observe tumor regression in an EML4-ALK positive NSCLC xenograft model with IPI-504 dosed twice weekly. This might be due to the fact that in vivo, the fusion protein is depleted for more than 48 h after a single injection of IPI-504 leading to the induction of apoptosis (FIGS. 12A and 12B). We also obtain evidence, that cells that have become resistant to ALK kinase inhibitors remain sensitive to IPI-504 suggesting a possible treatment for patients that relapse after successful TKI treatment.

In conclusion, this study suggest that NSCLC patients positive for EML4-ALK fusion proteins are likely to derive benefit from IPI-504 therapy and, potentially, from other HSP90 inhibitors. In addition, Lin and coworkers recently reported the detection of EML4-ALK fusion in breast and colorectal carcinomas (Lin E, et al. (2009) Mol Cancer Res 7: 1466-76). Therefore, the presence of ALK oncogenic activation in these tumor types can also predict response to IPI-504 in breast and colorectal cancer patient subpopulations.

### Experimental Design for Example 4: Materials and Methods Cell Lines and Compounds

H3122 cell were obtained from the NIH (Rockville, Md.), H1650 and BT-474 cells were from ATCC (Manassas, Va.). All cell lines were tested for mycoplasma and cultured in RPMI 1640 supplemented with 10% heat-inactivated fetal bovine serum. IPI-504 was synthesized at Infinity (Ge et al., 2006). TAE-684 and PF-02341066 (referred to as PF-1066) were purchased from Chemietek (Indianapolis, Ind.).

### Generation of the 293FT^{EML4-ALK} Cell Line

EML4-ALK variant 1 open reading frame clone and the kinase domain-dead version (K589M) were synthesized by GenScript based on GenBank accession number sequence AB274722. Recombination was done via Gateway LR clonase reaction (Invitrogen, Carlsbad, Calif.) pLenti6.2/R4R2/DEST vector along with a cytomegalovirus promoter vector according to the manufacturer's instructions. The resulting vector was used to generate recombinant lentiviral particles using Virapower reagent (Invitrogen, Carlsbad, Calif.). 293FT cells were infected via polybrene mediation with the variant 1 EML4-ALK-expressing lentiviral particles. Cells were selected using blasticidin. The same protocol was used to generate 293FT cells stably expressing the kinase dead (K589M) variant 1 EML4-ALK protein.

### Generation of PF-1066 Resistant Cells

H3122 cells were mutagenized overnight with 0.64 mM N-ethyl N-ethylurea (ENU), washed and cultured in 500 nM PF-1066 for 3 weeks. Short Tandem Repeat sequencing (RADIL, Columbia, Mo.) was used to confirm that the resistant cells (H3122R) were derived from H3122 cells.

### Cell Growth and Viability Studies

H3122 were seeded at 4,000 cells/well in 96 well plates and incubated with increasing concentrations of IPI-504 or PF-02341066 for 72 h. Growth inhibition studies were performed using Cell Titer Glo (Promega, Madison, Wis.). Viability studies were performed by trypan blue exclusion and quantitated using the Countess cell counter (Invitrogen, Carlsbad, Calif.). The data were normalized to DMSO controls to generate growth inhibition GI₅₀ values.

### Immunoblot, Immunoprecipitation and ELISA Analyses

Cells were treated for the indicated time with IPI-504 and lysed in ice-cold cell lysis buffer (Cell Signaling, Beverly, Mass.) containing protease inhibitors and HALT phosphatase inhibitor (Pierce, Rockford, Ill.). Lysates were clarified by centrifugation at 14,000 g at 4° C. and protein concentration determined by BCA method (Pierce Rockford, Ill.). Samples were boiled for 5 minutes in sample buffer, resolved on 4-12% BIS-TRIS gels, and transferred onto PVDF membrane. Blots were probed with antibodies to ALK, phospho-ALK (Tyr1604), AKT, phospho-AKT (Ser473), ERK, phospho-ERK (Thr202/Tyr204), pSTAT3 (Tyr705), EGFR, cleaved-PARP (all from Cell Signaling, Beverly, Mass.), HER2 (Abcam, Cambridge Mass.), and GAPDH (Santa Cruz Biotech). ALK and pALK level were also monitored using an ELISA (RnD Systems, Minneapolis, Minn.) according to supplier instructions. For immunoprecipitation, 1 mg of pre-cleared cell lysate was incubated overnight at 4 C with 2 ug Hsp90□ (9D2; Stressgen). Protein G beads were added for 4 h on a rotator at 4 C and beads were washed extensively with cold lysis buffer. 2× reducing sample buffer was added, sample was boiled and run on a 4-12% Bis-Tris SDS gel. Proteins were transferred to PVDF and probed with either an ALK or an Hsp90 antibody overnight at 4 C followed by detection with an HRP-linked secondary antibody.

### Xenograft Studies

All in vivo studies were conducted in accordance with the Institutional Animal Care and Use Committee (IACUC) guidelines. PF-02341066 was prepared at 2.5 mg/ml in sterile water for injection, pH 4-5 and stored at −20° C. IPI-504 was prepared in 5 mM Citrate, 20 mM Ascorbate, 0.244 mM EDTA in 0.9% Saline, pH 3.3 and stored at −80° C. Five to six (5-6) week old male NCR nu/nu athymic mice were purchased from Taconic Farms (Hudson, N.Y.). Five million (5×10⁶) NCI-H3122 cells in serum-free RPMI 1640 were implanted subcutaneously into the right rear flanks of mice and treatment was started when tumors reached an average volume of 170 mm³. Vehicle, IPI-504 50 mg/kg or 75 mg/kg intraperitoneal (IP) was administered two times per week at a volume of 8 ml/kg. PF-02341066 37.5 mg/kg oral (PO) was administered QDX5 (5 days on, 2 days off) at a volume of 15 ml/kg. Tumors were measured once per week with digital calipers and tumor volume was determined using the formula: (length×width)/2. Results are presented as average tumor volume±standard error of the mean (SEM) in mm³. For in vivo analysis of 293FT^{YFP} or 293FT^{EML4-ALK} engineered cell lines, cell lines were produced as described above. Ten million (10×10⁶) cells of each cell line in serum-free DMEM mixed with matrigel (BD Biosciences) 1:1 were implanted into the right rear flanks of 5-6 week old male NCR nu/nu mice and treatment was initiated once tumors had reached ˜250-300 mm³. IPI-504 or vehicle was administered twice per week (BIW) IP at 75 mg/kg at a volume of 8 ml/kg. Tumor volumes were assessed as described above.

### Example 5

### Efficacy of IPI-504 in Patients with Squamous Cell Carcinoma

This example describes the results from a clinical trial that assessed the efficacy of IPI-504 in patients with squamous cell carcinoma (SCC).

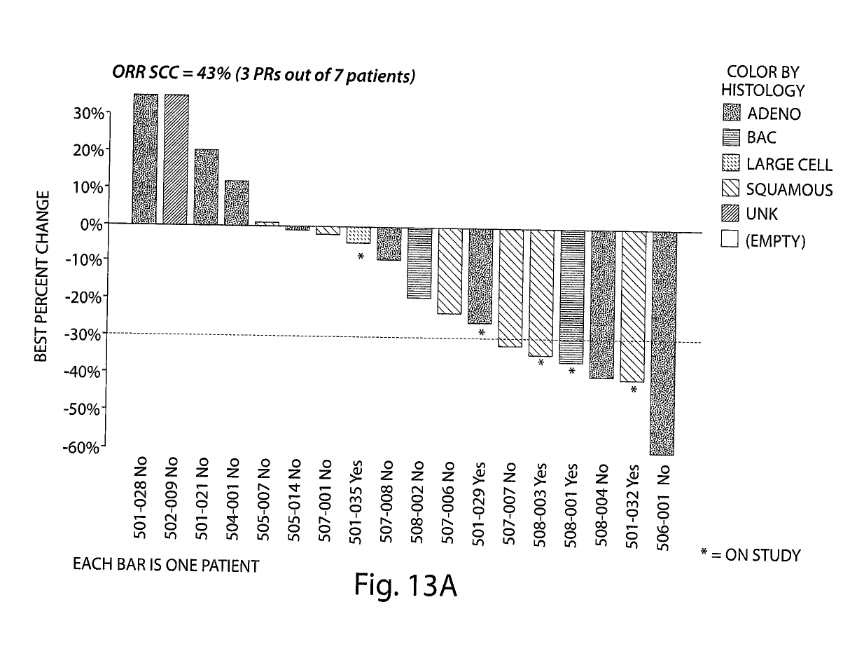

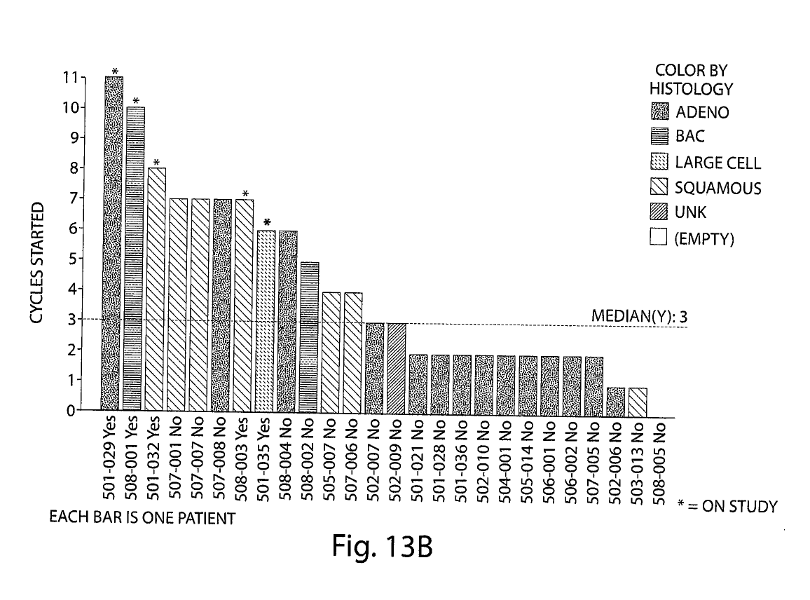

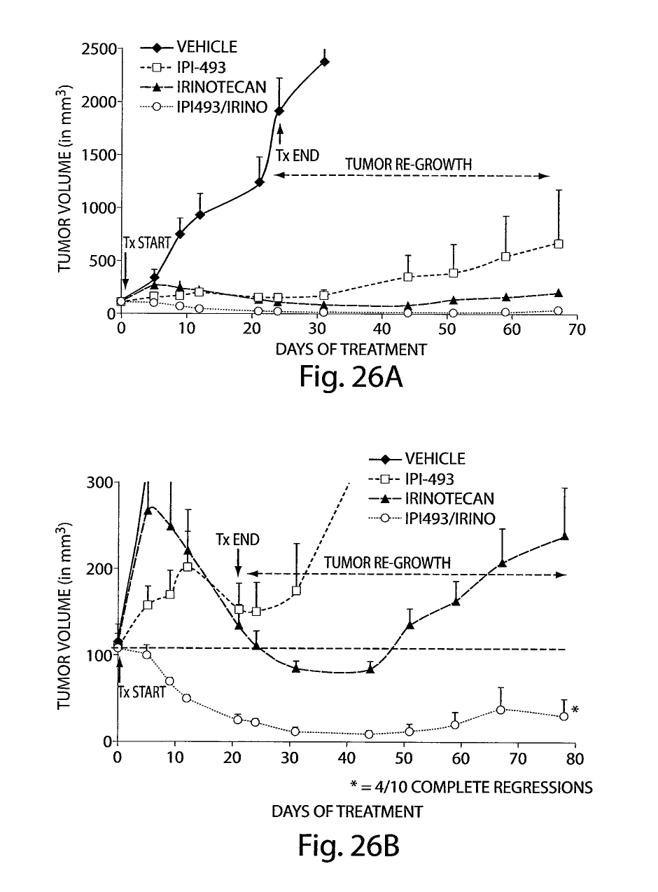

FIGS. 13A-13B depict waterfall plots showing responses to IPI-504 according to cancer subtypes analyzed by histology. The cancers examined were adenocarcinoma (shown as #1), bronchioloalveolar carcinoma (BAC) (shown as #2), large cell lung cancer (shown as #3), squamous cell carcinoma (shown as #4), unknown (shown as #5) and control (shown as #6). Each bar represents one patient. The y-axis of FIG. 13A represents % of tumor volume change from baseline. Patients with SCC showed an objective response rate (ORR) to IPI-504 of 43% with 3 out of 7 patients showing a partial response (PR) (FIG. 13A). FIG. 26B shows the efficacy of IPI-504 in SCC patients after therapeutic cycles started.

### Example 6

### Efficacy of IPI-504, Alone or in Combination with Docetaxel, in Smokers

This example describes the results from a clinical trial that assessed the efficacy of IPI-504, alone or in combination with docetaxel, in smoker patients.

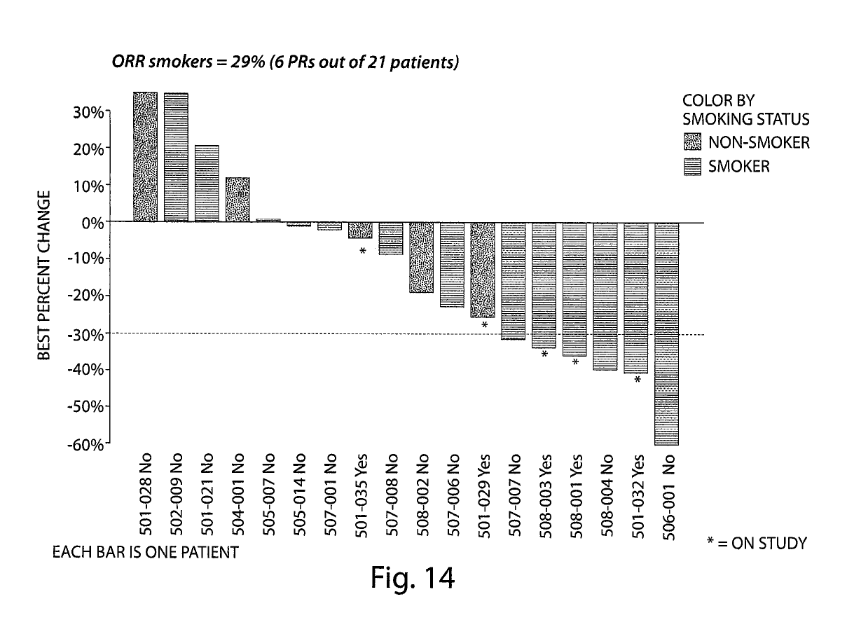

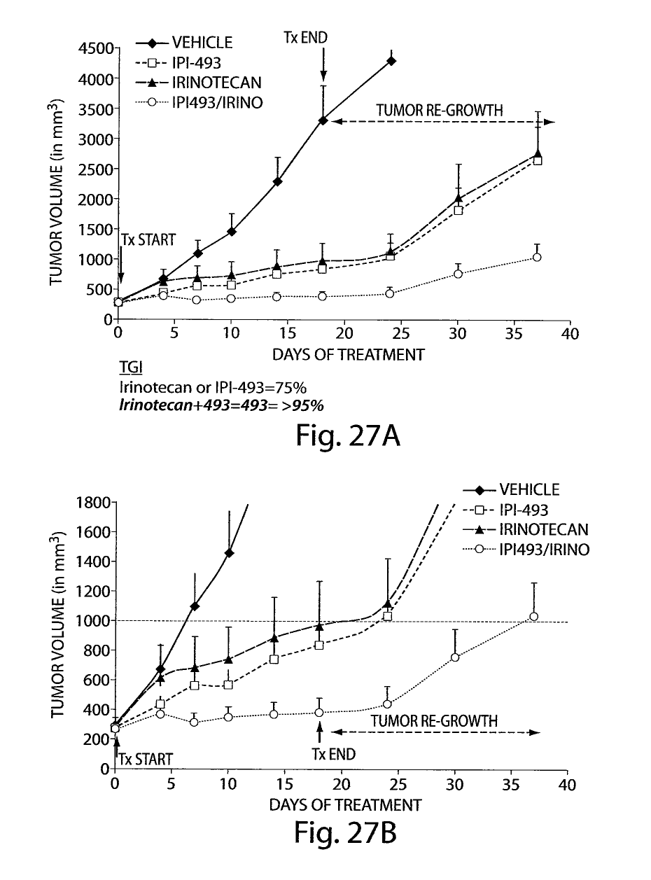

FIG. 27 depicts a waterfall plot showing responses to IPI-504 according to smoking status. The y-axis of FIG. 27 represents % of tumor volume change from baseline. Each bar represents one patient. Smoker patients showed an objective response rate (ORR) to IPI-504 of 29% with 6 out of 21 patients showing a partial response (PR) (FIG. 14).

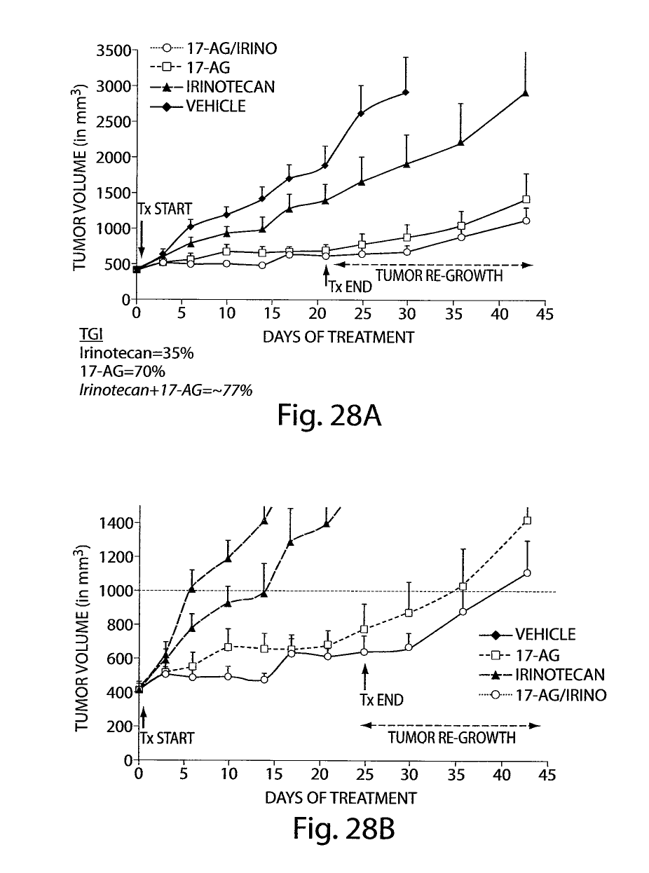

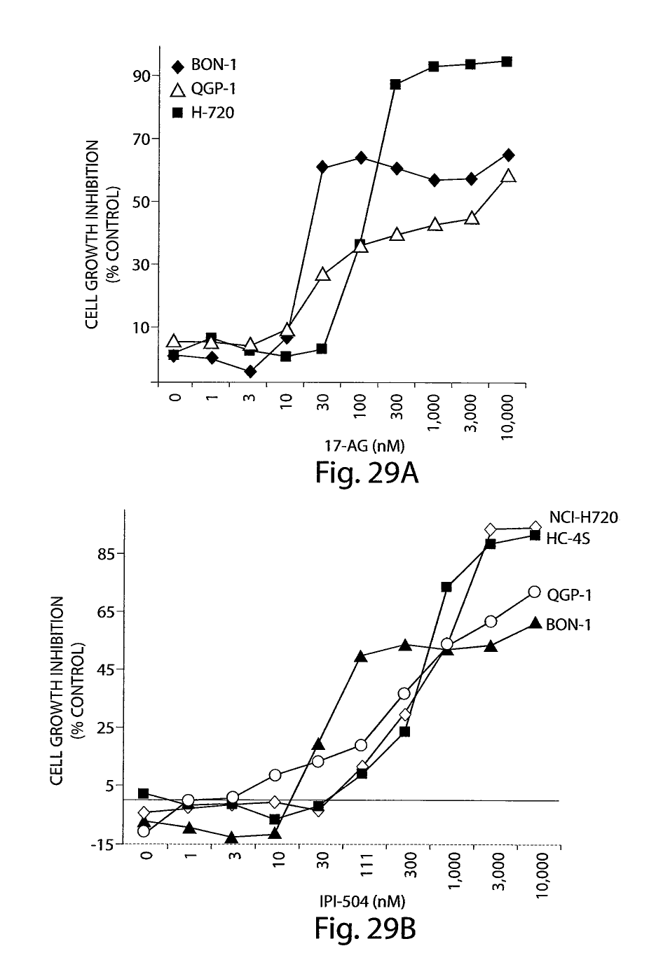

Next, the relationship between tobacco exposure and efficacy in NSCLC and SCC was evaluated. The results are shown in FIGS. 28-29.

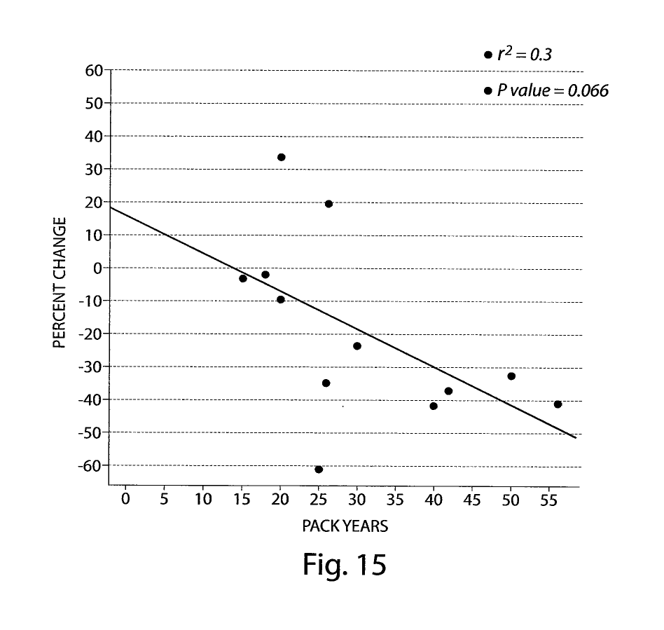

FIG. 15 depicts a graph showing increased efficacy of IPI-504 determined by % decrease in tumor volume as the tobacco exposure (assessed by number of pack years) increased in patients with NSCLC. The y-axis represents % of tumor volume change from baseline.

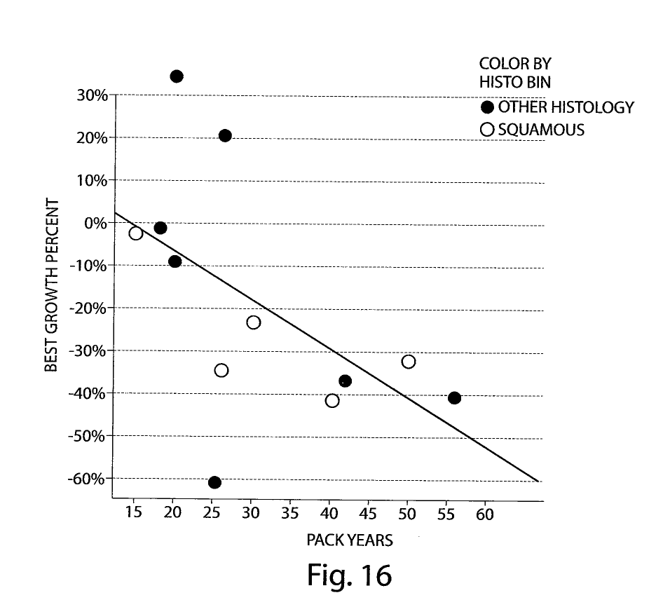

FIG. 16 depicts a graph showing increased efficacy of IPI-504 determined by % decrease in tumor volume as the tobacco exposure (assessed by number of pack years) increased in patients with SCC and other lung cancer histologies. The y-axis represents % of tumor volume change from baseline.

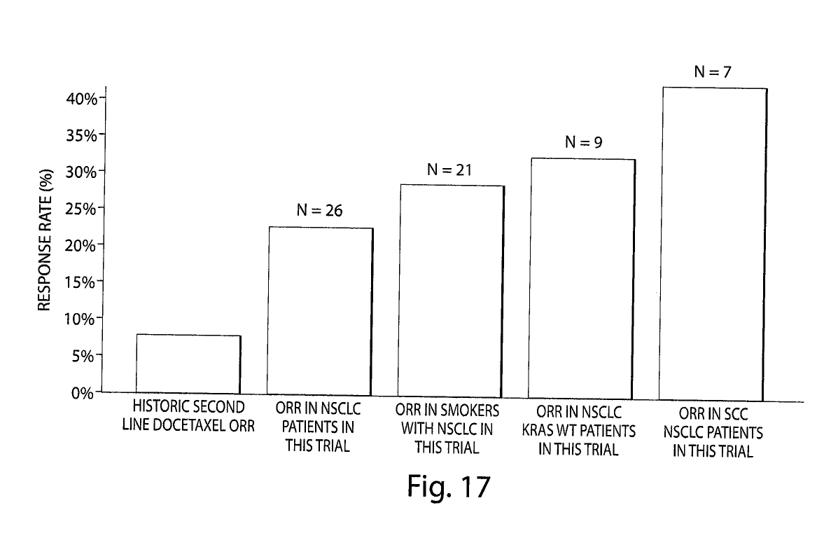

FIG. 17 is a bar graph summarizing the efficacy of the combination of IPI-504 and docetaxel in patients with NSCLC. The y-axis represents the % change in response rate (ORR) in the following patient populations:

1) Patients having docetaxel as second line therapy;

2) NSCLC patients in this trial treated with IPI-504 and docetaxel;

3) NSCLC patients, who were smokers, in this trial treated with IPI-504 and docetaxel;

4) NSCLC patients, who are KRAS wild-type, in this trial treated with IPI-504 and docetaxel; and

5) SCC/NSCLC patients in this trial treated with IPI-504 and docetaxel.

These results show an interesting association between smoking status and tumor shrinkage in response to IPI-504, alone or in combination with docetaxel. The combination of IPI-504 and docetaxel has activity in NSCLC. These results thus enable appropriate patient selection in patients to be treated with HSP90 inhibitors, such as IPI-504. Among the factors to be considered include one or more of tumor histology (e.g., NSCLC or SCC), smoking status, HSP90 expression and/or ALK or KRAS status.

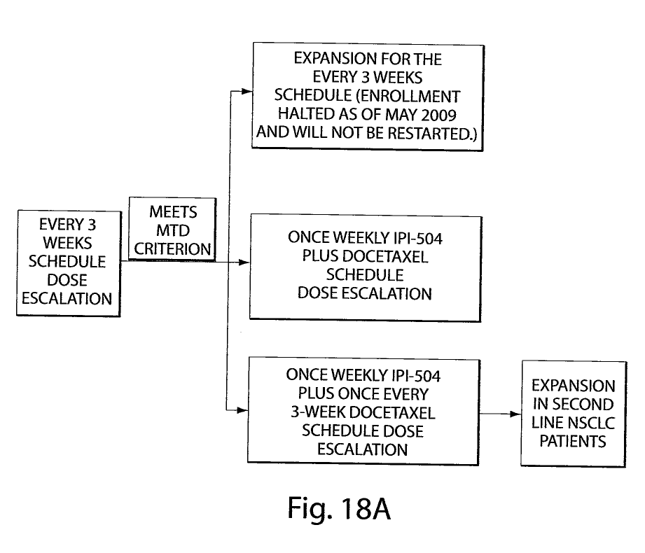

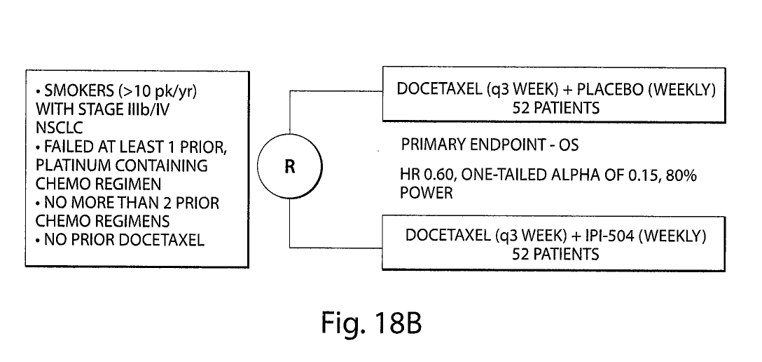

Flow chart summarizing the study designs of two clinical trials evaluating the combination of IPI-504 and docetaxel are summarized in FIGS. 18A-18B.

FIG. 18A summarizes a clinical study of the combination of IPI-504 at 300 mg/m2 in a 3-week schedule and having docetaxel administered once a week. The combination was well tolerated with no unexpected safety observations. As to the NSCLC subset, 26 NSCLC patients were treated with weekly ILI-504 (and either weekly or once every three weeks with docetaxel). None of the patients had prior docetaxel treatment. The ORR was 23% with 6 out of 26 patients showing a partial response.

FIG. 18B summarizes the design of a randomized, placebo-controlled Phase 2 clinical trial of docetaxel with or without IPI-504 in a 2^{nd}-3^{rd} line of treatment for NSCLC.

### Example 7

### B-Raf and K-Ras Mutations in Colorectal Cancer (CRC)

This example shows that IPI-493 demonstrates good efficacy in CRC harboring kRAS or bRAF mutations and further demonstrate that MAPK pathway activity can be a valuable marker of IPI-493 sensitivity. Moreover, the combination of IPI-493 with Irinotecan was superior to either agent alone.

### Background/Summary

Colorectal cancer (CRC) is the third most common form of cancer in the United States and worldwide with greater than 50,000 and 700,000 cancer-related deaths annually, respectively. Greater than 110,000 new diagnoses and 45,000 deaths expected in 2009 (US); >300,000 and 100,000 worldwide, respectively. Approximately, a 5 yr survival rate is expected for less than 10% of the patients with mCRC.

The standard of care (SOC) for advanced CRC includes multi-agent combination therapy with:
5-Fluorouracil (5FU-TS inhibitor); Irinotecan (Topo I poison); Oxaliplatin (DNA adducts)
Erbitux and Vectabix (monoclonal Abs against EGFR) are also FDA approved for Tx of metastatic CRC
1st line
FOLFOX: 5-Fluorouracil+Leucovorin+Oxaliplatin
FOLFIRI: 5-Fluorouracil+Leucovorin+Irinotecan
2nd line⋄Normally the alternate to the 1st line therapy
3+ line⋄irinotecan +/−CTX or biologic
Irinotecan inclusion with CTX after failure
Erbitux and Vectabix (monoclonal Abs against EGFR) are FDA approved for Tx of advanced metastatic CRC (KRAS wt)
Erbitux in combination with Irinotecan for Irinotecan-refractory pts
Vectabix as a single agent

Approximately 40% of cases can contain mutations in the small GTPase kRAS and ˜10-25% in its downstream effector, the cytosolic kinase bRAF, which are mutually exclusive. In addition, in CRC mutant bRAF represents a sub-set of patients distinct from that of mutant kRAS. Mutant bRAF has previously been shown in melanoma to be a very sensitive “client” of the molecular chaperone HSP90. HSP90 is a chaperone protein that maintains the conformation and activity of diverse cellular proteins referred to herein as “HSP90 clients.” Many HSP90 clients are oncoproteins. Mutant BRAF has been shown to be a sensitive HSP90 client in melanoma and CRC. Activated c-RAF (which is thought to be the important RAF family member in KRAS mutant backgrounds) is also a HSP90 client.

IPI-493 is an oral HSP90 inhibitor that is currently in a phase 1 dose escalation clinical trial. To investigate the potency of IPI-493 in CRC, we first performed growth inhibition (GI) studies on a CRC cell line panel. IPI-493 demonstrated GI50's in the range of 10-100 nM in cell lines harboring either kRAS or bRAF mutations. These GI values were also confirmed in colony forming assays. To explore the in vivo potency of IPI-493, several mutant bRAF (Colo201, Colo205, Colo741, HT55) and kRAS (HCT-116, SW480, DuDu-1), as well as a wild-type kRAS/bRAF (Colo320HSR, NCI-H716, SNU-C1, C2BBe1) xenograft models were developed. In regards to efficacy, administration of IPI-493 as a single agent at 100 mg/kg (mpk) three times weekly demonstrated dramatic effects in all of the mutant models tested, with tumor growth inhibition (TGI) values between 70 and 90% and regression in one mutant BRAF model. Importantly, when IPI-493 was evaluated in combination with Irinotecan (a standard of care in late stage CRC), the combination of the two drugs showed superior efficacy versus either drug alone, causing overall tumor regression, and complete regressions in 4 of 10 animals that lasted >70 days.

Interestingly, when IPI-493 efficacy was explored in the wild-type kRAS/bRAF background, no detactable effect on TGI was observed. Together with the significant effects observed in mutant kRAS/bRAF models, these results suggest activation of the MAPK pathway can be an important marker of sensitivity to HSP90 inhibition. FIG. 19 summarizes the MAPK (RAS-RAF-MEK-Erk) pathway.

To further investigate a potential correlation between the effect of IPI-493 on molecular components of the RAS-RAF-ERK pathway and efficacy, pharmacodynamic analysis was performed on mutant bRAF (Colo205, Colo201) and wild-type (Colo320HSR) xenograft models after a single 100 mpk dose. The data show that IPI-493 administration resulted in downregulation of the activity of both bRAF and MEK (downstream target of bRAF) ˜24 hr post dose in mutant kRAS or bRAF models, as assessed by decreased phosphorylation of both proteins (p-bRAF, p-MEK). In addition, IPI-493 also elicited an increase in cleaved caspase 3 (a marker of apoptosis) within the same time frame, further suggesting a correlation between the two events. In contrast, wild-type kRAS/bRAF Colo320HSR model demonstrated low baseline levels of both p-bRAF and p-MEK and little effect of IPI-493 administration. Moreover, when p-MEK activity was assessed for all the CRC xengrafts, there was a clear delineation of sensitivity between mutant and wild type models, further supporting the hypothesis that MAPK pathway activity can be a surrogate for IPI-493 efficacy. Collectively, these results show that IPI-493 demonstrates good efficacy in CRC harboring kRAS or bRAF mutations and further demonstrate that MAPK pathway activity can be a valuable marker of IPI-493 sensitivity. Moreover, the combination of IPI-493 with Irinotecan was superior to either agent alone. Altogether, these provide rationale for a clinical path in this disease.

### Preparation of an Amorphous Molecular Dispersion of 17-AG

To a 4:1 mixture of acetone (160 mL) and ethanol 190 proof USP/NF grade (40 ml) was added HPMC-AS-HG (1 g) in a single portion (acetone can be used as an alternative to the acetone:ethanol mixture to dissolve the polymer and 17-AG). The mixture was stirred at 60° C. until the dissolution of the polymer was complete (ca 30 minutes). 17-AG (1 g) was added in portions over the course of 10 minutes to provide an opaque purple mixture. The resulting solution (1% w/v solids) was stirred at 60° C. for 30 minutes.

The homogeneous purple solution was then concentrated and the solvent was removed under high vacuum at 50° C. to provide a solid dispersion. The dispersion confirmed by cross-polarization microscopy to be substantially amorphous. The dispersion was then crushed to a powder using a mortar and a pestle and dried under high vacuum at 30° C. for 24 hrs.

Preparation of a suspension of this solid amorphous dispersion was made by levigating the solid with glycerol with a spatula followed by homogenization in a 1% hydroxyethylcellulose solution in a high speed homogenizer for 10 minutes to provide in vehicle (1% hydroxyethylcellulose, 5% glycerol). The suspension was used to administer 17-AG in the below described in vivo experiments.

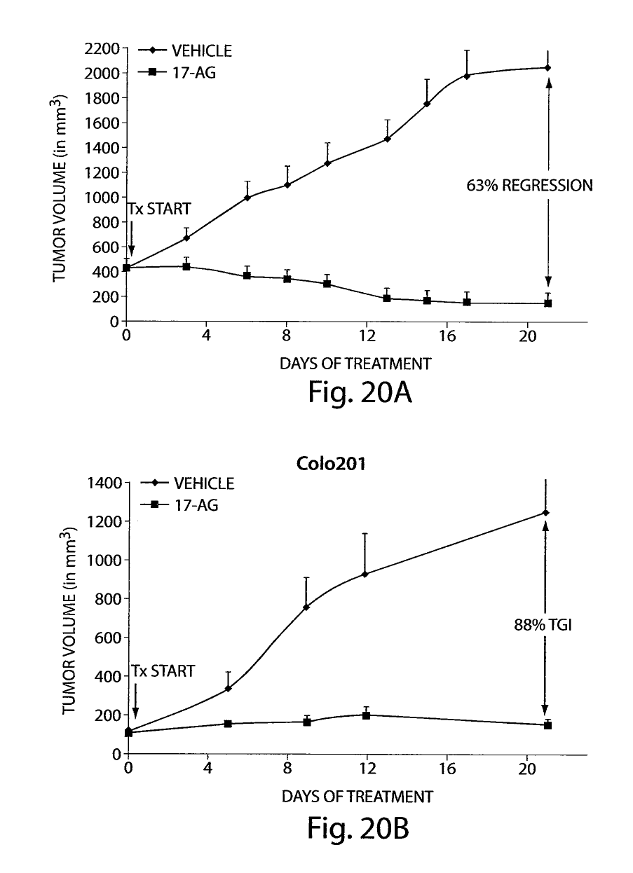

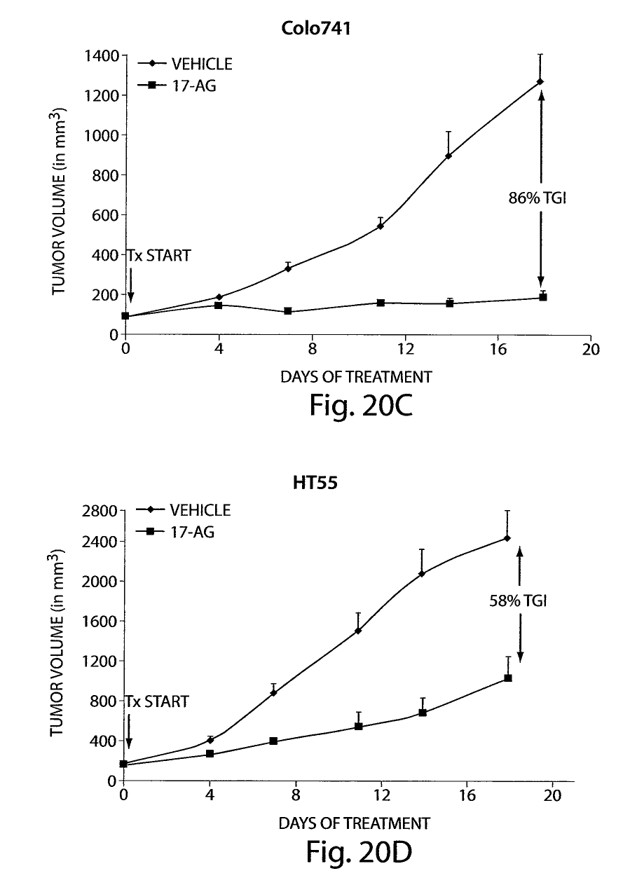

### Experiment 1. Table 8 and FIGS. 20A-20D

In the first set of studies, several different colorectal adenocarcinoma cell line models which contain either a V600E or Y581F activating mutation in the cyotsolic serine/threonine kinase BRAF were employed: Colo205 (V600E) (FIG. 20A), Colo201 (V600E) (FIG. 20B), Colo741 (V600E) (FIG. 20C), and HT55 (Y581F) (FIG. 20D). 5-6 week old Nu/Nu mice were implanted subcutaneously with 10×10⁶ cells for each cell line used. Dosing commenced after tumors had reached approximately 150-200 mm³. Dosing was by oral gavage and the dosing schedule was three times weekly (M, W, F) with a suspension of 50% 17-AG in HPMC-AS-HG dispersion in vehicle (1% hydroxyethylcellulose, 5% glycerol) at a dose level of 100 mg/kg 17-AG as compared to vehicle control. Dosing was carried out for ˜3 weeks (9 doses). At the end of the treatment schedule, maximum tumor growth inhibition (TGI) ranging between 58 and 94% was observed comparing 17-AG treated animals to animals treated with the vehicle control.

Table 8 summarizes the activity of IPI-504 and IPI-493 in CRC cell lines in vitro. The GI-50 concentration is provided as a function of mutational status.

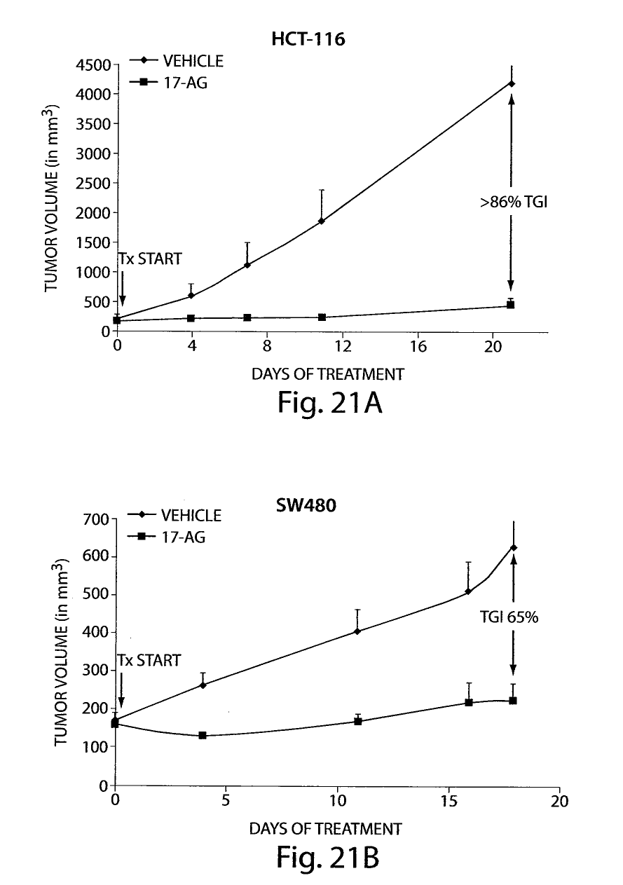

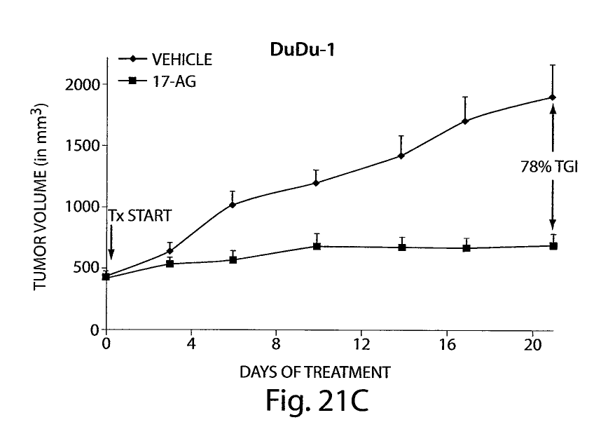

### Experiment 2. FIGS. 21A-21C

In the second set of studies, the HCT-116 (FIG. 21A) and SW-480 cell lines (FIG. 21B) and DuDu-1 (derived from a primary tumor) (FIG. 21C) containing activating mutations in the small GTPase RAS (G13D, G12V and G12V respectively) were used. As described above, 5-6 week old Nu/Nu mice were implanted subcutaneously with 10×10⁶ cells for each cell line used. For the primary DuDu-1 model, cells were harvested from tumors that were propagated in Nu/Nu mice and the appropriate cell number was used implant into naïve Nu/Nu mice for the 17-AG studies. Dosing commenced after implanted cells reached about 150-200 mm³ and was by performed by oral gavage on the dosing schedule of three times weekly (M, W, F) with a suspension of 50% 17-AG in HPMC-AS-HG dispersion in vehicle (1% hydroxyethylcellulose, 5% glycerol) at a dose level of 100 mg/kg 17-AG as compared to vehicle control. After dosing, 65-86% maximum reduction in tumor volume was seen in the dosing arms as compared to vehicle treated animals for the different models.

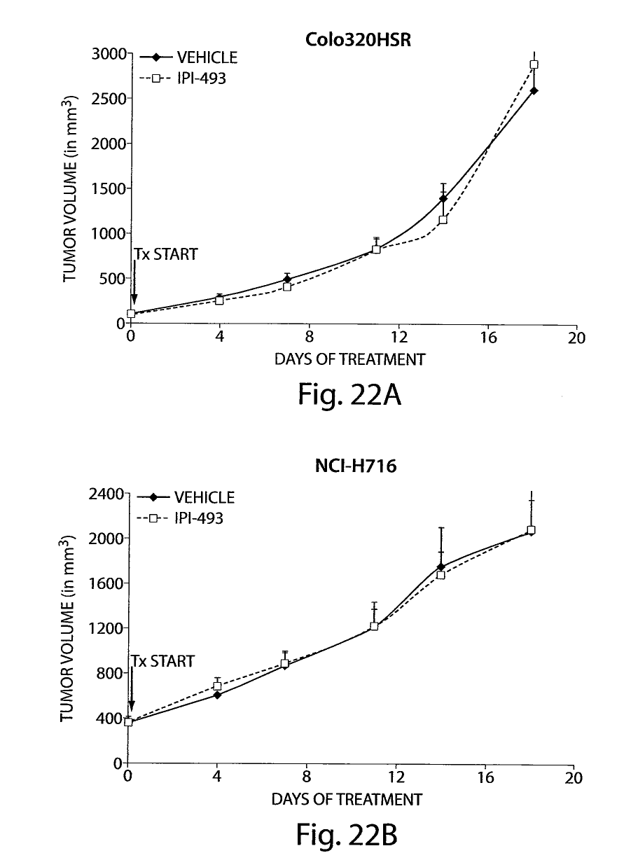

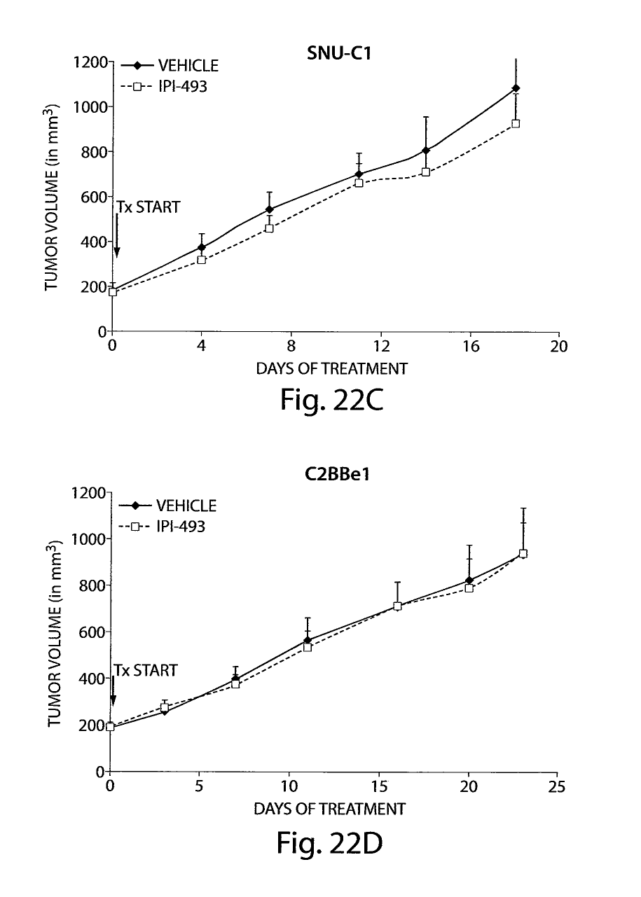

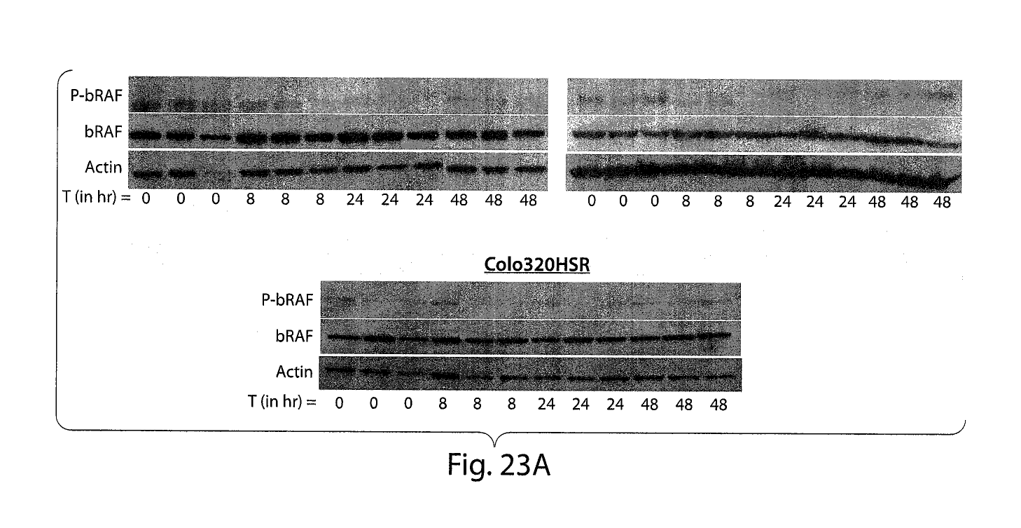

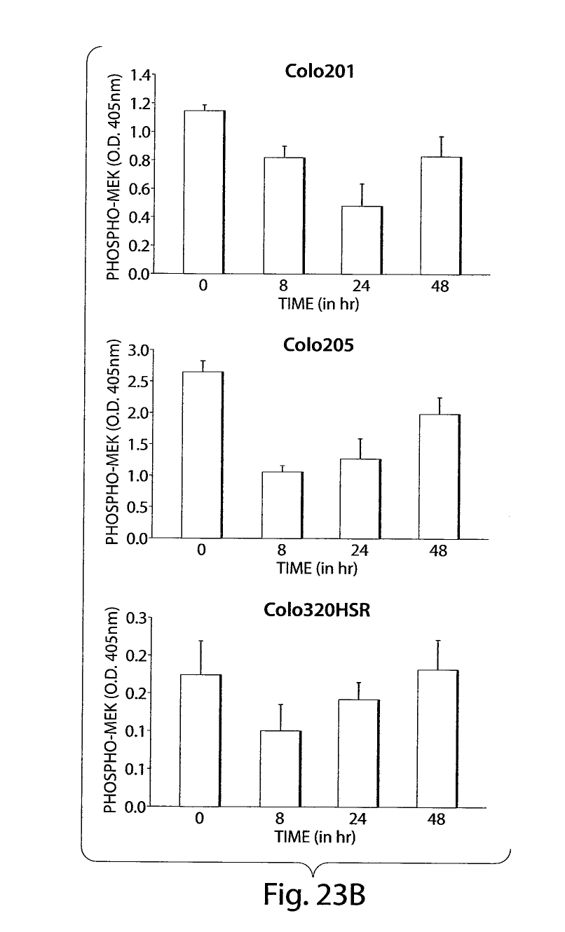

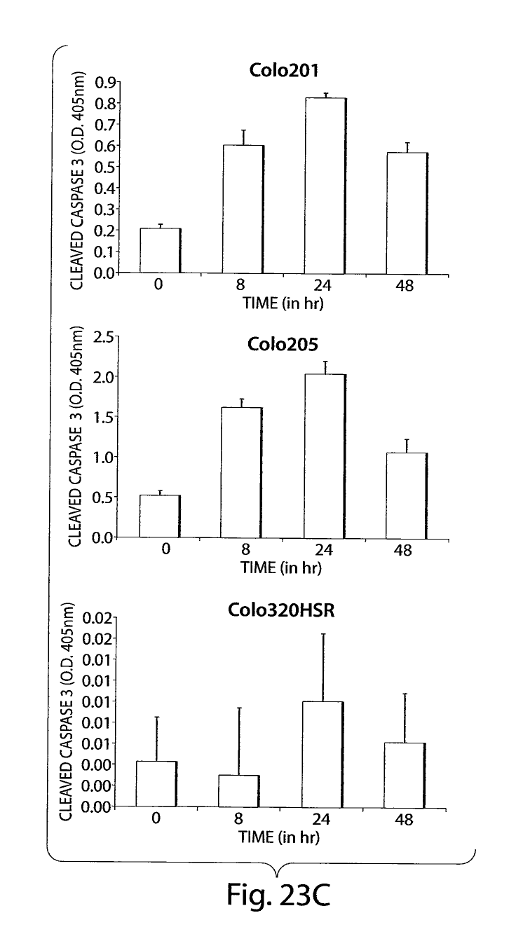

### Experiment 3. FIGS. 22A-22D and 23A-23C

In the third set of studies, the Colo320HSR (FIG. 22A), NC1—H716 (FIG. 22B), SNU-C1 (FIG. 22C) and C2BBe1 (FIG. 22D) cell lines were employed all of which are wild type for both KRAS and BRAF. 5-6 week old Nu/Nu mice were implanted subcutaneously with 10×10⁶ cells for each cell line used and dosing by oral gavage commenced after tumors had reached approximately 150-200 mm³ on a dosing schedule of three times weekly (M, W, F) with a suspension of 50% 17-AG in HPMC-AS-HG dispersion in vehicle (1% hydroxyethylcellulose, 5% glycerol) at a dose level of 100 mg/kg 17-AG (IPI-493) as compared to vehicle control. At the end of the treatment period, no detectable difference in tumor volume was observed between the 17-AG (IPI-493) and vehicle treated animals for each CRC cell line wild type for both KRAS and BRAF.

FIG. 23A shows a panel of immunoblots depicting a time dependent decrease in phosphorylated BRAF in mutant Colo 201 and Colo 205 xenografts upon a single dose of IPI-493 (100 mpk). Similar changes were observed in KRAS mutant models. Minimal changes in phosphorylated BRAF activity were detected in wild type Colo320HSR.

FIG. 23B shows a panel of bar graphs depicting a time dependent decrease in phosphorylated MEK in mutant Colo 201 and Colo 205 xenografts. Similar changes were observed in KRAS mutant models. Minimal changes in phosphorylated BRAF activity were detected in wild type Colo320HSR upon a single dose of IPI-493 (100 mpk).

FIG. 23C shows a panel of bar graphs depicting a time dependent increase in cleaved caspase 3 activity in mutant Colo 201 and Colo 205 xenografts (correlating with the decrease on phosphor MEK). Minimal changes were detected in wild type Colo320HSR upon a single dose of IPI-493 (100 mpk).

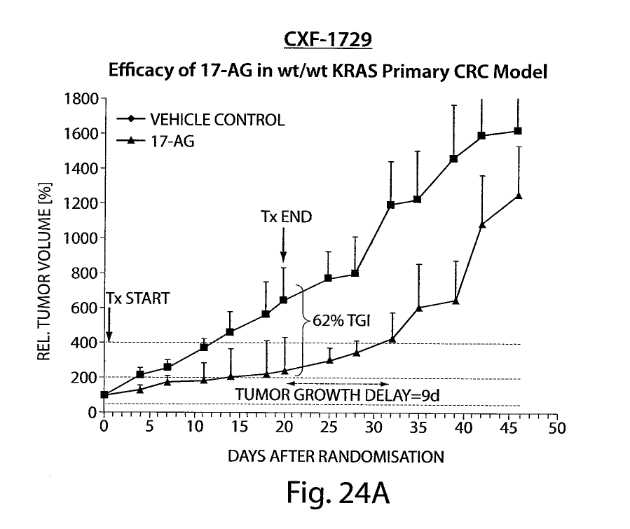

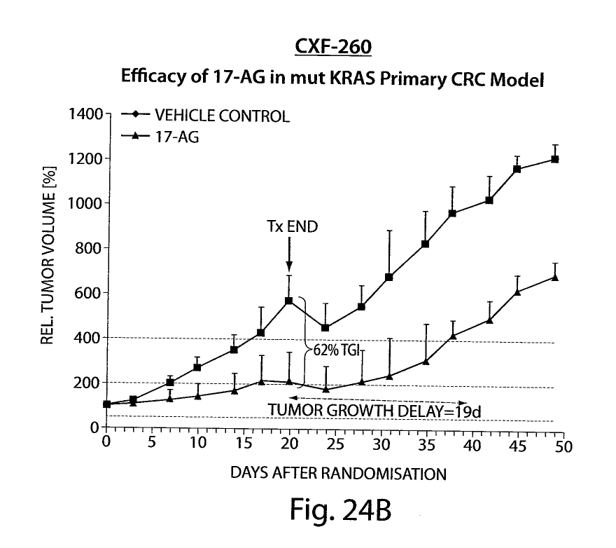

### Experiment 4. FIGS. 24A-24B

In the fourth set of studies, xenograft studies were performed (Oncotest GmbH, Freiberg, Germany). Briefly, two xenograft models derived from primary patient samples by direct transplant and propagation in nude mice, CXF-1729 (wild-type BRAF, wild-type KRAS) (FIG. 24A) and CXF-260 (wild-type BRAF, mutant KRAS (G12V)) (FIG. 24B), were grown in nude mice. Once tumor volumes had reached approximately 150-200 mm³, mice from each model were randomized into treatment groups, either vehicle or a suspension of 50% 17-AG in HPMC-AS-HG dispersion in vehicle (1% hydroxyethylcellulose, 5% glycerol), and dosed via oral gavage on schedule of 90 mg/kg, three times weekly (M-W-F) for three weeks and tumor volumes monitored. Once the three week dosing period had ended, animals were monitored for an additional 24-26 days to follow tumor growth delay. The results demonstrate that while on drug, both models, CXF-1729 and CXF-260, responded similarly to 17-AG treatment showing >60% tumor growth inhibition (TGI). Following the treatment phase, the CXF-1729 model demonstrated a nine day delay in tumor growth (time to tumor volume doubling post treatment cessation) while the CXF-260 model demonstrated a nineteen day tumor growth delay.

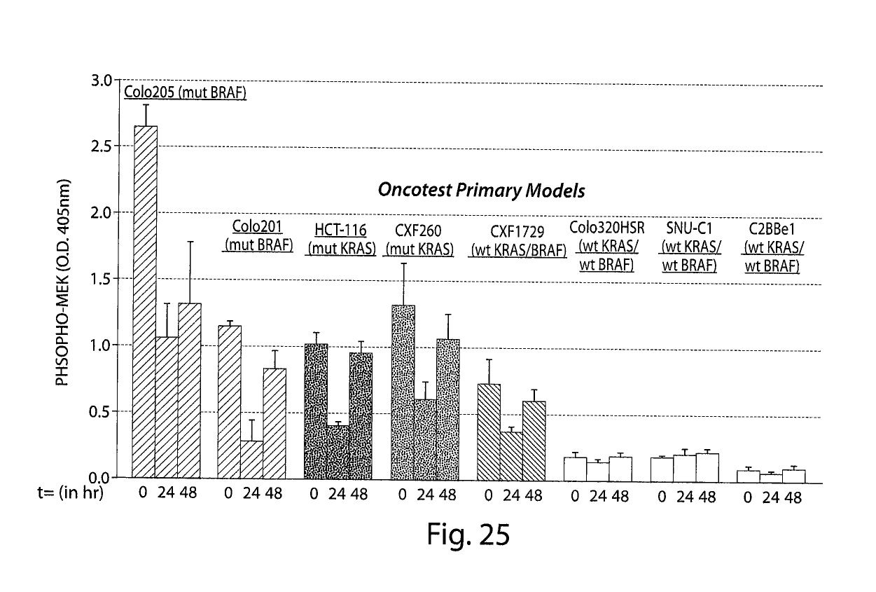

### Experiment 5. FIG. 25

In the fifth set of studies, select colorectal tumor models were established; mutant BRAF (Colo201, Colo205), mutant KRAS (HCT-116, CXF-260) and wild-type BRAF/KRAS (CXF-1729, Colo320HSR, SNU-C1) (FIG. 25). Once tumors had reached approximately 200 mm³, animals were either left untreated (T=0) or administered a single oral dose of a suspension of 50% 17-AG in HPMC-AS-HG dispersion in vehicle (1% hydroxyethylcellulose, 5% glycerol) at 100 mg/kg and then tumors were harvested at twenty-four (T=24) and forty-eight (T=48) hours following treatment and flash frozen in liquid nitrogen. Tumor cell lysates were prepared from frozen tumor tissue for each xenograft sample and the activity (phosphorylation) of MEK1 (a surrogate marker of the activity of the MAP Kinase pathway-MAPK) was assessed by ELISA assay. The results of these studies demonstrate that for the tumor models tested, the activity of the MAPK pathway correlates very well with sensitivity to 17-AG treatment as assessed by tumor growth inhibition.

### Experiment 6. FIGS. 26A and 26B

In the sixth set of studies, subcutaneous xenografts of Colo201 (mutant BRAF) were established by implantation of 10×10⁶ cells into the right flank of nude mice. Once tumors reached approximately 100-150 mm³, animals were randomized into treatment groups and administered either vehicle or a suspension of 50% 17-AG in HPMC-AS-HG dispersion in vehicle (1% hydroxyethylcellulose, 5% glycerol) (100 mg/kg, M-W-F, 3 weeks), Irinotecan (100 mg/kg, Q7D, 3 weeks) or the combination of a suspension of 50% 17-AG in HPMC-AS-HG dispersion in vehicle (1% hydroxyethylcellulose, 5% glycerol) (50 mg/kg, M-W-F) and Irinotecan (75 mg/kg, Q7D) for three weeks, and tumor volume measured twice per week (FIG. 26A; FIG. 26B is a zoomed-in section of FIG. 26A). At the end of the dosing period, animals were monitored for an additional 57 days to follow tumor growth delay. The outcome of these studies demonstrate that compared to 17-AG or Irinotecan single agent administration, the combination of 17-AG plus Irinotecan showed dramatically increased efficacy with an average 75% tumor regression and four out of ten complete responses (complete disappearance of a palpable tumor) during the treatment phase which was maintained throughout the tumor growth delay phase (end of study=78 days total, treatment+post treatment phases). In contrast, single agent administration of either 17-AG or Irinitecan only resulted in tumor growth inhibition while on drug (TGI=˜88% for both drug treatments), with no complete responses in either treatment arm of the study. Similar results to those described above for the Colo201 model were also observed in two separate colorectal models (HCT-116 and DuDu1, both G12V mutant KRAS) (Experiments 7 and 8).

### Experiment 7. FIGS. 27A and 27B

In the seventh set of studies, subcutaneous xenografts of HCT-116 (G13D mutant KRAS) colorectal tumor model were established by implantation of 10×10⁶ cells into the right flank of nude mice. Once tumors reached approximately 100-150 mm³, animals were randomized into treatment groups and administered either vehicle or a suspension of 50% 17-AG in HPMC-AS-HG dispersion in vehicle (1% hydroxyethylcellulose, 5% glycerol) (100 mg/kg, M-W-F, 3 weeks), Irinotecan (100 mg/kg, Q7D, 3 weeks) or the combination of a suspension of 50% 17-AG in HPMC-AS-HG dispersion in vehicle (1% hydroxyethylcellulose, 5% glycerol) (50 mg/kg, M-W-F) and Irinotecan (75 mg/kg, Q7D) for three weeks, and tumor volume measured twice per week (FIG. 27A; FIG. 27B is a zoomed-in section of FIG. 27A). At the end of the dosing period, animals were monitored for an additional 21 days to follow tumor growth delay. The outcome of these studies demonstrate that while on drug, compared to 17-AG or Irinotecan single agent administration, the combination of 17-AG plus Irinotecan demonstrated increased efficacy with an average tumor growth inhibition of ˜90%. In addition, during the re-growth phase of the study the 17-AG plus Irinotecan combination resulted in an approximate seventeen day delay in tumor progression. In contrast, single agent administration of either 17-AG or Irinotecan only resulted in tumor growth inhibition of ˜75% while on drug, with no delay in tumor progression in either single agent treatment arm of the study.

### Experiment 8. FIGS. 28A and 28B

In the eighth set of studies, subcutaneous xenografts of DuDu-1 (G12V mutant KRAS) patient-derived tumor colorectal model were established by implantation of 10×10⁶ cells into the right flank of nude mice. Once tumors reached approximately 100-150 mm³, animals were randomized into treatment groups and administered either vehicle or a suspension of 50% 17-AG in HPMC-AS-HG dispersion in vehicle (1% hydroxyethylcellulose, 5% glycerol) (100 mg/kg, M-W-F, 3 weeks), Irinotecan (100 mg/kg, Q7D, 3 weeks) or the combination of 17-AG (50 mg/kg, M-W-F) and Irinotecan (75 mg/kg, Q7D) for three weeks, and tumor volume measured twice per week (FIG. 28A; FIG. 28B is a zoomed-in section of FIG. 28A). At the end of the dosing period, animals were monitored for an additional 24 days to follow tumor growth delay. The outcome of these studies demonstrate that while on drug, compared to 17-AG or Irinotecan single agent administration, the combination of 17-AG plus Irinotecan demonstrated increased efficacy with an average tumor growth inhibition (TGI) of 77%. In addition, during the re-growth phase of the study the 17-AG plus Irinotecan combination resulted in an approximate sixteen day delay in tumor progression. In contrast, single agent administration of Irinotecan was relatively ineffective resulting in only tumor growth inhibition of ˜35% while on drug and no tumor growth delay. Interestingly, 17-AG administration alone resulted in 70% TGI and a delay of ten days in tumor progression once drug administration was stopped.

Experiment 9: Activity of the Novel Hsp90 Inhibitor IPI-493 in Models of Colorectal Cancer Correlates with Ras Pathway Activation

Heat-shock protein 90 (Hsp90) has emerged as an attractive target in cancer due to its role in maintaining the activity and stability of a variety of oncoproteins, including HER2, BCR-ABL, EML4-ALK and mutant EGFR. Infinity is developing two novel Hsp90 inhibitors, IPI-504 (IV administered) and IPI-493 (orally administered). IPI-504 is currently being evaluated in multiple phase 2 clinical trials; IPI-493 is being evaluated in two phase 1 trials. To investigate the activity of IPI-493 in colorectal cancer (CRC), we performed in vitro growth inhibition (GI) studies on a CRC cell line panel. IPI-493 demonstrated GI₅₀s in the range of 10-100 nM in cell lines harboring either kRAS or bRAF mutations. To explore the in vivo potency of IPI-493, several mutant bRAF, mutant kRAS, as well as a wild-type kRAS/bRAF xenograft models were developed. Administration of IPI-493 at 100 mg/kg three times weekly demonstrated dramatic effects in all of the mutant models tested, with tumor growth inhibition (TGI) values between 70 and 90% and regression in one mutant bRAF model. Importantly, when IPI-493 was evaluated in combination with irinotecan, the combination showed superior efficacy versus either drug alone, which led to overall tumor regression and/or complete regressions in 4 of 10 animals. Interestingly, in the wild-type kRAS/bRAF models, IPI-493 administration did not lead to tumor growth inhibition. These results suggest that activation of the MAPK pathway may predispose these cells to sensitivity to HSP90 inhibition. To investigate the effect of IPI-493 on MAPK pathway activity, we performed pharmacodynamic analysis after a single dose of IPI-493 in multiple xenograft models differing in their RAF/RAS mutation status. In mutant bRAF models, pathway activity was high, and IPI-493 administration resulted in downregulation of the activity of both bRAF and MEK. In models containing no mutations in kRAS or bRAF, we detect low baseline levels of both p-bRAF and p-MEK and little effect of IPI-493 administration. When Ras pathway activity in all CRC xenografts was compared with IPI-493 efficacy, there was a clear c-orrelation between pathway activation and tumor growth inhibition by IPI-493. Our finding that Ras pathway activation predisposes CRC cells to sensitivity to IPI-493 and our combination data with -irinotecan provide a clear rationale for Hsp90 inhibitors in colorectal cancer.

### Conclusion

It was observed from the above described experiments that the Hsp90 inhibitor 17-AG demonstrates dramatic efficacy in both in vitro and in vivo models of KRAS and BRAF mutant CRC. In contrast, the majority of the models wt/wt for both KRAS and BRAF exhibited little to no sensitivity to Hsp90 inhibition. It was also observed that the combination of the Hsp90 inhibitor 17-AG and irinotecan (SOC in CRC) demonstrates efficacy over either agent administered alone.

Pathway analysis of tumors from mutant K-Ras/B-Raf and wt/wt models demonstrated that MAPK pathway activity is a good predictor of Hsp90 sensitivity. For example, all “sensitive” models displayed increased baseline MAPK pathway activity whereas no detectable efficacy was observed in models that displayed very low MAPK pathway activity. Analysis of MAPK pathway (RAS-RAF-MEK) activity can offer a clinical strategy to predict IPI-493 sensitivity.

These data demonstrate that HSP90 inhibition is comparable to SOC and the combination of an HSP90i with SOC could be a more efficacious approach for treatment of mCRC

### Example 8

### Effectiveness of Combination of an HSP90 Inhibitor and an mTOR Kinase Inhibitor for Treating Ras-driven Malignancies

The mTOR kinase is frequently deregulated in human cancer due to genetic alterations in various tumor suppressors and oncogenes including PTEN, TSC1/2, LKB, NF1, PI3K and RAS (Menon, S. et al. (2008) Oncogene 27 Suppl 2, S43-51; Sabatini, D. M. (2006) Nat Rev Cancer 6, 729-734). Consequently, mTOR inhibitors have been evaluated as potential cancer therapies in the clinic (Chiang, G. G. et al. (2007) Trends Mol Med 13, 433-442). While these inhibitors exhibit efficacy in a subset of tumor-types, responses are typically cytostatic and temporary, suggesting that mTOR inhibitors might be more effective when combined with other agents (Dancey, J. (2010) Nat Rev Clin Oncol. 7, 209-219). Potent therapeutic effects of combining an mTOR inhibitor with agents that induce ER stress, e.g., the HSP90 inhibitor IPI-504, are demonstrated herein. Rapamycin/IPI-504 treatment causes tumor regression in a KrasG12D/p53 genetically engineered model of non-small cell lung cancer (NSCLC) (data not shown). These findings reveal a promising strategy for developing therapies based on the combination of an HSP90 inhibitor and an mTOR inhibitor for Ras-driven malignancies.

To investigate the potential therapeutic efficacy of combination therapy of rapamycin/IPI-504 in a tumor driven by an activating mutation in RAS, a genetically engineered model was used in which NSCLC is driven by compound mutations in KRAS and p53 (Jackson, E. L., et al. (2005) Cancer Res 65, 10280-10288). In this model lung adenocarcinomas are induced by intranasal administration of adenoviral Cre, which causes the concomitant expression of a single KrasG12D allele and loss of p53 (herein referred to as LSLKrasG12D/+; p53fl/fl mice).

Administration of rapamycin and IPI-504 in combination causes tumor regression (data not shown).

Notably, while combined MEK and PI3K inhibitors have been shown to promote tumor regression in murine NSCLCs harboring the KrasG12D mutation alone (Engelman, J. A., et al. (2008) Nat Med 14, 1351-1356), to date no targeted therapy has been shown to promote the regression of the more aggressive KrasG12D, p53-deficient tumors. These results underscore the significance of this finding and its potential impact on therapeutic development in KRAS driven NSCLC in humans.

Future studies should reveal whether the therapeutic effects of this combination can extend to other Ras-driven and/or mTOR-driven cancers. While mTOR inhibitors exhibit anti-tumor activity in some cancers, there has been a concerted effort to enhance the efficacy and utility of these agents (reviewed in Dancey, J. (2010) Nat Rev Clin Oncol. 7, 209-219). One strategy has been to develop more potent mTOR or dual (mTOR/PI3K) inhibitors. Several second-generation compounds are currently in development and clinical trials will ultimately reveal whether they are more efficacious. However, a second approach has been to identify combination therapies that can convert the generally cytostatic effect of mTOR inhibitors to a cytotoxic response. A promising strategy for developing a potent mTOR inhibitor-based combination therapy in KRAS-driven lung cancers is disclosed herein.

While these studies provide compelling data to support the clinical investigation of combined rapamycin/IPI-504 therapy, they also serve as a foundation for developing combinations with other related agents. For example, a more potent mTOR inhibitor can enhance the therapeutic efficacy of this combination. Similarly, there are currently several structurally unrelated Hsp90 inhibitors in clinical development, which should provide an array of compounds that can differ in efficacy and/or in toxicity. However, the potential utility of these agents can be overlooked if they are assessed exclusively as mono-therapies in genetically heterogeneous tumors using tumor regression as an endpoint.

Our findings demonstrate that these agents can potently synergize when combined, converging on basic cell biological processes (ER stress and autophagy), and selectively kill tumor cells in two robust animal tumor models that are refractory to single targeted agents.

### Example 9

### HSP90 Inhibitors Inhibit the Proliferation of Neuroendocrine and Carcinoid Cell Lines In Vitro

### Materials and Methods

Cell lines BON-1 (a metastatic cell originating from the pancreas), H-720 (a carcinoid cell originating from the lung), QGP-1 (originating from a carcinoma of pancreatic islet cells) and HC45 (a carcinoid cell originating from the ileum) were grown in RPMI-1640 medium supplemented with 10% fetal bovine serum, 1 μg/ml streptomycin and 1 μg/ml penicillin. All cell lines were tested for mycoplasma and maintained at 37° C. in 5% CO2 atmosphere.

Cells were seeded at 10,000 cells/well in 96 well plates for 24 hours and subsequently incubated with increasing concentrations of IPI-504 for 72 hrs, and viability studies were performed using the vital mitochondrial function stain Cell Titer Glow kit (Promega, Madison, Wis.). The data were normalized with respect to DMSO vehicle control to generate growth inhibition GI50 values.

### Background

Gastroenteropancreatic neuroendocrine tumors (GEP-NET, also called carcinoids) were originally seen as a homogenous group of neoplasms, but advances in the molecular characterization of GEP-NET led to a more complex classification published by the WHO in 2000. With a reported incidence of 2-3:100,000 these tumors are relatively rare, but the 5-year survival rate is only about 67%. For localized tumors, producing an excess of biogenic amines and hormones, systemic symptoms are limited by the rapid hepatic clearance of these molecules. However, metastasized tumors often have incapacitating symptoms, including diarrhea, flushing, wheezing and skin rashes.

The treatment of choice for localized tumors is still surgical resection. However, about 80% of patients have already developed liver or lymph-node metastases upon presentation. In the advanced stages, the medical treatment options are still poor. Although tumor-related symptoms are often well controlled by somatostatin-analogues (i.e., lanreotide and octreotide), which are sometimes combined with interferon-α, an effective inhibitor of tumor growth is not available at this time. Combinations of etoposide plus cisplatin, or streptozocin plus 5-FU or doxorubicin are used in chemotherapy treatment, however response rates are a disappointing 0-30%. Thus, effective new treatment strategies are urgently needed.

Heat shock protein 90 (Hsp90), an emerging target for the treatment of cancer, is a highly expressed protein chaperone that associates with many “client” proteins implicated in oncogenesis. Indeed, numerous Hsp90 client proteins are kinases or transcription factors involved in cellular proliferation, angiogenesis, invasion, and metastasis. Hsp90 has been shown to be over-expressed in a wide range of tumor types including breast, endometrial, ovarian, colon, lung and prostate (Ciocca D R, et al. (2005) Cell Stress Chaperones. 10(2):86-103). Noteworthy, several of the proteins that are known to be over expressed in GEP-NET are regulated by Hsp90, including EGFR, ERbB2, IGF1-R and AKT (Höpfner M, et al (2008) WJG. 14(16):2461; Pitt S C, et al. (2009) Am J Transl Res. 1(3):291-299).

Inhibition of Hsp90 leads to rapid degradation of client proteins through the ubiquitin-proteasome pathway (McDonough H, et al. (2003) Cell Stress Chaperones. 8(4):303-308). Studies have demonstrated the activity of Hsp90 inhibitors in multiple models of solid (e.g., lung, breast, prostate, pancreatic, melanoma) and hematologic (e.g., chronic myelogenous leukemia, multiple myeloma) cancers. The in vitro effect of HSP90 inhibitors, such as IPI-504, on the growth of several GEP-NET cell lines, and its mechanism of action.

### Results

The graphs in FIGS. 29A and 29B show the percent growth inhibition for three cell lines (BON-1, QGP-1 and H-720) for various concentrations of 17-AG (IPI-493) and IPI-504. The four neuroendocrine cell lines treated with increasing concentrations of IPI-504 for 72 hours showed a dose dependent decrease in cell proliferation. The growth inhibition of cell lines NCI-H720 and HC-45 reached a maximum of 100%, suggesting a cytotoxic effect of IPI-504, whereas cell lines BON-1 and QGP-1 reached a growth inhibition plateau at 60%, suggesting a cytostatic effect of the drug (FIG. 29A).

In addition, GI50 values (i.e., the concentration of compound needed to reduce the growth of treated cells to half that of untreated cells) for 17-AG, IPI-504, SNX-2112 and NVP-AUY922 are provided in Table 6. All carcinoid cell lines tested were sensitive to IPI-504 with GI₅₀ values between 10 nM and 1 μM Table 6).

TABLE 6
GI50 values (nm)

| | BON-1 | H-720 | QGP-1 | HC-45 |
|---|---|---|---|---|
| 17-AG | 19 | 189 | 10 | 88 |
| Compound 2 | 49 | 930 | 302 | 705 |
| (IPI-504) | | | | |
| SNX-2112 | 35 | 133 | 25 | 224 |
| NVP-AUY922 | 13 | 68 | 3 | 28 |

Thus, IPI-504 and IPI-493 (17-AG) inhibit the proliferation of neuroendocrine cell lines by induction of apoptosis and cell cycle arrest.

### Example 10

### Xenograft Studies of BON-1 Cells

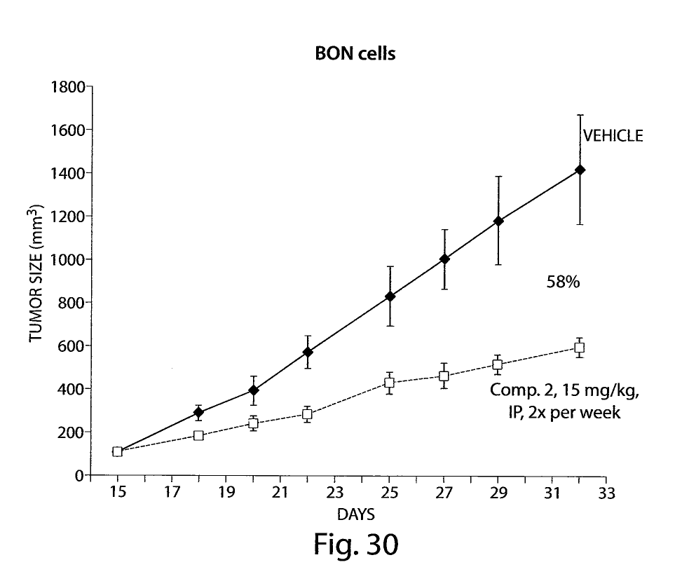

Six to eight week old male NCr nude athymic (nu-/nu-) mice (Taconic Farms, Hudson, N.Y.), were maintained in accordance with the Institutional Animal Care and Use Committee guidelines. Xenographs were generated by injecting 5×106 BON-1 cells into the flanks of 40 mice. IPI-504 (also referred to herein as IPI-504) (15 mg/kg) or vehicle were administered i.p. twice per week (n=10 per arm), and tumor xenograft size was monitored twice weekly with calipers. Results are presented as means and SEM. As shown in FIG. 30, mice treated with IPI-504 exhibited a 58% reduction in tumor size compared with vehicle at the end of the study.

### Example 11

### IPI-504 Inhibits the Hsp90 Client Protein IGF-1R

### Materials and Methods

BON-1 and H-720 cell lysates were hybridized to R&D Systems' Human Phospho-Receptor Tyrosine Kinase (RTK) Arrays (Catalog #ARY001) according to the manufacturer's instructions. In the array, each RTK is spotted in duplicate. Hybridization signals at the corners serve as controls. The array revealed that BON-1 and H-720 cells show constitutive phosphorylation of IR and IGF1R receptors. Treatment with 1 μM IPI-504 or 17-AG overnight inhibited this constitutive phosphorylation.

### Results

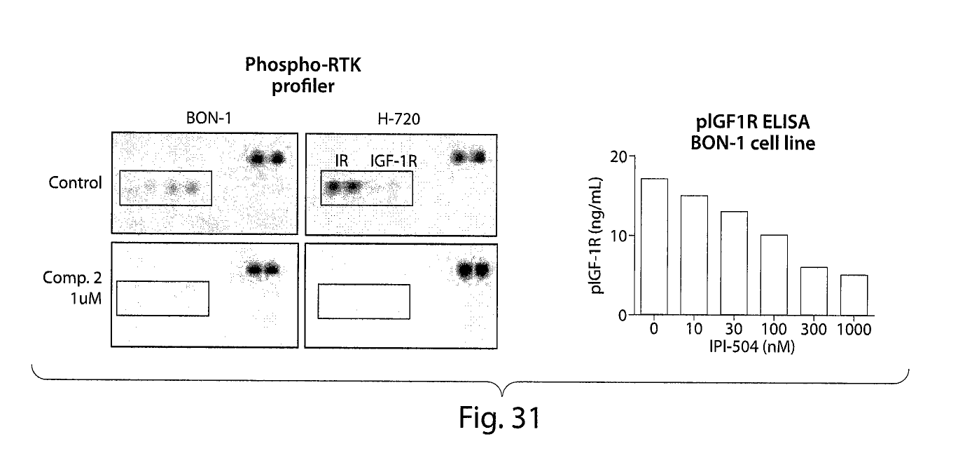

Insulin-like Growth Factor 1 Receptor (IGF-1R) is over expressed in gastroenteropancreatic neuroendocrine tumors (GEP-NET) cells, and it is also a client protein of Hsp90. BON-1 cells were incubated with increasing concentrations of IPI-504 for 24 hours, and levels of phosphorylated IGF-1R were monitored in cell lysates using a phospho-IGF-1R ELISA according to the manufacturer' protocol (cat #7820, Tyr 1131). As shown in FIG. 31, upon treatment with IPI-504, phospho-IGF-1R is degraded in BON-1 cells in a dose-dependent manner. The EC50 of the protein degradation and the in vitro growth inhibitory activity of IPI-504 are similar (−50 nM), suggesting that the anti-tumor activity of IPI-504 could be due, in part, to the inactivation of this growth factor receptor.

Thus, the growth factor receptor, IGF-1R, is constitutively activated in the BON-1 cell line. Treatment with IPI-504 decreases the amount of phospho-IGF-1R in a dose dependent manner. The IC50 for this process matches the growth inhibition of BON-1 cells by IPI-504. Therefore, inhibition of IGF-1R phosphorylation can be a possible mechanism of action by IPI-504.

### Example 12

### Additive Effect of Combined Hsp90 Inhibition and mTOR or Akt Inhibition in Neuroendocrine Cell Lines

### Materials and Methods

BON-1 cells were incubated for either 6 hours or 24 hours with 1 uM of IPI-504, 100 nM rapamycin (Sigma) or the combination of both. 50 ug of cell lysate was immunoblotted for pAKT, total AKT, pS6, total S6, pERK 1/2 (Cell Signaling), IGF-1Rb, Hsp70, and b-actin (Santa Cruz). Image analysis and band quantization were performed with the Bio-Rad Versa Doc system. The expression of GADPH was used as a control for protein loading.

### Results

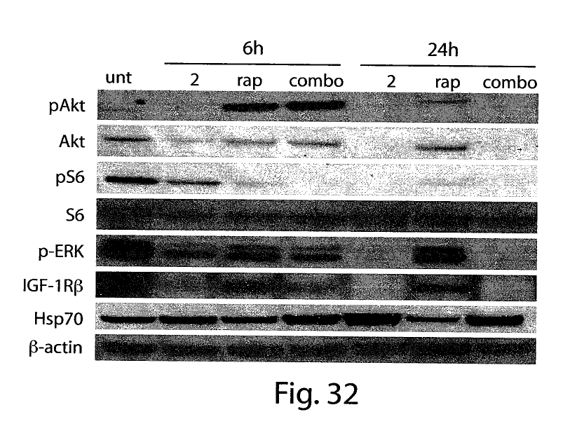

Since the PI3K/Akt/mTOR pathway is activated in neuroendocrine tumors, GEP-NET cells were treated with IPI-504 and drugs which inhibit the AKT/mTOR pathway to look for combination effects. In one experiment, BON-1 cells were incubated for 6 or 24 hours with 1 μM IPI-504, 100 nM rapamycin or the combination of both. Fifty μg of cell lysate was immunoblotted for pAKT, total AKT, pS6, IGF-1Rβ, Hsp70, and β-actin. Rapamycin inhibition leads to an increase in AKT phosphorylation whereas IPI-504 incubation leads to AKT degradation. The combination of IPI-504 and rapamycin exhibited additive effects (FIG. 32). These data indicate that IPI-504 and rapamycin work additively together to inhibit S6 kinase activation downstream of mTOR.

### Discussion

Through the use of established drugs like somatostatin analogues, great progress has been made in controlling the often debilitating hypersecretion syndrome encountered in patients with metastasized GEP-NETs (Panzuto F, et al. (2006) Ann. Oncol. 17(3):461-466). However, cytostatic therapy regimens aimed at slowing tumor progression, or inducing remission have had limited success (Kouvaraki M A, et al. (2004) J Clin Oncol. 22(23):4762-4771). Deregulated Hsp90 is known to be important for tumor survival and progression (Mahalingam D, et al. (2009) Br J. Cancer. 100(10):1523-1529; Ciocca D R, et al. (2005) Cell Stress Chaperones. 10(2):86-103), and several proteins deregulated in GEP-NETs are at least partially controlled by Hsp90 (Höpfner M, et al (2008) WJG. 14(16):2461; Pitt S C, et al. (2009) Am J Transl Res. 1(3):291-299). In this study, inhibition of Hsp90 by IPI-504 is shown to inhibit neuroendocrine tumor cells, and thus provides a promising approach for novel GEP-NET treatment options.

Since GEP-NETs are very heterogeneous, and include both slow growing and fast growing, aggressive tumors (Klöppel G, et al. (2004) Annals of the New York Academy of Sciences. 1014(Gastroenteropancreatic Neuroendocrine Tumor Disease: Molecular and Cell Biological Aspects):13-27), it is important to evaluate different representatives of this tumor entity when testing new therapeutic agents. Therefore, five cell lines with different growth rates and origins were tested. The IC₅₀ values of IPI-504 in the different cell lines did not show a clear correlation to the doubling times of the cells, indicating that characteristics other than the growth rate might be more important in determining sensitivity towards Hsp90 inhibition.

Treatment of GEP-NET cell lines with IPI-504 led to a time- and dose-dependent reduction in cell growth by inducing cell cycle arrest and/or apoptosis. In BON-1 and CM cells the anti-proliferative effect of IPI-504 correlated with a reduction in protein levels of the IGF-1 receptor, in good agreement with earlier publications were we reported that the IGF-1 receptor plays an important role in the survival and proliferation of GEP-NETs (Höpfner M, et al. (2006) Endocr. Relat. Cancer. 13(1):135-149). Additionally, several proteins in the PI3K/AKT/mTOR pathway, which are thought to be tightly regulated by the IGF-1 receptor in neuroendocrine tumor cells (von Wichert G, et al. (2005) Oncogene 24(7):1284-1289), were down regulated as a consequence of Hsp90 inhibition. This most likely happens not only as a consequence of IGF-1R down regulation, but also as a direct influence of Hsp90 inhibition. AKT is known to be associated with Hsp90 as its chaperone to prevent degradation. Treatment of GEP-NETs with IPI-504 not only decreased the amount of active, phosphorylated AKT, but also led to a reduction of total AKT, indicating an increase in ubiquitination of this protein. Furthermore the ribosomal protein S6 (rpS6), which is further downstream in the PI3K-AKT pathway, is also down regulated and directly influenced by Hsp90 (Kim T, et al. (2006) Mol. Biol. Cell. 17(2):824-833).

Next the combinations of IPI-504 with other targeted cancer therapeutics were evaluated. The protein mTORC1 is part of the PI3K-AKT pathway and closely interacts with AKT (Sarbassov D D, et al. (2005) Science 307(5712):1098-1101). A phase II study has recently been completed investigating the effect of an mTORC1 inhibitor with or without octreotide in patients with pancreatic neuroendocrine tumors, yielding promising results. Studies showing that mTORC1 inhibition leads to upregulation of AKT via loss of feedback inhibition (O'Reilly K E, et al. (2006) Cancer Res. 66(3):1500-1508) make mTORC1 inhibitors even more attractive as combination partners for drugs targeting other proteins within the PI3K/AKT pathway. As a consequence, several new compounds are being studied as dual PI3K/mTORC1 inhibitors. We therefore combined IPI-504 with the mTORC1 inhibitor, rapamycin, and found strong additive antiproliferative effects. These results are important for two at least two reasons. First, many in vitro and in vivo studies using chemotherapeutic agents for targeted therapy in cancer have only shown modest anti-neoplastic effects, mainly resulting in slowing down the disease. This is believed to be due to escape-mechanisms of the cells, which are more pronounced when only a single molecule is targeted. Thus, by targeting a growth-pathway simultaneously at multiple sites, as shown in our results, might result in a better tumor-response in vivo. Second, by combining two agents, one can hope to diminish adverse events in the clinical setting.

Recently, several studies indicated that Hsp90 can be important on an extracellular level, where it could influence cell motility (Sidera K, et al. (2008) Cell Cycle. 7(11):1564-1568). A monoclonal antibody selectively targeting extracellular Hsp90 has been shown to decrease the formation of metastatic lesions in different types of cancer in vitro (Stellas D, et al. (2007) Clin. Cancer Res. 13(6):1831-1838). IPI-504 potently inhibits the migration of gastrointestinal neuroendocrine tumor cells, thus supporting the concept of its role in metastatic diseases.

In summary, Hsp90 inhibition is an attractive target in GEP-NETs. Combination treatments in our study showed promising additive effects, and metastatic disease seems to be an especially promising target for this new therapeutic option.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1

ALK (anaplastic lymphoma kinase) sequences
ALK wild type mRNA
Ref Seq: NM_004304.3 GI: 29029631

| | |
|---|---|
| GGGGGCGGCA GCGGTGGTAG CAGCTGGTAC CTCCCGCCGC CTCTGTTCGG | 50 |
| AGGGTCGCGG GGCACCGAGG TGCTTTCCGG CCGCCCTCTG GTCGGCCACC | 100 |
| CAAAGCCGCG GGCGCTGATG ATGGGTGAGG AGGGGGCGGC AAGATTTCGG | 150 |
| GCGCCCCTGC CCTGAACGCC CTCAGCTGCT GCCGCCGGGG CCGCTCCAGT | 200 |
| GCCTGCGAAC TCTGAGGAGC CGAGGCGCCG GTGAGAGCAA GGACGCTGCA | 250 |
| AACTTGCGCA GCGCGGGGGC TGGGATTCAC GCCCAGAAGT TCAGCAGGCA | 300 |
| GACAGTCCGA AGCCTTCCCG CAGCGGAGAG ATAGCTTGAG GGTGCGCAAG | 350 |
| ACGGCAGCCT CCGCCCTCGG TTCCCGCCCA GACCGGGCAG AAGAGCTTGG | 400 |
| AGGAGCCAAA AGGAACGCAA AAGGCGGCCA GGACAGCGTG CAGCAGCTGG | 450 |
| GAGCCGCCGT TCTCAGCCTT AAAAGTTGCA GAGATTGGAG GCTGCCCCGA | 500 |
| GAGGGGACAG ACCCCAGCTC CGACTGCGGG GGGCAGGAGA GGACGGTACC | 550 |
| CAACTGCCAC CTCCCTTCAA CCATAGTAGT TCCTCTGTAC CGAGCGCAGC | 600 |
| GAGCTACAGA CGGGGGCGCG GCACTCGGCG CGGAGAGCGG GAGGCTCAAG | 650 |
| GTCCCAGCCA GTGAGCCCAG TGTGCTTGAG TGTCTCTGGA CTCGCCCCTG | 700 |
| AGCTTCCAGG TCTGTTTCAT TTAGACTCCT GCTCGCCTCC GTGCAGTTGG | 750 |
| GGGAAAGCAA GAGACTTGCG CGCACGCACA GTCCTCTGGA GATCAGGTGG | 800 |
| AAGGAGCCGC TGGGTACCAA GGACTGTTCA GAGCCTCTTC CCATCTCGGG | 850 |
| GAGAGCGAAG GGTGAGGCTG GGCCCGGAGA GCAGTGTAAA CGGCCTCCTC | 900 |
| CGGCGGGATG GGAGCCATCG GGCTCCTGTG GCTCCTGCCG CTGCTGCTTT | 950 |
| CCACGGCAGC TGTGGGCTCC GGGATGGGGA CCGGCCAGCG CGCGGGCTCC | 1000 |
| CCAGCTGCGG GGCCGCCGCT GCAGCCCCGG GAGCCACTCA GCTACTCGCG | 1050 |
| CCTGCAGAGG AAGAGTCTGG CAGTTGACTT CGTGGTGCCC TCGCTCTTCC | 1100 |
| GTGTCTACGC CCGGGACCTA CTGCTGCCAC CATCCTCCTC GGAGCTGAAG | 1150 |
| GCTGGCAGGC CCGAGGCCCG CGGCTCGCTA GCTCTGGACT GCGCCCCGCT | 1200 |
| GCTCAGGTTG CTGGGGCCGG CGCCGGGGGT CTCCTGGACC GCCGGTTCAC | 1250 |
| CAGCCCCGGC AGAGGCCCGG ACGCTGTCCA GGGTGCTGAA GGGCGGCTCC | 1300 |
| GTGCGCAAGC TCCGGCGTGC CAAGCAGTTG GTGCTGGAGC TGGGCGAGGA | 1350 |
| GGCGATCTTG GAGGGTTGCG TCGGGCCCCC CGGGGAGGCG GCTGTGGGGC | 1400 |
| TGCTCCAGTT CAATCTCAGC GAGCTGTTCA GTTGGTGGAT TCGCCAAGGC | 1450 |
| GAAGGGCGAC TGAGGATCCG CCTGATGCCC GAGAAGAAGG CGTCGGAAGT | 1500 |
| GGGCAGAGAG GGAAGGCTGT CCGCGGCAAT TCGCGCCTCC CAGCCCCGCC | 1550 |
| TTCTCTTCCA GATCTTCGGG ACTGGTCATA GCTCCTTGGA ATCACCAACA | 1600 |
| AACATGCCTT CTCCTTCTCC TGATTATTTT ACATGGAATC TCACCTGGAT | 1650 |
| AATGAAAGAC TCCTTCCCTT TCCTGTCTCA TCGCAGCCGA TATGGTCTGG | 1700 |
| AGTGCAGCTT TGACTTCCCC TGTGAGCTGG AGTATTCCCC TCCACTGCAT | 1750 |
| GACCTCAGGA ACCAGAGCTG GTCCTGGCGC CGCATCCCCT CCGAGGAGGC | 1800 |
| CTCCCAGATG GACTTGCTGG ATGGGCCTGG GGCAGAGCGT TCTAAGGAGA | 1850 |
| TGCCCAGAGG CTCCTTTCTC CTTCTCAACA CCTCAGCTGA CTCCAAGCAC | 1900 |
| ACCATCCTGA GTCCGTGGAT GAGGAGCAGC AGTGAGCACT GCACACTGGC | 1950 |
| CGTCTCGGTG CACAGGCACC TGCAGCCCTC TGGAAGGTAC ATTGCCCAGC | 2000 |
| TGCTGCCCCA CAACGAGGCT GCAAGAGAGA TCCTCCTGAT GCCCACTCCA | 2050 |
| GGGAAGCATG GTTGGACAGT GCTCCAGGGA AGAATCGGGC GTCCAGACAA | 2100 |
| CCCATTTCGA GTGGCCCTGG AATACATCTC CAGTGGAAAC CGCAGCTTGT | 2150 |
| CTGCAGTGGA CTTCTTTGCC CTGAAGAACT GCAGTGAAGG AACATCCCCA | 2200 |
| GGCTCCAAGA TGGCCCTGCA GAGCTCCTTC ACTTGTTGGA ATGGGACAGT | 2250 |
| CCTCCAGCTT GGGCAGGCCT GTGACTTCCA CCAGGACTGT GCCCAGGGAG | 2300 |
| AAGATGAGAG CCAGATGTGC CGGAAACTGC CTGTGGGTTT TTACTGCAAC | 2350 |
| TTTGAAGATG GCTTCTGTGG CTGGACCCAA GGCACACTGT CACCCCACAC | 2400 |
| TCCTCAATGG CAGGTCAGGA CCCTAAAGGA TGCCCGGTTC CAGGACCACC | 2450 |
| AAGACCATGC TCTATTGCTC AGTACCACTG ATGTCCCCGC TTCTGAAAGT | 2500 |
| GCTACAGTGA CCAGTGCTAC GTTTCCTGCA CCGATCAAGA GCTCTCCATG | 2550 |
| TGAGCTCCGA ATGTCCTGGC TCATTCGTGG AGTCTTGAGG GGAAACGTGT | 2600 |
| CCTTGGTGCT AGTGGAGAAC AAAACCGGGA AGGAGCAAGG CAGGATGGTC | 2650 |
| TGGCATGTCG CCGCCTATGA AGGCTTGAGC CTGTGGCAGT GGATGGTGTT | 2700 |
| GCCTCTCCTC GATGTGTCTG ACAGGTTCTG GCTGCAGATG GTCGCATGGT | 2750 |
| GGGGACAAGG ATCCAGAGCC ATCGTGGCTT TTGACAATAT CTCCATCAGC | 2800 |
| CTGGACTGCT ACCTCACCAT TAGCGGAGAG GACAAGATCC TGCAGAATAC | 2850 |
| AGCACCCAAA TCAAGAAACC TGTTTGAGAG AAACCCAAAC AAGGAGCTGA | 2900 |
| AACCCGGGGA AAATTCACCA AGACAGACCC CCATCTTTGA CCCTACAGTT | 2950 |
| CATTGGCTGT TCACCACATG TGGGGCCAGC GGGCCCCATG GCCCCACCCA | 3000 |
| GGCACAGTGC AACAACGCCT ACCAGAACTC CAACCTGAGC GTGGAGGTGG | 3050 |
| GGAGCGAGGG CCCCCTGAAA GGCATCCAGA TCTGGAAGGT GCCAGCCACC | 3100 |
| GACACCTACA GCATCTCGGG CTACGGAGCT GCTGGCGGGA AAGGCGGGAA | 3150 |
| GAACACCATG ATGCGGTCCC ACGGCGTGTC TGTGCTGGGC ATCTTCAACC | 3200 |
| TGGAGAAGGA TGACATGCTG TACATCCTGG TTGGGCAGCA GGGAGAGGAC | 3250 |
| GCCTGCCCCA GTACAAACCA GTTAATCCAG AAAGTCTGCA TTGGAGAGAA | 3300 |
| CAATGTGATA GAAGAAGAAA TCCGTGTGAA CAGAAGCGTG CATGAGTGGG | 3350 |
| CAGGAGGCGG AGGAGGAGGG GGTGGAGCCA CCTACGTATT TAAGATGAAG | 3400 |
| GATGGAGTGC CGGTGCCCCT GATCATTGCA GCCGGAGGTG GTGGCAGGGC | 3450 |
| CTACGGGGCC AAGACAGACA CGTTCCACCC AGAGAGACTG GAGAATAACT | 3500 |
| CCTCGGTTCT AGGGCTAAAC GGCAATTCCG GAGCCGCAGG TGGTGGAGGT | 3550 |
| GGCTGGAATG ATAACACTTC CTTGCTCTGG GCCGGAAAAT CTTTGCAGGA | 3600 |
| GGGTGCCACC GGAGGACATT CCTGCCCCCA GGCCATGAAG AAGTGGGGGT | 3650 |
| GGGAGACAAG AGGGGGTTTC GGAGGGGGTG GAGGGGGGTG CTCCTCAGGT | 3700 |
| GGAGGAGGCG GAGGATATAT AGGCGGCAAT GCAGCCTCAA ACAATGACCC | 3750 |
| CGAAATGGAT GGGGAAGATG GGGTTTCCTT CATCAGTCCA CTGGGCATCC | 3800 |
| TGTACACCCC AGCTTTAAAA GTGATGGAAG GCCACGGGGA AGTGAATATT | 3850 |
| AAGCATTATC TAAACTGCAG TCACTGTGAG GTAGACGAAT GTCACATGGA | 3900 |
| CCCTGAAAGC CACAAGGTCA TCTGCTTCTG TGACCACGGG ACGGTGCTGG | 3950 |
| CTGAGGATGG CGTCTCCTGC ATTGTGTCAC CCACCCCGGA GCCACACCTG | 4000 |
| CCACTCTCGC TGATCCTCTC TGTGGTGACC TCTGCCCTCG TGGCCGCCCT | 4050 |
| GGTCCTGGCT TTCTCCGGCA TCATGATTGT GTACCGCCGG AAGCACCAGG | 4100 |
| AGCTGCAAGC CATGCAGATG GAGCTGCAGA GCCCTGAGTA CAAGCTGAGC | 4150 |
| AAGCTCCGCA CCTCGACCAT CATGACCGAC TACAACCCCA ACTACTGCTT | 4200 |
| TGCTGGCAAG ACCTCCTCCA TCAGTGACCT GAAGGAGGTG CCGCGGAAAA | 4250 |
| ACATCACCCT CATTCGGGGT CTGGGCCATG GCGCCTTTGG GGAGGTGTAT | 4300 |
| GAAGGCCAGG TGTCCGGAAT GCCCAACGAC CCAAGCCCCC TGCAAGTGGC | 4350 |
| TGTGAAGACG CTGCCTGAAG TGTGCTCTGA ACAGGACGAA CTGGATTTCC | 4400 |
| TCATGGAAGC CCTGATCATC AGCAAATTCA ACCACCAGAA CATTGTTCGC | 4450 |
| TGCATTGGGG TGAGCCTGCA ATCCCTGCCC CGGTTCATCC TGCTGGAGCT | 4500 |
| CATGGCGGGG GGAGACCTCA AGTCCTTCCT CCGAGAGACC CGCCCTCGCC | 4550 |
| CGAGCCAGCC CTCCTCCCTG GCCATGCTGG ACCTTCTGCA CGTGGCTCGG | 4600 |
| GACATTGCCT GTGGCTGTCA GTATTTGGAG GAAAACCACT TCATCCACCG | 4650 |
| AGACATTGCT GCCAGAAACT GCCTCTTGAC CTGTCCAGGC CCTGGAAGAG | 4700 |
| TGGCCAAGAT TGGAGACTTC GGGATGGCCC GAGACATCTA CAGGGCGAGC | 4750 |
| TACTATAGAA AGGGAGGCTG TGCCATGCTG CCAGTTAAGT GGATGCCCCC | 4800 |
| AGAGGCCTTC ATGGAAGGAA TATTCACTTC TAAAACAGAC ACATGGTCCT | 4850 |
| TTGGAGTGCT GCTATGGGAA ATCTTTTCTC TTGGATATAT GCCATACCCC | 4900 |
| AGCAAAAGCA ACCAGGAAGT TCTGGAGTTT GTCACCAGTG GAGGCCGGAT | 4950 |
| GGACCCACCC AAGAACTGCC CTGGGCCTGT ATACCGGATA ATGACTCAGT | 5000 |
| GCTGGCAACA TCAGCCTGAA GACAGGCCCA ACTTTGCCAT CATTTTGGAG | 5050 |
| AGGATTGAAT ACTGCACCCA GGACCCGGAT GTAATCAACA CCGCTTTGCC | 5100 |
| GATAGAATAT GGTCCACTTG TGGAAGAGGA AGAGAAAGTG CCTGTGAGGC | 5150 |
| CCAAGGACCC TGAGGGGGTT CCTCCTCTCC TGGTCTCTCA ACAGGCAAAA | 5200 |
| CGGGAGGAGG AGCGCAGCCC AGCTGCCCCA CCACCTCTGC CTACCACCTC | 5250 |
| CTCTGGCAAG GCTGCAAAGA AACCCACAGC TGCAGAGATC TCTGTTCGAG | 5300 |
| TCCCTAGAGG GCCGGCCGTG GAAGGGGGAC ACGTGAATAT GGCATTCTCT | 5350 |
| CAGTCCAACC CTCCTTCGGA GTTGCACAAG GTCCACGGAT CCAGAAACAA | 5400 |
| GCCCACCAGC TTGTGGAACC CAACGTACGG CTCCTGGTTT ACAGAGAAAC | 5450 |
| CCACCAAAAA GAATAATCCT ATAGCAAAGA AGGAGCCACA CGACAGGGGT | 5500 |
| AACCTGGGGC TGGAGGGAAG CTGTACTGTC CCACCTAACG TTGCAACTGG | 5550 |
| GAGACTTCCG GGGGCCTCAC TGCTCCTAGA GCCCTCTTCG CTGACTGCCA | 5600 |
| ATATGAAGGA GGTACCTCTG TTCAGGCTAC GTCACTTCCC TTGTGGGAAT | 5650 |
| GTCAATTACG GCTACCAGCA ACAGGGCTTG CCCTTAGAAG CCGCTACTGC | 5700 |
| CCCTGGAGCT GGTCATTACG AGGATACCAT TCTGAAAAGC AAGAATAGCA | 5750 |
| TGAACCAGCC TGGGCCCTGA GCTCGGTCGC ACACTCACTT CTCTTCCTTG | 5800 |
| GGATCCCTAA GACCGTGGAG GAGAGAGAGG CAATGGCTCC TTCACAAACC | 5850 |
| AGAGACCAAA TGTCACGTTT TGTTTTGTGC CAACCTATTT TGAAGTACCA | 5900 |
| CCAAAAAAGC TGTATTTTGA AAATGCTTTA GAAAGGTTTT GAGCATGGGT | 5950 |
| TCATCCTATT CTTTCGAAAG AAGAAAATAT CATAAAAATG AGTGATAAAT | 6000 |
| ACAAGGCCCA GATGTGGTTG CATAAGGTTT TTATGCATGT TTGTTGTATA | 6050 |
| CTTCCTTATG CTTCTTTCAA ATTGTGTGTG CTCTGCTTCA ATGTAGTCAG | 6100 |
| AATTAGCTGC TTCTATGTTT CATAGTTGGG GTCATAGATG TTTCCTTGCC | 6150 |
| TTGTTGATGT GGACATGAGC CATTTGAGGG GAGAGGGAAC GGAAATAAAG | 6200 |
| GAGTTATTTG TAATGACTAA aa (SEQ ID NO: 1) | |

ALK wild type protein sequence
Ref seq NP_004295

| | |
|---|---|
| 1 | mgaigllwll plllstaavg sgmgtgqrag spaagpplqp replsysrlq rkslavdfvv |
| 61 | pslfrvyard lllppsssel kagrpeargs laldcapllr llgpapgvsw tagspapaea |
| 121 | rtlsrvlkgg svrklrrakq lvlelgeeai legcvgppge aavgllqfnl selfswwirq |
| 181 | gegrlrirlm pekkasevgr egrlsaaira sqprllfqif gtghsslesp tnmpspspdy |
| 241 | ftwnltwimk dsfpflshrs ryglecsfdf pceleysppl hdlrnqswsw rripseeasq |
| 301 | mdlldgpgae rskemprgsf lllntsadsk htilspwmrs ssehctlays vhrhlqpsgr |
| 361 | yiaqllphne aareillmpt pgkhgwtvlq grigrpdnpf rvaleyissg nrslsavdff |
| 421 | alkncsegts pgskmalqss ftcwngtvlq lgqacdfhqd caggedesqm crklpvgfyc |
| 481 | nfedgfcgwt qgtlsphtpq wqvrtlkdar fqdhqdhall lsttdvpase satvtsatfp |
| 541 | apiksspcel rmswlirgvl rgnvslvlve nktgkeqgrm vwhvaayegl slwqwmvlpl |
| 601 | ldvsdrfwlq mvawwgqgsr aivafdnisi sldcyltisg edkilqntap ksrnlfernp |
| 661 | nkelkpgens prqtpifdpt vhwlfttcga sgphgptqaq cnnayqnsnl svevgsegpl |
| 721 | kgiqiwkvpa tdtysisgyg aaggkggknt mmrshgvsvl gifnlekddm lyilvgqqge |
| 781 | dacpstngli qkvcigennv ieeeirvnrs vhewaggggg gggatyvfkm kdgvpvplii |
| 841 | aaggggrayg aktdtfhper lennssvlgl ngnsgaaggg ggwndntsll wagkslqega |
| 901 | tgghscpqam kkwgwetrgg fggggggcss ggggggyigg naasnndpem dgedgvsfis |
| 961 | plgilytpal kvmeghgevn ikhylncshc evdechmdpe shkvicfcdh gtvlaedgvs |
| 1021 | civsptpeph lplslilsvv tsalvaalvl afsgimivyr rkhgelqamq melqspeykl |
| 1081 | sklrtstimt dynpnycfag ktssisdlke vprknitlir glghgafgev yegqvsgmpn |
| 1141 | dpsplqvavk tlpevcseqd eldflmeali iskfnhqniv rcigvslqsl prfillelma |
| 1201 | ggdlksflre trprpsqpss lamldllhva rdiacgcqyl eenhfihrdi aarnclltcp |
| 1261 | gpgrvakigd fgmardiyra syyrkggcam lpvkwmppea fmegiftskt dtwsfgvllw |
| 1321 | eifslgympy psksnqevle fvtsggrmdp pkncpgpvyr imtqcwqhqp edrpnfaiil |
| 1381 | erieyctqdp dvintalpie ygplveeeek vpvrpkdpeg vppllvsqqa kreeerspaa |
| 1441 | ppplpttssg kaakkptaae isvrvprgpa vegghvnmaf sqsnppselh kvhgsrnkpt |
| 1501 | slwnptygsw ftekptkknn piakkephdr gnlglegsct vppnvatgrl pgasllleps |
| 1561 | sltanmkevp lfrlrhfpcg nvnygyqqqg lpleaatapg aghyedtilk sknsmnqpgp |
| | (SEQ ID NO: 2) |

mRNA for fusion protein EML4-ALK variant 1

| | |
|---|---|
| ggcggcgcgg cgcggcgctc gcggctgctg cctgggaggg aggccgggca | 50 |
| ggcggctgag cggcgcggct ctcaacgtga cggggaagtg gttcgggcgg | 100 |
| ccgcggctta ctaccccagg gcgaacggac ggacgacgga ggcgggagcc | 150 |
| ggtagccgag ccgggcgacc tagagaacga gcgggtcagg ctcagcgtcg | 200 |
| gccactctgt cggtccgctg aatgaagtgc ccgcccctct gagcccggag | 250 |
| cccggcgctt tccccgcaag atggacggtt tcgccggcag tctcgatgat | 300 |
| agtatttctg ctgcaagtac ttctgatgtt caagatcgcc tgtcagctct | 350 |
| tgagtcacga gttcagcaac aagaagatga aatcactgtg ctaaaggcgg | 400 |
| ctttggctga tgttttgagg cgtcttgcaa tctctgaaga tcatgtggcc | 450 |
| tcagtgaaaa aatcagtctc aagtaaaggc caaccaagcc ctcgagcagt | 500 |
| tattcccatg tcctgtataa ccaatggaag tggtgcaaac agaaaaccaa | 550 |
| gtcataccag tgctgtctca attgcaggaa aagaaactct ttcatctgct | 600 |
| gctaaaagtg gtacagaaaa aaagaaagaa aaaccacaag gacagagaga | 650 |
| aaaaaaagag gaatctcatt ctaatgatca aagtccacaa attcgagcat | 700 |
| caccttctcc ccagccctct tcacaacctc tccaaataca cagacaaact | 750 |
| ccagaaagca agaatgctac tcccaccaaa agcataaaac gaccatcacc | 800 |
| agctgaaaag tcacataatt cttgggaaaa ttcagatgat agccgtaata | 850 |
| aattgtcgaa aataccttca acacccaaat taataccaaa agttaccaaa | 900 |
| actgcagaca agcataaaga tgtcatcatc aaccaagaag gagaatatat | 950 |
| taaaatgttt atgcgcggtc ggccaattac catgttcatt ccttccgatg | 1000 |
| ttgacaacta tgatgacatc agaacggaac tgcctcctga gaagctcaaa | 1050 |
| ctggagtggg catatggtta tcgaggaaag gactgtagag ctaatgttta | 1100 |
| ccttcttccg accggggaaa tagtttattt cattgcatca gtagtagtac | 1150 |
| tatttaatta tgaggagaga actcagcgac actacctggg ccatacagac | 1200 |
| tgtgtgaaat gccttgctat acatcctgac aaaattagga ttgcaactgg | 1250 |
| acagatagct ggcgtggata aagatggaag gcctctacaa ccccacgtca | 1300 |
| gagtgtggga ttctgttact ctatccacac tgcagattat tggacttggc | 1350 |
| acttttgagc gtggagtagg atgcctggat ttttcaaaag cagattcagg | 1400 |
| tgttcattta tgtgttattg atgactccaa tgagcatatg cttactgtat | 1450 |
| gggactggca gaagaaagca aaaggagcag aaataaagac aacaaatgaa | 1500 |
| gttgttttgg ctgtggagtt tcacccaaca gatgcaaata ccataattac | 1550 |
| atgcggtaaa tctcatattt tcttctggac ctggagcggc aattcactaa | 1600 |
| caagaaaaca gggaattttt gggaaatatg aaaagccaaa atttgtgcag | 1650 |
| tgtttagcat tcttggggaa tggagatgtt cttactggag actcaggtgg | 1700 |
| agtcatgctt atatggagca aaactactgt agagcccaca cctgggaaag | 1750 |
| gacctaaAGT GTACCGCCGG AAGCACCAGG AGCTGCAAGC CATGCAGATG | 1800 |
| GAGCTGCAGA GCCCTGAGTA CAAGCTGAGC AAGCTCCGCA CCTCGACCAT | 1850 |
| CATGACCGAC TACAACCCCA ACTACTGCTT TGCTGGCAAG ACCTCCTCCA | 1900 |
| TCAGTGACCT GAAGGAGGTG CCGCGGAAAA ACATCACCCT CATTCGGGGT | 1950 |
| CTGGGCCATG GaGCCTTTGG GGAGGTGTAT GAAGGCCAGG TGTCCGGAAT | 2000 |
| GCCCAACGAC CCAAGCCCCC TGCAAGTGGC TGTGAAGACG CTGCCTGAAG | 2050 |
| TGTGCTCTGA ACAGGACGAA CTGGATTTCC TCATGGAAGC CCTGATCATC | 2100 |
| AGCAAATTCA ACCACCAGAA CATTGTTCGC TGCATTGGGG TGAGCCTGCA | 2150 |
| ATCCCTGCCC CGGTTCATCC TGCTGGAGCT CATGGCGGGG GGAGACCTCA | 2200 |
| AGTCCTTCCT CCGAGAGACC CGCCCTCGCC CGAGCCAGCC CTCCTCCCTG | 2250 |
| GCCATGCTGG ACCTTCTGCA CGTGGCTCGG GACATTGCCT GTGGCTGTCA | 2300 |
| GTATTTGGAG GAAAACCACT TCATCCACCG AGACATTGCT GCCAGAAACT | 2350 |
| GCCTCTTGAC CTGTCCAGGC CCTGGAAGAG TGGCCAAGAT TGGAGACTTC | 2400 |
| GGGATGGCCC GAGACATCTA CAGGGCGAGC TACTATAGAA AGGGAGGCTG | 2450 |
| TGCCATGCTG CCAGTTAAGT GGATGCCCCC AGAGGCCTTC ATGGAAGGAA | 2500 |
| TATTCACTTC TAAAACAGAC ACATGGTCCT TTGGAGTGCT GCTATGGGAA | 2550 |
| ATCTTTTCTC TTGGATATAT GCCATACCCC AGCAAAAGCA ACCAGGAAGT | 2600 |
| TCTGGAGTTT GTCACCAGTG GAGGCCGGAT GGACCCACCC AAGAACTGCC | 2650 |
| CTGGGCCTGT ATACCGGATA ATGACTCAGT GCTGGCAACA TCAGCCTGAA | 2700 |
| GACAGGCCCA ACTTTGCCAT CATTTTGGAG AGGATTGAAT ACTGCACCCA | 2750 |
| GGACCCGGAT GTAATCAACA CCGCTTTGCC GATAGAATAT GGTCCACTTG | 2800 |
| TGGAAGAGGA AGAGAAAGTG CCTGTGAGGC CCAAGGACCC TGAGGGGGTT | 2850 |
| CCTCCTCTCC TGGTCTCTCA ACAGGCAAAA CGGGAGGAGG AGCGCAGCCC | 2900 |
| AGCTGCCCCA CCACCTCTGC CTACCACCTC CTCTGGCAAG GCTGCAAAGA | 2950 |
| AACCCACAGC TGCAGAGgTC TCTGTTCGAG TCCCTAGAGG GCCGGCCGTG | 3000 |
| GAAGGGGGAC ACGTGAATAT GGCATTCTCT CAGTCCAACC CTCCTTCGGA | 3050 |
| GTTGCACAgG GTCCACGGAT CCAGAAACAA GCCCACCAGC TTGTGGAACC | 3100 |
| CAACGTACGG CTCCTGGTTT ACAGAGAAAC CCACCAAAAA GAATAATCCT | 3150 |
| ATAGCAAAGA AGGAGCCACA CGAgAGGGGT AACCTGGGGC TGGAGGGAAG | 3200 |
| CTGTACTGTC CCACCTAACG TTGCAACTGG GAGACTTCCG GGGGCCTCAC | 3250 |
| TGCTCCTAGA GCCCTCTTCG CTGACTGCCA ATATGAAGGA GGTACCTCTG | 3300 |
| TTCAGGCTAC GTCACTTCCC TTGTGGGAAT GTCAATTACG GCTACCAGCA | 3350 |
| ACAGGGCTTG CCCTTAGAAG CCGCTACTGC CCCTGGAGCT GGTCATTACG | 3400 |
| AGGATACCAT TCTGAAAAGC AAGAATAGCA TGAACCAGCC TGGGCCCTGA | 3450 |
| GCTCGGTCaC ACACTCACTT CTCTTCCTTG GGATCCCTAA GACCGTGGAG | 3500 |
| GAGAGAGAGG CAATcaatGG CTCCTTCACA AACCAGAGAC CAAATGTCAC | 3550 |
| GTTTTGTTTT GTGCCAACCT ATTTTGAAGT ACCACCAAAA AAGCTGTATT | 3600 |
| TTGAAAATGC TTTAGAAAGG TTTTGAGCAT GGGTTCATCC TATTCTTTCG | 3650 |
| AAAGAAGAAA ATATCATAAA AATGAGTGAT AAATACAAGG CCCAGATGTG | 3700 |
| GTTGCATAAG GTTTTTATGC ATGTTTGTTG TATACTTCCT TATGCTTCTT | 3750 |
| TtAAATTGTG TGTGCTCTGC TTCAATGTAG TCAGAATTAG CTGCTTCTAT | 3800 |
| GTTTCATAGT TGGGGTCATA GATGTTTCCT TGCCTTGTTG ATGTGGACAT | 3850 |
| GAGCCATTTG AGGGGAGAGG GAACGGAAAT AAAGGAGTTA TTTGTAATGA | 3900 |
| aaaaaaaaaa aaaaaaaaaa aaaaaa (SEQ ID NO: 3) | |

mRNA for fusion protein EML4-ALK variant 2

| | |
|---|---|
| gGCGGCGCGG CGCGGCGCTC GCGGCTGCTG CCTGGGAGGG AGGCCGGGCA | 50 |
| GGCGGCTGAG CGGCGCGGCT CTCAACGTGA CGGGGAAGTG GTTCGGGCGG | 100 |
| CCGCGGCTTA CTACCCCAGG GCGAACGGAC GGACGACGGA GGCGGGAGCC | 150 |
| GGTAGCCGAG CCGGGCGACC TAGAGAACGA GCGGGTCAGG CTCAGCGTCG | 200 |
| GCCACTCTGT CGGTCCGCTG AATGAAGTGC CCGCCCCTCT gAGCCCGGAG | 250 |
| CCCGGCGCTT TCCCCGCAAG ATGGACGGTT TCGCCGGCAG TCTCGATGAT | 300 |
| AGTATTTCTG CTGCAAGTAC TTCTGATGTT CAAGATCGCC TGTCAGCTCT | 350 |
| TGAGTCACGA GTTCAGCAAC AAGAAGATGA AATCACTGTG CTAAAGGCGG | 400 |
| CTTTGGCTGA TGTTTTGAGG CGTCTTGCAA TCTCTGAAGA TCATGTGGCC | 450 |
| TCAGTGAAAA AATCAGTCTC AAGTAAAGGC CAACCAAGCC CTCGAGCAGT | 500 |
| TATTCCCATG TCCTGTATAA CCAATGGAAG TGGTGCAAAC AGAAAACCAA | 550 |
| GTCATACCAG TGCTGTCTCA ATTGCAGGAA AAGAAACTCT TTCATCTGCT | 600 |
| GCTAAAAGTG GTACAGAAAA AAAGAAAGAA AAACCACAAG GACAGAGAGA | 650 |
| AAAAAAAGAG GAATCTCATT CTAATGATCA AAGTCCACAA ATTCGAGCAT | 700 |
| CACCTTCTCC CCAGCCCTCT TCACAACCTC TCCAAATACA CAGACAAACT | 750 |
| CCAGAAAGCA AGAATGCTAC TCCCACCAAA AGCATAAAAC GACCATCACC | 800 |
| AGCTGAAAAG TCACATAATT CTTGGGAAAA TTCAGATGAT AGCCGTAATA | 850 |
| AATTGTCGAA AATACCTTCA ACACCCAAAT TAATACCAAA AGTTACCAAA | 900 |
| ACTGCAGACA AGCATAAAGA TGTCATCATC AACCAAGAAG GAGAATATAT | 950 |
| TAAAATGTTT ATGCGCGGTC GGCCAATTAC CATGTTCATT CCTTCCGATG | 1000 |
| TTGACAACTA TGATGACATC AGAACGGAAC TGCCTCCTGA GAAGCTCAAA | 1050 |
| CTGGAGTGGG CATATGGTTA TCGAGGAAAG GACTGTAGAG CTAATGTTTA | 1100 |
| CCTTCTTCCG ACCGGGgAAA TAGTTTATTT CATTGCATCA GTAGTAGTAC | 1150 |
| TATTTAATTA TGAGGAGAGA ACTCAGCGAC ACTACCTGGG CCATACAGAC | 1200 |
| TGTGTGAAAT GCCTTGCTAT ACATCCTGAC AAAATTAGGA TTGCAACTGG | 1250 |
| ACAGATAGCT GGCGTGGATA AAGATGGAAG GCCTCTACAA CCCCACGTCA | 1300 |
| GAGTGTGGGA TTCTGTTACT CTATCCACAC TGCAGATTAT TGGACTTGGC | 1350 |
| ACTTTTGAGC GTGGAGTAGG ATGCCTGGAT TTTTCAAAAG CAGATTCAGG | 1400 |
| TGTTCATTTA TGTgTTATTG ATGACTCCAA TGAGCATATG CTTACTGTAT | 1450 |
| GGGACTGGCA GAAGAAAGCA AAAGGAGCAG AAATAAAGAC AACAAATGAA | 1500 |
| GTTGTTTTGG CTGTGGAGTT TCACCCAACA GATGCAAATA CCATAATTAC | 1550 |
| ATGCGGTAAA TCTCATATTT TCTTCTGGAC CTGGAGCGGC AATTCACTAA | 1600 |
| CAAGAAAACA GGGAATTTTT GGGAAATATG AAAAGCCAAA ATTTGTGCAG | 1650 |
| TGTTTAGCAT TCTTGGGGAA TGGAGATGTT CTTACTGGAG ACTCAGGTGG | 1700 |
| AGTCATGCTT ATATGGAGCA AAACTACTGT AGAGCCCACA CCTGGGAAAG | 1750 |
| GACCTAAAGG TGTATATCAA ATCAGCAAAC AAATCAAAGC TCATGATGGC | 1800 |
| AGTGTGTTCA CACTTTGTCA GATGAGAAAT GGGATGTTAT TAACTGGAGG | 1850 |
| AGGGAAAGAC AGAAAAATAA TTCTGTGGGA TCATGATCTG AATCCTGAAA | 1900 |
| GAGAAATAGA GGTTCCTGAT CAGTATGGCA CAATCAGAGC TGTAGCAGAA | 1950 |
| GGAAAGGCAG ATCAATTTTT AGTAGGCACA TCACGAAACT TTATTTTACG | 2000 |
| AGGAACATTT AATGATGGCT TCCAAATAGA AGTACAGGGT CATACAGATG | 2050 |
| AGCTTTGGGG TCTTGCCACA CATCCCTTCA AAGATTTGCT CTTGACATGT | 2100 |
| GCTCAGGACA GGCAGGTGTG CCTGTGGAAC TCAATGGAAC ACAGGCTGGA | 2150 |
| ATGGACCAGG CTGGTAGATG AACCAGGACA CTGTGCAGAT TTTCATCCAA | 2200 |
| GTGGCACAGT GGTGGCCATA GGAACGCACT CAGGCAGGTG GTTTGTTCTG | 2250 |
| GATGCAGAAA CCAGAGATCT AGTTTCTATC CACACAGACG GGAATGAACA | 2300 |
| GCTCTCTGTG ATGCGCTACT CAATAGATGG TACCTTCCTG GCTGTAGGAT | 2350 |
| CTCATGACAA CTTTATTTAC CTCTATGTAG TCTCTGAAAA TGGAAGAAAA | 2400 |
| TATAGCAGAT ATGGAAGGTG CACTGGACAT TCCAGCTACA TCACACACCT | 2450 |
| TGACTGGTCC CCAGACAACA AGTATATAAT GTCTAACTCG GGAGACTATG | 2500 |
| AAATATTGTA CTtgtaccgc cggaagcacc aggagctgca agccatgcag | 2550 |
| atggagctgc agagccctga gtacaagctg agcaagctcc gcacctcgac | 2600 |
| catcatgacc gactacaacc ccaactactg ctttgctggc aagacctcct | 2650 |
| ccatcagtga cctgaaggag gtgccgcgga aaaacatcac cctcattcgg | 2700 |
| ggtctgggcc atggagcctt tggggaggtg tatgaaggcc aggtgtccgg | 2750 |
| aatgcccaac gacccaagcc ccctgcaagt ggctgtgaag acgctgcctg | 2800 |
| aagtgtgctc tgaacaggac gaactggatt tcctcatgga agccctgatc | 2850 |
| atcagcaaat tcaaccacca gaacattgtt cgctgcattg gggtgagcct | 2900 |
| gcaatccctg ccccggttca tcctgctgga gctcatggcg gggggagacc | 2950 |
| tcaagtcctt cctccgagag acccgccctc gcccgagcca gccctcctcc | 3000 |
| ctggccatgc tggaccttct gcacgtggct cgggacattg cctgtggctg | 3050 |
| tcagtatttg gaggaaaacc acttcatcca ccgagacatt gctgccagaa | 3100 |
| actgcctctt gacctgtcca ggccctggaa gagtggccaa gattggagac | 3150 |
| ttcgggatgg cccgagacat ctacagggcg agctactata gaaagggagg | 3200 |
| ctgtgccatg ctgccagtta agtggatgcc cccagaggcc ttcatggaag | 3250 |
| gaatattcac ttctaaaaca gacacatggt cctttggagt gctgctatgg | 3300 |
| gaaatctttt ctcttggata tatgccatac cccagcaaaa gcaaccagga | 3350 |
| agttctggag tttgtcacca gtggaggccg gatggaccca cccaagaact | 3400 |
| gccctgggcc tgtataccgg ataatgactc agtgctggca acatcagcct | 3450 |
| gaagacaggc ccaactttgc catcattttg gagaggattg aatactgcac | 3500 |
| ccaggacccg gatgtaatca acaccgcttt gccgatagaa tatggtccac | 3550 |
| ttgtggaaga ggaagagaaa gtgcctgtga ggcccaagga ccctgagggg | 3600 |
| gttcctcctc tcctggtctc tcaacaggca aaacgggagg aggagcgcag | 3650 |
| cccagctgcc ccaccacctc tgcctaccac ctcctctggc aaggctgcaa | 3700 |
| agaaacccac agctgcagag gtctctgttc gagtccctag agggccggcc | 3750 |
| gtggaagggg gacacgtgaa tatggcattc tctcagtcca accctccttc | 3800 |
| ggagttgcac agggtccacg gatccagaaa caagcccacc agcttgtgga | 3850 |
| acccaacgta cggctcctgg tttacagaga aacccaccaa aaagaataat | 3900 |
| cctatagcaa agaaggagcc acacgagagg ggtaacctgg ggctggaggg | 3950 |
| aagctgtact gtcccaccta acgttgcaac tgggagactt ccgggggcct | 4000 |
| cactgctcct agagccctct tcgctgactg ccaatatgaa ggaggtacct | 4050 |
| ctgttcaggc tacgtcactt cccttgtggg aatgtcaatt acggctacca | 4100 |
| gcaacagggc ttgcccttag aagccgctac tgcccctgga gctggtcatt | 4150 |
| acgaggatac cattctgaaa agcaagaata gcatgaacca gcctgggccc | 4200 |
| tgagctcggt cacacactca cttctcttcc ttgggatccc taagaccgtg | 4250 |
| gaggagagag aggcaatcaa tggctccttc acaaaccaga gaccaaatgt | 4300 |
| cacgttttgt tttgtgccaa cctattttga agtaccacca aaaaagctgt | 4350 |
| attttgaaaa tgctttagaa aggttttgag catgggttca tcctattctt | 4400 |
| tcgaaagaag aaaatatcat aaaaatgagt gataaataca aggcccagat | 4450 |
| gtggttgcat aaggttttta tgcatgtttg ttgtatactt ccttatgctt | 4500 |
| cttttaaatt gtgtgtgctc tgcttcaatg tagtcagaat tagctgcttc | 4550 |
| tatgtttcat agttggggtc atagatgttt ccttgccttg ttgatgtgga | 4600 |
| catgagccat ttgaggggag agggaacgga aataaaggag ttatttgtaa | 4650 |
| tgaaaaaaaa aaaaaaaaaa aaaaaaaaa (SEQ ID NO: 4) | |

mRNA for fusion protein EML4-ALK variant 3 splicing isoform a

| | |
|---|---|
| actctgtcgg tccgctgaat gaagtgcccg cccctctaag cccggagccc | 50 |
| ggcgctttcc ccgcaagatg gacggtttcg ccggcagtct cgatgatagt | 100 |
| atttctgctg caagtacttc tgatgttcaa gatcgcctgt cagctcttga | 150 |
| gtcacgagtt cagcaacaag aagatgaaat cactgtgcta aaggcggctt | 200 |
| tggctgatgt tttgaggcgt cttgcaatct ctgaagatca tgtggcctca | 250 |
| gtgaaaaaat cagtctcaag taaaggccaa ccaagccctc gagcagttat | 300 |
| tcccatgtcc tgtataacca atggaagtgg tgcaaacaga aaaccaagtc | 350 |
| ataccagtgc tgtctcaatt gcaggaaaag aaactctttc atctgctgct | 400 |
| aaaagtggta cagaaaaaaa gaaagaaaaa ccacaaggac agagagaaaa | 450 |
| aaaagaggaa tctcattcta atgatcaaag tccacaaatt cgagcatcac | 500 |
| cttctcccca gccctcttca caacctctcc aaatacacag acaaactcca | 550 |
| gaaagcaaga atgctactcc caccaaaagc ataaaacgac catcaccagc | 600 |
| tgaaaagtca cataattctt gggaaaattc agatgatagc cgtaataaat | 650 |
| tgtcgaaaat accttcaaca cccaaattaa taccaaaagt taccaaaact | 700 |
| gcagacaagc ataaagatgt catcatcaac caAGTGTACC GCCGGAAGCA | 750 |
| CCAGGAGCTG CAAGCCATGC AGATGGAGCT GCAGAGCCCT GAGTACAAGC | 800 |
| TGAGCAAGCT CCGCACCTCG ACCATCATGA CCGACTACAA CCCCAACTAC | 850 |
| TGCTTTGCTG GCAAGACCTC CTCCATCAGT GACCTGAAGG AGGTGCCGCG | 900 |
| GAAAAACATC ACCCTCATTC GGGGTCTGGG CCATGGaGCC TTTGGGGAGG | 950 |
| TGTATGAAGG CCAGGTGTCC GGAATGCCCA ACGACCCAAG CCCCCTGCAA | 1000 |
| GTGGCTGTGA AGACGCTGCC TGAAGTGTGC TCTGAACAGG ACGAACTGGA | 1050 |
| TTTCCTCATG GAAGCCCTGA TCATCAGCAA ATTCAACCAC CAGAACATTG | 1100 |
| TTCGCTGCAT TGGGGTGAGC CTGCAATCCC TGCCCCGGTT CATCCTGCTG | 1150 |
| GAGCTCATGG CGGGGGGAGA CCTCAAGTCC TTCCTCCGAG AGACCCGCCC | 1200 |
| TCGCCCGAGC CAGCCCTCCT CCCTGGCCAT GCTGGACCTT CTGCACGTGG | 1250 |
| CTCGGGACAT TGCCTGTGGC TGTCAGTATT TGGAGGAAAA CCACTTCATC | 1300 |
| CACCGAGACA TTGCTGCCAG AAACTGCCTC TTGACCTGTC CAGGCCCTGG | 1350 |
| AAGAGTGGCC AAGATTGGAG ACTTCGGGAT GGCCCGAGAC ATCTACAGGG | 1400 |
| CGAGCTACTA TAGAAAGGGA GGCTGTGCCA TGCTGCCAGT TAAGTGGATG | 1450 |
| CCCCCAGAGG CCTTCATGGA AGGAATATTC ACTTCTAAAA CAGACACATG | 1500 |
| GTCCTTTGGA GTGCTGCTAT GGGAAATCTT TTCTCTTGGA TATATGCCAT | 1550 |
| ACCCCAGCAA AAGCAACCAG GAAGTTCTGG AGTTTGTCAC CAGTGGAGGC | 1600 |
| CGGATGGACC CACCCAAGAA CTGCCCTGGG CCTGTATACC GGATAATGAC | 1650 |
| TCAGTGCTGG CAACATCAGC CTGAAGACAG GCCCAACTTT GCCATCATTT | 1700 |
| TGGAGAGGAT TGAATACTGC ACCCAGGACC CGGATGTAAT CAACACCGCT | 1750 |
| TTGCCGATAG AATATGGTCC ACTTGTGGAA GAGGAAGAGA AAGTGCCTGT | 1800 |
| GAGGCCCAAG GACCCTGAGG GGGTTCCTCC TCTCCTGGTC TCTCAACAGG | 1850 |
| CAAAACGGGA GGAGGAGCGC AGCCCAGCTG CCCCACCACC TCTGCCTACC | 1900 |
| ACCTCCTCTG GCAAGGCTGC AAAGAAACCC ACAGCTGCAG AGgTCTCTGT | 1950 |
| TCGAGTCCCT AGAGGGCCGG CCGTGGAAGG GGGACACGTG AATATGGCAT | 2000 |
| TCTCTCAGTC CAACCCTCCT TCGGAGTTGC ACAgGGTCCA CGGATCCAGA | 2050 |
| AACAAGCCCA CCAGCTTGTG GAACCCAACG TACGGCTCCT GGTTTACAGA | 2100 |
| GAAACCCACC AAAAAGAATA ATCCTATAGC AAAGAAGGAG CCACACGAgA | 2150 |
| GGGGTAACCT GGGGCTGGAG GGAAGCTGTA CTGTCCCACC TAACGTTGCA | 2200 |
| ACTGGGAGAC TTCCGGGGGC CTCACTGCTC CTAGAGCCCT CTTCGCTGAC | 2250 |
| TGCCAATATG AAGGAGGTAC CTCTGTTCAG GCTACGTCAC TTCCCTTGTG | 2300 |
| GGAATGTCAA TTACGGCTAC CAGCAACAGG GCTTGCCCTT AGAAGCCGCT | 2350 |
| ACTGCCCCTG GAGCTGGTCA TTACGAGGAT ACCATTCTGA AAAGCAAGAA | 2400 |
| TAGCATGAAC CAGCCTGGGC CCTGAGCTCG GTCGCACACT CACTTCTCTT | 2450 |
| CCTTGGGATC CCTAAGACCG TGG (SEQ ID NO: 5) | |

mRNA for fusion protein EML4-ALK variant 3 splicing isoform b

| | |
|---|---|
| actctgtcgg tccgctgaat gaagtgcccg cccctctaag cccggagccc | 50 |
| ggcgctttcc ccgcaagatg gacggtttcg ccggcagtct cgatgatagt | 100 |
| atttctgctg caagtacttc tgatgttcaa gatcgcctgt cagctcttga | 150 |
| gtcacgagtt cagcaacaag aagatgaaat cactgtgcta aaggcggctt | 200 |
| tggctgatgt tttgaggcgt cttgcaatct ctgaagatca tgtggcctca | 250 |
| gtgaaaaaat cagtctcaag taaaggccaa ccaagccctc gagcagttat | 300 |
| tcccatgtcc tgtataacca atggaagtgg tgcaaacaga aaaccaagtc | 350 |
| ataccagtgc tgtctcaatt gcaggaaaag aaactctttc atctgctgct | 400 |
| aaaagtggta cagaaaaaaa gaaagaaaaa ccacaaggac agagagaaaa | 450 |
| aaaagaggaa tctcattcta atgatcaaag tccacaaatt cgagcatcac | 500 |
| cttctcccca gccctcttca caacctctcc aaatacacag acaaactcca | 550 |
| gaaagcaaga atgctactcc caccaaaagc ataaaacgac catcaccagc | 600 |
| tgaaaagtca cataattctt gggaaaattc agatgatagc cgtaataaat | 650 |
| tgtcgaaaat accttcaaca cccaaattaa taccaaaagt taccaaaact | 700 |
| gcagacaagc ataaagatgt catcatcaac caagcaaaaa tgtcaactcg | 750 |
| cgaaaaaaac agccaAGTGT ACCGCCGGAA GCACCAGGAG CTGCAAGCCA | 800 |
| TGCAGATGGA GCTGCAGAGC CCTGAGTACA AGCTGAGCAA GCTCCGCACC | 850 |
| TCGACCATCA TGACCGACTA CAACCCCAAC TACTGCTTTG CTGGCAAGAC | 900 |
| CTCCTCCATC AGTGACCTGA AGGAGGTGCC GCGGAAAAAC ATCACCCTCA | 950 |
| TTCGGGGTCT GGGCCATGGa GCCTTTGGGG AGGTGTATGA AGGCCAGGTG | 1000 |
| TCCGGAATGC CCAACGACCC AAGCCCCCTG CAAGTGGCTG TGAAGACGCT | 1050 |
| GCCTGAAGTG TGCTCTGAAC AGGACGAACT GGATTTCCTC ATGGAAGCCC | 1100 |
| TGATCATCAG CAAATTCAAC CACCAGAACA TTGTTCGCTG CATTGGGGTG | 1150 |
| AGCCTGCAAT CCCTGCCCCG GTTCATCCTG CTGGAGCTCA TGGCGGGGGG | 1200 |
| AGACCTCAAG TCCTTCCTCC GAGAGACCCG CCCTCGCCCG AGCCAGCCCT | 1250 |
| CCTCCCTGGC CATGCTGGAC CTTCTGCACG TGGCTCGGGA CATTGCCTGT | 1300 |
| GGCTGTCAGT ATTTGGAGGA AAACCACTTC ATCCACCGAG ACATTGCTGC | 1350 |
| CAGAAACTGC CTCTTGACCT GTCCAGGCCC TGGAAGAGTG GCCAAGATTG | 1400 |
| GAGACTTCGG GATGGCCCGA GACATCTACA GGGCGAGCTA CTATAGAAAG | 1450 |
| GGAGGCTGTG CCATGCTGCC AGTTAAGTGG ATGCCCCCAG AGGCCTTCAT | 1500 |
| GGAAGGAATA TTCACTTCTA AAACAGACAC ATGGTCCTTT GGAGTGCTGC | 1550 |
| TATGGGAAAT CTTTTCTCTT GGATATATGC CATACCCCAG CAAAAGCAAC | 1600 |
| CAGGAAGTTC TGGAGTTTGT CACCAGTGGA GGCCGGATGG ACCCACCCAA | 1650 |
| GAACTGCCCT GGGCCTGTAT ACCGGATAAT GACTCAGTGC TGGCAACATC | 1700 |
| AGCCTGAAGA CAGGCCCAAC TTTGCCATCA TTTTGGAGAG GATTGAATAC | 1750 |
| TGCACCCAGG ACCCGGATGT AATCAACACC GCTTTGCCGA TAGAATATGG | 1800 |
| TCCACTTGTG GAAGAGGAAG AGAAAGTGCC TGTGAGGCCC AAGGACCCTG | 1850 |
| AGGGGGTTCC TCCTCTCCTG GTCTCTCAAC AGGCAAAACG GGAGGAGGAG | 1900 |
| CGCAGCCCAG CTGCCCCACC ACCTCTGCCT ACCACCTCCT CTGGCAAGGC | 1950 |
| TGCAAAGAAA CCCACAGCTG CAGAGgTCTC TGTTCGAGTC CCTAGAGGGC | 2000 |
| CGGCCGTGGA AGGGGGACAC GTGAATATGG CATTCTCTCA GTCCAACCCT | 2050 |
| CCTTCGGAGT TGCACAgGGT CCACGGATCC AGAAACAAGC CCACCAGCTT | 2100 |
| GTGGAACCCA ACGTACGGCT CCTGGTTTAC AGAGAAACCC ACCAAAAAGA | 2150 |
| ATAATCCTAT AGCAAAGAAG GAGCCACACG AgAGGGGTAA CCTGGGGCTG | 2200 |
| GAGGGAAGCT GTACTGTCCC ACCTAACGTT GCAACTGGGA GACTTCCGGG | 2250 |
| GGCCTCACTG CTCCTAGAGC CCTCTTCGCT GACTGCCAAT ATGAAGGAGG | 2300 |
| TACCTCTGTT CAGGCTACGT CACTTCCCTT GTGGGAATGT CAATTACGGC | 2350 |
| TACCAGCAAC AGGGCTTGCC CTTAGAAGCC GCTACTGCCC CTGGAGCTGG | 2400 |
| TCATTACGAG GATACCATTC TGAAAAGCAA GAATAGCATG AACCAGCCTG | 2450 |
| GGCCCTGAGC TCGGTCGCAC ACTCACTTCT CTTCCTTGGG ATCCCTAAGA | 2500 |
| CCGTGG (SEQ ID NO: 6) | |

mRNA for fusion protein EML4-ALK variant 4

| | |
|---|---|
| ACTCTGTCGG TCCGCTGAAT GAAGTGCCCG CCCCTCTAAG CCCGGAGCCC | 50 |
| GGCGCTTTCC CCGCAAGATG GACGGTTTCG CCGGCAGTCT CGATGATAGT | 100 |
| ATTTCTGCTG CAAGTACTTC TGATGTTCAA GATCGCCTGT CAGCTCTTGA | 150 |
| GTCACGAGTT CAGCAACAAG AAGATGAAAT CACTGTGCTA AAGGCGGCTT | 200 |
| TGGCTGATGT TTTGAGGCGT CTTGCAATCT CTGAAGATCA TGTGGCCTCA | 250 |
| GTGAAAAAAT CAGTCTCAAG TAAAGGCCAA CCAAGCCCTC GAGCAGTTAT | 300 |
| TCCCATGTCC TGTATAACCA ATGGAAGTGG TGCAAACAGA AAACCAAGTC | 350 |
| ATACCAGTGC TGTCTCAATT GCAGGAAAAG AAACTCTTTC ATCTGCTGCT | 400 |
| AAAAGTGGTA CAGAAAAAAA GAAAGAAAAA CCACAAGGAC AGAGAGAAAA | 450 |
| AAAAGAGGAA TCTCATTCTA ATGATCAAAG TCCACAAATT CGAGCATCAC | 500 |
| CTTCTCCCCA GCCCTCTTCA CAACCTCTCC AAATACACAG ACAAACTCCA | 550 |
| GAAAGCAAGA ATGCTACTCC CACCAAAAGC ATAAAACGAC CATCACCAGC | 600 |
| TGAAAAGTCA CATAATTCTT GGGAAAATTC AGATGATAGC CGTAATAAAT | 650 |
| TGTCGAAAAT ACCTTCAACA CCCAAATTAA TACCAAAAGT TACCAAAACT | 700 |
| GCAGACAAGC ATAAAGATGT CATCATCAAC CAAGAAGGAG AATATATTAA | 750 |
| AATGTTTATG CGCGGTCGGC CAATTACCAT GTTCATTCCT TCCGATGTTG | 800 |
| ACAACTATGA TGACATCAGA ACGGAACTGC CTCCTGAGAA GCTCAAACTG | 850 |
| GAGTGGGCAT ATGGTTATCG AGGAAAGGAC TGTAGAGCTA ATGTTTACCT | 900 |
| TCTTCCGACC GGGgAAATAG TTTATTTCAT TGCATCAGTA GTAGTACTAT | 950 |
| TTAATTATGA GGAGAGAACT CAGCGACACT ACCTGGGCCA TACAGACTGT | 1000 |
| GTGAAATGCC TTGCTATACA TCCTGACAAA ATTAGGATTG CAACTGGACA | 1050 |
| GATAGCTGGC GTGGATAAAG ATGGAAGGCC TCTACAACCC CACGTCAGAG | 1100 |
| TGTGGGATTC TGTTACTCTA TCCACACTGC AGATTATTGG ACTTGGCACT | 1150 |
| TTTGAGCGTG GAGTAGGATG CCTGGATTTT TCAAAAGCAG ATTCAGGTGT | 1200 |
| TCATTTATGT gTTATTGATG ACTCCAATGA GCATATGCTT ACTGTATGGG | 1250 |
| ACTGGCAGAg GAAAGCAAAA GGAGCAGAAA TAAAGACAAC AAATGAAGTT | 1300 |
| GTTTTGGCTG TGGAGTTTCA CCCAACAGAT GCAAATACCA TAATTACATG | 1350 |
| CGGTAAATCT CATATTTTCT TCTGGACCTG GAGCGGCAAT TCACTAACAA | 1400 |
| GAAAACAGGG AATTTTTGGG AAATATGAAA AGCCAAAATT TGTGCAGTGT | 1450 |
| TTAGCATTCT TGGGGAATGG AGATGTTCTT ACTGGAGACT CAGGTGGAGT | 1500 |
| CATGCTTATA TGGAGCAAAA CTACTGTAGA GCCCACACCT GGGAAAGGAC | 1550 |
| CTAAAGGTGT ATATCAAATC AGCAAACAAA TCAAAGCTCA TGATGGCAGT | 1600 |
| GTGTTCACAC TTTGTCAGAT GAGAAATGGG ATGTTATTAA CTGGAGGAGG | 1650 |
| GAAAGACAGA AAAATAATTC TGTGGGATCA TGATCTGAAT CCTGAAAGAG | 1700 |
| AAATAGAGat atgctggatg agccctgagt acaagctgag caagctccgc | 1750 |
| acctcgacca tcatgaccga ctacaacccc aactactgct ttgctggcaa | 1800 |
| gacctcctcc atcagtgacc tgaaggaggt gccgcggaaa aacatcaccc | 1850 |
| tcattcgggg tctgggccat ggagcctttg gggaggtgta tgaaggccag | 1900 |
| gtgtccggaa tgcccaacga cccaagcccc ctgcaagtgg ctgtgaagac | 1950 |
| gctgcctgaa gtgtgctctg aacaggacga actggatttc ctcatggaag | 2000 |
| ccctgatcat cagcaaattc aaccaccaga acattgttcg ctgcattggg | 2050 |
| gtgagcctgc aatccctgcc ccggttcatc ctgctggagc tcatggcggg | 2100 |
| gggagacctc aagtccttcc tccgagagac ccgccctcgc ccgagccagc | 2150 |
| cctcctccct ggccatgctg gaccttctgc acgtggctcg ggacattgcc | 2200 |
| tgtggctgtc agtatttgga ggaaaaccac ttcatccacc gagacattgc | 2250 |
| tgccagaaac tgcctcttga cctgtccagg ccctggaaga gtggccaaga | 2300 |
| ttggagactt cgggatggcc cgagacatct acagggcgag ctactataga | 2350 |
| aagggaggct gtgccatgct gccagttaag tggatgcccc cagaggcctt | 2400 |
| catggaagga atattcactt ctaaaacaga cacatggtcc tttggagtgc | 2450 |
| tgctatggga aatcttttct cttggatata tgccataccc cagcaaaagc | 2500 |
| aaccaagaag ttctggagtt tgtcaccagt ggaggccgga tggacccacc | 2550 |
| caagaactgc cctgggcctg tataccggat aatgactcag tgctggcaac | 2600 |
| atcagcctga agacaggccc aactttgcca tcattttgga gaggattgaa | 2650 |
| tactgcaccc aggacccgga tgtaatcaac accgctttgc cgatagaata | 2700 |
| tggtccactt gtggaagagg aagagaaagt gcctgtgagg cccaaggacc | 2750 |
| ctgagggggt tcctcctctc ctggtctctc aacaggcaaa acgggaggag | 2800 |
| gagcgcagcc cagctgcccc accacctctg cctaccacct cctctggcaa | 2850 |
| ggctgcaaag aaacccacag ctgcagaggt ctctgttcga gtccctagag | 2900 |
| ggccggccgt ggaaggggga cacgtgaata tggcattctc tcagtccaac | 2950 |
| cctccttcgg agttgcacag ggtccacgga tccagaaaca agcccaccag | 3000 |
| cttgtggaac ccaacgtacg gctcctggtt tacagagaaa cccaccaaaa | 3050 |
| agaataatcc tatagcaaag aaggagccac acgagagggg taacctgggg | 3100 |
| ctggagggaa gctgtactgt cccacctaac gttgcaactg ggagacttcc | 3150 |
| gggggcctca ctgctcctag agccctcttc gctgactgcc aatatgaagg | 3200 |
| aggtacctct gttcaggcta cgtcacttcc cttgtgggaa tgtcaattac | 3250 |
| ggctaccagc aacagggctt gcccttagaa gccgctactg cccctggagc | 3300 |
| tggtcattac gaggatacca ttctgaaaag caagaatagc atgaaccagc | 3350 |
| ctgggccctg agctcggtcg cacactcact tctcttcctt gggatcccta | 3400 |
| agaccgtgg (SEQ ID NO: 7) | |

mRNA for fusion protein EML4-ALK variant 5 splicing isoform a

| | |
|---|---|
| actctgtcgg tccgctgaat gaagtgcccg cccctctaag cccggagccc | 50 |
| ggcgctttcc ccgcaagatg gacggtttcg ccggcagtct cgatgatagt | 100 |
| atttctgctg caagtacttc tgatgttcaa gatcgcctgt cagctcttga | 150 |
| gtcacgagtt cagcaacaag aagatgaaat cactgtgcta aaggcggctt | 200 |
| tggctgatgt tttgaggcgt cttgcaatct ctgaagatca tgtggcctca | 250 |
| gtgaaaaaat cagtctcaag taaAGTGTAC CGCCGGAAGC ACCAGGAGCT | 300 |
| GCAAGCCATG CAGATGGAGC TGCAGAGCCC TGAGTACAAG CTGAGCAAGC | 350 |
| TCCGCACCTC GACCATCATG ACCGACTACA ACCCCAACTA CTGCTTTGCT | 400 |
| GGCAAGACCT CCTCCATCAG TGACCTGAAG GAGGTGCCGC GGAAAAACAT | 450 |
| CACCCTCATT CGGGGTCTGG GCCATGGaGC CTTTGGGGAG GTGTATGAAG | 500 |
| GCCAGGTGTC CGGAATGCCC AACGACCCAA GCCCCCTGCA AGTGGCTGTG | 550 |
| AAGACGCTGC CTGAAGTGTG CTCTGAACAG GACGAACTGG ATTTCCTCAT | 600 |
| GGAAGCCCTG ATCATCAGCA AATTCAACCA CCAGAACATT GTTCGCTGCA | 650 |
| TTGGGGTGAG CCTGCAATCC CTGCCCCGGT TCATCCTGCT GGAGCTCATG | 700 |
| GCGGGGGGAG ACCTCAAGTC CTTCCTCCGA GAGACCCGCC CTCGCCCGAG | 750 |
| CCAGCCCTCC TCCCTGGCCA TGCTGGACCT TCTGCACGTG GCTCGGGACA | 800 |
| TTGCCTGTGG CTGTCAGTAT TTGGAGGAAA ACCACTTCAT CCACCGAGAC | 850 |
| ATTGCTGCCA GAAACTGCCT CTTGACCTGT CCAGGCCCTG GAAGAGTGGC | 900 |
| CAAGATTGGA GACTTCGGGA TGGCCCGAGA CATCTACAGG GCGAGCTACT | 950 |
| ATAGAAAGGG AGGCTGTGCC ATGCTGCCAG TTAAGTGGAT GCCCCCAGAG | 1000 |
| GCCTTCATGG AAGGAATATT CACTTCTAAA ACAGACACAT GGTCCTTTGG | 1050 |
| AGTGCTGCTA TGGGAAATCT TTTCTCTTGG ATATATGCCA TACCCCAGCA | 1100 |
| AAAGCAACCA GGAAGTTCTG GAGTTTGTCA CCAGTGGAGG CCGGATGGAC | 1150 |
| CCACCCAAGA ACTGCCCTGG GCCTGTATAC CGGATAATGA CTCAGTGCTG | 1200 |
| GCAACATCAG CCTGAAGACA GGCCCAACTT TGCCATCATT TTGGAGAGGA | 1250 |
| TTGAATACTG CACCCAGGAC CCGGATGTAA TCAACACCGC TTTGCCGATA | 1300 |
| GAATATGGTC CACTTGTGGA AGAGGAAGAG AAAGTGCCTG TGAGGCCCAA | 1350 |
| GGACCCTGAG GGGGTTCCTC CTCTCCTGGT CTCTCAACAG GCAAAACGGG | 1400 |
| AGGAGGAGCG CAGCCCAGCT GCCCCACCAC CTCTGCCTAC CACCTCCTCT | 1450 |
| GGCAAGGCTG CAAAGAAACC CACAGCTGCA GAGgTCTCTG TTCGAGTCCC | 1500 |
| TAGAGGGCCG GCCGTGGAAG GGGGACACGT GAATATGGCA TTCTCTCAGT | 1550 |
| CCAACCCTCC TTCGGAGTTG CACAgGGTCC ACGGATCCAG AAACAAGCCC | 1600 |
| ACCAGCTTGT GGAACCCAAC GTACGGCTCC TGGTTTACAG AGAAACCCAC | 1650 |
| CAAAAAGAAT AATCCTATAG CAAAGAAGGA GCCACACGAg AGGGGTAACC | 1700 |
| TGGGGCTGGA GGGAAGCTGT ACTGTCCCAC CTAACGTTGC AACTGGGAGA | 1750 |
| CTTCCGGGGG CCTCACTGCT CCTAGAGCCC TCTTCGCTGA CTGCCAATAT | 1800 |
| GAAGGAGGTA CCTCTGTTCA GGCTACGTCA CTTCCCTTGT GGGAATGTCA | 1850 |
| ATTACGGCTA CCAGCAACAG GGCTTGCCCT TAGAAGCCGC TACTGCCCCT | 1900 |
| GGAGCTGGTC ATTACGAGGA TACCATTCTG AAAAGCAAGA ATAGCATGAA | 1950 |
| CCAGCCTGGG CCCTGAGCTC GGTCGCACAC TCACTTCTCT TCCTTGGGAT | 2000 |
| CCCTAAGACC GTGG (SEQ ID NO: 8) | |

mRNA for fusion protein EML4-ALK variant 5 splicing isoform b

| | |
|---|---|
| actctgtcgg tccgctgaat gaagtgcccg cccctctaag cccggagccc | 50 |
| ggcgctttcc ccgcaagatg gacggtttcg ccggcagtct cgatgatagt | 100 |
| atttctgctg caagtacttc tgatgttcaa gatcgcctgt cagctcttga | 150 |
| gtcacgagtt cagcaacaag aagatgaaat cactgtgcta aaggcggctt | 200 |
| tggctgatgt tttgaggcgt cttgcaatct ctgaagatca tgtggcctca | 250 |
| gtgaaaaaat cagtctcaag tAAAGGTTCA GAGCTCAGGG GAGGATATGG | 300 |
| AGATCCAGGG AGGCTTCCTG TAGGAAGTGG CCTGTGTAGT GCTTCAAGGG | 350 |
| CCAGGCTGCC AGGCCATGTT GCAGCTGACC ACCCACCTGC AGTGTACCGC | 400 |
| CGGAAGCACC AGGAGCTGCA AGCCATGCAG ATGGAGCTGC AGAGCCCTGA | 450 |
| GTACAAGCTG AGCAAGCTCC GCACCTCGAC CATCATGACC GACTACAACC | 500 |
| CCAACTACTG CTTTGCTGGC AAGACCTCCT CCATCAGTGA CCTGAAGGAG | 550 |
| GTGCCGCGGA AAAACATCAC CCTCATTCGG GGTCTGGGCC ATGGaGCCTT | 600 |
| TGGGGAGGTG TATGAAGGCC AGGTGTCCGG AATGCCCAAC GACCCAAGCC | 650 |
| CCCTGCAAGT GGCTGTGAAG ACGCTGCCTG AAGTGTGCTC TGAACAGGAC | 700 |
| GAACTGGATT TCCTCATGGA AGCCCTGATC ATCAGCAAAT TCAACCACCA | 750 |
| GAACATTGTT CGCTGCATTG GGGTGAGCCT GCAATCCCTG CCCCGGTTCA | 800 |
| TCCTGCTGGA GCTCATGGCG GGGGGAGACC TCAAGTCCTT CCTCCGAGAG | 850 |
| ACCCGCCCTC GCCCGAGCCA GCCCTCCTCC CTGGCCATGC TGGACCTTCT | 900 |
| GCACGTGGCT CGGGACATTG CCTGTGGCTG TCAGTATTTG GAGGAAAACC | 950 |
| ACTTCATCCA CCGAGACATT GCTGCCAGAA ACTGCCTCTT GACCTGTCCA | 1000 |
| GGCCCTGGAA GAGTGGCCAA GATTGGAGAC TTCGGGATGG CCCGAGACAT | 1050 |
| CTACAGGGCG AGCTACTATA GAAAGGGAGG CTGTGCCATG CTGCCAGTTA | 1100 |
| AGTGGATGCC CCCAGAGGCC TTCATGGAAG GAATATTCAC TTCTAAAACA | 1150 |
| GACACATGGT CCTTTGGAGT GCTGCTATGG GAAATCTTTT CTCTTGGATA | 1200 |
| TATGCCATAC CCCAGCAAAA GCAACCAGGA AGTTCTGGAG TTTGTCACCA | 1250 |
| GTGGAGGCCG GATGGACCCA CCCAAGAACT GCCCTGGGCC TGTATACCGG | 1300 |
| ATAATGACTC AGTGCTGGCA ACATCAGCCT GAAGACAGGC CCAACTTTGC | 1350 |
| CATCATTTTG GAGAGGATTG AATACTGCAC CCAGGACCCG GATGTAATCA | 1400 |
| ACACCGCTTT GCCGATAGAA TATGGTCCAC TTGTGGAAGA GGAAGAGAAA | 1450 |
| GTGCCTGTGA GGCCCAAGGA CCCTGAGGGG GTTCCTCCTC TCCTGGTCTC | 1500 |
| TCAACAGGCA AAACGGGAGG AGGAGCGCAG CCCAGCTGCC CCACCACCTC | 1550 |
| TGCCTACCAC CTCCTCTGGC AAGGCTGCAA AGAAACCCAC AGCTGCAGAG | 1600 |
| gTCTCTGTTC GAGTCCCTAG AGGGCCGGCC GTGGAAGGGG GACACGTGAA | 1650 |
| TATGGCATTC TCTCAGTCCA ACCCTCCTTC GGAGTTGCAC AgGGTCCACG | 1700 |
| GATCCAGAAA CAAGCCCACC AGCTTGTGGA ACCCAACGTA CGGCTCCTGG | 1750 |
| TTTACAGAGA AACCCACCAA AAAGAATAAT CCTATAGCAA AGAAGGAGCC | 1800 |
| ACACGAgAGG GGTAACCTGG GGCTGGAGGG AAGCTGTACT GTCCCACCTA | 1850 |
| ACGTTGCAAC TGGGAGACTT CCGGGGGCCT CACTGCTCCT AGAGCCCTCT | 1900 |
| TCGCTGACTG CCAATATGAA GGAGGTACCT CTGTTCAGGC TACGTCACTT | 1950 |
| CCCTTGTGGG AATGTCAATT ACGGCTACCA GCAACAGGGC TTGCCCTTAG | 2000 |
| AAGCCGCTAC TGCCCCTGGA GCTGGTCATT ACGAGGATAC CATTCTGAAA | 2050 |
| AGCAAGAATA GCATGAACCA GCCTGGGCCC TGAGCTCGGT CGCACACTCA | 2100 |
| CTTCTCTTCC TTGGGATCCC TAAGACCGTG G (SEQ ID NO: 9) | |

EML4-ALK variant 6 mRNA for fusion protein EML4-ALK variant 6

| | |
|---|---|
| tactctgtcg gtccgctgaa tgaagtgccc gcccctctaa gcccggagcc | 50 |
| cggcgctttc cccgcaagat ggacggtttc gccggcagtc tcgatgatag | 100 |
| tatttctgct gcaagtactt ctgatgttca agatcgcctg tcagctcttg | 150 |
| agtcacgagt tcagcaacaa gaagatgaaa tcactgtgct aaaggcggct | 200 |
| ttggctgatg ttttgaggcg tcttgcaatc tctgaagatc atgtggcctc | 250 |
| agtgaaaaaa tcagtctcaa gtaaaggcca accaagccct cgagcagtta | 300 |
| ttcccatgtc ctgtataacc aatggaagtg gtgcaaacag aaaaccaagt | 350 |
| cataccagtg ctgtctcaat tgcaggaaaa gaaactcttt catctgctgc | 400 |
| taaaagtggt acagaaaaaa agaaagaaaa accacaagga cagagagaaa | 450 |
| aaaaagagga atctcattct aatgatcaaa gtccacaaat tcgagcatca | 500 |
| ccttctcccc agccctcttc acaacctctc caaatacaca gacaaactcc | 550 |
| agaaagcaag aatgctactc ccaccaaaag cataaaacga ccatcaccag | 600 |
| ctgaaaagtc acataattct tgggaaaatt cagatgatag ccgtaataaa | 650 |
| ttgtcgaaaa taccttcaac acccaaatta ataccaaaag ttaccaaaac | 700 |
| tgcagacaag cataaagatg tcatcatcaa ccaagaagga gaatatatta | 750 |
| aaatgtttat gcgcggtcgg ccaattacca tgttcattcc ttccgatgtt | 800 |
| gacaactatg atgacatcag aacggaactg cctcctgaga agctcaaact | 850 |
| ggagtgggca tatggttatc gaggaaagga ctgtagagct aatgtttacc | 900 |
| ttcttccgac cggggaaata gtttatttca ttgcatcagt agtagtacta | 950 |
| tttaattatg aggagagaac tcagcgacac tacctgggcc atacagactg | 1000 |
| tgtgaaatgc cttgctatac atcctgacaa aattaggatt gcaactggac | 1050 |
| agatagctgg cgtggataaa gatggaaggc ctctacaacc ccacgtcaga | 1100 |
| gtgtgggatt ctgttactct atccacactg cagattattg gacttggcac | 1150 |
| ttttgagcgt ggagtaggat gcctggattt ttcaaaagca gattcaggtg | 1200 |
| ttcatttatg tgttattgat gactccaatg agcatatgct tactgtatgg | 1250 |
| gactggcaga ggaaagcaaa aggagcagaa ataaagacaa caaatgaagt | 1300 |
| tgttttggct gtggagtttc acccaacaga tgcaaatacc ataattacat | 1350 |
| gcggtaaatc tcatattttc ttctggacct ggagcggcaa ttcactaaca | 1400 |
| agaaaacagg gaatttttgg gaaatatgaa aagccaaaat ttgtgcagtg | 1450 |
| tttagcattc ttggggaatg gagatgttct tactggagac tcaggtggag | 1500 |
| tcatgcttat atggagcaaa actactgtag agcccacacc tgggaaagga | 1550 |
| cctaaAGGAA GTGGCCTGTG TAGTGCTTCA AGGGCCAGGC TGCCAGGCCA | 1600 |
| TGTTGCAGCT GACCACCCAC CTGCAGTGTA CCGCCGGAAG CACCAGGAGC | 1650 |
| TGCAAGCCAT GCAGATGGAG CTGCAGAGCC CTGAGTACAA GCTGAGCAAG | 1700 |
| CTCCGCACCT CGACCATCAT GACCGACTAC AACCCCAACT ACTGCTTTGC | 1750 |
| TGGCAAGACC TCCTCCATCA GTGACCTGAA GGAGGTGCCG CGGAAAAACA | 1800 |
| TCACCCTCAT TCGGGGTCTG GGCCATGGaG CCTTTGGGGA GGTGTATGAA | 1850 |
| GGCCAGGTGT CCGGAATGCC CAACGACCCA AGCCCCCTGC AAGTGGCTGT | 1900 |
| GAAGACGCTG CCTGAAGTGT GCTCTGAACA GGACGAACTG GATTTCCTCA | 1950 |
| TGGAAGCCCT GATCATCAGC AAATTCAACC ACCAGAACAT TGTTCGCTGC | 2000 |
| ATTGGGGTGA GCCTGCAATC CCTGCCCCGG TTCATCCTGC TGGAGCTCAT | 2050 |
| GGCGGGGGGA GACCTCAAGT CCTTCCTCCG AGAGACCCGC CCTCGCCCGA | 2100 |
| GCCAGCCCTC CTCCCTGGCC ATGCTGGACC TTCTGCACGT GGCTCGGGAC | 2150 |
| ATTGCCTGTG GCTGTCAGTA TTTGGAGGAA AACCACTTCA TCCACCGAGA | 2200 |
| CATTGCTGCC AGAAACTGCC TCTTGACCTG TCCAGGCCCT GGAAGAGTGG | 2250 |
| CCAAGATTGG AGACTTCGGG ATGGCCCGAG ACATCTACAG GGCGAGCTAC | 2300 |
| TATAGAAAGG GAGGCTGTGC CATGCTGCCA GTTAAGTGGA TGCCCCCAGA | 2350 |
| GGCCTTCATG GAAGGAATAT TCACTTCTAA AACAGACACA TGGTCCTTTG | 2400 |
| GAGTGCTGCT ATGGGAAATC TTTTCTCTTG GATATATGCC ATACCCCAGC | 2450 |
| AAAAGCAACC AGGAAGTTCT GGAGTTTGTC ACCAGTGGAG GCCGGATGGA | 2500 |
| CCCACCCAAG AACTGCCCTG GGCCTGTATA CCGGATAATG ACTCAGTGCT | 2550 |
| GGCAACATCA GCCTGAAGAC AGGCCCAACT TTGCCATCAT TTTGGAGAGG | 2600 |
| ATTGAATACT GCACCCAGGA CCCGGATGTA ATCAACACCG CTTTGCCGAT | 2650 |
| AGAATATGGT CCACTTGTGG AAGAGGAAGA GAAAGTGCCT GTGAGGCCCA | 2700 |
| AGGACCCTGA GGGGGTTCCT CCTCTCCTGG TCTCTCAACA GGCAAAACGG | 2750 |
| GAGGAGGAGC GCAGCCCAGC TGCCCCACCA CCTCTGCCTA CCACCTCCTC | 2800 |
| TGGCAAGGCT GCAAAGAAAC CCACAGCTGC AGAGgTCTCT GTTCGAGTCC | 2850 |
| CTAGAGGGCC GGCCGTGGAA GGGGGACACG TGAATATGGC ATTCTCTCAG | 2900 |
| TCCAACCCTC CTTCGGAGTT GCACAgGGTC CACGGATCCA GAAACAAGCC | 2950 |
| CACCAGCTTG TGGAACCCAA CGTACGGCTC CTGGTTTACA GAGAAACCCA | 3000 |
| CCAAAAAGAA TAATCCTATA GCAAAGAAGG AGCCACACGA gAGGGGTAAC | 3050 |
| CTGGGGCTGG AGGGAAGCTG TACTGTCCCA CCTAACGTTG CAACTGGGAG | 3100 |
| ACTTCCGGGG GCCTCACTGC TCCTAGAGCC CTCTTCGCTG ACTGCCAATA | 3150 |
| TGAAGGAGGT ACCTCTGTTC AGGCTACGTC ACTTCCCTTG TGGGAATGTC | 3200 |
| AATTACGGCT ACCAGCAACA GGGCTTGCCC TTAGAAGCCG CTACTGCCCC | 3250 |
| TGGAGCTGGT CATTACGAGG ATACCATTCT GAAAAGCAAG AATAGCATGA | 3300 |
| ACCAGCCTGG GCCCTGAGCT CGGTCGCACA CTCACTTCTC TTCCTTGGGA | 3350 |
| TCCCTAAGAC CGTGG (SEQ ID NO: 10) | |

mRNA for fusion protein EML4-ALK variant 7

| | |
|---|---|
| tACTCTGTCG GTCCGCTGAA TGAAGTGCCC GCCCCTCTAA GCCCGGAGCC | 50 |
| CGGCGCTTTC CCCGCAAGAT GGACGGTTTC GCCGGCAGTC TCGATGATAG | 100 |
| TATTTCTGCT GCAAGTACTT CTGATGTTCA AGATCGCCTG TCAGCTCTTG | 150 |
| AGTCACGAGT TCAGCAACAA GAAGATGAAA TCACTGTGCT AAAGGCGGCT | 200 |
| TTGGCTGATG TTTTGAGGCG TCTTGCAATC TCTGAAGATC ATGTGGCCTC | 250 |
| AGTGAAAAAA TCAGTCTCAA GTAAAGGCCA ACCAAGCCCT CGAGCAGTTA | 300 |
| TTCCCATGTC CTGTATAACC AATGGAAGTG GTGCAAACAG AAAACCAAGT | 350 |
| CATACCAGTG CTGTCTCAAT TGCAGGAAAA GAAACTCTTT CATCTGCTGC | 400 |
| TAAAAGTGGT ACAGAAAAAA AGAAAGAAAA ACCACAAGGA CAGAGAGAAA | 450 |
| AAAAAGAGGA ATCTCATTCT AATGATCAAA GTCCACAAAT TCGAGCATCA | 500 |
| CCTTCTCCCC AGCCCTCTTC ACAACCTCTC CAAATACACA GACAAACTCC | 550 |
| AGAAAGCAAG AATGCTACTC CCACCAAAAG CATAAAACGA CCATCACCAG | 600 |
| CTGAAAAGTC ACATAATTCT TGGGAAAATT CAGATGATAG CCGTAATAAA | 650 |
| TTGTCGAAAA TACCTTCAAC ACCCAAATTA ATACCAAAAG TTACCAAAAC | 700 |
| TGCAGACAAG CATAAAGATG TCATCATCAA CCAAGAAGGA GAATATATTA | 750 |
| AAATGTTTAT GCGCGGTCGG CCAATTACCA TGTTCATTCC TTCCGATGTT | 800 |
| GACAACTATG ATGACATCAG AACGGAACTG CCTCCTGAGA AGCTCAAACT | 850 |
| GGAGTGGGCA TATGGTTATC GAGGAAAGGA CTGTAGAGCT AATGTTTACC | 900 |
| TTCTTCCGAC CGGGgAAATA GTTTATTTCA TTGCATCAGT AGTAGTACTA | 950 |
| TTTAATTATG AGGAGAGAAC TCAGCGACAC TACCTGGGCC ATACAGACTG | 1000 |
| TGTGAAATGC CTTGCTATAC ATCCTGACAA AATTAGGATT GCAACTGGAC | 1050 |
| AGATAGCTGG CGTGGATAAA GATGGAAGGC CTCTACAACC CCACGTCAGA | 1100 |
| GTGTGGGATT CTGTTACTCT ATCCACACTG CAGATTATTG GACTTGGCAC | 1150 |
| TTTTGAGCGT GGAGTAGGAT GCCTGGATTT TTCAAAAGCA GATTCAGGTG | 1200 |
| TTCATTTATG TgTTATTGAT GACTCCAATG AGCATATGCT TACTGTATGG | 1250 |
| GACTGGCAGA gGAAAGCAAA AGGAGCAGAA ATAAAGACAA CAAATGAAGT | 1300 |
| TGTTTTGGCT GTGGAGTTTC ACCCAACAGA TGCAAATACC ATAATTACAT | 1350 |
| GCGGTAAATC TCATATTTTC TTCTGGACCT GGAGCGGCAA TTCACTAACA | 1400 |
| AGAAAACAGG GAATTTTTGG GAAATATGAA AAGCCAAAAT TTGTGCAGTG | 1450 |
| TTTAGCATTC TTGGGGAATG GAGATGTTCT TACTGGAGAC TCAGGTGGAG | 1500 |
| TCATGCTTAT ATGGAGCAAA ACTACTGTAG AGCCCACACC TGGGAAAGGA | 1550 |
| CCTAAAGGTG TATATCAAAT CAGCAAACAA ATCAAAGCTC ATGATGGCAG | 1600 |
| TGTGTTCACA CTTTGTCAGA TGAGAAATGG GATGTTATTA ACTGGAGGAG | 1650 |
| GGAAAGACAG AAAAATAATT CTGTGGGATC ATGATCTGAA TCCTGAAAGA | 1700 |
| GAAATAGAGc accaggagct gcaagccatg cagatggagc tgcagagccc | 1750 |
| tgagtacaag ctgagcaagc tccgcacctc gaccatcatg accgactaca | 1800 |
| accccaacta ctgctttgct ggcaagacct cctccatcag tgacctgaag | 1850 |
| gaggtgccgc ggaaaaacat caccctcatt cggggtctgg gccatggagc | 1900 |
| ctttggggag gtgtatgaag gccaggtgtc cggaatgccc aacgacccaa | 1950 |
| gccccctgca agtggctgtg aagacgctgc ctgaagtgtg ctctgaacag | 2000 |
| gacgaactgg atttcctcat ggaagccctg atcatcagca aattcaacca | 2050 |
| ccagaacatt gttcgctgca ttggggtgag cctgcaatcc ctgccccggt | 2100 |
| tcatcctgct ggagctcatg gcggggggag acctcaagtc cttcctccga | 2150 |
| gagacccgcc ctcgcccgag ccagccctcc tccctggcca tgctggacct | 2200 |
| tctgcacgtg gctcgggaca ttgcctgtgg ctgtcagtat ttggaggaaa | 2250 |
| accacttcat ccaccgagac attgctgcca gaaactgcct cttgacctgt | 2300 |
| ccaggccctg gaagagtggc caagattgga gacttcggga tggcccgaga | 2350 |
| catctacagg gcgagctact atagaaaggg aggctgtgcc atgctgccag | 2400 |
| ttaagtggat gcccccagag gccttcatgg aaggaatatt cacttctaaa | 2450 |
| acagacacat ggtcctttgg agtgctgcta tgggaaatct tttctcttgg | 2500 |
| atatatgcca taccccagca aaagcaacca ggaagttctg gagtttgtca | 2550 |
| ccagtggagg ccggatggac ccacccaaga actgccctgg gcctgtatac | 2600 |
| cggataatga ctcagtgctg gcaacatcag cctgaagaca ggcccaactt | 2650 |
| tgccatcatt ttggagagga ttgaatactg cacccaggac ccggatgtaa | 2700 |
| tcaacaccgc tttgccgata gaatatggtc cacttgtgga agaggaagag | 2750 |
| aaagtgcctg tgaggcccaa ggaccctgag ggggttcctc ctctcctggt | 2800 |
| ctctcaacag gcaaaacggg aggaggagcg cagcccagct gccccaccac | 2850 |
| ctctgcctac cacctcctct ggcaaggctg caaagaaacc cacagctgca | 2900 |
| gaggtctctg ttcgagtccc tagagggccg gccgtggaag ggggacacgt | 2950 |
| gaatatggca ttctctcagt ccaaccctcc ttcggagttg cacaaggtcc | 3000 |
| acggatccag aaacaagccc accagcttgt ggaacccaac gtacggctcc | 3050 |
| tggtttacag agaaacccac caaaaagaat aatcctatag caaagaagga | 3100 |
| gccacacgac aggggtaacc tggggctgga gggaagctgt actgtcccac | 3150 |
| ctaacgttgc aactgggaga cttccggggg cctcactgct cctagagccc | 3200 |
| tcttcgctga ctgccaatat gaaggaggta cctctgttca ggctacgtca | 3250 |
| cttcccttgt gggaatgtca attacggcta ccagcaacag ggcttgccct | 3300 |
| tagaagccgc tactgcccct ggagctggtc attacgagga taccattctg | 3350 |
| aaaagcaaga atagcatgaa ccagcctggg ccctgagctc ggtcgcacac | 3400 |
| tcacttctct tccttgggat ccctaagacc gtgga (SEQ ID NO: 11) | |

mRNA for fusion protein KIF5B-ALK

| | |
|---|---|
| TGCGAGAAAG ATGGCGGACC TGGCCGAGTG CAACATCAAA GTGATGTGTC | 50 |
| GCTTCAGACC TCTCAACGAG TCTGAAGTGA ACCGCGGCGA CAAGTACATC | 100 |
| GCCAAGTTTC AGGGAGAAGA CACGGTCGTG ATCGCGTCCA AGCCTTATGC | 150 |
| ATTTGATCGG GTGTTCCAGT CAAGCACATC TCAAGAGCAA GTGTATAATG | 200 |
| ACTGTGCAAA GAAGATTGTT AAAGATGTAC TTGAAGGATA TAATGGAACA | 250 |
| ATATTTGCAT ATGGACAAAC ATCCTCTGGG AAGACACACA CAATGGAGGG | 300 |
| TAAACTTCAT GATCCAGAAG GCATGGGAAT TATTCCAAGA ATAGTGCAAG | 350 |
| ATATTTTTAA TTATATTTAC TCCATGGATG AAAATTTGGA ATTTCATATT | 400 |
| AAGGTTTCAT ATTTTGAAAT ATATTTGGAT AAGATAAGGG ACCTGTTAGA | 450 |
| TGTTTCAAAG ACCAACCTTT CAGTTCATGA AGACAAAAAC CGAGTTCCCT | 500 |
| ATGTAAAGGG GTGCACAGAG CGTTTTGTAT GTAGTCCAGA TGAAGTTATG | 550 |
| GATACCATAG ATGAAGGAAA ATCCAACAGA CATGTAGCAG TTACAAATAT | 600 |
| GAATGAACAT AGCTCTAGGA GTCACAGTAT ATTTCTTATT AATGTCAAAC | 650 |
| AAGAGAACAC ACAAACGGAA CAAAAGCTGA GTGGAAAACT TTATCTGGTT | 700 |
| GATTTAGCTG GTAGTGAAAA GGTTAGTAAA ACTGGAGCTG AAGGTGCTGT | 750 |
| GCTGGATGAA GCTAAAAACA TCAACAAGTC ACTTTCTGCT CTTGGAAATG | 800 |
| TTATTTCTGC TTTGGCTGAG GGTAGTACAT ATGTTCCATA TCGAGATAGT | 850 |
| AAAATGACAA GAATCCTTCA AGATTCATTA GGTGGCAACT GTAGAACCAC | 900 |
| TATTGTAATT TGCTGCTCTC CATCATCATA CAATGAGTCT GAAACAAAAT | 950 |
| CTACACTCTT ATTTGGCCAA AGGGCCAAAA CAATTAAGAA CACAGTTTGT | 1000 |
| GTCAATGTGG AGTTAACTGC AGAACAGTGG AAAAAGAAGT ATGAAAAAGA | 1050 |
| AAAAGAAAAA AATAAGATCC TGCGGAACAC TATTCAGTGG CTTGAAAATG | 1100 |
| AGCTCAACAG ATGGCGTAAT GGGGAGACGG TGCCTATTGA TGAACAGTTT | 1150 |
| GACAAAGAGA AAGCCAACTT GGAAGCTTTC ACAGTGGATA AAGATATTAC | 1200 |
| TCTTACCAAT GATAAACCAG CAACCGCAAT TGGAGTTATA GGAAATTTTA | 1250 |
| CTGATGCTGA AAGAAGAAAG TGTGAAGAAG AAATTGCTAA ATTATACAAA | 1300 |
| CAGCTTGATG ACAAGGATGA AGAAATTAAC CAGCAAAGTC AACTGGTAGA | 1350 |
| GAAACTGAAG ACGCAAATGT TGGATCAGGA GGAGCTTTTG GCATCTACCA | 1400 |
| GAAGGGATCA AGACAATATG CAAGCTGAGC TGAATCGCCT TCAAGCAGAA | 1450 |
| AATGATGCCT CTAAAGAAGA AGTGAAAGAA GTTTTACAGG CCCTAGAAGA | 1500 |
| ACTTGCTGTC AATTATGATC AGAAGTCTCA GGAAGTTGAA GACAAAACTA | 1550 |
| AGGAATATGA ATTGCTTAGT GATGAATTGA ATCAGAAATC GGCAACTTTA | 1600 |
| GCGAGTATAG ATGCTGAGCT TCAGAAACTT AAGGAAATGA CCAACCACCA | 1650 |
| GAAAAAACGA GCAGCTGAGA TGATGGCATC TTTACTAAAA GACCTTGCAG | 1700 |
| AAATAGGAAT TGCTGTGGGA AATAATGATG TAAAGCAGCC TGAGGGAACT | 1750 |
| GGCATGATAG ATGAAGAGTT CACTGTTGCA AGACTCTACA TTAGCAAAAT | 1800 |
| GAAGTCAGAA GTAAAAACCA TGGTGAAACG TTGCAAGCAG TTAGAAAGCA | 1850 |
| CACAAACTGA GAGCAACAAA AAAATGGAAG AAAATGAAAA GGAGTTAGCA | 1900 |
| GCATGTCAGC TTCGTATCTC TCAACATGAA GCCAAAATCA AGTCATTGAC | 1950 |
| TGAATACCTT CAAAATGTGG AACAAAAGAA AAGACAGTTG GAGGAATCTG | 2000 |
| TCGATGCCCT CAGTGAAGAA CTAGTCCAGC TTCGAGCACA AGAGAAAGTC | 2050 |
| CATGAAATGG AAAAGGAGCA CTTAAATAAG GTTCAGACTG CAAATGAAGT | 2100 |
| TAAGCAAGCT GTTGAACAGC AGATCCAGAG CCATAGAGAA ACTCATCAAA | 2150 |
| AACAGATCAG TAGTTTGAGA GATGAAGTAG AAGCAAAAGC AAAACTTATT | 2200 |
| ACTGATCTTC AAGACCAAAA CCAGAAAATG ATGTTAGAGC AGGAACGTCT | 2250 |
| AAGAGTAGAA CATGAGAAGT TGAAAGCCAC AGATCAGGAA AAGAGCAGAA | 2300 |
| AACTACATGA ACTTACGGTT ATGCAAGATA GACGAGAACA AGCAAGACAA | 2350 |
| GACTTGAAGG GTTTGGAAGA GACAGTGGCA AAAGAACTTC AGACTTTACA | 2400 |
| CAACCTGCGC AAACTCTTTG TTCAGGACCT GGCTACAAGA GTTAAAAAGA | 2450 |
| GTGCTGAGAT TGATTCTGAT GACACCGGAG GCAGCGCTGC TCAGAAGCAA | 2500 |
| AAAATCTCCT TTCTTGAAAA TAATCTTGAA CAGCTCACTA AAGTGCACAA | 2550 |
| ACAGTTGGTA CGTGATAATG CAGATCTCCG CTGTGAACTT CCTAAGTTGG | 2600 |
| AAAAGCGACT TCGAGCTACA GCTGAGAGAG TGAAAGCTTT GGAATCAGCA | 2650 |
| CTGAAAGAAG CTAAAGAAAA TGCATCTCGT GATCGCAAAC GCTATCAGCA | 2700 |
| AGAAGTAGAT CGCATAAAGG AAGCAGTCAG GTCAAAGAAT ATGGCCAGAA | 2750 |
| GAGGGCATTC TGCACAGATT Gtgtaccgcc ggaagcacca ggagctgcaa | 2800 |
| gccatgcaga tggagctgca gagccctgag tacaagctga gcaagctccg | 2850 |
| cacctcgacc atcatgaccg actacaaccc caactactgc tttgctggca | 2900 |
| agacctcctc catcagtgac ctgaaggagg tgccgcggaa aaacatcacc | 2950 |
| ctcattcggg gtctgggcca tggcgccttt ggggaggtgt atgaaggcca | 3000 |
| ggtgtccgga atgcccaacg acccaagccc cctgcaagtg gctgtgaaga | 3050 |
| cgctgcctga agtgtgctct gaacaggacg aactggattt cctcatggaa | 3100 |
| gccctgatca tcagcaaatt caaccaccag aacattgttc gctgcattgg | 3150 |
| ggtgagcctg caatccctgc cccggttcat cctgctggag ctcatggcgg | 3200 |
| ggggagacct caagtccttc ctccgagaga cccgccctcg cccgagccag | 3250 |
| ccctcctccc tggccatgct ggaccttctg cacgtggctc gggacattgc | 3300 |
| ctgtggctgt cagtatttgg aggaaaacca cttcatccac cgagacattg | 3350 |
| ctgccagaaa ctgcctcttg acctgtccag gccctggaag agtggccaag | 3400 |
| attggagact tcgggatggc ccgagacatc tacagggcga gctactatag | 3450 |
| aaagggaggc tgtgccatgc tgccagttaa gtggatgccc ccagaggcct | 3500 |
| tcatggaagg aatattcact tctaaaacag acacatggtc ctttggagtg | 3550 |
| ctgctatggg aaatcttttc tcttggatat atgccatacc ccagcaaaag | 3600 |
| caaccaggaa gttctggagt ttgtcaccag tggaggccgg atggacccac | 3650 |
| ccaagaactg ccctgggcct gtataccgga taatgactca gtgctggcaa | 3700 |
| catcagcctg aagacaggcc caactttgcc atcattttgg agaggattga | 3750 |
| atactgcacc caggacccgg atgtaatcaa caccgctttg ccgatagaat | 3800 |
| atggtccact tgtggaagag gaagagaaag tgcctgtgag gcccaaggac | 3850 |
| cctgaggggg ttcctcctct cctggtctct caacaggcaa aacgggagga | 3900 |
| ggagcgcagc ccagctgccc caccacctct gcctaccacc tcctctggca | 3950 |
| aggctgcaaa gaaacccaca gctgcagagg tctctgttcg agtccctaga | 4000 |
| gggccggccg tggaaggggg acacgtgaat atggcattct ctcagtccaa | 4050 |
| ccctccttcg gagttgcaca aggtccacgg atccagaaac aagcccacca | 4100 |
| gcttgtggaa cccaacgtac ggctcctggt ttacagagaa acccaccaaa | 4150 |
| aagaataatc ctatagcaaa gaaggagcca cacgacaggg gtaacctggg | 4200 |
| gctggaggga agctgtactg tcccacctaa cgttgcaact gggagacttc | 4250 |
| cgggggcctc actgctccta gagccctctt cgctgactgc caatatgaag | 4300 |
| gaggtacctc tgttcaggct acgtcacttc ccttgtggga atgtcaatta | 4350 |
| cggctaccag caacagggct tgcccttaga agccgctact gcccctggag | 4400 |
| ctggtcatta cgaggatacc attctgaaaa gcaagaatag catgaaccag | 4450 |
| cctgggccct gagctcggtc gcacactca (SEQ ID NO: 12) | |

mRNA for TRK-fused gene/anaplastic large cell lymphoma kinase
(TFG/ALK fusion) extra long form

| | |
|---|---|
| atgaacggac agttggatct aagtgggaag ctaatcatca aagctcaact | 50 |
| tggggaggat attcggcgaa ttcctattca taatgaagat attacttatg | 100 |
| atgaattagt gctaatgatg caacgagttt tcagaggaaa acttctgagt | 150 |
| aatgatgaag taacaataaa gtataaagat gaagatggag atcttataac | 200 |
| aatttttgat agttctgacc tttcctttgc aattcagtgc agtaggatac | 250 |
| tgaaactgac attatttgtt aatggccagc caagacccct tgaatcaagt | 300 |
| caggtgaaat atctccgtcg agaactgata gaacttcgaa ataaagtgaa | 350 |
| tcgtttattg gatagcttgg aaccacctgg agaaccagga ccttccacca | 400 |
| atattcctga aaatgatact gtggatggta gggaagaaaa gtctgcttct | 450 |
| gattcttctg gaaaacagtc tactcaggtt atggcagcaa gtatgtctgc | 500 |
| ttttgatcct ttaaaaaacc aagatgaaat caataaaaat gttatgtcag | 550 |
| cgtttggctt aacagatgat caggtttcag ggccacccag tgctcctgca | 600 |
| gaagatcgtt caggaacacc cgacagcatt gcttcctcct cctcagcagc | 650 |
| tcacccacca ggcgttcagc cacagcagcc accatataca ggagctcaga | 700 |
| ctcaagcagg tcagattgaA GTGTACCGCC GGAAGCACCA GGAGCTGCAA | 750 |
| GCCATGCAGA TGGAGCTGCA GAGCCCTGAG TACAAGCTGA GCAAGCTCCG | 800 |
| CACCTCGACC ATCATGACCG ACTACAACCC CAACTACTGC TTTGCTGGCA | 850 |
| AGACCTCCTC CATCAGTGAC CTGAAGGAGG TGCCGCGGAA AAACATCACC | 900 |
| CTCATTCGGG GTCTGGGCCA TGGCGCCTTT GGGGAGGTGT ATGAAGGCCA | 950 |
| GGTGTCCGGA ATGCCCAACG ACCCAAGCCC CCTGCAAGTG GCTGTGAAGA | 1000 |
| CGCTGCCTGA AGTGTGCTCT GAACAGGACG AACTGGATTT CCTCATGGAA | 1050 |
| GCCCTGATCA TCAGCAAATT CAACCACCAG AACATTGTTC GCTGCATTGG | 1100 |
| GGTGAGCCTG CAATCCCTGC CCCGGTTCAT CCTGCTGGAG CTCATGGCGG | 1150 |
| GGGGAGACCT CAAGTCCTTC CTCCGAGAGA CCCGCCCTCG CCCGAGCCAG | 1200 |
| CCCTCCTCCC TGGCCATGCT GGACCTTCTG CACGTGGCTC GGGACATTGC | 1250 |
| CTGTGGCTGT CAGTATTTGG AGGAAAACCA CTTCATCCAC CGAGACATTG | 1300 |
| CTGCCAGAAA CTGCCTCTTG ACCTGTCCAG GCCCTGGAAG AGTGGCCAAG | 1350 |
| ATTGGAGACT TCGGGATGGC CCGAGACATC TACAGGGCGA GCTACTATAG | 1400 |
| AAAGGGAGGC TGTGCCATGC TGCCAGTTAA GTGGATGCCC CCAGAGGCCT | 1450 |
| TCATGGAAGG AATATTCACT TCTAAAACAG ACACATGGTC CTTTGGAGTG | 1500 |
| CTGCTATGGG AAATCTTTTC TCTTGGATAT ATGCCATACC CCAGCAAAAG | 1550 |
| CAACCAGGAA GTTCTGGAGT TTGTCACCAG TGGAGGCCGG ATGGACCCAC | 1600 |
| CCAAGAACTG CCCTGGGCCT GTATACCGGA TAATGACTCA GTGCTGGCAA | 1650 |
| CATCAGCCTG AAGACAGGCC CAACTTTGCC ATCATTTTGG AGAGGATTGA | 1700 |
| ATACTGCACC CAGGACCCGG ATGTAATCAA CACCGCTTTG CCGATAGAAT | 1750 |
| ATGGTCCACT TGTGGAAGAG GAAGAGAAAG TGCCTGTGAG GCCCAAGGAC | 1800 |
| CCTGAGGGGG TTCCTCCTCT CCTGGTCTCT CAACAGGCAA AACGGGAGGA | 1850 |
| GGAGCGCAGC CCAGCTGCCC CACCACCTCT GCCTACCACC TCCTCTGGCA | 1900 |
| AGGCTGCAAA GAAACCCACA GCTGCAGAGg TCTCTGTTCG AGTCCCTAGA | 1950 |
| GGGCCGGCCG TGGAAGGGGG ACACGTGAAT ATGGCATTCT CTCAGTCCAA | 2000 |
| CCCTCCTTCG GAGTTGCACA AGGTCCACGG ATCCAGAAAC AAGCCCACCA | 2050 |
| GCTTGTGGAA CCCAACGTAC GGCTCCTGGT TTACAGAGAA ACCCACCAAA | 2100 |
| AAGAATAATC CTATAGCAAA GAAGGAGCCA CACGACAGGG GTAACCTGGG | 2150 |
| GCTGGAGGGA AGCTGTACTG TCCCACCTAA CGTTGCAACT GGGAGACTTC | 2200 |
| CGGGGGCCTC ACTGCTCCTA GAGCCCTCTT CGCTGACTGC CAATATGAAG | 2250 |
| GAGGTACCTC TGTTCAGGCT ACGTCACTTC CCTTGTGGGA ATGTCAATTA | 2300 |
| CGGCTACCAG CAACAGGGCT TGCCCTTAGA AGCCGCTACT GCCCCTGGAG | 2350 |
| CTGGTCATTA CGAGGATACC ATTCTGAAAA GCAAGAATAG CATGAACCAG | 2400 |
| CCTGGGCCCT Ga (SEQ ID NO: 13) | |

mRNA for nucleophosmin-anaplastic lymphoma kinase fusion
protein (NPM/ALK)

| | |
|---|---|
| atggaagatt cgatggacat ggacatgagc cccctgaggc cccagaacta | 50 |
| tcttttcggt tgtgaactaa aggccgacaa agattatcac tttaaggtgg | 100 |
| ataatgatga aaatgagcac cagttatctt taagaacggt cagtttaggg | 150 |
| gctggtgcaa aggatgagtt gcacattgtt gaagcagagg caatgaatta | 200 |
| cgaaggcagt ccaattaaag taacactggc aactttgaaa atgtctgtac | 250 |
| agccaacggt ttcccttggg ggctttgaaa taacaccacc agtggtctta | 300 |
| aggttgaagt gtggttcagg gccagtgcat attagtggac agcacttagt | 350 |
| AGTGTACCGC CGGAAGCACC AGGAGCTGCA AGCCATGCAG ATGGAGCTGC | 400 |
| AGAGCCCTGA GTACAAGCTG AGCAAGCTCC GCACCTCGAC CATCATGACC | 450 |
| GACTACAACC CCAACTACTG CTTTGCTGGC AAGACCTCCT CCATCAGTGA | 500 |
| CCTGAAGGAG GTGCCGCGGA AAAACATCAC CCTCATTCGG GGTCTGGGCC | 550 |
| ATGGCGCCTT TGGGGAGGTG TATGAAGGCC AGGTGTCCGG AATGCCCAAC | 600 |
| GACCCAAGCC CCCTGCAAGT GGCTGTGAAG ACGCTGCCTG AAGTGTGCTC | 650 |
| TGAACAGGAC GAACTGGATT TCCTCATGGA AGCCCTGATC ATCAGCAAAT | 700 |
| TCAACCACCA GAACATTGTT CGCTGCATTG GGGTGAGCCT GCAATCCCTG | 750 |
| CCCCGGTTCA TCCTGCTGGA GCTCATGGCG GGGGGAGACC TCAAGTCCTT | 800 |
| CCTCCGAGAG ACCCGCCCTC GCCCGAGCCA GCCCTCCTCC CTGGCCATGC | 850 |
| TGGACCTTCT GCACGTGGCT CGGGACATTG CCTGTGGCTG TCAGTATTTG | 900 |
| GAGGAAAACC ACTTCATCCA CCGAGACATT GCTGCCAGAA ACTGCCTCTT | 950 |
| GACCTGTCCA GGCCCTGGAA GAGTGGCCAA GATTGGAGAC TTCGGGATGG | 1000 |
| CCCGAGACAT CTACAGGGCG AGCTACTATA GAAAGGGAGG CTGTGCCATG | 1050 |
| CTGCCAGTTA AGTGGATGCC CCCAGAGGCC TTCATGGAAG GAATATTCAC | 1100 |
| TTCTAAAACA GACACATGGT CCTTTGGAGT GCTGCTATGG GAAATCTTTT | 1150 |
| CTCTTGGATA TATGCCATAC CCCAGCAAAA GCAACCAGGA AGTTCTGGAG | 1200 |
| TTTGTCACCA GTGGAGGCCG GATGGACCCA CCCAAGAACT GCCCTGGGCC | 1250 |
| TGTATACCGG ATAATGACTC AGTGCTGGCA ACATCAGCCT GAAGACAGGC | 1300 |
| CCAACTTTGC CATCATTTTG GAGAGGATTG AATACTGCAC CCAGGACCCG | 1350 |
| GATGTAATCA ACACCGCTTT GCCGATAGAA TATGGTCCAC TTGTGGAAGA | 1400 |
| GGAAGAGAAA GTGCCTGTGA GGCCCAAGGA CCCTGAGGGG GTTCCTCCTC | 1450 |
| TCCTGGTCTC TCAACAGGCA AAACGGGAGG AGGAGCGCAG CCCAGCTGCC | 1500 |
| CCACCACCTC TGCCTACCAC CTCCTCTGGC AAGGCTGCAA AGAAACCCAC | 1550 |
| AGCTGCAGAG gTCTCTGTTC GAGTCCCTAG AGGGCCGGCC GTGGAAGGGG | 1600 |
| GACACGTGAA TATGGCATTC TCTCAGTCCA ACCCTCCTTC GGAGTTGCAC | 1650 |
| AAGGTCCACG GATCCAGAAA CAAGCCCACC AGCTTGTGGA ACCCAACGTA | 1700 |
| CGGCTCCTGG TTTACAGAGA AACCCACCAA AAAGAATAAT CCTATAGCAA | 1750 |
| AGAAGGAGCC ACACGACAGG GGTAACCTGG GGCTGGAGGG AAGCTGTACT | 1800 |
| GTCCCACCTA ACGTTGCAAC TGGGAGACTT CCGGGGGCCT CACTGCTCCT | 1850 |
| AGAGCCCTCT TCGCTGACTG CCAATATGAA GGAGGTACCT CTGTTCAGGC | 1900 |
| TACGTCACTT CCCTTGTGGG AATGTCAATT ACGGCTACCA GCAACAGGGC | 1950 |
| TTGCCCTTAG AAGCCGCTAC TGCCCCTGGA GCTGGTCATT ACGAGGATAC | 2000 |
| CATTCTGAAA AGCAAGAATA GCATGAACCA GCCTGGGCCC TGa | |

ALK point mutations:
F1245I/L; L1204F; A1200V; L1196M; I1170S; T1151M; R1275Q; F1174V/C/L; T1087I; K1062M

TABLE 2
Demographics, Baseline Characteristics and Chemotherapy Treatment History by EGFR, KRAS and ALK Genotypes

| | | | | | | | ALK Status | |
|---|---|---|---|---|---|---|---|---|
| | | | EGFR Status | | KRAS Status | | (n = 15) | |
| | | | (n = 68) | | (n = 38) | | No Rearrangement | Rearrangement |
| | | Total | Wild Type | Mutant | Wild Type | Mutant | Detected | Detected |
| Number of Patients | | 76 | 40 | 28 | 26 | 12 | 12 | 3 |
| Age (years) | Median | 64.0 | 63.0 | 66.0 | 61.0 | 65.0 | 65.5 | 48.0 |
| | Range | 31-82 | 31-79 | 44-82 | 31-81 | 52-76 | 48-76 | 31-58 |
| Sex (n [%]) | Female | 48 (63) | 22 (55) | 20 (71) | 17 (65) | 7 (58) | 9 (75) | 1 (33) |
| | Male | 28 (37) | 18 (45) | 8 (29) | 9 (35) | 5 (42) | 3 (25) | 2 (67) |
| Race (n [%]) | Asian | 11 (14) | 6 (15) | 5 (18) | 4 (15) | 0 | 2 (17) | 1 (33) |
| | Black or African | 4 (5) | 2 (5) | 2 (7) | 2 (8) | 0 | 0 | 0 |
| | American | | | | | | | |
| | White | 61 (80) | 32 (80) | 21 (75) | 20 (77) | 12 (100) | 10 (83) | 2 (67) |
| Smoking Status (n | Current Smoker | 0 | 0 | 0 | 0 | 0 | 0 | 0 |
| [%]) | Never Smoked | 34 (45) | 13 (33) | 17 (61) | 13 (50) | 0 | 3 (25) | 3 (100) |
| | Previous Smoker | 42 (55) | 27 (68) | 11 (39) | 13 (50) | 12 (100) | 9 (75) | 0 |
| Months since | Median | 27.5 | 24.6 | 37.2 | 25.7 | 20.6 | 28.5 | 29.7 |
| Diagnosis | Range | 8-120 | 8-120 | 11-108 | 10-120 | 11-71 | 11-71 | 10-120 |
| Histology (n [%]) | Adenocarcinoma | 59 (78) | 31 (78) | 23 (82) | 21 (81) | 10 (83) | 11 (92) | 3 (100) |
| | Brochoalveolar | 4 (5) | 2 (5) | 2 (7) | 0 | 1 (8) | 0 | 0 |
| | Large cell | 2 (3) | 2 (5) | 0 | 1 (4) | 1 (8) | 0 | 0 |
| | Squamous | 6 (8) | 4 (10) | 1 (4) | 3 (12) | 0 | 1 (8) | 0 |
| | Unspecified NSCLC | 5 (7) | 1 (3) | 2 (7) | 1 (4) | 0 | 0 | 0 |
| # of prior treatment | Median | 4.0 | 4.0 | 3.0 | 3.0 | 3.5 | 4.0 | 3.0 |
| regimens for NSCLC | Range | 1-11 | 1-7 | 1-11 | 1-6 | 2-7 | 2-7 | 3-5 |
| Best prior response to | CR | 1 (1) | 0 | 1 (4) | 1 (4) | 0 | 0 | 0 |
| TKI treatment | PR | 18 (24) | 2 (5) | 14 (50) | 3 (12) | 1 (8) | 1 (8) | 0 |
| Total Months on TKI | Median | 1.8 | 1.5 | 10.5 | 1.7 | 1.2 | 1.9 | 0.0 |
| prior to study | Range | 0-61 | 0-25 | 0-61 | 0-61 | 0-16 | 0-16 | 0-1 |

TABLE 3
Most Frequent Adverse Events
(≧15% of Patients with Any Adverse Event)

| | | |
|---|---|---|
| Patients | Patients with | |
| with | Grade 1 or | Patients with |
| Any Event | 2 Event | ≧Grade 3 Event |
| n (%) | n (%) | n (%) |

MedDRA Preferred Term^{a}

| | | | |
|---|---|---|---|
| Fatigue | 44 (57.9) | 41 (53.9) | 6 (7.9) |
| Nausea | 43 (56.6) | 41 (53.9) | 6 (7.9) |
| Diarrhoea | 40 (52.6) | 37 (48.7) | 8 (10.5) |
| Vomiting | 28 (36.8) | 25 (32.9) | 6 (7.9) |
| Cough | 24 (31.6) | 24 (31.6) | 2 (2.6) |
| Urine colour abnormal | 22 (28.9) | 22 (28.9) | 0 (0.0) |
| Anorexia | 19 (25.0) | 18 (23.7) | 4 (5.3) |
| Arthralgia | 19 (25.0) | 17 (22.4) | 2 (2.6) |
| Myalgia | 19 (25.0) | 18 (23.7) | 1 (1.3) |
| Headache | 19 (25.0) | 19 (25.0) | 0 (0.0) |
| Abdominal pain | 18 (23.7) | 18 (23.7) | 1 (1.3) |
| Constipation | 18 (23.7) | 18 (23.7) | 2 (2.6) |
| Dyspnoea | 18 (23.7) | 15 (19.7) | 6 (7.9) |
| Back pain | 16 (21.1) | 16 (21.1) | 0 (0.0) |
| Infusion site pain | 15 (19.7) | 15 (19.7) | 0 (0.0) |
| Dehydration | 14 (18.4) | 11 (14.5) | 3 (3.9) |
| Musculoskeletal chest pain | 13 (17.1) | 11 (14.5) | 3 (3.9) |
| Pyrexia | 12 (15.8) | 12 (15.8) | 0 (0.0) |
| Vision blurred | 12 (15.8) | 12 (15.8) | 0 (0.0) |
| Insomnia | 12 (15.8) | 12 (15.8) | 0 (0.0) |
| Dizziness | 12 (15.8) | 12 (15.8) | 0 (0.0) |

Liver Function Tests^{a}

| | | | |
|---|---|---|---|
| Alkaline phosphatase | 47 (61.8) | 43 (56.6) | 4 (5.3) |
| AST | 37 (48.7) | 30 (39.5) | 7 (9.2) |
| ALT | 31 (40.8) | 26 (34.2) | 5 (6.6) |
| Total bilirubin | 3 (3.9) | 3 (3.9) | 0 (0.0) |

^{a}Maximum post-baseline grade based on laboratory values

TABLE 4
Clinical Efficacy of IPI-504 Based on Molecular Characterization

| | | | | | | ALK Status | |
|---|---|---|---|---|---|---|---|
| | | EGFR Status | | KRAS Status | | (n = 15) | |
| | | (n = 68) | | (n = 38) | | No Rearrangement | Rearrangement |
| | Total | Wild Type | Mutant | Wild Type | Mutant | Detected | Detected |
| Number of Patients (n [%]) | 76 | 40 (53) | 28 (37) | 26 (34) | 12 (16) | 12 (16) | 3 (4) |
| Objective Response Rate | 5 (7) | 4 (10) | 1 (4) | 3 (12)* | 0 (0) | 1 (8) | 2 (67) |
| (All PRs) (n [%]) | | | | | | | |
| RECIST Stable Disease or better for at | 18 (24) | 10 (25) | 6 (21) | 4 (15) | 5 (42) | 3 (25) | 3 (100) |
| least 3 months (n [%]) | | | | | | | |
| Median PFS | 2.86 | 2.86 | 2.76 | 2.86 | 3.91 | 2.43 | Unable to |
| (95% CI) | (2.43-4.18) | (1.18-5.33) | (2.40-3.91) | (1.22-10.20) | (1.12-4.18) | (1.13-5.33) | determine |

*Two of the three KRAS wild-type responders were ALK rearranged; the third was confirmed ALK wild-type.

TABLE 5

KRAS Ref Seq mRNA
>NM_033360.2
ggccgcggcggcggaggcagcagcggcggcggcagtggcggcggcgaaggtggcggcggctcggccagtac
tcccggcccccgccatttcggactgggagcgagcgcggcgcaggcactgaaggcggcggcggggccagagg
ctcagcggctcccaggtgcgggagagaggcctgctgaaaatgactgaatataaacttgtggtagttggagc
tggtggcgtaggcaagagtgccttgacgatacagctaattcagaatcattttgtggacgaatatgatccaa
caatagaggattcctacaggaagcaagtagtaattgatggagaaacctgtctcttggatattctcgacaca
gcaggtcaagaggagtacagtgcaatgagggaccagtacatgaggactggggagggctttctttgtgtatt
tgccataaataatactaaatcatttgaagatattcaccattatagagaacaaattaaaagagttaaggact
ctgaagatgtacctatggtcctagtaggaaataaatgtgatttgccttctagaacagtagacacaaaacag
gctcaggacttagcaagaagttatggaattccttttattgaaacatcagcaaagacaagacagagagtgga
ggatgctttttatacattggtgagggagatccgacaatacagattgaaaaaaatcagcaaagaagaaaaga
ctcctggctgtgtgaaaattaaaaaatgcattataatgtaatctgggtgttgatgatgccttctatacatt
agttcgagaaattcgaaaacataaagaaaagatgagcaaagatggtaaaaagaagaaaaagaagtcaaaga
caaagtgtgtaattatgtaaatacaatttgtacttttttcttaaggcatactagtacaagtggtaattttt
gtacattacactaaattattagcatttgttttagcattacctaatttttttcctgctccatgcagactgtt
agcttttaccttaaatgcttattttaaaatgacagtggaagtttttttttcctctaagtgccagtattccc
agagttttggtttttgaactagcaatgcctgtgaaaaagaaactgaatacctaagatttctgtcttggggt
ttttggtgcatgcagttgattacttcttatttttcttaccaattgtgaatgttggtgtgaaacaaattaat
gaagcttttgaatcatccctattctgtgttttatctagtcacataaatggattaattactaatttcagttg
agaccttctaattggtttttactgaaacattgagggaacacaaatttatgggcttcctgatgatgattctt
ctaggcatcatgtcctatagtttgtcatccctgatgaatgtaaagttacactgttcacaaaggttttgtct
cctttccactgctattagtcatggtcactctccccaaaatattatattttttctataaaaagaaaaaaatg
gaaaaaaattacaaggcaatggaaactattataaggccatttccttttcacattagataaattactataaa
gactcctaatagcttttcctgttaaggcagacccagtatgaaatggggattattatagcaaccattttggg
gctatatttacatgctactaaatttttataataattgaaaagattttaacaagtataaaaaattctcatag
gaattaaatgtagtctccctgtgtcagactgctctttcatagtataactttaaatcttttcttcaacttga
gtctttgaagatagttttaattctgcttgtgacattaaaagattatttgggccagttatagcttattaggg
ttgaagagaccaaggttgcaaggccaggccctgtgtgaacctttgagctttcatagagagtttcacagcat
ggactgtgtccccacggtcatccagtgttgtcatgcattggttagtcaaaatggggagggactagggcagt
ttggatagctcaacaagatacaatctcactctgtggtggtcctgctgacaaatcaagagcattgcttttgt
ttcttaagaaaacaaactcttttttaaaaattacttttaaatattaactcaaaagttgagattttggggtg
gtggtgtgccaagacattaattttttttttaaacaatgaagtgaaaaagttttacaatctctaggtttggc
tagttctcttaacactggttaaattaacattgcataaacacttttcaagtctgatccatatttaataatgc
tttaaaataaaaataaaaacaatccttttgataaatttaaaatgttacttattttaaaataaatgaagtga
gatggcatggtgaggtgaaagtatcactggactaggaagaaggtgacttaggttctagataggtgtctttt
aggactctgattttgaggacatcacttactatccatttcttcatgttaaaagaagtcatctcaaactctta
gttttttttttttacaactatgtaatttatattccatttacataaggatacacttatttgtcaagctcagc
acaatctgtaaatttttaacctatgttacaccatcttcagtgccagtcttgggcaaaattgtgcaagaggt
gaagtttatatttgaatatccattctcgttttaggactcttcttccatattagtgtcatcttgcctcccta
ccttccacatgccccatgacttgatgcagttttaatacttgtaattcccctaaccataagatttactgctg
ctgtggatatctccatgaagttttcccactgagtcacatcagaaatgccctacatcttatttcctcagggc
tcaagagaatctgacagataccataaagggatttgacctaatcactaattttcaggtggtggctgatgctt
tgaacatctctttgctgcccaatccattagcgacagtaggatttttcaaacctggtatgaatagacagaac
cctatccagtggaaggagaatttaataaagatagtgctgaaagaattccttaggtaatctataactaggac
tactcctggtaacagtaatacattccattgttttagtaaccagaaatcttcatgcaatgaaaaatacttta
attcatgaagcttactttttttttttggtgtcagagtctcgctcttgtcacccaggctggaatgcagtggc
gccatctcagctcactgcaacctccatctcccaggttcaagcgattctcgtgcctcggcctcctgagtagc
tgggattacaggcgtgtgccactacactcaactaatttttgtatttttaggagagacggggtttcaccctg
ttggccaggctggtctcgaactcctgacctcaagtgattcacccaccttggcctcataaacctgttttgca
gaactcatttattcagcaaatatttattgagtgcctaccagatgccagtcaccgcacaaggcactgggtat
atggtatccccaaacaagagacataatcccggtccttaggtagtgctagtgtggtctgtaatatcttacta
aggcctttggtatacgacccagagataacacgatgcgtattttagttttgcaaagaaggggtttggtctct
gtgccagctctataattgttttgctacgattccactgaaactcttcgatcaagctactttatgtaaatcac
ttcattgttttaaaggaataaacttgattatattgtttttttatttggcataactgtgattcttttaggac
aattactgtacacattaaggtgtatgtcagatattcatattgacccaaatgtgtaatattccagttttctc
tgcataagtaattaaaatatacttaaaaattaatagttttatctgggtacaaataaacaggtgcctgaact
agttcacagacaaggaaacttctatgtaaaaatcactatgatttctgaattgctatgtgaaactacagatc
tttggaacactgtttaggtagggtgttaagacttacacagtacctcgtttctacacagagaaagaaatggc
catacttcaggaactgcagtgcttatgaggggatatttaggcctcttgaatttttgatgtagatgggcatt
tttttaaggtagtggttaattacctttatgtgaactttgaatggtttaacaaaagatttgtttttgtagag
attttaaagggggagaattctagaaataaatgttacctaattattacagccttaaagacaaaaatccttgt
tgaagtttttttaaaaaaagctaaattacatagacttaggcattaacatgtttgtggaagaatatagcaga
cgtatattgtatcatttgagtgaatgttcccaagtaggcattctaggctctatttaactgagtcacactgc
ataggaatttagaacctaacttttataggttatcaaaactgttgtcaccattgcacaattttgtcctaata
tatacatagaaactttgtggggcatgttaagttacagtttgcacaagttcatctcatttgtattccattga
ttttttttttcttctaaacattttttcttcaaacagtatataactttttttaggggatttttttttagaca
gcaaaaactatctgaagatttccatttgtcaaaaagtaatgatttcttgataattgtgtagtaatgttttt
tagaacccagcagttaccttaaagctgaatttatatttagtaacttctgtgttaatactggatagcatgaa
ttctgcattgagaaactgaatagctgtcataaaatgaaactttctttctaaagaaagatactcacatgagt
tcttgaagaatagtcataactagattaagatctgtgttttagtttaatagtttgaagtgcctgtttgggat
aatgataggtaatttagatgaatttaggggaaaaaaaagttatctgcagatatgttgagggcccatctctc
cccccacacccccacagagctaactgggttacagtgttttatccgaaagtttccaattccactgtcttgtg
ttttcatgttgaaaatacttttgcatttttcctttgagtgccaatttcttactagtactatttcttaatgt
aacatgtttacctggaatgtattttaactatttttgtatagtgtaaactgaaacatgcacattttgtacat
tgtgctttcttttgtgggacatatgcagtgtgatccagttgttttccatcatttggttgcgctgacctagg
aatgttggtcatatcaaacattaaaaatgaccactcttttaattgaaattaacttttaaatgtttatagga
gtatgtgctgtgaagtgatctaaaatttgtaatatttttgtcatgaactgtactactcctaattattgtaa
tgtaataaaaatagttacagtgacaaaaaaaaaaaaaaa (SEQ ID NO: 14)
HRAS Ref Seq mRNA
>NM_176795.3
tgccctgcgcccgcaacccgagccgcacccgccgcggacggagcccatgcgcggggcgaaccgcgcgcccc
cgcccccgccccgccccggcctcggccccggccctggccccgggggcagtcgcgcctgtgaacggtggggc
aggagaccctgtaggaggaccccgggccgcaggcccctgaggagcgatgacggaatataagctggtggtgg
tgggcgccggcggtgtgggcaagagtgcgctgaccatccagctgatccagaaccattttgtggacgaatac
gaccccactatagaggattcctaccggaagcaggtggtcattgatggggagacgtgcctgttggacatccg
gataccgccggccaggaggagtacagcgccatgcgggaccagtacatgcgcaccggggagggcttcctgtg
tgtgtttgccatcaacaacaccaagtcttttgaggacatccaccagtacagggagcagatcaaacgggtga
aggactcggatgacgtgcccatggtgctggtggggaacaagtgtgacctggctgcacgcactgtggaatct
cggcaggctcaggacctcgcccgaagctacggcatcccctacatcgagacctcggccaagacccggcaggg
cagccgctctggctctagctccagctccgggaccctctgggaccccccgggacccatgtgacccagcggcc
cctcgcgctggagtggaggatgccttctacacgttggtgcgtgagatccggcagcacaagctgcggaagct
gaaccctcctgatgagagtggccccggctgcatgagctgcaagtgtgtgctctcctgacgcaggtgagggg
gactcccagggcggccgccacgcccaccggatgaccccggctccccgcccctgccggtctcctggcctgcg
gtcagcagcctcccttgtgccccgcccagcacaagctcaggacatggaggtgccggatgcaggaaggaggt
gcagacggaaggaggaggaaggaaggacggaagcaaggaaggaaggaagggctgctggagcccagtcaccc
cgggaccgtgggccgaggtgactgcagaccctcccagggaggctgtgcacagactgtcttgaacatcccaa
atgccaccggaaccccagcccttagctcccctcccaggcctctgtgggcccttgtcgggcacagatgggat
cacagtaaattattggatggtcttgaaaaaaaaaaaaaaaaaa (SEQ ID NO: 15)
NRAS Ref Seq mRNA
>NM_002524.3
gaaacgtcccgtgtgggaggggcgggtctgggtgcggcctgccgcatgactcgtggttcggaggcccacgt
ggccggggcggggactcaggcgcctggggcgccgactgattacgtagcgggcggggccggaagtgccgctc
cttggtgggggctgttcatggcggttccggggtctccaacatttttcccggctgtggtcctaaatctgtcc
aaagcagaggcagtggagcttgaggttcttgctggtgtgaaatgactgagtacaaactggtggtggttgga
gcaggtggtgttgggaaaagcgcactgacaatccagctaatccagaaccactttgtagatgaatatgatcc
caccatagaggattcttacagaaaacaagtggttatagatggtgaaacctgtttgttggacatactggata
cagctggacaagaagagtacagtgccatgagagaccaatacatgaggacaggcgaaggcttcctctgtgta
tttgccatcaataatagcaagtcatttgcggatattaacctctacagggagcagattaagcgagtaaaaga
ctcggatgatgtacctatggtgctagtgggaaacaagtgtgatttgccaacaaggacagttgatacaaaac
aagcccacgaactggccaagagttacgggattccattcattgaaacctcagccaagaccagacagggtgtt
gaagatgctttttacacactggtaagagaaatacgccagtaccgaatgaaaaaactcaacagcagtgatga
tgggactcagggttgtatgggattgccatgtgtggtgatgtaacaagatacttttaaagttttgtcagaaa
agagccactttcaagctgcactgacaccctggtcctgacttccctggaggagaagtattcctgttgctgtc
ttcagtctcacagagaagctcctgctacttccccagctctcagtagtttagtacaataatctctatttgag
aagttctcagaataactacctcctcacttggctgtctgaccagagaatgcacctcttgttactccctgtta
tttttctgccctgggttcttccacagcacaaacacacctctgccaccccaggtttttcatctgaaaagcag
ttcatgtctgaaacagagaaccaaaccgcaaacgtgaaattctattgaaaacagtgtcttgagctctaaag
tagcaactgctggtgattttttttttctttttactgttgaacttagaactatgctaatttttggagaaatg
tcataaattactgttttgccaagaatatagttattattgctgtttggtttgtttataatgttatcggctct
attctctaaactggcatctgctctagattcataaatacaaaaatgaatactgaattttgagtctatcctag
tcttcacaactttgacgtaattaaatccaactttcacagtgaagtgcctttttcctagaagtggtttgtag
acttcctttataatatttcagtggaatagatgtctcaaaaatccttatgcatgaaatgaatgtctgagata
cgtctgtgacttatctaccattgaaggaaagctatatctatttgagagcagatgccattttgtacatgtat
gaaattggttttccagaggcctgttttggggctttcccaggagaaagatgaaactgaaagcacatgaataa
tttcacttaataatttttacctaatctccacttttttcataggttactacctatacaatgtatgtaatttg
tttcccctagcttactgataaacctaatattcaatgaacttccatttgtattcaaatttgtgtcataccag
aaagctctacatttgcagatgttcaaatattgtaaaactttggtgcattgttatttaatagctgtgatcag
tgattttcaaacctcaaatatagtatattaacaaattacattttcactgtatatcatggtatcttaatgat
gtatataattgccttcaatccccttctcaccccaccctctacagcttcccccacagcaataggggcttgat
tatttcagttgagtaaagcatggtgctaatggaccagggtcacagtttcaaaacttgaacaatccagttag
catcacagagaaagaaattcttctgcatttgctcattgcaccagtaactccagctagtaattttgctagg
tagctgcagttagccctgcaaggaaagaagaggtcagttagcacaaaccctttaccatgactggaaaactc
agtatcacgtatttaaacatttttttttcttttagccatgtagaaactctaaattaagccaatattctcat
ttgagaatgaggatgtctcagctgagaaacgttttaaattctctttattcataatgttctttgaagggttt
aaaacaagatgttgataaatctaagctgatgagtttgctcaaaacaggaagttgaaattgttgagacagga
atggaaaatataattaattgatacctatgaggatttggaggcttggcattttaatttgcagataat
accctggtaattctcatgaaaaatagacttggataacttttgataaaagactaattccaaaatggccactt
tgttcctgtctttaatatctaaatacttactgaggtcctccatcttctatattatgaattttcatttatta
agcaaatgtcatattaccttgaaattcagaagagaagaaacatatactgtgtccagagtataatgaacctg
cagagttgtgcttcttactgctaattctgggagctttcacagtactgtcatcatttgtaaatggaaattct
gcttttctgtttctgctccttctggagcagtgctactctgtaattttcctgaggcttatcacctcagtcat
ttcttttttaaatgtctgtgactggcagtgattctttttcttaaaaatctattaaatttgatgtcaaatta
gggagaaagatagttactcatcttgggctcttgtgccaatagcccttgtatgtatgtacttagagttttcc
aagtatgttctaagcacagaagtttctaaatggggccaaaattcagacttgagtatgttctttgaatacct
taagaagttacaattagccgggcatggtggcccgtgcctgtagtcccagctacttgagaggctgaggcagg
agaatcacttcaacccaggaggtggaggttacagtgagcagagatcgtgccactgcactccagcctgggtg
acaagagagacttgtctccaaaaaaaaagttacacctaggtgtgaattttggcacaaaggagtgacaaact
tatagttaaaagctgaataacttcagtgtggtataaaacgtggtttttaggctatgtttgtgattgctgaa
aagaattctagtttacctcaaaatccttctctttccccaaattaagtgcctggccagctgtcataaattac
atattccttttggtttttttaaaggttacatgttcaagagtgaaaataagatgttctgtctgaaggctacc
atgccggatctgtaaatgaacctgttaaatgctgtatttgctccaacggcttactatagaatgttacttaa
tacaatatcatacttattacaatttttactataggagtgtaataggtaaaattaatctctattttagtggg
cccatgtttagtctttcaccatcctttaaactgctgtgaatttttttgtcatgacttgaaagca
aggatagagaaacactttagagatatgtggggtttttttaccattccagagcttgtgagcataatcatatt
tgctttatatttatagtcatgaactcctaagttggcagctacaaccaagaaccaaaaaatggtgcgttctg
cttcttgtaattcatctctgctaataaattataagaagcaaggaaaattagggaaaatattttatttggat
ggtttctataaacaagggactataattcttgtacattatttttcatctttgctgtttctttgagcagtcta
atgtgccacacaattatctaaggtatttgttttctataagaattgttttaaaagtattcttgttac
cagagtagttgtattatatttcaaaacgtaagatgatttttaaaagcctgagtactgacctaagatggaat
tgtatgaactctgctctggagggaggggaggatgtccgtggaagttgtaagacttttatttttttgtgcca
tcaaatataggtaaaaataattgtgcaattctgctgtttaaacaggaactattggcctccttggccctaaa
tggaagggccgatattttaagttgattattttattgtaaattaatccaacctagttctttttaatttggtt
gaatgttttttcttgttaaatgatgtttaaaaaataaaaactggaagttcttggcttagtcataat
tcttatattca (SEQ ID NO: 16)
BRAF mRNA
>gi|75516779|gb|BC101757.1| Homo sapiens v-raf murine
sarcoma viral oncogene homolog B1, mRNA (cDNA clone MGC: 126806
IMAGE: 8069263), complete cds
GGCCCCGGCTCTCGGTTATAAGATGGCGGCGCTGAGCGGTGGCGGTGGTGGCGGCGCGGAGCCGGGCCAG
GCTCTGTTCAACGGGGACATGGAGCCCGAGGCCGGCGCCGGCGCCGGCGCCGCGGCCTCTTCGGCTGCGG
ACCCTGCCATTCCGGAGGAGGTGTGGAATATCAAACAAATGATTAAGTTGACACAGGAACATATAGAGGC
CCTATTGGACAAATTTGGTGGGGAGCATAATCCACCATCAATATATCTGGAGGCCTATGAAGAATACACC
AGCAAGCTAGATGCACTCCAACAAAGAGAACAACAGTTATTGGAATCTCTGGGGAACGGAACTGATTTTT
CTGTTTCTAGCTCTGCATCAATGGATACCGTTACATCTTCTTCCTCTTCTAGCCTTTCAGTGCTACCTTC
ATCTCTTTCAGTTTTTCAAAATCCCACAGATGTGGCACGGAGCAACCCCAAGTCACCACAAAAACCTATC
GTTAGAGTCTTCCTGCCCAACAAACAGAGGACAGTGGTACCTGCAAGGTGTGGAGTTACAGTCCGAGACA
GTCTAAAGAAAGCACTGATGATGAGAGGTCTAATCCCAGAGTGCTGTGCTGTTTACAGAATTCAGGATGG
AGAGAAGAAACCAATTGGTTGGGACACTGATATTTCCTGGCTTACTGGAGAAGAATTGCATGTGGAAGTG
TTGGAGAATGTTCCACTTACAACACACAACTTTGTACGAAAAACGTTTTTCACCTTAGCATTTTGTGACT
TTTGTCGAAAGCTGCTTTTCCAGGGTTTCCGCTGTCAAACATGTGGTTATAAATTTCACCAGCGTTGTAG
TACAGAAGTTCCACTGATGTGTGTTAATTATGACCAACTTGATTTGCTGTTTGTCTCCAAGTTCTTTGAA
CACCACCCAATACCACAGGAAGAGGCGTCCTTAGCAGAGACTGCCCTAACATCTGGATCATCCCCTTCCG
CACCCGCCTCGGACTCTATTGGGCCCCAAATTCTCACCAGTCCGTCTCCTTCAAAATCCATTCCAATTCC
ACAGCCCTTCCGACCAGCAGATGAAGATCATCGAAATCAATTTGGGCAACGAGACCGATCCTCATCAGCT
CCCAATGTGCATATAAACACAATAGAACCTGTCAATATTGATGACTTGATTAGAGACCAAGGATTTCGTG
GTGATGGAGGATCAACCACAGGTTTGTCTGCTACCCCCCCTGCCTCATTACCTGGCTCACTAACTAACGT
GAAAGCCTTACAGAAATCTCCAGGACCTCAGCGAGAAAGGAAGTCATCTTCATCCTCAGAAGACAGGAAT
CGAATGAAAACACTTGGTAGACGGGACTCGAGTGATGATTGGGAGATTCCTGATGGGCAGATTACAGTGG
GACAAAGAATTGGATCTGGATCATTTGGAACAGTCTACAAGGGAAAGTGGCATGGTGATGTGGCAGTGAA
AATGTTGAATGTGACAGCACCTACACCTCAGCAGTTACAAGCCTTCAAAAATGAAGTAGGAGTACTCAGG
AAAACACGACATGTGAATATCCTACTCTTCATGGGCTATTCCACAAAGCCACAACTGGCTATTGTTACCC
AGTGGTGTGAGGGCTCCAGCTTGTATCACCATCTCCATATCATTGAGACCAAATTTGAGATGA
TCAAACTTATAGATATTGCACGACAGACTGCACAGGGCATGGATTACTTACACGCCAAGTCAA
TCATCCACAGAGACCTCAAGAGTAATAATATATTTCTTCATGAAGACCTCACAGTAAAAATAG
GTGATTTTGGTCTAGCTACAGTGAAATCTCGATGGAGTGGGTCCCATCAGTTTGAACAGTTGT
CTGGATCCATTTTGTGGATGGCACCAGAAGTCATCAGAATGCAAGATAAAAATCCATACAGCT
TTCAGTCAGATGTATATGCATTTGGAATTGTTCTGTATGAATTGATGACTGGACAGTTACCTTA
TTCAAACATCAACAACAGGGACCAGATAATTTTTATGGTGGGACGAGGATACCTGTCTCCAGA
TCTCAGTAAGGTACGGAGTAACTGTCCAAAAGCCATGAAGAGATTAATGGCAGAGTGCCTCA
AAAAGAAAAGAGATGGAGACCACTCTTTCCCCAAATTCTCGCCTCTATTGAGCTGCTGGCCCG
CTCATTGCCAAAAATTCACCGCAGTGCATCAGAACCCTCCTTGAATCGGGCTGGTTTCCAAAC
AGAGGATTTTAGTCTATATGCTTGTGCTTCTCCAAAAACACCCATCCAGGCAGGGGGATATGG
TGCGTTTCCTGTCCACTGAAACAAATGAGTGAGAGAGTTCAGGAGAGTAGCAACAAAAG
GAA (SEQ ID NO: 17)
RAF1(CRAF) mRNA
>gi|35841|emb|X03484.1| Human mRNA for raf oncogene
CCGAATGTGACCGCCTCCCGCTCCCTCACCCGCCGCGGGGAGGAGGAGCGGGCGAGAAGCTGCCG
CCGAACGACAGGACGTTGGGGCGGCCTGGCTCCCTCAGGTTTAAGAATTGTTTAAGCTGCATCAA
TGGAGCACATACAGGGAGCTTGGAAGACGATCAGCAATGGTTTTGGATTCAAAGATGCCGTGTTT
GATGGCTCCAGCTGCATCTCTCCTACAATAGTTCAGCAGTTTGGCTATCAGCGCCGGGCATCAGA
TGATGGCAAACTCACAGATCCTTCTAAGACAAGCAACACTATCCGTGTTTTCTTGCCGAACAAGC
AAAGAACAGTGGTCAATGTGCGAAATGGAATGAGCTTGCATGACTGCCTTATGAAAGCACTCAAG
GTGAGGGGCCTGCAACCAGAGTGCTGTGCAGTGTTCAGACTTCTCCACGAACACAAAGGTAAAAA
AGCACGCTTAGATTGGAATACTGATGCTGCGTCTTTGATTGGAGAAGAACTTCAAGTAGATTTCC
TGGATCATGTTCCCCTCACAACACACAACTTTGCTCGGAAGACGTTCCTGAAGCTTGCCTTCTGT
GACATCTGTCAGAAATTCCTGCTCAATGGATTTCGATGTCAGACTTGTGGCTACAAATTTCATGA
GCACTGTAGCACCAAAGTACCTACTATGTGTGTGGACTGGAGTAACATCAGACAACTCTTATTGT
TTCCAAATTCCACTATTGGTGATAGTGGAGTCCCAGCACTACCTTCTTTGACTATGCGTCGTATG
CGAGAGTCTGTTTCCAGGATGCCTGTTAGTTCTCAGCACAGATATTCTACACCTCACGCCTTCAC
CTTTAACACCTCCAGTCCCTCATCTGAAGGTTCCCTCTCCCAGAGGCAGAGGTCGACATCCACAC
CTAATGTCCACATGGTCAGCACCACGCTGCCTGTGGACAGCAGGATGATTGAGGATGCAATTCGA
AGTCACAGCGAATCAGCCTCACCTTCAGCCCTGTCCAGTAGCCCCAACAATCTGAGCCCAACAGG
CTGGTCACAGCCGAAAACCCCCGTGCCAGCACAAAGAGAGCGGGCACCAGTATCTGGGACCCAGG
AGAAAAACAAAATTAGGCCTCGTGGACAGAGAGATTCAAGCTATTATTGGGAAATAGAAGCCAGT
GAAGTGATGCTGTCCACTCGGATTGGGTCAGGCTCTTTTGGAACTGTTTATAAGGGTAAATGGCA
CGGAGATGTTGCAGTAAAGATCCTAAAGGTTGTCGACCCAACCCCAGAGCAATTCCAGGCCTTCA
GGAATGAGGTGGCTGTTCTGCGCAAAACACGGCATGTGAACATTCTGCTTTTCATGGGGTACATG
ACAAAGGACAACCTGGCAATTGTGACCCAGTGGTGCGAGGGCAGCAGCCTCTACAAACACCTGCA
TGTCCAGGAGACCAAGTTTCAGATGTTCCAGCTAATTGACATTGCCCGGCAGACGGCTCAGGGAA
TGGACTATTTGCATGCAAAGAACATCATCCATAGAGACATGAAATCCAACAATATATTTCTCCAT
GAAGGCTTAACAGTGAAAATTGGAGATTTTGGTTTGGCAACAGTAAAGTCACGCTGGAGTGGTTC
TCAGCAGGTTGAACAACCTACTGGCTCTGTCCTCTGGATGGCCCCAGAGGTGATCCGAATGCAGG
ATAACAACCCATTCAGTTTCCAGTCGGATGTCTACTCCTATGGCATCGTATTGTATGAACTGATG
ACGGGGGAGCTTCCTTATTCTCACATCAACAACCGAGATCAGATCATCTTCATGGTGGGCCGAGG
ATATGCCTCCCCAGATCTTAGTAAGCTATATAAGAACTGCCCCAAAGCAATGAAGAGGCTGGTAG
CTGACTGTGTGAAGAAAGTAAAGGAAGAGAGGCCTCTTTTTCCCCAGATCCTGTCTTCCATTGAG
CTGCTCCAACACTCTCTACCGAAGATCAACCGGAGCGCTTCCGAGCCATCCTTGCATCGGGCAGC
CCACACTGAGGATATCAATGCTTGCACGCTGACCACGTCCCCGAGGCTGCCTGTCTTCTAGTTGA
CTTTGCACCTGTCTTCAGGCTGCCAGGGGAGGAGGAGAAGCCAGCAGGCACCACTTTTCTGCTCC
CTTTCTCCAGAGGCAGAACACATGTTTTCAGAGAAGCTCTGCTAAGGACCTTCTAGACTGCTCAC
AGGGCCTTAACTTCATGTTGCCTTCTTTTCTATCCCTTTGGGCCCTGGGAGAAGGAAGCCATTTG
CAGTGCTGGTGTGTCCTGCTCCCTCCCCACATTCCCCATGCTCAAGGCCCAGCCTTCTGTAGATG
CGCAAGTGGATGTTGATGGTAGTACAAAAAGCAGGGGCCCAGCCCCAGCTGTTGGCTACATGAGT
ATTTAGAGGAAGTAAGGTAGCAGGCAGTCCAGCCCTGATGTGGAGACACATGGGATTTTGGAAAT
CAGCTTCTGGAGGAATGCATGTCACAGGCGGGACTTTCTTCAGAGAGTGGTGCAGCGCCAGACAT
TTTGCACATAAGGCACCAAACAGCCCAGGACTGCCGAGACTCTGGCCGCCCGAAGGAGCCTGCTT
TGGTACTATGGAACTTTTCTTAGGGGACACGTCCTCCTTTCACAGCTTCTAAGGTGTCCAGTGCA
TTGGGATGGTTTTCCAGGCAAGGCACTCGGCCAATCCGCATCTCAGCCCTCTCAGGAGCAGTCTT
CCATCATGCTGAATTTTGTCTTCCAGGAGCTGCCCCTATGGGGCGGGCCGCAGGGCCAGCCTGTT
TCTCTAACAAACAAACAAACAAACAGCCTTGTTTCTCTAGTCACATCATGTGTATACAAGGAAGC
CAGGAATACAGGTTTTCTTGATGATTTGGGTTTTAATTTTGTTTTTATTGCACCTGACAAAATAC
AGTTATCTGATGGTCCCTCAATTATGTTATTTTAATAAAATAAATTAAATTT (SEQ ID NO: 18)
ARAF mRNA
>gi|28820|emb|X04790.1| Human mRNA for A-raf-1 oncogene
TGACCCAATAAGGGTGGAAGGCTGAGTCCCGCAGAGCCAATAACGAGAGTCCGAGAGGCGACGGA
GGCGGACTCTGTGAGGAAACAAGAAGAGAGGCCCAAGATGGAGACGGCGGCGGCTGTAGCGGCGT
GACAGGAGCCCCATGGCACCTGCCCAGCCCCACCTCAGCCCATCTTGACAAAATCTAAGGCTCCA
TGGAGCCACCACGGGGCCCCCCTGCCAATGGGGCCGAGCCATCCCGGGCAGTGGGCACCGTCAAA
GTATACCTGCCCAACAAGCAACGCACGGTGGTGACTGTCCGGGATGGCATGAGTGTCTACGACTC
TCTAGACAAGGCCCTGAAGGTGCGGGGTCTAAATCAGGACTGCTGTGTGGTCTACCGACTCATCA
AGGGACGAAAGACGGTCACTGCCTGGGACACAGCCATTGCTCCCCTGGATGGCGAGGAGCTCATT
GTCGAGGTCCTTGAAGATGTCCCGCTGACCATGCACAATTTTGTACGGAAGACCTTCTTCAGCCT
GGCGTTCTGTGACTTCTGCCTTAAGTTTCTGTTCCATGGCTTCCGTTGCCAAACCTGTGGCTACA
AGTTCCACCAGCATTGTTCCTCCAAGGTCCCCACAGTCTGTGTTGACATGAGTACCAACCGCCAA
CAGTTCTACCACAGTGTCCAGGATTTGTCCGGAGGCTCCAGACAGCATGAGGCTCCCTCGAACCG
CCCCCTGAATGAGTTGCTAACCCCCCAGGGTCCCAGCCCCCGCACCCAGCACTGTGACCCGGAGC
ACTTCCCCTTCCCTGCCCCAGCCAATGCCCCCCTACAGCGCATCCGCTCCACGTCCACTCCCAAC
GTCCATATGGTCAGCACCACGGCCCCCATGGACTCCAACCTCATCCAGCTCACTGGCCAGAGTTT
CAGCACTGATGCTGCCGGTAGTAGAGGAGGTAGTGATGGAACCCCCCGGGGGAGCCCCAGCCCAG
CCAGCGTGTCCTCGGGGAGGAAGTCCCCACATTCCAAGTCACCAGCAGAGCAGCGCGAGCGGAAG
TCCTTGGCCGATGACAAGAAGAAAGTGAAGAACCTGGGGTACCGGGANTCAGGCTATTACTGGGA
GGTACCACCCAGTGAGGTGCAGCTGCTGAAGAGGATCGGGACGGGCTCGTTTGGCACCGTGTTTC
GAGGGCGGTGGCATGGCGATGTGGCCGTGAAGGTGCTCAAGGTGTCCCAGCCCACAGCTGAGCAG
GCCCAGGCTTTCAAGAATGAGATGCAGGTGCTCAGGAAGACGCGACATGTCAACATCTTGCTGTT
TATGGGCTTCATGACCCGGCCGGGATTTGCCATCATCACACAGTGGTGTGAGGGCTCCAGCCTCT
ACCATCACCTGCATGTGGCCGACACACGCTTCGACATGGTCCAGCTCATCGACGTGGCCCGGCAG
ACTGCCCAGGGCATGGACTACCTCCATGCCAAGAACATCATCCACCGAGATCTCAAGTCTAACAA
CATCTTCCTACATGAGGGGCTCACGGTGAAGATCGGTGACTTTGGCTTGGCCACAGTGAAGACTC
GATGGAGCGGGGCCCAGCCCTTGGAGCAGCCCTCAGGATCTGTGCTGTGGATGGCAGCTGAGGTG
ATCCGTATGCAGGACCCGAACCCCTACAGCTTCCAGTCAGACGTCTATGCCTACGGGGTTGTGCT
CTACGAGCTTATGACTGGCTCACTGCCTTACAGCCACATTGGCTGCCGTGACCAGATTATCTTTA
TGGTGGGCCGTGGCTATCTGTCCCCGGACCTCAGCAAAATCTCCAGCAACTGCCCCAAGGCCATG
CGGCGCCTGCTGTCTGACTGCCTCAAGTTCCAGCGGGAGGAGCGGCCCCTCTTCCCCCAGATCCT
GGCCACAATTGAGCTGCTGCAACGGTCACTCCCCAAGATTGAGCGGAGTGCCTCGGAACCCTCCT
TGCACCGCACCCAGGCCGATGAGTTGCCTGCCTGCCTACTCAGCGCAGCCCGCCTTGTGCCTTAG
GCCCCGCCCAAGCCACCAGGGAGCCAATCTCAGCCCTCCACGCCAAGGAGCCTTGCCCACCAGCC
AATCAATGTTCGTCTCTGCCCTGATGCTGCCTCAGGATCCCCCATTCCCCACCCTGGGAGATGAG
GGGGTCCCCATGTGCTTTTCCAGTTCTTCTGGAATTGGGGGACCCCCGCCAAAGACTGAGCCCCC
TGTCTCCTCCATCATTTGGTTTCCTCTTGGCTTTGGGGATACTTCTAAATTTTGGGAGCTCCTCC
ATCTCCAATGGCTGGGATTTGTGGCAGGGATTCCACTCAGAACCTCTCTGGAATTTGTGCCTGAT
GTGCCTTCCACTGGATTTTGGGGTTCCCAGCACCCCATGTGGATTTTGGGGGGTCCCTTTTGTGT
CTCCCCCGCCATTCAAGGACTCCTCTCTTTCTTCACCAAGAAGCACAGAATTC (SEQ ID NO: 19)
Protein Sequences
>KRAS
MTEYKLVVVGAGGVGKSALTIQLIQNHFVDEYDPTIEDSYRKQVVIDGETCLLDILDTAG
QEEYSAMRDQYMRTGEGFLCVFAINNTKSFEDIHHYREQIKRVKDSEDVPMVLVGNKC
DLPSRTVDTKQAQDLARSYGIPFIETSAKTRQGVDDAFYTLVREIRKHKEKMSKDGKKK
KKKSKTKCVIM* (SEQ ID NO: 20)
>HRAS
MTEYKLVVVGAGGVGKSALTIQLIQNHFVDEYDPTIEDSYRKQVVIDGETCLLDILDTAG
QEEYSAMRDQYMRTGEGFLCVFAINNTKSFEDIHQYREQIKRVKDSDDVPMVLVGNKC
DLAARTVESRQAQDLARSYGIPYIETSAKTRQGVEDAFYTLVREIRQHKLRKLNPPDESG
PGCMSCKCVLS* (SEQ ID NO: 21)
>NRAS
MTEYKLVVVGAGGVGKSALTIQLIQNHFVDEYDPTIEDSYRKQVVIDGETCLLDILDTAG
QEEYSAMRDQYMRTGEGFLCVFAINNSKSFADINLYREQIKRVKDSDDVPMVLVGNKC
DLPTRTVDTKQAHELAKSYGIPFIETSAKTRQGVEDAFYTLVREIRQYRMKKLNSSDDGT
QGCMGLPCVVM* (SEQ ID NO: 22)
>BRAF
MAALSGGGGGGAEPGQALFNGDMEPEAGAGAGAAASSAADPAIPEEVWNIKQMIKLTQ
EHIEALLDKFGGEHNPPSIYLEAYEEYTSKLDALQQREQQLLESLGNGTDFSVSSSASMD
TVTSSSSSSLSVLPSSLSVFQNPTDVARSNPKSPQKPIVRVFLPNKQRTVVPARCGVTVRD
SLKKALMMRGLIPECCAVYRIQDGEKKPIGWDTDISWLTGEELHVEVLENVPLTTHNFV
RKTFFTLAFCDFCRKLLFQGFRCQTCGYKFHQRCSTEVPLMCVNYDQLDLLFVSKFFEH
HPIPQEEASLAETALTSGSSPSAPASDSIGPQILTSPSPSKSIPIPQPFRPADEDHRNQFGQRD
RSSSAPNVHINTIEPVNIDDLIRDQGFRGDGGSTTGLSATPPASLPGSLTNVKALQKSPGPQ
RERKSSSSSEDRNRMKTLGRRDSSDDWEIPDGQITVGQRIGSGSFGTVYKGKWHGDVAV
KMLNVTAPTPQQLQAFKNEVGVLRKTRHVNILLFMGYSTKPQLAIVTQWCEGSSLYHHL
HIIETKFEMIKLIDIARQTAQGMDYLHAKSIIHRDLKSNNIFLHEDLTVKIGDFGLATVKSR
WSGSHQFEQLSGSILWMAPEVIRMQDKNPYSFQSDVYAFGIVLYELMTGQLPYSNIN
NRDQIIFMVGRGYLSPDLSKVRSNCPKAMKRLMAECLKKKRDERPLFPQILASIELLARS
LPKIHRSASEPSLNRAGFQTEDFSLYACASPKTPIQAGGYGAFPVH* (SEQ ID NO: 23)
>ARAF
MEPPRGPPANGAEPSRAVGTVKVYLPNKQRTVVTVRDGMSVYDSLDKALKVRGLNQD
CCVVYRLIKGRKTVTAWDTAIAPLDGEELIVEVLEDVPLTMHNFVRKTFFSLAFCDFCLK
FLFHGFRCQTCGYKFHQHCSSKVPTVCVDMSTNRQQFYHSVQDLSGGSRQHEAPSNRPL
NELLTPQGPSPRTQHCDPEHFPFPAPANAPLQRIRSTSTPNVHMVSTTAPMDSNLIQLTGQ
SFSTDAAGSRGGSDGTPRGSPSPASVSSGRKSPHSKSPAEQRERKSLADDKKKVKNLGYR
DSGYYWEVPPSEVQLLKRIGTGSFGTVFRGRWHGDVAVKVLKVSQPTAEQAQAFKNEM
QVLRKTRHVNILLFMGFMTRPGFAIITQWCEGSSLYHHLHVADTRFDMVQLIDVARQTA
QGMDYLHAKNIIHRDLKSNNIFLHEGLTVKIGDFGLATVKTRWSGAQPLEQPSGSVLWM
AAEVIRMQDPNPYSFQSDVYAYGVVLYELMTGSLPYSHIGCRDQIIFMVGRGYLSPDLSK
ISSNCPKAMRRLLSDCLKFQREERPLFPQILATIELLQRSLPKIERSASEPSLHRTQADELPA
CLLSAARLVP* (SEQ ID NO: 24)
>RAF1
MEHIQGAWKTISNGFGFKDAVFDGSSCISPTIVQQFGYQRRASDDGKLTDPSKTSNTIRVF
LPNKQRTVVNVRNGMSLHDCLMKALKVRGLQPECCAVFRLLHEHKGKKARLDWNTD
AASLIGEELQVDFLDHVPLTTHNFARKTFLKLAFCDICQKFLLNGFRCQTCGYKFHEHCS
TKVPTMCVDWSNIRQLLLFPNSTIGDSGVPALPSLTMRRMRESVSRMPVSSQHRYSTPHA
FTFNTSSPSSEGSLSQRQRSTSTPNVHMVSTTLPVDSRMIEDAIRSHSESASPSALSSSPNNL
SPTGWSQPKTPVPAQRERAPVSGTQEKNIRPRGQRDSSYYWEIEASEVMLSTRIGSGSFG
TVYKGKWHGDVAVKILKVVDPTPQFQAFRNEVAVLRKTRHVNILLFMGYMTKDNLAIV
TQWCEGSSLYKHLHVQETFQMFQLIDIARQTAQGMDYLHAKNIIHRDMKSNNIFLHEGL
TVKIGDFGLATVKSRWSGSQQVEQPTGSVLWMAPEVIRMQDNNPFSFQSDVYSYGIVLY
ELMTGELPYSHINNRDQIIFMVGRGYASPDLSKLYKNCPKAMKRLVADCVKKVKEERPL
FPQILSSIELLQHSLPKINRSASEPSLHRAAHTEDINACTLTTSPRLPVF* (SEQ ID NO: 25)
>MKNK1
MVSSQKLEKPIEMGSSEPLPIADGDRRRKKKRRGRATDSLPGKFEDMYKLTSELLGEGA
YAKVQGAVSLQNGKEYAVKIIEKQAGHSRSRVFREVETLYQCQGNKNILELIEFFEDDTR
FYLVFEKLQGGSILAHIQKQKHFNEREASRVVRDVAAALDFLHTKDKVSLCHLGWSAM
APSGLTAAPTSLGSSDPPTSASQVAGTTGIAHRDLKPENILCESPEKVSPVKICDFDLGSG
MKLNNSCTPITTPELTTPCGSAEYMAPEVVEVFTDQATFYDKRCDLWSLGVVLYIMLSG
YPPFVGHCGADCGWDRGEVCRVCQNKLFEIQEGKYEFPDKDWAHISSEAKDLISKLLVR
DAKQRLSAAQVLQHPWVQGQAPEKGLPTPQVLQRNSSTMDLTLFAAEAIALNRQLSQH
EENELAEEPEALADGLCSMKLSPPCKSRLARRRALAQAGRGEDRSPPTAL* (SEQ ID NO: 26)
The four RSK genes:
>RPS6KA1
MPLAQLKEPWPLMELVPLDPENGQTSGEEAGLQPSKDEGVLKEISITHHVKAGSEKADPS
HFELLKVLGQGSFGKVFLVRKVTRPDSGHLYAMKVLKKATLKVRDRVRTKMERDILAD
VNHPFVVKLHYAFQTEGKLYLILDFLRGGDLFTRLSKEVMFTEEDVKFYLAELALGLDH
LHSLGIIYRDLKPENILLDEEGHIKLTDFGLSKEAIDHEKKAYSFCGTVEYMAPEVVNRQG
HSHSADWWSYGVLMFEMLTGSLPFQGKDRKEMTLILKAKLGMPQFLSTEAQSLLRALF
KRNPANRLGSGPDGAEEIKRHVFYSTIDNKLYRREITPPFKPAVAQPDDTFYFDTEFTSRT
PKDSPGIPPSAGAHQLFRGFSFVATGLMEDDGKPRAPQAPLHSVVQQLHGKNLVFSDGY
VVKETIGVGSYSECKRCVHKATNMEYAVKVIDKSKRDPSEEIEILLRYGQHPNIITLKDV
YDDGKHVYLVTELMRGGELLDKILRQKFFSEREASFVLHTIGKTVEYLHSQGVVHRDLK
PSNILYVDESGNPECLRICDFGFAKQLRAENGLLMTPCYTANFVAPEVLKRQGYDEGCDI
WSLGILLYTMLAGYTPFANGPSDTPEEILTRIGSGKFTLSGGNWNTVSETAKDLVSKMLH
VDPHQRLTAKQVLQHPWVTQKDKLPQSQLSHQDLQLVKGAMAATYSALNSSKPTPQLK
PIESSILAQRRVRKLPSTTL* (SEQ ID NO: 27)
>RPS6KA2
MDLSMKKFAVRRFFSVYLRRKSRSKSSSLSRLEEEGVVKEIDISHHVKEGFEKADPSQFE
LLKVLGQGSYGKVFLVRKVKGSDAGQLYAMKVLKKATLKVRDRVRSKMERDILAEVN
HPFIVKLHYAFQTEGKLYLILDFLRGGDLFTRLSKEVMFTEEDVKFYLAELALALDHLHS
LGIIYRDLKPENILLDEEGHIKITDFGLSKEAIDHDKRAYSFCGTIEYMAPEVVNRRGHTQS
ADWWSFGVLMFEMLTGSLPFQGKDRKETMALILKAKLGMPQFLSGEAQSLLRALFKRN
PCNRLGAGIDGVEEIKRHPFFVTIDWNTLYRKEIKPPFKPAVGRPEDTFHFDPEFTARTPT
DSPGVPPSANAHHLFRGFSFVASSLIQEPSQQDLHKVPVHPIVQQLHGNNIHFTDGYEIKE
DIGVGSYSVCKRCVHKATDTEYAVKIIDKSKRDPSEEIEILLRYGQHPNIITLKDVYDDGK
FVYLVMELMRGGELLDRILRQRYFSEREASDVLCTITKTMDYLHSQGVVHRDLKPSNIL
YRDESGSPESIRVCDFGFAKQLRAGNGLLMTPCYTANFVAPEVLKRQGYDAACDIWSLG
ILLYTMLAGFTPFANGPDDTPEEILARIGSGKYALSGGNWDSISDAAKDVVSKMLHVDPH
QRLTAMQVLKHPWVVNREYLSPNQLSRQDVHLVKGAMAATYFALNRTPQAPRLEPVLS
SNLAQRRGMKRLTSTRL* (SEQ ID NO: 28)
>RPS6KA3
MPLAQLADPWQKMAVESPSDSAENGQQIMDEPMGEEEINPQTEEVSIKEIAITHHVKEGH
EKADPSQFELLKVLGQGSFGKVFLVKKISGSDARQLYAMKVLKKATLKVRDRVRTKME
RDILVEVNHPFIVKLHYAFQTEGKLYLILDFLRGGDLFTRLSKEVMFTEEDVKFYLAELA
LALDHLHSLGIIYRDLKPENILLDEEGHIKLTDFGLSKESIDHEKKAYSFCGTVEYMAPEV
VNRRGHTQSADWWSFGVLMFEMLTGTLPFQGKDRKETMTMILKAKLGMPQFLSPEAQS
LLRMLFKRNPANRLGAGPDGVEEIKRHSFFSTIDWNKLYRREIHPPFKPATGRPEDTFYFD
PEFTAKTPKDSPGIPPSANAHQLFRGFSFVAITSDDESQAMQTVGVHSIVQQLHRNSIQFT
DGYEVKEDIGVGSYSVCKRCIHKATNMEFAVKIIDKSKRDPTEEIEILLRYGQHPNIITLKD
VYDDGKYVYVVTELMKGGELLDKILRQKFFSEREASAVLFTITKTVEYLHAQGVVHRDL
KPSNILYVDESGNPESIRICDFGFAKQLRAENGLLMTPCYTANFVAPEVLKRQGYDAACD
IWSLGVLLYTMLTGYTPFANGPDDTPEEILARIGSGKFSLSGGYWNSVSDTAKDLVSKML
HVDPHQRLTAALVLRHPWIVHWDQLPQYQLNRQDAPHLVKGAMAATYSALNRNQSPV
LEPVGRSTLAQRRGIKKITSTAL* (SEQ ID NO: 29)
>RPS6KA4
MGDEDDDESCAVELRITEANLTGHEEKVSVENFELLKVLGTGAYGKVFLVRKAGGHDA
GKLYAMKVLRKAALVQRAKTQEHTRTERSVLELVRQAPFLVTLHYAFQTDAKLHLILD
YVSGGEMFTHLYQRQYFKEAEVRVYGGEIVLALEHLHKLGIIYRDLKLENVLLDSEGHIV
LTDFGLSKEFLTEEKERTFSFCGTIEYMAPEIIRSKTGHGKAVDWWSLGILLFELLTGASPF
TLEGERNTQAEVSRRILKCSPPFPPRIGPVAQDLLQRLLCKDPKKRLGAGPQGAQEVRNH
PFFQGLDWVALAARKIPAPFRPQIRSELDVGNFAEEFTRLEPVYSPPGSPPPGDPRIFQGYS
FVAPSILFDHNNAVMTDGLEAPGAGDRPGRAAVARSAMMQDSPFFQQYELDLREPALG
QGSFSVCRRCRQRQSGQEFAVKILSRRLEANTQREVAALRLCQSHPNVVNLHEVHHDQL
HTYLVLELLRGGELLEHIRKKRHFSESEASQILRSLVSAVSFMHEEAGVVHRDLKPENILY
ADDTPGAPVKIIDFGFARLRPQSPGVPMQTPCFTLQYAAPELLAQQGYDESCDLWSLGVI
LYMMLSGQVPFQGASGQGGQSQAAEIMCKIREGRFSLDGEAWQGVSEEAKELVRGLLT
VDPAKRLKLEGLRGSSWLQDGSARSSPPLRTPDVLESSGPAVRSGLNATFMAFNRGKRE
GFFLKSVENAPLAKRRKQKLRSATASRRGSPAPANPGRAPVASKGAPRRANGPL
PPS* (SEQ ID NO: 30)
>ETS1
MKAAVDLKPTLTIIKTEKVDLELFPSPDMECADVPLLTPSSKEMMSQALKATFSGFTKEQ
QRLGIPKDPRQWTETHVRDWVMWAVNEFSLKGVDFQKFCMNGAALCALGKDCFLELA
PDFVGDILWEHLEILQKEDVKPYQVNGVNPAYPESRYTSDYFISYGIEHAQCVPPSEFSEP
SFITESYQTLHPISSEELLSLKYENDYPSVILRDPLQTDTLQNDYFAIKQEVVTPDNMCMG
RTSRGKLGGQDSFESIESYDSCDRLTQSWSSQSSFNSLQRVPSYDSFDSEDYPAALPNHKP
KGTFKDYVRDRADLNKDKPVIPAAALAGYTGSGPIQLWQFLLELLTDKSCQSFISWTGD
GWEFKLSDPDEVARRWGKRKNKPKMNYEKLSRGLRYYYDKNIIHKTAGKRYVYRFVC
DLQSLLGYTPEELHAMLDVKPDADE* (SEQ ID NO: 31)
>ELK1
MDPSVTLWQFLLQLLREQGNGHIISWTSRDGGEFKLVDAEEVARLWGLRKNKTNMNYD
KLSRALRYYYDKNIIRKVSGQKFVYKFVSYPEVAGCSTEDCPPQPEVSVTSTMPNVAPAA
IHAAPGDTVSGKPGTPKGAGMAGPGGLARSSRNEYMRSGLYSTFTIQSLQPQPPPHPRPA
VVLPNAAPAGAAAPPSGSRSTSPSPLEACLEAEEAGLPLQVILTPPEAPNLKSEELNVEPG
LGRALPPEVKVEGPKEELEVAGERGFVPETTKAEPEVPPQEGVPARLPAVVMDTAGQAG
GHAASSPEISQPQKGRKPRDLELPLSPSLLGGPGPERTPGSGSGSGLQAPGPALTPSLLPTH
TLTPVLLTPSSLPPSIHFWSTLSPIAPRSPAKLSFQFPSSGSAQVHIPSISVDGLSTPVVLSPG
PQKP* (SEQ ID NO: 32)
>SH2D1A (SAP)
MDAVAVYHGKISRETGEKLLLATGLDGSYLLRDSESVPGVYCLCVLYHGYIYTYRVSQT
ETGSWSAETAPGVHKRYFRKIKNLISAFQKPDQGIVIPLQYPVEKKSSARSTQGTTGIRED
PDVCLKAP* (SEQ ID NO: 33)

Mutations
RAS family (KRAS, HRAS and NRAS): mutations in codon 12, 13 and 61, i.e. G12A, G12N, G12R, G12C, G12S, G12V, G13N and Q61R.
BRAF: mutations in codon 600, i.e. V600E.

TABLE 8

| | | Colo | Colo | Colo | HT- | HCT- | SW- | SW- | Colo | HT- | NCI- | SNU- | SW- |
|---|---|---|---|---|---|---|---|---|---|---|---|---|---|
| | | 205 | 201 | 741 | 29 | 116 | 480 | 620 | 320 | 55 | H716 | C1 | 48 |
| GI50 (nM) | 17- | 9 +/− 3 | >10 uM | >10 uM | 10 | 78 | 49 | 48 +/− 54 | >1.6 uM | NA | NA | NA | 15 +/− 4 |
| | AAG | | | | | | | | | | | | |
| | IPI-504 | 8 +/− 2 | >10 uM | >10 uM | 6 | 88 | 42 | 51 +/− 43 | >1.6 uM | 54 | 66 | 117 | 13 +/− 4 |
| | IPI-493 | 7 +/− 3 | >10 uM | >10 uM | 2 | 34 | 30 | 13 +/− 5 | >1.6 uM | 28 | 35 | 25 | 7 +/− 1 |
| Mutational | KRAS | wt | wt | wt | wt | Mut | Mut | Mut | wt | wt | wt | wt | wt |
| Status | | | | | | G13D | G12V | G12V | | | | | |
| | BRAF | Mut | Mut | Mut | Mut | wt | wt | wt | wt | Mut | wt | wt | wt |
| | | V600E | V600E | V600E | V600E | | | | | N581Y | | | |
| | EGFR | wt | wt | wt | wt | wt | wt | wt | wt | wt | wt | wt | G719S |

SUPPLEMENTAL TABLE 1

| Pt ID | Genzyme EGFR | D × S EGFR | D × S KRAS | D × S BRAF | Snapshot | Oncomap | ALK FISH |
|---|---|---|---|---|---|---|---|
| 1 | Mutant | | | | | | |
| 2 | Mutant | | | | | | |
| 3 | Mutant | | | | | | |
| 4 | Mutant | Mutant | | | | | |
| 5 | Mutant | | | | | | |
| 6 | Mutant | | | | | | |
| 7 | Mutant | | | | | | |
| 8 | Mutant | Mutant | | | | | |
| 9 | Mutant | | | | | | |
| 10 | Mutant | | Wild Type | | EGFR & KRAS | EGFR | |
| 11 | Mutant | | | | | | |
| 12 | Mutant | Mutant | Wild Type | | EGFR | | |
| 13 | Mutant | | | | | | |
| 14 | Mutant | Mutant | Wild Type | | EGFR & PIK3CA | | |
| 15 | Mutant | | | | | | |
| 16 | Mutant | | | | | | |
| 17 | Mutant | | | | | | |
| 18 | Mutant | | | | EGFR | | |
| 19 | Mutant | | | | | | |
| 20 | Mutant | | | | | | |
| 21 | Mutant | | | | EGFR & TP53 | | No Rearrangement Detected |
| 22 | Mutant | | | | | | |
| 23 | Mutant | | | | | | |
| 24 | Mutant | | | | | | |
| 52 | Wild Type | | | | KRAS | | No Rearrangement Detected |
| 53 | Wild Type | | | | | | |
| 54 | Wild Type | | | | KRAS | | No Rearrangement Detected |
| 55 | Wild Type | | | | | | |
| 56 | Wild Type | Wild Type | Wild Type | Wild Type | | | |
| 57 | Wild Type | Mutant | Wild Type | | EGFR | Wild Type | |
| 58 | Wild Type | | | | | | |
| 59 | Wild Type | | Mutant | | KRAS | KRAS | No Rearrangement Detected |
| 60 | Wild Type | | Mutant | | KRAS | KRAS | |
| 61 | Wild Type | | Mutant | | KRAS | KRAS | No Rearrangement Detected |
| 62 | Wild Type | | Wild Type | | | | No Rearrangement Detected |
| 63 | Wild Type | Wild Type | Mutant | | KRAS | Wild Type | |
| 64 | Wild Type | Wild Type | Wild Type | Wild Type | Wild Type | Wild Type | No Rearrangement Detected |
| 65 | Wild Type | Wild Type | Wild Type | Wild Type | | Wild Type | Rearrangement Detected |
| 66 | Wild Type | | Mutant | | | | |
| 67 | | | | | | | |
| 68 | | | | | | | |
| 69 | | | | | Wild Type | | No Rearrangement Detected |
| 70 | | Mutant | Wild Type | | | | |
| 71 | | | | | | | |
| 72 | | | | | | | |
| 73 | | | | | | | |
| 74 | | | | | | | |
| 75 | | | | | | | |
| 76 | | Mutant | | | | | |

What is claimed is:

1. A method of determining the responsiveness of, a tumor or a cancer cell, or a subject having or at risk of having the tumor or cancer cell, to a treatment comprising an HSP90 inhibitor, said method comprising:
(i) detecting an alteration in an an ALK, a MAPK pathway and/or EGFR gene or gene product in the tumor or cancer cell; and/or
(ii) evaluating one or more of: a) the tumor or cancer cell histology, b) the subject's smoking status, or c) the level or expression of HSP90 in the tumor or cancer cell, thereby determining the responsiveness of the tumor or the cancer cell to the treatment comprising the HSP90 inhibitor.

2. A method of identifying a subject having, or at risk of having, a cancer or tumor, as having a likelihood to respond to a treatment comprising an HSP90 inhibitor, said method comprising one, two, three or four of the following:
(i) detecting the presence or absence of an alteration in an an ALK, a MAPK pathway and/or EGF gene or gene in a sample from the subject;
(ii) detecting the presence or absence of a cancerous histology in a sample from the subject;
(iii) determining the subject's smoking status; or
(iv) determining the level or expression of HSP90 in a sample from the subject, thereby identifying the subject as being likely or unlikely to respond to the treatment comprising the HSP90 inhibitor.

3. A method of monitoring the efficacy, or predicting the efficacy, of a treatment comprising an HSP90 inhibitor, of a cancer or tumor in a subject, said method comprising
(i) detecting the presence or absence of an alteration in an an ALK, a MAPK pathway and/or EGFR gene or gene product, in a sample obtained from the subject; and/or
(ii) evaluating one or more of: a) the presence or absence of a cancerous histology in a sample from the subject; b) the subject's smoking status; or c) the level or expression of HSP90; and
(iii) comparing the detected alteration or evaluation in (i) and/or (ii) to a reference sample, wherein the extent of the difference in the alteration or evaluation detected in the sample in relation to the reference sample is indicative of, or predictive of, the efficacy of the treatment.

4. The method of any of claims 1-3, wherein one or more of following is indicative of an increased likelihood to respond to a treatment comprising the HSP90 inhibitor: (i) detecting presence of non-small cell lung cancer, squamous cell or colorectal cancer cells or tissue in said histology; (ii) identifying the subject as a smoker, e.g., having a smoking history of at least 5, 10, 15 or more pack years; or (iii) detecting an elevated level or expression of HSP90.

5. The method of claim 2, wherein detection of, or the presence of, the alteration in an ALK gene or gene product is indicative that the tumor, the cancer cell, or the subject has an increased likelihood to respond to a treatment comprising the HSP90 inhibitor.

6. The method of claim 2, wherein the MAPK pathway gene or gene product is chosen from one or more of H-Ras, N-Ras, K-Ras, A-Raf, B-Raf (BRAF), C-Raf, Mek, or Erk.

7. The method of claim 2, further comprising detection of an alteration in one or more gene products chosen from PIK3CA, PTEN, AKT, TP53 (p53), CTNNB1 (beta-catenin), APC, KIT, JAK2, NOTCH, FLT3, RSK, ETS, ELK-1, or SAP-1.

8. The method of claim 6, wherein the detection of, or the presence of, the alteration in a MAPK pathway gene or gene product is indicative that the tumor or cancer cell has an increased likelihood to respond to a treatment comprising the HSP90 inhibitor as a single agent.

9. The method of claim 6, wherein the detection of, or the presence of, the alteration in a MAPK pathway gene or gene product is indicative that the tumor or cancer cell has an increased likelihood to respond to a treatment comprising the HSP90 inhibitor in combination with a second agent.

10. The method of claim 5, wherein the detection of, or the presence of, the alteration in an ALK gene or gene product is indicative of an increased likelihood to respond to a treatment comprising an HSP90 inhibitor as a single agent or in combination, to inhibit, reduce, or treat a NSCLC tumor or cancer cell.

11. The method of claim 6, wherein the detection of, or the presence of, the alteration in a K-Ras gene or gene product, is indicative of an increased likelihood to respond to therapy comprising an HSP90 inhibitor as a single agent or in combination, to inhibit, reduce, or treat a colorectal tumor or cancer cell.

12. The method of claim 6, wherein the detection of, or the presence of, the alteration in a B-Raf gene or gene product, is indicative of an increased likelihood to respond to therapy comprising an HSP90 inhibitor as a single agent or in combination, to inhibit, reduce, or treat a colorectal tumor or cancer cell.

13. The method of claim 6, wherein the detection of, or the presence of, the alteration in a K-Ras gene or gene product, optionally in combination with detecting an alteration in a a p53 gene or gene product, is indicative of an increased likelihood to respond to a treatment comprising an HSP90 inhibitor and an mTOR inhibitor, to inhibit, reduce, or treat a NSCLC tumor or cancer cell.

14. The method of any of claims 1-3, further comprising treating or preventing a cancer or tumor harboring an alteration in the ALK and/or the MAPK pathway gene or gene product, said treatment comprising administering to a subject in need of HSP90 treatment, an HSP90 inhibitor, as a single agent or in combination.

15. A method of treating a subject having, or at risk of having, a cancer or tumor harboring an ALK or MAPK pathway alteration, comprising administering to a subject identified as likely to benefit from, or being considered or evaluated for, an HSP90 inhibitor treatment, an HSP90 inhibitor, as a single agent or in combination, in an amount sufficient to reduce or inhibit the tumor cell growth, and/or treat or prevent the cancer, in the subject.

16. The method of claim 15, wherein the subject is identified as having, one or more of: a history of smoking; elevated level or expression of HSP90; NSCLC (e.g., relapsed and/or refractory NSCLC); NSCLC or SCC cells or tumors; or is experiencing disease progression during or after receiving at least one prior chemotherapeutic regimen; is an NSCLC patient experiencing disease progression during or after receiving at least one prior platinum-containing chemotherapeutic regimen.

17. The method of claim 3, further comprising altering a dose or dosage schedule of an HSP90 inhibitor, alone or in combination, in response to the difference detected, wherein the presence of an alteration in the ALK, a MAPK pathway and/or EGFR gene or gene product, or the presence of cancerous cells or tissues, in the sample obtained from the subject during treatment with the HSP90 inhibitor, or after treatment has been discontinued, is indicative of the need to increase in dose or frequency of administration of the HSP90 inhibitor, as a single agent or in combination.

18. The method of claim 5, wherein the alteration in the ALK gene or gene product comprises an ALK gene rearrangement, EML4-ALK fusion, an KIF5B-ALK fusion, a TGF-ALK fusion, an NPM-ALK fusion, or an ALK point mutatios including one or more of F12451/L, L1204F, A1200V, L1196M, 11170S, T1151M, R1275Q, F1174V/C/L, T1087I, or K1062M.

19. The method of claim 6, wherein the alteration in the MAPK gene or gene products is chosen from one or more mutant K-Ras or B-Raf polynucleotide molecules, or the polypeptides listed in Table 5.

20. The method of claim 19, wherein the one or more K-Ras mutation are chosen from one or more of KRAS_G12C, KRAS_G12R, KRAS_G12D, KRAS_G12A, KRAS_G12S, KRAS_G12V, KRAS_G13D, KRAS_G13S, KRAS_G13C, KRAS_G13V, KRAS_Q61H, KRAS_Q61R, KRAS_Q61P, KRAS_Q61L, KRAS_Q61K, KRAS_Q61E, KRAS_A59T or KRAS_G12F.

21. The method of claim 6, wherein the alteration in the MAPK pathway gene or gene product is chosen from BRAF_D594G, BRAF_D594V, BRAF_F468C, BRAF_F595L, BRAF_G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E, BRAF_K601N, BRAF_L597Q, BRAF_L597R, BRAF_L597S, BRAF_L597V, BRAF_T599I, BRAF_V600E, BRAF_V600K, BRAF_V600L, or BRAF_V600R.

22. The method of any of claims 1-3, wherein the alteration is detected by one or more of: nucleic acid hybridization assay, amplification-based assays, sequencing, screening analysis, metaphase cytogenetic analysis by standard karyotype methods, FISH, spectral karyotyping or MFISH, and/or comparative genomic hybridization, or in situ hybridization.

23. The method of claim 2, wherein the method further comprises one or more of: determining whether the subject with an an ALK, a MAPK pathway and/or EGFR mutation positive cancer is likely to respond to treatment comprising the HSP90 inhibitor; altering the course of therapy, dosing, treatment schedule or time course, combination therapies; determining the time course of the cancer in the subject; or determining the probability of a significant event in the subject.

24. The method of any of claims 1-3 or 15, wherein the cancer cell or tumor identified or treated is chosen from one or more of lung cancer, small cell lung cancer (SCLC), non-small cell lung cancer (NSCLC), or squamous cell cancer (SCC), colorectal cancer (CRC), breast cancer, medulloblastoma, chondrosarcoma, osteosarcoma, pancreatic cancer, ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, prostate cancer, anaplastic large cell lymphoma, neuroblastoma, neuroendocrine or carcinoid.

25. The method of any of claims 1-3 or 15, wherein the HSP90 inhibitor is chosen from one or more of IPI-493, IPI-504, 17-AAG (also known as tanespimycin or CNF-1010), BIIB-021 (CNF-2024), BIIB-028, AUY-922 (also known as VER-49009), SNX-5422, STA-9090, AT-13387, XL-888, MPC-3100, CU-0305, 17-DMAG, CNF-1010, Macbecin (e.g., Macbecin I, Macbecin II), CCT-018159, CCT-129397, PU-H71, or PF-04928473 (SNX-2112).

26. The method of claim 14, wherein the HSP90 inhibitor is administered in combination with an mTOR inhibitor chosen from one or more of rapamycin, temsirolimus (TORISEL®), everolimus (RAD001, AFINITOR®), ridaforolimus, AP23573, AZD8055, BEZ235, BGT226, XL765, PF-4691502, GDC0980, SF1126 or OSI-027.

27. The method of claim 14, wherein the HSP90 inhibitor is administered in combination with an ALK kinase inhibitor, a tyrosine kinase inhibitor, a taxoid, or a topoisomerase inhibitor.

28. The method of claim 14, wherein the HSP90 inhibitor is administered in combination with one or more other therapeutic modalities chosen from anti-cancer agents, surgical or radiation procedures.

29. A method of treating a subject having a functional or non-functional neuroendocrine tumor, comprising administering to the subject an Hsp90 inhibitor in an amount sufficient to reduce or inhibit the tumor growth, thereby treating the neuroendocrine tumor, wherein the neuroendocrine tumor is chosen from one or more of: a pancreatic endocrine tumor; a neuroendocrine lung tumor; or a neuroendocrine cancer from the adrenal medulla, the pituitary, the parathyroids, thyroid endocrine islets, pancreatic endocrine islets, or dispersed endocrine cells in the respiratory or gastrointestinal tract.

30. A kit, or assay, for determining the chemosensitivity of a cancer patient to treatment with an HSP90 inhibitor, comprising a reagent that specifically binds to one or more alterations of an an ALK, a MAPK pathway and/or EGFR gene or gene product, optionally in combination with one or more of PIK3CA, PTEN, AKT, TP53, CTNNB1 (beta-catenin), APC, KIT, JAK2, NOTCH, or FLT3.
